(19) United States
(12) Patent Application Publication
(10) Pub. No.: US 2024/0209357 A1
(43) Pub. Date: Jun. 27, 2024

(54) DNA CONSTRUCTS COMPRISING ALTERNATIVE PROMOTERS

(71) Applicant: ETH ZÜRICH, Zürich (CH)

(72) Inventors: Yaakov Benenson, Binningen (CH); Jlten Doshi, Rajkot (IN)

(73) Assignee: ETH ZÜRICH, Zürich (CH)

(21) Appl. No.: 18/039,578

(22) PCT Filed: Dec. 1, 2021
(86) PCT No.: PCT/EP2021/083846

§ 371 Date: May 31, 2023

(30) Foreign Application Priority Data
Dec. 1, 2020 (EP) 20020580.5

(51) Int. Cl. C12N 15/113; C12N 15/86

(52) U.S. Cl. CPC C12N 15/113; C12N 15/86; C12N 2310/14; C12N 2740/15043

(57) ABSTRACT

The invention relates to the fields of biomolecular computing and/or synthetic biology. In particular, the invention relates to a DNA construct that comprises a first promoter, at least one further promoter and an output sequence, wherein each of said promoters comprises a transcription start site and/or is suitable for initiating transcription, wherein the initiation of transcription from the first promoter is enabled by a first transcriptional regulatory state and the initiation of transcription from each of said further promoter(s) is enabled by a respective further transcriptional regulatory state, and wherein said DNA construct yields an effective amount of an output RNA in a eukaryotic cell, when said first transcriptional regulatory state and/or any of the respective further transcriptional regulatory states is present in said cell, wherein said output RNA comprises a sequence corresponding to said output sequence. Furthermore, the invention relates to medical and/or diagnostic uses of the inventive DNA construct of the invention, e.g., for detecting, killing and/or manipulating different types of eukaryotic target cells in a subject and/or in a tissue sample.

### CROSS-REFERENCE TO RELATED APPLICATIONS

This application is the U.S. National Stage application, pursuant to 35 U.S.C. § 371, of PCT International Patent Application No. PCT/EP2021/083846, filed Dec. 1, 2021, designating the European Patent Office and published in English, which claims priority under 35 U.S.C. §§ 119 and 365 to European Patent Application No. 20020580.5, filed Dec. 1, 2020. The contents of each of the aforementioned applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jan. 9, 2024, is named AD3598_PCT_BS.txt and is 76,534 bytes in size.

The invention relates to the fields of biomolecular computing and/or synthetic biology. In particular, the invention relates to a DNA construct that comprises a first promoter, at least one further promoter and an output sequence, wherein each of said promoters comprises a transcription start site and/or is suitable for initiating transcription, wherein the initiation of transcription from the first promoter is enabled by a first transcriptional regulatory state and the initiation of transcription from each of said further promoter(s) is enabled by a respective further transcriptional regulatory state, and wherein said DNA construct yields an effective amount of an output RNA in a eukaryotic cell, when said first transcriptional regulatory state and/or any of the respective further transcriptional regulatory states is present in said cell, wherein said output RNA comprises a sequence corresponding to said output sequence. Furthermore, the invention relates to medical and/or diagnostic uses of the inventive DNA construct of the invention, e.g., for detecting, killing and/or manipulating different types of eukaryotic target cells in a subject and/or in a tissue sample.

Research in biomolecular computing and synthetic biology (Bashor et al., 2019; Benenson, 2012; Elowitz and Leibler, 2000; Gardner et al., 2000; Ham et al., 2008; Xie and Fussenegger, 2018; You et al., 2004) has enabled, over the last two decades, a variety of gene circuit architectures capable of implementing complex logic in mammalian cells. An OR logic program generates high output when at least one of the inputs to the program is active, and it is key to addressing heterogeneous cell populations. For example, there are several subtypes of each kind of cancer. A therapeutic genetic classifier circuit that targets more than one cancer subtype while generating the same output is desirable. For this, a genetic classifier circuit that can implement an OR logic at a transcriptional and/or post-transcriptional level can be very useful, especially when the molecular inputs that differentiate the subtypes can act directly or indirectly at the promoter level.

So far, OR logic between transcriptional inputs has often been trivially implemented with two distinct genetic constructs, each receiving its own set of inputs (Buchler et al., 2003). Experimentally, however, this often results in multi-valued logic with twice the output obtained when both constructs are active compared to a single active construct (Kramer et al., 2004; Rinaudo et al., 2007). So, there is an, often undesired, output expression imbalance across different input conditions.

Additionally, the large redundant genetic footprint (Lapique and Benenson, 2018) makes it impractical for translation to clinically-relevant viral vectors. In fact, the presence of redundant information increases the payload size which prevents such constructs to be packaged into viral vectors or used as a therapeutic product. The redundancy issue has been tackled by use of recombinases which, however, is not desired at the moment for therapy (Lapique and Benenson, 2018), at least for safety reasons. Previously-described post-transcriptional OR logic with miRNA inputs functions not as a single gate but a superposition of NOR and NOT gates (Mohammadi et al., 2017). RNA interference was also shown to support logic operations between transcription factors, but at the expense of high circuit complexity (Leisner et al., 2010).

There is also an ongoing quest toward universal, potentially indefinitely scalable logic in living cells. However, the gap between the state of the art and the desired functionality is particularly acute when it comes to multiple transcriptional inputs, which carry perhaps the most information about cell state and therefore have the most promise as inputs to application-relevant gene circuits.

While initially thought of as straightforward (Buchler et al., 2003), implementing transcriptional OR gates at a single promoter level is challenging due to the fact that secondary interactions between different transcriptional inputs, or synergy, is the rule rather than the exception (Angelici et al., 2016; Donahue et al., 2020; Lin et al., 1990; Lohmueller et al., 2012). A similar observation was made in prokaryotes (Cox et al., 2007), and eventually a robust prokaryotic OR gate was designed via “dual promoters” driving the expression of the same coding sequence from distinct RNA Polymerase binding sites (Tamsir et al., 2011). However, it is entirely unclear whether dual promoters would be functional in eukaryotes, e.g. due to the commonly observed secondary interactions of transcriptional inputs. Interestingly, higher eukaryotes are often faced with their own requirements to implement OR logic when the same gene is to be expressed in a number of different cell lineages or under distinct sets of inducible conditions. This is often naturally implemented via multiple alternative promoters regulating alternatively spliced first exons of a gene. Usually, in nature, only one of these promoters actively transcribes the gene at any given time, generating otherwise identical transcripts with different first exons, which can be either protein-coding, e.g., in ABL1 or CIITA (Nickerson et al., 2001), generating protein isoforms; or non-coding, resulting in the exact same protein, e.g., the FURIN gene (Ayoubi and VanDeVen, 1996). However, it has not been explored whether this natural phenomenon which is part of an extremely complex regulation of cellular behavior could be exploited in synthetic DNA constructs, i.e. in tools for technical applications.

Furthermore, OR logic has been implemented at the DNA level using recombinases (Bonnet et al., 2013), however it is unidirectional meaning that once the logic circuit encounters an input, the output will get defined and remain such even when the input signal is removed. This evidently drastically reduces the versatility of such an approach.

In summary, there is no practicable way available yet that implements OR logic at transcriptional and/or post-transcriptional levels, i.e., with reduced complexity. The lack of suitable tools constitutes a significant obstacle when trying to cope with heterogeneous cell populations, e.g. in therapeutic and/or diagnostic applications.

Thus, there is still a need for improved means and methods for analyzing and/or manipulating eukaryotic cells, in particular, for detecting and/or manipulating different eukaryotic cell types and/or states.

Accordingly, the invention relates to a DNA construct comprising in 5′ to 3′ direction the following DNA sequence elements:
a first promoter (P₁);
n further promoter(s) (Pₙ), wherein n≥1, e.g. P₂, P₂ and P₃, or P₂, P₃ and P₄; and an output sequence,
wherein each of said promoters comprises a transcription start site and/or is suitable for initiating transcription,
wherein initiation of transcription from P₁ is enabled by a first transcriptional regulatory state (TS₁) and initiation of transcription from each of said further promoter(s) (Pₙ) is enabled by a respective further transcriptional regulatory state (TSₙ), e.g. P₂ is enabled by TS₂, and
wherein said DNA construct yields an effective amount of an output RNA in a eukaryotic cell, preferably a mammalian cell, when said TS₁ and/or any of the respective TSₙ (e.g. for P₁ and P₂: TS₁, TS₂, or TS₁ and TS₂), is present in said cell,
wherein said output RNA comprises a sequence corresponding to said output sequence.

In particular, a first type of said output RNA (RNA₁) may be obtained when transcription is initiated from P₁ (i.e because TS₁ is present), and a respective further type of said output RNA (RNAₙ, e.g. RNA₂) may be obtained when transcription is initiated from a respective Pₙ, e.g P₂, (i.e because the respective TSₙ, e.g. TS₂ is present), wherein each type of said output RNA comprises a sequence corresponding to said output sequence.

In particular, the amount of an output RNA present in a cell refers to the total (i.e. cumulative) amount of all types of an output RNA present in said cell, at least of all useful output RNA types as described herein, in particular because each type of an output RNA comprises an identical sequence, i.e. a sequence corresponding to said output sequence.

Preferably, said DNA construct does not yield an effective amount of said output RNA when neither TS₁ nor any of TSₙ is present in said cell.

The invention is, at least partly, based on the surprising discovery that mRNAs encoding a desired output protein could be independently produced in mammalian cells by each one of two or more alternative promoters comprised in the same DNA construct. As illustrated in the appended Examples, a high amount of the output protein was obtained regardless of which of the promoters was activated by a corresponding transcriptional activator.

It was entirely unexpected that an OR gate-like logic that is operational in eukaryotic cells could be achieved by a single DNA construct. It is highly advantageous to achieve an OR gate-like logic with a single DNA construct of the invention which is robust and simple compared to multiple constructs or highly complex and bulky constructs. In particular, a single inventive DNA construct provided herein can be more easily integrated into target cells, e.g. because it can be more easily packaged into a viral vector, compared to multiple constructs that have to be packaged into different vectors or very large constructs that are not efficiently packaged into viral vectors at all, and still enable OR gate-like logic operations. Thus, the inventive DNA constructs provided herein allow to reduce the payload size for DNA-based applications. In particular, the present invention is advantageous for DNA-based therapeutic and/or diagnostic applications, because the delivery of genetic material into cells is often still the major bottle neck for such applications.

Furthermore, for treating and/or diagnosing many diseases, such as, inter alia, cancer, it is important to recognize cells in different states and/or different subtypes of cells. This is greatly facilitated by the DNA constructs of the invention, at least because due to their rather compact size, they can be more easily incorporated into more target cells and due to the presence of alternative promoters they can recognize different cell types and/or states.

Thus, the DNA constructs of the invention can be used as classifiers to distinguish target cells (e.g. abnormal and/or malignant cells) from non-target cells (e.g. normal or benign cells). In particular, the inventive DNA constructs provide an improved classification, at least because they are capable of OR gate-like logic operations which allows to recognize different cell types and/or states, such as, inter alia, different subtypes of a cancer.

Furthermore, the DNA constructs of the present invention, as illustrated in the appended Examples, provide a scalable and expandable platform which can provide further logic operations as described herein.

In particular, as described herein, and as illustrated in the appended Examples, a single alternative promoter may function as an AND gate such that transcription is only initiated when two or more conditions (e.g. transcriptional activators) are present.

Furthermore, as described herein, and as illustrated in the appended Examples, the DNA construct of the invention allows to produce different types of output RNAs (e.g. mRNA isoforms) that are subject to different post-transcriptional regulatory mechanisms and/or factors. This allows further logic operations such as “AND NOT”. In particular, the DNA construct of the invention may comprise features that allow alternative splicing which further improves the implementation of “AND NOT” logic operations, at least because undesired intervening sequences can be removed from the output RNAs which increases the flexibility and options, as described herein. The implementation of alternative splicing further provides the possibility of producing different types of an output protein (e.g. different protein isoforms that are translated from different respective mRNA isoforms) based on the activity of the respective promoters, as described herein. Thus, the inventive DNA constructs provided herein can enable further AND-gate logic operations and/or AND NOT gate-like logic operations in addition to the central OR-gate like logic operation, as illustrated in the appended Examples. Thus, the DNA constructs of the invention may form a normal-form-like logic circuit, e.g. a disjunctive normal form-like (AND-OR) logic circuit.

For example, in some embodiments, the DNA construct described herein yields an effective amount of an output RNA according to complex logic formulae such as: (TS1a AND TS1b AND NOT PTS1) OR (TS2a AND TS2b AND NOT PTS2). However, this example is merely illustrative and the inventive DNA construct provided herein can provide many different logic operations dependent on which DNA sequence elements it comprises, as described herein.

The DNA construct of the invention may be also considered a “DNA cassette”, as commonly understood in the art. Thus, the terms “DNA construct” and “DNA cassette” may be used interchangeably herein. In particular, a DNA construct or DNA cassette, as used herein and in the context of the invention, refers to a sequence of DNA (deoxyribonucleic acid) (or a sequence of DNA nucleotides), and thus be may also considered a DNA polynucleotide. Furthermore, the DNA construct of the invention may comprise or consist of DNA analogues and/or modified DNA (e.g. chemically modified DNA such as, inter alia, methylated DNA), at least as long as said DNA construct has the desired functionality as described herein. Preferably, the DNA construct of the invention is comprised of at least 50%, 60%, 70%, 80%, 90%, 95%, 99% or 100%, preferably at least 90% deoxyribonucleic acid. Preferably, the DNA construct of the invention is double-stranded (dsDNA). However, the DNA construct of the invention may be also single-stranded (ssDNA), e.g. when the coding stand or the antisense strand of the DNA construct is comprised in an Adeno Associated Virus (AAV) vector. Furthermore, the invention encompasses an RNA that comprises a sequence corresponding to the DNA construct of the invention and/or that comprises a sequence that is complementary to the sequence of the DNA construct of the invention, e.g. an RNA that is comprised in a retrovirus, e.g. a lentiviral vector. Thus, the double stranded DNA construct of the invention may be also formed in a cell upon delivery of a ssDNA or RNA vector that comprises a sequence corresponding to the DNA construct of the invention into the cell. For example, an AAV vector contains a partially single stranded DNA that is converted into a double stranded DNA in the cell and a Lentiviral vector packages an RNA payload that is converted into a DNA sequence in the cell via reverse transcription.

The DNA construct of the invention comprises DNA sequence elements that are arranged in 5′ to 3′ direction, in particular wherein the sequence of the DNA construct of the invention corresponds to the coding strand (sense strand) of said DNA construct. In particular, the DNA construct of the invention may comprise more than one DNA sequence element of a certain type. For example, the DNA construct of the invention comprises at least two promoters (P), i.e. a first promoter (P₁) and n further promoter(s) (Pₙ), wherein n≥1.

Furthermore, the DNA construct of the invention is functional in a eukaryotic cell, i.e. it yields an effective amount of an output RNA in a eukaryotic cell under certain conditions, e.g. in cells of a certain type and/or in a certain state. Evidently, this functionality can be assayed with cells that comprise the DNA construct of the invention, e.g. cells into which the DNA construct of the invention has been introduced.

In particular, as illustrated in the appended Examples, the DNA construct of the invention may yield an effective amount of an output RNA in a eukaryotic cell under more than one condition, e.g. under more than one condition of a certain type (e.g. when one or more transcriptional regulatory states are present). For example, at least two transcriptional regulatory states (TS), i.e. a first transcriptional regulatory state (TS₁) and n further transcriptional regulatory states (TSₙ) may enable the initiation of transcription such that an effective amount of an output RNA is obtained.

In particular herein, an individual feature of a certain type may be linked to at least one other individual feature of a different type. Such linked features are also called “respective” features herein. For example, individual DNA sequence elements of a certain type (e.g. individual promoters) may be linked to other individual DNA sequence elements of a different type (e.g. individual alternative first exons) and/or an individual cellular condition of a certain type (e.g. individual transcriptional regulatory states).

As used herein, and in the context of the invention, respective individual features are designated by the same number (e.g. 1, 2, 3, etc.), a corresponding letter (e.g. a, b, c, etc.) or n, wherein “n” means at least a further one (n≥1, in addition to 1 or a), and thus “n” may stand for a number greater than 1, e.g., 2, 3, 4, 5, etc., or a letter in the Latin alphabet after a, e.g. b, c, d, e, etc. For example n may be 1, 2, 3, 4, 5, 6, 7, 8, 9, or 10, preferably 1, 2, 3, or 4, more preferably 1 or 2, which means that there may be, for example 2, 3, 4, 5, 6, 7, 8, 9, 10 or 11, preferably 2, 3, 4, or 5, more preferably 2 or 3 features of a certain type (e.g. promoters, alternative first exons, 5′ splice sites, transcriptional regulatory states, and/or post-transcriptional regulatory states, etc.) present and/or involved.

Usually, numbers are used prior to letters, e.g. the individual promoters may be P₁, P₂, P₃ etc., or Pₙ. However, when a feature (or the abbreviation thereof) already includes a number (e.g. an alternative first exon (E1), letters are used to designate the individual features of the same type (e.g. E1ₐ, E1_{b}, E1_{c}, etc., or E1ₙ). Furthermore, when features of the same type (e.g. transcription factors (TFs)) are first grouped into individual groups (e.g. TF₁, TF₂, TF₃, etc.), then the individual elements of the individual groups are designated by numbers (e.g. the individual TFs of the first group of TFs (TF₁) may be TF₁ₐ, TF_{1b}, TF_{1c}, etc.).

As an illustrative example, E1ₐ and/or TF₁ₐ (or TF₁ₙ) may be respective features of P₁; and E1ₙ and/or TFₙₐ (or TFₙₙ) may be respective features of Pₙ.

As described above, a certain type of a feature may comprise more than one individual feature (element). As further described above, a certain type of a feature may relate to (i) a DNA sequence element that may be comprised in the DNA construct of the invention or (ii) a cellular condition that may control the amount of an output RNA obtained in a eukaryotic cell comprising the DNA construct of the invention.

For example, different types of DNA sequence elements that may be comprised in the DNA construct of the invention, wherein any of them may contain more than one element, may be: promoters (P), spacers, unique sequences (US), alternative first exons (E1), alternative 5′ splice sites (5′ss), and/or further intronic sequences. Of note, the term “alternative” may be omitted herein, i.e. in the context of expressions such as (alternative) promoters, (alternative) first exons, and (alternative) 5′ splice sites, without changing the meaning of the expression.

Furthermore, different types of cellular conditions that may control the amount of an output RNA obtained in a eukaryotic cell, wherein any of them may contain more than one element, may be, for example: transcriptional regulatory states (TS), post-transcriptional regulatory states (TS), transcription factors (TF), antisense RNAs, and/or abnormal and/or malignant cell types and/or states (AC).

Furthermore, an output RNA produced and/or obtained by the DNA construct of the invention may comprise more than one type of an output RNA, as further described herein, and as illustrated in the appended Examples. Thus, an output RNA may be also considered a respective feature, e.g. it may be linked to a respective promoter and/or a respective post-transcriptional regulatory state. However, as further described herein, the amount of an output RNA (e.g. an effective amount of an output RNA), refers, in particular, to the total (i.e. cumulative) amount of all types of output RNAs the are obtained in a cell, as further described herein.

For example, the initiation of transcription from a certain promoter (P) is enabled by a respective transcriptional regulatory state (TS). Thus, transcription initiation from the first promoter (P₁) is enabled by TS₁, and transcription initiation from a further promoter (Pₙ), e.g. P₂, is enabled by the respective further transcriptional regulatory state (TSₙ), e.g. TS₂.

Thus, P₁ may produce a respective output RNA (RNA₁), and a further promoter (Pa, e.g., P₂) may produce a further respective output RNA (RNAₙ, e.g., RNA₂). Nonetheless, it is usually considered, in context of the invention, that an effective amount of an output RNA is obtained when an effective amount of at least RNA₁ and/or another one of RNAₙ, e.g RNA₂, is present in the cell and/or when the total (i.e. cumulative) amount of all types of an output RNA in the cell corresponds to an effective amount, in particular, wherein RNA₁ and any of RNAₙ, e.g. RNA₂, comprise a common output sequence (or common second exon), as described herein.

This principle of respective features may be applied to any respective features herein.

For example, a certain promoter, e.g. P₁, may comprise at least one binding site for at least one TF or a certain number of TFs from a respective group of TFs, e.g. TF₁, such as TF₁ₐ and/TF_{1b}. As another example, a certain promoter, e.g. P₁, may be the next promoter upstream of a respective alternative first exon (e.g. E1ₐ) and a respective 5′ splice site (e.g. 5′ss₁), wherein said promoter produces a respective output RNA that comprises a sequence corresponding to the respective first alternative first exon when the respective transcriptional regulatory state (e.g. TS₁) is present, and wherein said output RNA may be controlled by a respective post-transcriptional regulatory state (e.g. PTS₁), as described herein.

A promoter, as used herein, is a sequence of DNA to which proteins can bind that initiate transcription of an RNA from the DNA downstream of and/or overlapping with the promoter, i.e. from the transcription start site (TSS), wherein the transcription start site (TSS) corresponds to the first nucleotide of the transcribed RNA. As used herein, an RNA produced by a promoter (e.g. a certain type of an output RNA) refers, in particular, to the RNA that is transcribed by the activity of said promoter.

In particular, the TSS is located downstream of the promoter, preferably nearby the promoter, or more preferably, in the context of the present invention, the TSS is comprised within the promoter, in particular wherein said TSS is near the 3′ end or at the 3′ end of the promoter. Thus, in some cases a part of the sequence of a promoter may lie 3′ (downstream) of the TSS, but in any case the promoter region to which RNA Polymerase can bind to initiate transcription (i.e. the RNA polymerase binding site) is 5′ (upstream) of the TSS. Any TSS that is upstream of an RNA Polymerase binding site of a promoter is not considered, in the context of the present invention, a TSS that belongs to that promoter and/or that is comprised in that promoter. Whether a TSS is downstream of a promoter (and i.e. near the promoter) or whether a TSS is comprised in a promoter (i.e. near the 3′ end or at the 3′end of the promoter) may not make a technical difference but is, in particular, an issue of definition. Since, in the context of the invention, a promoter should be suitable for initiating transcription, and transcription is initiated at a TSS, a promoter is preferably defined herein such that it comprises the TSS, as described herein. However, it would be also suitable to specify that the inventive DNA construct may comprise a respective TSS downstream of each promoter, e.g. a TSS₁ downstream of P₁ (but upstream of all Pₙ such as P₂), and a TSS₂ downstream of P₂, etc.

Yet, in the context of the present invention, and as just described, a promoter preferably comprises a TSS, in particular wherein said TSS is near the 3′ end or at the 3′ end of the promoter. The size of a promoter is not particularly limited, but it may be about 15 to 2000 bp. The transcription start sites (TSSs) that belong to the different promoters and/or that are comprised in the different promoters of the invention may be structurally identical, similar or different. In particular, a TSS corresponds to the first (i.e. most 5′) nucleotide of the respective type of an output RNA that is produced by the promoter to which said TSS belongs and/or in which said TSS is comprised, wherein said first nucleotide may be the first nucleotide of the 5′ untranslated region of said output RNA type or the first nucleotide of the start codon of at least one output protein that is encoded by said output RNA type.

The promoters comprised in the DNA construct of the invention may be considered alternative promoters, at least, because each promoter comprises its own transcriptional start site (TSS), and/or is, in principle, suitable for initiating transcription and producing an output RNA, i.e. because it may allow the binding of proteins, as described herein. Preferably, at least one, preferably each, of said promoters comprises a binding site for an RNA polymerase. Preferably herein, the RNA polymerase is RNA polymerase II.

However, the fact that each promoter may be suitable for initiating transcription and producing an output RNA does not mean that a promoter constitutively initiates transcription or produces an output RNA in any condition. To the contrary, the activity (i.e. the initiation of transcription) of a promoter, in the context of the invention, is regulated, i.e., it is enabled by a respective transcriptional regulatory state (TS), as described herein.

Furthermore, a promoter may contain specific DNA sequences such as response elements (binding sites) that provide a secure initial binding site for RNA polymerase and for proteins called transcription factors (TFs) that contribute in recruiting the RNA polymerase.

A promoter may further work in concert with other regulatory regions of the DNA (e.g. enhancers or insulators) to control the transcription initiation and/or the amount of the transcribed (produced) RNA. Said other regulatory regions may be comprised in the DNA construct of the invention, or, in particular, they may be comprised in the genome of a cell into which the DNA construct of the invention may be integrated (e.g. by means of a viral vector or a transposon system).

For example, a promoter may comprise a core promoter, wherein said core promoter comprises a transcription start site (TSS), and upstream thereof a binding site for an RNA polymerase, in particular RNA polymerase II (e.g. to produce a messenger RNA and/or a microRNAs), and at least one general transcription factor binding site (response element), e.g. a TATA box, and/or a B recognition element, and, preferably upstream of the core promoter, at least one binding site for a specific transcription factor, as described herein. In particular, the TATA box and/or B recognition element may be within about 20 to about 50 bp of the TSS.

In particular herein, the activity of a promoter refers to the initiation of transcription from said promoter. Furthermore, when a promoter is active, i.e. when transcription from said promoter is initiated, an output RNA is produced by said promoter, i.e. an output RNA is transcribed from the respective TSS (e.g. within said promoter) and the DNA sequence directly downstream of the TSS comprising at least an output sequence, as described herein. Thus, as described herein, a certain promoter may produce a certain type of an output RNA that comprises a unique sequence and downstream thereof a common sequence (or second exon) that is shared between different output RNA types (i.e. a sequence corresponding to the output sequence or second exon of the DNA construct of the invention). Furthermore, as described herein, the initiation of transcription from a certain promoter is enabled by a respective transcriptional regulatory state.

In the context of the invention, a transcriptional regulatory state may be a characteristic of a eukaryotic cell which comprises the DNA construct of the invention and/or in which the DNA construct of the invention is functional. Thus, a certain transcriptional regulatory state, e.g. TS₁, or any of TSₙ such as TS₂ or TS₃ may be associated with and/or reflect a certain cell type and/or cell state. Hence, a cell comprising a certain transcriptional regulatory state enables the initiation of transcription from a respective promoter comprised in the DNA construct of the invention. In other words, said promoter is active in said cell.

Thus, a certain promoter comprised in the DNA construct of the invention may be active in a certain cell type and/or state, which means that an output RNA may be produced in a cell of said type and/or in said state. Thus, at least one or each of the promoters comprised in the DNA construct of the invention may be a cell type and/or cell state specific-promoter.

The cell type and/or state and the associated transcription regulatory state, as used herein and in the context of the invention, is not particularly limited. For example, a certain cell type and/or state may refer, inter alia, to a differentiated cell state; a stem cell state; a disease cell state; a certain generic cell type such as, inter alia, a lymphocyte or a B-cell; a certain generic abnormal and/or malignant cell type such, inter alia, a leukemic cell; a certain generic healthy cell type such, inter alia, a non-malignant or benign lymphocyte; a cell type with a specific molecular characteristic such as, inter alia, a B-cell that has a certain genetic mutation or a B-cell that shows a certain biomarker or combination of biomarkers; a specific stem cell type, such as, inter alia, a hematopoietic stem cell, or a specific cell type during development, such as, inter alia, a hemogenic endothelial cell. This list is merely illustrative and in no way exhaustive. In fact, a certain cell type and/or state may relate to any certain generic or specific cell of virtually any tissue and/or any certain cell state of any type of cell.

Preferably, a certain transcriptional regulatory state, i.e. the presence of a certain transcriptional regulatory state, may be associated with and/or reflect a disease cell state, i.e. an abnormal and/or malignant type and/or state of a cell, e.g. a cell of a certain tissue type. Thus, a certain promoter comprised in the DNA construct of the invention may be active in an abnormal and/or malignant cell of a certain type. This is further described herein, i.e. context of the inventive medical and/or diagnostic uses of the DNA construct of the invention.

Furthermore, a transcriptional regulatory state, as used herein and in the context of the present invention, may comprise any mechanism and/or factor that can contribute to and/or, preferably, regulate the initiation of transcription from a respective promoter. An individual mechanism and/or factor may promote or inhibit said initiation of transcription. Yet, a certain transcriptional regulatory state is considered present herein, when initiation of the respective promoter is enabled, and, in particular, an effective amount of a respective output RNA is produced (i.e. transcribed). Thus, when a certain transcriptional regulatory state is considered present, the corresponding mechanisms and/or factors must, overall (in total and/or in combination), enable the transcription initiation from the respective promoter. When this is not the case, said certain transcriptional regulatory state is usually considered absent herein.

Mechanisms and/or factors that contribute to and/or regulate the initiation of transcription from a promoter include RNA polymerases (e.g. RNA polymerase II), general transcription factors (e.g. TFIIA, TFIIB, TFIID, TFIIE, TFIIF, TFIIE and/or TFIIH), cell type and/or state specific transcription factors (e.g. TFs from a respective group of TFs, as described herein; illustrative examples include erythroid lineage-specific TFs (e.g., inter alia, GATA1); pluripotent stem cell-specific TFs (e.g., inter alia, OCT4), or a liver-specific TFs (e.g., inter alia, HNF4), epigenetic modifiers (e.g. histone acetylases, histone demethylases, SWI/SNF complex, DNA methyltransferases etc.), the location of the promoter (and the DNA construct) in the cell (e.g. episomal, or in an overall transcriptionally active or inactive chromosomal region, and/or a certain topologically associating domain) and epigenetic modifications at the respective promoter sequence and/or associated enhancer sequences (e.g. histone modifications such as, inter alia, mono-, di- or trimethylation or acetylation of H3K4, H3K27 or H3K9, and/or DNA modifications such as DNA methylation or DNA hydroxymethylation).

It is also possible that a certain transcriptional regulatory state, post-transcriptional regulatory state and/or cell type and/or state is associated with and/or reflects a certain transcriptional regulatory state and/or post-transcriptional regulatory state in the past of a cell, e.g. a previous cell type and/or state of the cell and/or a cell from which said cell is derived. This is possible because certain mechanisms and/or factors, e.g. epigenetic mechanisms and/or epigenetic modifiers, can impart a memory to cells that may persist across many cell divisions (and that may be associated with transcriptional and/or post-transcription regulation).

A certain cell type and/or state and/or the presence of a certain transcriptional regulatory mechanism and/or factor (e.g. the presence of a certain transcription factor) may be readily determined and/or defined for reference purposes by the person skilled in the art by any means known in the art e.g. by the analysis of biomarkers (e.g. DNA variants or mutations, the expression of certain characteristic proteins, RNAs, and/or functional characteristics, e.g. the presence of a certain biological function such as a certain enzymatic reaction), genomics and/or transcriptomics (e.g. DNA and/or RNA sequencing, DNA and/or RNA microarrays, multiplexed in situ hybridization), proteomics (e.g. mass spectrometry, flow cytometry and/or mass cytometry), epigenomics (e.g. 5′mC DNA sequencing and/or ChIP-seq) live-cell imaging, and/or functional in vitro and/or in vivo assays. In vivo assays should be only carried out when necessary, and only in suitable animal models such as, inter alia, mice, rats, fish, flies, and/or monkeys.

Thus, the person skilled in the art has no difficulties to define reference transcriptional regulatory programs and/or reference cell types and/or states.

Furthermore, the DNA construct of the invention (or RNA or single-stranded DNA that corresponds to the sense (coding) and/or antisense (non-coding) strand of the inventive DNA construct) can be transiently or stably introduced into reference cells and/or cells of interest by means known in the art without any difficulties, e.g. as described herein and/or illustrated in the appended Examples, for example, by viral transduction (e.g. by an adeno-associated virus (AAV) vector, a lentiviral vector and/or an Adenoviral vector), transfection (e.g. lipofection), electroporation, and/or the use of transposons (e.g. piggyBac and/or sleeping beauty).

Furthermore, the presence of an output RNA and/or output protein can be easily determined by means known in the art, e.g. as described herein and/or illustrated in the appended Examples, for example, by RT-PCR, RNA sequencing, RNA microarrays, in-situ hybridization, Western blot, ELISA, immunofluorescence, mass spectrometry etc. Furthermore, the presence of fluorescent or luminescent output proteins may be further determined by optical methods such as flow cytometry and/or imaging (e.g. microscopy).

Hence, it can be easily determined whether a certain promoter is active in a certain reference cell that comprises a certain transcriptional regulatory state (e.g. a reference cell of a certain type and/or in a certain state) by ordinary means.

Thus, the person skilled in the art can select and/or modify a promoter of the inventive DNA construct provided herein by routine experimentation such that said promoter is active when a respective transcriptional regulatory state is present.

Furthermore, rational design principles can be applied for selecting and/or modifying promoters. For example, when it is desired or expected that a certain transcriptional regulatory state comprises a certain transcription factor, e.g. a TF from certain group of transcription factors as described herein, a respective promoter of the inventive DNA construct may comprise respective binding sites (response elements) to which said TF(s) can bind.

Thus, a certain transcriptional regulatory state, i.e. the presence of a certain transcriptional regulatory state, may comprise the presence of at least one transcription factor (TF) from a respective group of transcription factors, e.g.,
wherein TS₁ comprises the presence of at least one TF from a first group of TFs (TF₁), e.g. TF₁ₐ and/or TF_{1b}, and/or
a TSₙ, e.g. TS₂, comprises the presence of at least one TF from a further group of TFs (TFₙ, e.g., TF₂), for example, TFₙₐ and/or TF_{nb}, e.g. TF₂ₐ and/or TF_{2b}.

However, an individual transcription factor may be contained in one or more of said groups of TFs.

In particular, a certain TF is considered present when a higher amount of the TF compared to a control cell that does not express said TF is present, and/or when adding a similar amount of the TF (e.g. by forced expression from a plasmid) affects the amount of the respective RNA that is produced (positive control experiment).

In particular, the first transcriptional regulatory state (TS₁) may comprises the presence of at least a certain number of, e.g. two, TFs from a first group of TFs (TF₁), e.g. TF₁ₐ and TF_{1b}, and/or any of the further transcriptional regulatory state(s) (TSₙ) may comprise the presence of at least a certain number of, e.g. two, TFs from a respective further group of transcription factors (TFₙ, e.g., TF₂), for example, TFₙₐ and TF_{nb}, e.g. TF₂ₐ and TF_{2b}.

Thus, P₁ may comprise at least one binding site for at least one TF or a certain number of TFs from TF₁, e.g. TF₁ₐ, or TF₁ₐ and TF_{1b}; and/or any of Pₙ, e.g. P₂, may comprise at least one binding site for at least one TF or a certain number of TFs from a respective TFₙ, e.g. TF₂, for example, TFₙₐ, or TFₙₐ and TF_{nb}, e.g. TF₂a, or TF₂ₐ and TF_{2b}.

Thus, as illustrated in the appended Examples, a promoter of the DNA construct of the invention may be designed such that transcription is initiated from said promoter when more than one, e.g. 2, 3, or 4 TFs are present, e.g. when said promoter comprises at least one binding site for each of said TFs. In other words, it is possible that a promoter is activated in a synergistic way by more than one TF.

However, this principle is not limited to TFs, but any two or more mechanisms and/or factors, as described herein, may act synergistically to activate a certain promoter of the DNA construct. Thus, the activation of any one of the promoters of the inventive DNA construct (e.g. by two or more mechanisms and/or factors of the same respective transcriptional regulatory state, e.g. comprising two or more TFs from the same respective group of TFs) may follow an AND gate-like logic.

However, as described herein, the production of an output RNA by the different (alternative) promoters of the DNA construct of the invention (which are activated by different respective transcriptional regulatory states, e.g., comprising different respective groups of TFs) rather follows an OR gate-like logic.

Thus, when an AND gate-like logic at the individual promoter level is combined with the OR gate-like logic at the DNA construct level (i.e. based on the alternative promoters), disjunctive normal form-like logic operations (OR of ANDs) may be achieved such as, inter alia, (TF₁ₐ AND TF_{1b}) OR (TF₂ₐ AND TF_{2b}).

Furthermore, as illustrated in the appended Examples, the DNA construct of the invention may be designed such that it is further capable of AND NOT-gate like logic operations. In particular herein, the NOT logic is implemented at the post-transcriptional level, i.e. at the level of the produced mRNA, e.g. by means of antisense RNAs as described herein. In particular herein, the AND NOT logic refers to the production of a certain type of an output RNA that is not inhibited and/or degraded by a respective post-transcriptional regulatory state, as described herein.

Furthermore, a NOT gate-like logic may be implemented in the inventive DNA construct provided herein such that any produced output RNA is controlled by the same post-transcriptional regulatory state, i.e. when said post-transcriptional regulatory state (e.g. comprising one or more antisense RNAs) targets a sequence in the output RNA that corresponds to a sequence that is comprised in the common output sequence (e.g. the 3′ UTR) of the DNA construct of the invention, as described herein.

In some embodiments, the DNA construct of the invention comprises between at least one, preferably each, pair of promoters (e.g. P₁ and P₂, and/or P₂ and P₃) a respective spacer sequence (spacer). In particular, said spacer may disrupt or abrogate interactions between the promoters, preferably such that they do not initiate transcription in a synergistic manner or such that a downstream promoter is not inhibited by an active upstream promoter, as illustrated in the appended Examples. Thus, spacers may further improve the OR-gate like logic of the inventive DNA constructs.

Furthermore, each spacer may comprise a respective unique sequence (US), e.g. the spacer between P₁ and P₂ (the first spacer) may comprise a first unique sequence (US₁), and the spacer between P₂ and P₃ (the second spacer) may comprise a US2, and so forth.

Such a DNA construct can produce different types of output RNAs, for example, wherein only an output RNA produced by P₁ comprises a sequence corresponding to US1 (e.g. in the 5′ UTR). Thus, the different types of output RNAs may be subject to different respective post-transcriptional regulatory states (PTS). In other words, unique sequences in spacers may enable specific NOT-gate like logic operations.

For example, when transcription is initiated from P₁, an output RNA is produced that comprises sequences corresponding to all unique sequences including US₁, e.g. US₁ and US₂. However, when transcription is initiated from P₂, an output RNA is produced that comprises sequences corresponding to all unique sequences except US₁, e.g. US₂. Thus, in this example, only the output RNA produced by P₁ is subject to a respective post-transcriptional regulatory state that targets US₁ (e.g. PTS₁), whereas the output RNAs produced by P₁ or P₂ are subject to a respective PTS that targets US₂ (e.g. PTS₂). As described herein, a post-transcriptional regulatory state preferably leads to the degradation of a respective (target) output RNA and/or inhibits translation of a protein encoded by a respective (target) output RNA.

Thus, a DNA construct comprising in 5′ to 3′ direction P₁, US₁, P₂, US₂, and an output sequence may yield an effective amount of an output RNA under the following conditions: (i) TS₁ is present, PTS₁ is absent and PTS₂ is absent; or (ii) TS₂ is present and PTS₂ is absent.

Thus, such a DNA construct is capable of, at least some, complex logic operations.

However, the flexibility of such a DNA construct can be still improved. In particular, it may be desirable that the respective output RNA produced by a certain promoter (e.g. P₁) does not contain sequences corresponding to other promoter(s) and/or unique sequences that are downstream of said promoter (e.g. P₂ and/or US₂).

Thus, the DNA construct of the invention may further comprise in 5′ to 3′ direction between said first promoter (P₁) and the last promoter of said further promoter(s) (Pₙ) a first alternative first exon (E1ₐ) and a first alternative 5′ splice site (5′ss₁), and
between said last promoter and said output sequence a branch point (BP) and a 3′ splice site (3′ss), wherein said output sequence is or comprises a second Exon (E2).

Since said DNA construct comprises at least one 5′ss, a BP and a 3′ss, and thus can produce an output RNA (i.e. a pre-RNAs such as a pre-mRNA) that is subject to RNA splicing, said DNA construct is thus further called, more specifically, herein a “splicing DNA construct”. Thus, the inventive splicing DNA construct(s) provided herein, refer, in particular, to preferred embodiments of the DNA construct of the invention.

The terms “splice site” and “splice signal” are used interchangeably herein. Furthermore, the terms “branch point” and “branch site” are used interchangeably herein.

In particular, said 5′ss₁, BP and 3′ss enable the removal of the sequence between the 5′ss₂ and the 3′ss (including the sequence corresponding to P₂) contained in a respective output RNA produced by P₁. Thus, in that example, an output RNA produced by P₁ comprises in 5′ to 3′ direction a sequence corresponding to E1ₐ and E2 (but neither P₂ nor any sequence between P₂ and the 3′ss such as an US₂), whereas an output RNA produced by P₂ comprises a sequence corresponding to the common output sequence (E2) without E1ₐ.

In general, the DNA construct of the invention (including splicing DNA constructs and non-splicing DNA constructs) produces an effective amount of an output RNA in a eukaryotic cell under certain conditions, as described herein, i.e. (i) when the initiation of transcription from at least one of the promoters comprised in the DNA construct is enabled, and (ii) when the respective produced output RNA is not degraded and/or the translation of an output protein encoded by the respective produced output RNA is not inhibited.

The term “an output RNA”, as used herein and in the context of the present invention, refers to the output RNA that can be produced by any of the promoters contained in the inventive DNA construct. In particular, “an output RNA” encompasses all RNA molecules (i.e. different types of an output RNA) that comprise a sequence corresponding to the output sequence (which may be or comprise the “second exon” (E2)), as described herein. Thus, the amount of an output RNA obtained in a cell refers, in particular, and as described herein, to the total (i.e. cumulative) amount of all types of output RNAs, at least of all useful output RNA types, that are obtained in a cell, i.e. regardless of the promoter by which the output RNAs are produced. Furthermore, an output RNA may comprise a transcribed pre-RNA (which is not or not fully spliced) and, the respective spliced RNA, at least, pre-RNAs and/or spliced RNAs that are useful, as described herein, e.g. pre-RNAs and/or spliced RNAs that encode an output protein (i.e. in-frame).

In context of the invention, it may be specified, e.g., how a certain output RNA comprising a sequence corresponding to a respective first alternative first exon (e.g. E1ₐ) is produced (i.e. by a respective promoter; e.g. P₁) and/or controlled (i.e. by a respective post-transcriptional regulatory state, e.g. PTS₁). However, an “effective amount of an output RNA” (i.e. the output of the DNA construct), encompasses, in particular, all types of output RNAs present in the cell that are useful, e.g. output RNAs that comprise a sequence encoding an output protein (i.e. in-frame) (coding sequence (CDS)), no matter if the corresponding CDS is fully contained in the output sequence (or second exon alone) or partly in an alternative first exon and partly in the second exon and/or output RNAs that are functional themselves (e.g. as antisense RNAs that may control the cellular behavior and/or state), as described herein.

Preferably herein and in the context of the present invention, an output RNA is a messenger RNA (mRNA), and a pre-RNA is a pre-mRNA. In some embodiments, an output RNA is a non-coding RNA such as a long non-coding RNA or a microRNA-containing RNA.

In particular herein, and in the context of the present invention, the yield of an effective amount of an output RNA in a cell (which comprises a DNA construct of the invention) may correspond to the presence of an at least 1.5-, 2-, 4-, 6-, 8-, 10-, 15-, 20-, 30-, 40-, 60-, 80-, 100-, 150-, or 200-fold, preferably at least 10-fold, higher amount of the output RNA in said cell compared to the amount of the output RNA present in a cell comprising the same DNA construct in a condition under which no effective amount of an output RNA is obtained (e.g. a control cell and/or control condition), e.g. when none of the respective transcriptional regulatory states is present to initiate transcription from any of the promoters contained in said DNA construct.

Furthermore, the effective amount of an output RNA may correspond to (and/or is translated into), an effective amount of at least one output protein, i.e. at least one reporter protein and/or effector protein that is encoded by said output RNA.

Thus, the yield of an effective amount of an output RNA (that encodes at least one output protein) and/or output protein in a cell (which comprises a DNA construct of the invention) may further correspond herein and in context of the present invention to the presence of an at least 1.5-, 2-, 4-, 6-, 8-, 10-, 15-, 20-, 30-, 40-, 60-, 80-, 100-, 150-, or 200-fold, preferably at least 10-fold, higher amount of an output protein that is encoded by said output RNA in said cell compared to the amount of said output protein present in a cell comprising the same DNA construct in a condition under which no effective amount of said output RNA is obtained (e.g. a control cell and/or control condition), e.g. when none of the respective transcriptional regulatory states is present to initiate transcription from any of the promoters contained in said DNA construct.

A control cell and/or condition may refer to a reference cell and/or condition, as described herein, for which it is known that the DNA construct of the invention does not yield an effective amount of an output RNA and/or corresponding output protein therein.

When the DNA construct of the invention can produce (under certain conditions) an output RNA that encodes at least one output protein, the yield of an effective amount of an output RNA in a cell (which comprises said DNA construct of the invention) may correspond preferably to the presence of an at least 1.5-, 2-, 4-, 6-, 8-, 10-, 15-, 20-, 30-, 40-, 60-, 80-, 100-, 150-, or 200-fold, preferably at least 10-fold, higher amount of an output protein that is encoded by said output RNA in said cell compared to the amount of said output protein present in a cell comprising the same DNA construct in a condition under which no effective amount of said output RNA is obtained, e.g. when none of the respective transcriptional regulatory states is present to initiate transcription from any of the promoters contained in said DNA construct.

Furthermore, a similar concept may be applied for determining how well the DNA construct of the invention can distinguish between conditions in which an effective amount of an output RNA should be produced, and conditions in which no effective amount of an output RNA should be produced, e.g. according to the respective logic formula, for example, as illustrated, in the appended Examples, and as further described herein. For example, the DNA construct of the invention may yield an at least 1.5-, 2-, 4-, 6-, 8-, 10-, 15-, 20-, 30-, 40-, 60-, 80-, 100-, 150- or 200-fold, preferably at least 10-fold, higher amount of an output RNA and/or corresponding output protein in at least one, preferably each, condition in which an effective amount of an output RNA should be produced compared to at least one, preferably each, condition in which no effective amount of an output RNA should be produced (e.g. according to the respective logic formula).

Furthermore, the DNA construct of the invention may contain a transcription termination sequence downstream of the output sequence (or second exon), or near the 3′ end or at the 3′ end of the output sequence (or second exon). In particular, said transcription termination sequence comprises a polyadenylation signal. Transcription termination sequences are well known in the art, and illustrated in the appended Examples. For example, a transcriptional termination sequence may comprise a rabbit beta globin polyadenylation signal.

Thus, the output RNA produced and/or obtained (including different types of output RNA) by the DNA construct of the invention may comprise a poly-A tail at the 3′ end.

As used herein, a sequence contained in an output RNA (i.e. an RNA sequence) that corresponds to a sequence contained in the inventive DNA construct (i.e. a DNA sequence), is usually the same sequence as said DNA sequence (i.e. as the coding strand thereof) except that any thymine (T) in the corresponding DNA sequence is an uracil (U) in said RNA sequence. The same is true vice versa, e.g. for a DNA sequence in a DNA construct corresponding to a sequence in an output RNA. Furthermore, the person skilled in the art immediately understands from context whether a certain sequence is contained in a DNA or RNA, and thus whether any T has to be replaced by a U, or vice versa.

DNA and RNA sequences should be read, in the context of the present invention, in 5′ to 3′ direction. Furthermore, a sequence that is 5′ of another sequence, e.g. in a DNA construct or an output RNA, may be also specified as “upstream” of said other sequence herein. Furthermore, a sequence that is 3′ of another sequence, e.g. in a DNA construct or an output RNA, may be also specified as “downstream” of said other sequence herein.

An inventive DNA construct that does not contain at least one 5′ss, a BP and a 3′ss, as described herein, which is also called a “non-splicing DNA construct” herein, does not have a typical exon/intron structure. Thus, the sequence between two promoters in non-splicing DNA constructs is called a “spacer”, wherein a spacer may contain a unique sequence (US), as described herein, and the sequence contained in any output RNA produced by any promoter contained in a non-splicing DNA construct (common sequence) is called “output sequence” herein.

Although, in an inventive splicing DNA construct, as described herein, the sequence between two promoters may still be considered a “spacer” which may contain a unique sequence (US) and/or which may disrupt or abrogate interactions between the promoters, as described herein, usually a more precise terminology is used in the context of splicing DNA constructs herein, e.g. in the following. The spacer of a splicing DNA construct comprises, in particular, in 5′ to 3′ direction an alternative first exon (E1), a respective alternative 5′ss, and optionally a respective further intronic sequence, wherein the alternative first exon (E1) may comprise a unique sequence (US). Since the “output sequence” in a splicing DNA construct is downstream of the 3′ss, it refers to or comprises, in particular, a common second exon which is contained in any output RNA produced by any promoter contained in the splicing DNA construct.

As used herein, and in the context of the invention, a second exon may be an ordinary exon as commonly understood in the art (i.e. without any internal intronic sequence) or it may comprise one or more intronic sequences in the middle (i.e. not at any end) and thus may be a “split exon” which may be also considered a bunch of exons. Furthermore, the output sequence as used herein and in the context of the invention, may refer to or comprise (in the context of the inventive splicing DNA constructs provided herein), an ordinary second exon as commonly understood in the art, a split second exon (including the intronic sequence(s) therein), or the exonic part of a split second exon. Preferably herein and in the context of the invention, the second exon is an ordinary exon and the output sequence is or comprises an ordinary second exon.

Similarly, as used herein, and in the context of the invention, an alternative first exon may be an ordinary exon as commonly understood in the art (i.e. without any internal intronic sequence) or it may comprise one or more intronic sequences in the middle (i.e. not at any end) and thus may be a “split exon” which may be also considered a bunch of exons. Preferably herein and in the context of the invention, an alternative first exon is an ordinary exon.

Furthermore, the splicing DNA construct of the invention may further comprise additional exons, introns, branch points and/or splice sites, as long as the DNA construct is functional as described herein. Thus, the alternative first exon(s), as used herein, is/are preferably but not necessarily the “first” exon(s) in 5′ to 3′ direction. For example, the alternative first exon(s) may be preceded by at least one additional other exon and thus they may be middle exon(s). Furthermore, the second exon, as used herein, is not necessarily the “second” exon in 5′ to 3′ direction but it may be, e.g., the last exon. Thus, the terms “first exon” and “second exon” should not be understood in a strict and/or narrow sense herein and in the context of the present invention. However, herein and in the context of the present invention, the second exon is, in particular, downstream of all alternative first exons.

Furthermore, the inventive splicing DNA construct of invention may further comprise in 5′ to 3′ direction between said last promoter and said branch point (BP) the last alternative first exon of n respective further alternative first exons (E1ₙ) and the last alternative 5′ splice site of n respective further 5′ splice sites (5′ssₙ).

Furthermore, said inventive splicing DNA construct may further comprise in 5′ to 3′ direction between said first alternative 5′ splice site (5′ss₁) and said last promoter at least one further group of elements, wherein each of said groups of elements comprises in 5′ to 3′ direction:
a further one of said n further promoter(s) (Pₙ),
a further one of said n respective further alternative first exons (E1ₙ), and
a further one of said n respective alternative 5′ splice site (5′ssₙ).

Preferably herein, the last alternative 5′ splice site in an inventive splicing DNA construct of the invention is weaker than another 5′ splice sites contained in said DNA construct, and more preferably, said last alternative 5′ splice site is the weakest one among all 5′ splice sites contained in said DNA construct.

The person skilled in the art can easily determine the strength of a splice site, e.g. a 5′ splice, as well as the effects of modulating the 5′ splice sites strength(s), for example with respect to the amount of the different types of output RNAs and/or corresponding proteins obtained, e.g. as demonstrated in the appended Examples (see, e.g. Table 5).

Furthermore, a splicing enhancer sequence may be inserted between at least one 5′ss and the 3′ss (i.e. an intron) of the DNA construct of the invention, preferably into at least one further intronic sequence. Preferably, a splicing enhancer sequence may be inserted into another intron than the last intron. For example, the splicing enhancer sequence may be inserted into the most 5′ intron. Suitable splicing enhancer sequences are well known in the art and described in the appended Examples. Furthermore, a certain splicing enhancer and/or the functionality of a certain splicing enhancer may be or may be not associated with a certain transcriptional regulatory state, a certain post-transcriptional regulatory state, and/or a certain cell type and/or state, as described herein.

Furthermore, the length between any 5′ss and a 3′ss (i.e. introns) may be tuned, e.g. by modifying the size of intermediate promoters and/or, preferably, further intronic sequences. This may further improve the splicing of the output RNA, and/or the performance of the DNA construct of the invention. In particular, guidelines for modifying the length of DNA sequence elements such as, inter alia, further intronic sequences are disclosed herein, e.g. in the appended Examples.

Furthermore, a stop codon may be inserted between any (or each) 5′ss and the next downstream promoter of the inventive splicing DNA construct, e.g. between 5′ss₁ and P₂, and/or between 5′ss₂ and P₃. In particular, a stop codon may prevent the translation of an undesired protein from a mis-spliced RNA, as illustrated in the appended Examples.

In particular herein, i.e. in the context of an inventive splicing DNA construct, the branch point (BP) and the 3′ splice site (3′ss) enable the removal of the sequence(s) between at least one 5′ splice site (e.g. 5′ss₂) and the 3′ splice site contained in an output RNA, i.e. a pre-RNA, produced by any of the promoters contained in an inventive splicing DNA construct provided herein. Preferably said BP and 3′ss enable at least the removal of the sequence between the 5′ss that is most 5′ (“upstream”) in the output RNA and said 3′ss, and/or said BP and 3′ss enable the removal of the sequence(s) between each 5′ss in the output RNA and the 3′ss. In other words, the BP and 3′ss used in the context of the invention should be able to engage in RNA splicing.

In particular herein, i.e. in the context of an inventive splicing DNA construct, the first alternative 5′ splice site (5′ss₁) enables the removal of the sequence between said 5′ss₁ and the 3′ss (i.e. intron) contained in an output RNA, i.e. a pre-RNA, produced by P₁ contained in an inventive splicing DNA construct provided herein, and, in particular, each of the further alternative 5′ splice sites (if there are any) (e.g. 5′ss₂) enables the removal of the sequence between the respective further 5′ splice site (e.g. 5′ss₂) and the 3′ss (i.e. intron) contained in an output RNA i.e. a pre-RNA, produced by a promoter that is 5′ (“upstream”) of said further 5′ splice site (e.g. P₁ or P₂), i.e. the closest promoter 5′ (“upstream”) of said 5′ splice site (e.g. P₂). In other words, a 5′ss used in the context of the invention should be able to engage in RNA splicing.

It is well known in the art that a 5′ splice and a 3′ splice site usually specify the sequence that is removed by splicing between said 5′ss and said 3′ss (i.e. an intron), wherein the 5′ss and the 3′ss form the borders of the intron. In particular, the sequence that is removed between a 5′ss and a 3′ss may contain at the 5′ end a part of the 5′ss (the intronic part of the 5′ss) and at the 3′ss a part of the 3′ss (the intronic part of the 3′ss). Thus, the sequence that is removed between a 5′ss and a 3′ss in context of the invention may comprise part of the 5′ss and part of the 3′ss.

Furthermore, the splicing DNA construct of the invention may comprise a polypyrimidine tract between the branchpoint and the 3′ss.

Thus, i.e. in the context of an inventive splicing DNA construct, an output RNA further comprises, in particular, 5′ of the sequence corresponding to the second Exon (E2)
(i) a sequence corresponding to the first alternative first Exon (E1ₐ), when said output RNA is produced by P₁, and/or
(ii) a sequence corresponding to one of said further alternative first exon(s), e.g. E1_{b}, when said output RNA is produced by a promoter that is 5′ of the respective further alternative first exon (e.g. P₁ or P₂), i.e. the closest promoter 5′ of said further alternative first exon (e.g. P₂).

Preferably, i.e. in the context of an inventive splicing DNA construct, an output RNA does not comprise (at least not to a large and/or undesired extent)
(i) a sequence corresponding to the sequence between the 5′ss₁ and the 3′ss, when said output RNA is produced by P₁, and/or
(ii) a sequence corresponding to the sequence between a further 5′ splice site (e.g. 5′ss₂) and said 3′ss, when said output RNA is produced by a promoter that is 5′ of said further 5′ splice site (e.g. P₁ or P₂), i.e. the closest promoter 5′ of said 5′ splice site (e.g. P₂).

The inventive splicing DNA construct provided herein has the further advantage, as illustrated in the appended Examples, that different types of output RNAs can be produced by the different alternative promoters contained in said DNA construct, wherein a certain type of an output RNA may enable the production of a certain type of an output protein.

In the inventive non-splicing DNA constructs, the entire sequence encoding an output protein (CDS) including the start codon should be downstream of the last promoter to avoid that an intermediate promoter (or other undesired intermediate sequence) is co-translated, and a potentially undesired fusion-protein is obtained as an output protein. Thus, the same output protein is normally obtained with non-splicing DNA constructs, no matter from which promoter the output RNA encoding said output protein has been produced.

Although, the same output protein can be also obtained with the inventive splicing DNA constructs provided herein in such a way, if desired, the inventive splicing DNA constructs are more flexible and further allow the production of different useful types of an output protein.

In particular, an inventive splicing DNA construct may comprise in each alternative first exon a start codon, wherein the stop codon is contained in the common second exon. When the sequence between a 5′ss (i.e. the most upstream 5′ss) and the 3′ss in a certain output RNA (i.e. a pre-RNA produced by an upstream promoter) is removed by splicing, the start codon in an alternative first exon and the stop codon in the second exon may form an open reading frame (ORF) that is translated into a certain output protein. Since the output RNA obtained may encompass different types of output RNAs, wherein each type comprises a certain alternative first exon, dependent by which promoter it has been produced, different ORFs can be generated, and accordingly, different output proteins can be obtained. This is particularly helpful for understanding which promoters were active in the cell (and thus which transcriptional regulatory states were present), and/or for generating modified output proteins (which may have different functionalities) dependent on which promoters were active.

Thus, in the context of the present invention, the output RNA may comprise at least one sequence encoding at least one output protein, wherein the coding sequence (CDS) of said at least one output protein is/are partially or fully contained in the output sequence (i.e. in the context of the splicing DNA constructs in the second exon). Said at least one output protein may comprise a reporter protein, e.g. a fluorescent protein or a luminogenic or chromogenic enzyme, and/or an effector protein, e.g. a toxic protein, an enzyme, a cytokine, an immunomodulator, a membrane protein and/or a membrane-bound receptor.

Furthermore, an inventive DNA construct comprising at least one coding sequence, may further comprise upstream (i.e. directly adjacent) of at least one or each coding sequence a Kozak sequence. Suitable Kozak sequences are well known in the art and described in the appended Examples.

Thus, in particular, in the context of the inventive non-splicing DNA constructs, each of the CDS is fully contained in the output sequence.

Thus, in particular, i.e. the context of the inventive splicing DNA constructs, a CDS that is partially comprised in the second exon corresponds to or is part of at least one open reading frame (ORF), wherein the start codon of the ORF(s) is contained in at least one, preferably each, alternative first exon comprised in the DNA construct and the common stop codon of said ORF(s) is contained in the second exon. This may result in various useful output fusion-proteins (dependent on which promoters were active). In particular, each of said fusion proteins may comprise a basic output protein, e.g. a reporter protein and/or an effector protein as described herein, that is encoded by the common second exon, wherein said basic output protein is fused N-terminally to a certain peptide (e.g. a tag), wherein different peptides/tag are encoded by different alternative first exons. For example, an alternative first exon may comprise a sequence that encodes a peptide that controls the localization of a protein to which it is fused (localization tag, e.g. a nuclear localization sequence), and/or a sequence that encodes a peptide that controls the stability of a protein to which it is fused (stability tag, e.g. a PEST sequence, or an inducible degron). Furthermore, an alternative first exon may comprise a sequence that encodes any other tag or site which is known to affect the function, stability and/or function of a protein to which it is fused, e.g. a sequence encoding a protease cleavage site.

It is also possible, as illustrated in the appended Examples, that the common second exon comprises the part of a CDS that encodes the C-terminal part of an output protein, wherein said C-terminal part is common to different variants of said output protein, e.g. a fluorescent protein, and wherein each of the alternative first exons comprises a certain variant of the part of the CDS that encodes the N-terminal part of said output protein, wherein said N-terminal part is different in said variants and, preferably, specifies the properties of said output protein variants. For example, the derivatives (variants) of green fluorescent protein (GFP) such as CFP, Cerulean, Cerulean 2, Cerulean 3, Turquoise, Turquoise 2, BFP, SBFP2, YFP, Citrine, Venus, eGFP, Dendra2, etc., may comprise a common or similar C-terminal part and a variable N-terminal part. Since these different GFP variants may comprise different fluorescent properties (e.g. SBFP2, Cerulean and Citrine), they are suitable for analyzing which promoters have been active. For example, when E1ₐ comprises the part of the CDS that encodes the N-terminal part of SBFP2, E1_{b} comprises the part of the CDS that encodes the N-terminal part of Cerulean, E1_{c} comprises the part of the CDS that encodes the N-terminal part of Citrine, and E2 comprises the CDS that encodes the common part of SBFP2, Cerulean and Citrine, P₁ may produce an output RNA that encodes SBFP2, P₂ may produce an output RNA that encodes Cerulean, and P₃ may produce an output RNA that encodes Citrine.

Evidently, when the CDS of one or more output proteins is split up into different exons, the complete CDS should be in-frame, at least upon splicing, such that the desired output protein(s) is/are produced.

It is also possible that the common output sequence (or second exon) comprised in the inventive DNA construct provided herein contains a sequence that encodes a certain peptide (e.g. a tag) as described herein, for example, a peptide that controls the localization of a protein to which it is fused (localization tag, e.g. a nuclear localization sequence), and/or a sequence that encodes a peptide that controls the stability of a protein to which it is fused (e.g. an inducible degron). This may be useful to impart a further common functionality to all output protein types that may be obtained.

In the context of the inventive splicing DNA constructs provided herein, an alternative first exon may contain a 5′ untranslated region (5′ UTR) and downstream thereof a coding sequence or part of a coding sequence (CDS), as described herein. Preferably, the sequence(s) corresponding to the sequence(s) of the respective output RNA that may be targeted by a respective post-transcriptional regulatory state, as described herein, e.g. a unique sequence, an antisense RNA binding site, and/or an RNA-binding protein binding site, are contained in the 5′UTR of the respective alternative first exon.

Furthermore, a 5′ UTR may have a certain secondary structure and/or impart a certain secondary structure to the output RNA comprising said 5′ UTR. It is known in the art that the secondary structure of an RNA may affect the stability of the RNA and/or the translation efficiency. Thus, different types of output RNAs (produced by the respective promoters) may have different secondary structures which allows to further control the amount of an output RNA and/or an output protein encoded by an output RNA (and/or a certain type of an output RNA or corresponding output protein) that is obtained.

It is also possible that the common output sequence (or second exon) comprised in the inventive DNA construct provided herein contains a sequence that corresponds to a sequence of the output RNA (i.e. of all types of the output RNA) that may be targeted by a certain post-transcriptional regulatory state, e.g. an antisense RNA binding site, and/or an RNA-binding protein binding site, as described herein. This may be useful when it is desired that all types of the output RNA are degraded and/or translationally inhibited by a certain post-transcriptional regulatory state, regardless of which promoters are active and/or which transcriptional regulatory states are present in the cell. In particular, the sequence that may be targeted by a certain post-transcriptional regulatory state may be downstream of a coding sequence or the 3′ part of a coding sequence (CDS) within the common output sequence (or second exon), and hence, it may be contained in the 3′ untranslated region (3′ UTR).

Although it is preferred, in the context of the present invention, that an output RNA comprises a sequence that encodes an output protein, this does not have to be necessarily the case. For example, an output RNA itself may be detected in a cell by means known in the art, e.g., inter alia, by RT-PCR, RNA-sequencing, FISH, in situ hybridization and/or northern blot. Furthermore, an output RNA may be non-coding and may itself control the cellular behavior and/or state, e.g., it may be an long non-coding RNA (lncRNA) or an microRNA (miRNA) and act as an antisense RNA, and/or modulate transcription of certain genes, modulate the translation of certain mRNAs and/or modulate the activity of certain proteins.

However, employing at least one output protein, as described herein, may provide more flexibility and options and is, especially, more suitable for live cell applications, e.g. for selectively manipulating and/or killing target cells and/or for detecting target cells in vivo, as described herein.

As described herein, a certain type of an output RNA comprising a respective alternative first exon, and that is, in particular, produced by a respective promoter of the DNA construct of the invention, may be controlled by a respective post-transcriptional state. Thus, as illustrated in the appended Examples, the DNA construct of the invention may allow AND gate-like logic, or, preferably, AND NOT gate-like logic that is implemented at different molecular layers: the first “input” is the initiation of transcription from a certain promoter that is enabled by a respective transcriptional regulatory state and the production of a corresponding output RNA, as described herein, and the second “input” is the stability of the respective output RNA and/or the translation of an output protein encoded by the respective output RNA that is controlled by a respective post-transcriptional regulatory state. Preferably herein and in context of the invention, a post-transcriptional regulatory state degrades the respective output RNA and/or inhibits translation of an output protein encoded by the respective output RNA. Thus, this implementation preferably results in an AND NOT gate-like logic, i.e. the production of a certain type of an output RNA by a respective promoter that is enabled by the presence of a respective transcriptional regulatory state AND NOT the degradation and/or translation inhibition of said output RNA by the presence of a respective transcriptional regulatory yields an effective amount of said output RNA and/or an output protein encoded by said output RNA, and hence of an output RNA and/or output protein in general. This principle may be also considered in an abstracted and/or simplified (e.g. Boolean logic-like) way. At the level of one type of output RNA it may be noted: TS⁽¹⁾ AND NOT respective PTS⁽¹⁾=respective output RNA and/or protein⁽¹⁾; wherein 1 means “present” and 0 means “absent”.

Thus, the same may be noted like: TS⁽¹⁾ AND respective PTS⁽⁰⁾=respective output RNA and/or protein⁽¹⁾

Thus, at the level of an inventive DNA construct, it may be noted, e.g.: TS₁⁽¹⁾ AND NOT PTS₁⁽¹⁾ OR TS₂⁽¹⁾ AND NOT PTS₂⁽¹⁾=output RNA and/or protein⁽¹⁾.

The same may be noted like: TS₁⁽¹⁾ AND PTS₁⁽⁰⁾ OR TS₂⁽¹⁾ AND PTS₂⁽⁰⁾=output RNA and/or protein⁽¹⁾.

Furthermore, this type of AND gate-like logic or, preferably, AND NOT gate-like logic may be combined with the AND gate-like logic at the level of transcription initiation from a certain promoter, as described herein.

The various logic operations are further described in more detail herein.

Thus, in context of the inventive splicing DNA construct provided herein, an output RNA comprising a sequence corresponding to the first alternative first exon (E1ₐ), i.e. 5′ of the sequence corresponding to E2, may be controlled by a first post-transcriptional regulatory state (PTS₁), and/or an output RNA comprising a sequence corresponding to a further alternative first exon (E1ₙ, e.g., E1₂), i.e. 5′ of the sequence corresponding to E2, may be controlled by a respective further post-transcriptional regulatory state (PTSₙ, e.g., PTS₂).

In particular, an output RNA comprising a sequence corresponding to the first alternative first exon (E1ₐ) may be translationally inhibited and/or degraded by a first post-transcriptional regulatory state (PTS₁), and/or an output RNA comprising a sequence corresponding to a further alternative first exon (E1ₙ, e.g., E1₂) may be translationally inhibited and/or degraded by a respective further post-transcriptional regulatory state (PTSₙ, e.g., PTS₂).

Therefore, an inventive splicing DNA construct may yield, e.g. in some embodiments, an effective amount of an output RNA and/or output protein encoded by an output RNA in the eukaryotic cell, when
(i) at least one transcriptional regulatory state, i.e. TS₁ and/or any of TSₙ, e.g. TS₁, is present in said cell such that an output RNA from at least one respective promoter contained in said DNA construct, e.g. P₁, is produced; and
(ii) none of the respective post-transcriptional regulatory state(s) (e.g. PTS₁) is present in said cell such that the produced output RNA comprising the respective alternative first exon(s) (e.g. E1ₐ) is not translationally inhibited or degraded, e.g. such that not all different types of output RNAs (produced by the different respective promoters) are translationally inhibited or degraded, and hence, an effective amount of at least one type of an output RNA is obtained.

Preferably, said inventive splicing DNA construct, e.g. in said embodiments, may not yield an effective amount of an output RNA and/or output protein encoded by an output RNA in said eukaryotic cell, when
(i) no transcriptional regulatory state, e.g. neither TS₁ nor TS₂, that is capable of inducing transcription from the respective promoter(s) contained in said DNA construct, e.g. P₁ and P₂, is present in said cell, and/or
(ii) each post-transcriptional regulatory state, e.g. PTS₁ and PTS₂, that is capable of translationally inhibiting and/or degrading the output RNA produced by the respective promoters contained in said DNA construct, e.g. P₁ and P₂, is present in said cell, e.g. such that all different types of output RNAs (produced by the different respective promoters and/or containing different sequences corresponding to the respective alternative first exon(s)) are translationally inhibited or degraded, and hence, no effective amount of any output RNA is obtained.

In the context of the invention, a post-transcriptional regulatory state may be a characteristic of a eukaryotic cell which comprises the DNA construct of the invention and/or in which the DNA construct of the invention is functional. Thus, a certain post-transcriptional regulatory state, e.g. PTS₁, or any of PTSₙ such as PTS₂ or PTS₃ may be associated with and/or reflect a certain cell type and/or cell state. Hence, a cell comprising a certain post-transcriptional regulatory state (i) may promote the stability of a respective output RNA and/or the translation of an output protein from a respective output RNA, or, preferably, cause the degradation of a respective output RNA and/or inhibit the translation of an output protein from a respective output RNA. In other words, for example in case (ii), said output RNA is degraded and/or the translation of an output protein encoded by said output RNA is inhibited in said cell.

The cell type and/or state and the associated post-transcription regulatory state, as used herein and in the context of the invention, is not particularly limited, as described herein, e.g., as described herein in context of the cell type and/or state and the associated transcriptional regulatory state.

However, since a certain transcriptional regulatory state enables transcription initiation from a respective promoter (i.e. it is a positive regulator), and a respective post-transcriptional regulatory state preferably degrades the respective produced RNA and/or inhibits the translation of an output protein of the respective produced RNA (i.e. it is preferably a negative regulator), a certain transcriptional regulatory state, e.g. TS₁, and a respective post-transcriptional regulatory state, e.g. PTS₁, are, in the context of the present invention, preferably not comprised in the same cell type and/or cell state, in which an effective amount of an output RNA is desired to be obtained (e.g. a target cell). Thus, a certain cell type and/or cell state in which an effective amount of an RNA output is desired to be obtained (e.g. a target cell), as used herein and in context of the invention, does preferably not comprise the presence of a certain transcriptional regulatory state, e.g. TS₁, and the presence of a respective post-transcriptional regulatory state, e.g. PTS₁.

Conversely, a certain cell type and/or cell state, e.g., in which an effective amount of an RNA output is desired to be obtained (e.g. a target cell), as used herein and in context of the invention may preferably comprise
(i) the presence of a certain transcriptional regulatory state, e.g. TS₁, for example, the presence of at least one or a certain number of transcription factor(s) (TFs) from a respective group of TFs, e.g., TF₁, or TF₁ and TF₂; and/or
(ii) the absence of a certain, i.e. respective, post-transcriptional regulatory state, e.g. PTS₁, for example, the absence of at least one or a certain number of antisense RNA(s) (AR) from a respective group of ARs, e.g., AR₁, or AR₁ and AR₂.

For example, the absence of a certain post-transcriptional regulatory state may be associated with and/or reflect a disease cell state, i.e. an abnormal and/or malignant state of a cell, e.g. a cell of a certain tissue type.

Thus, the presence of a certain post-transcriptional regulatory state may be preferably associated with and/or reflect an, i.e. respective, non-disease (healthy) cell state, i.e. a normal and/or benign state of a cell, e.g. a cell of a certain tissue type. This is further described herein, i.e. context of the inventive medical and/or diagnostic uses of the DNA construct of the invention.

Furthermore, a post-transcriptional regulatory state, as used herein and in the context of the present invention, may comprise any mechanism and/or factor that can contribute to and/or, preferably, regulate the stability of a respective output RNA and/or the translation of an output protein encoded by a respective output RNA. An individual mechanism and/or factor may promote or inhibit the stability of an output RNA and/or the translation of a corresponding output protein. Yet, a certain post-transcriptional regulatory state is preferably considered present herein, when a respective output RNA is degraded and/or the translation of an output protein encoded by a respective output RNA is inhibited, and, in particular, no effective amount of a respective output RNA and/or output protein encoded by a respective output RNA is obtained (i.e. due to degradation and/or translational inhibition of the respective output RNA). Thus, when a certain post-transcriptional regulatory state is considered present, the corresponding mechanisms and/or factors must, in preferred embodiments, overall (in total and/or in combination), degrade the respective output RNA and/or inhibit translation of an output protein encoded by the respective output RNA. When this is not the case, said certain post-transcriptional regulatory state is preferably considered absent herein. Of note, this may be different, i.e. inversed, in some embodiments in which a post-transcriptional regulatory state promotes the stability of the respective output RNA, and/or promotes the translation of an output protein from the respective output RNA.

Of note, as used herein, translation of “an” output protein from a respective output RNA, comprises, in particular, the translation of “at least one” or “each” protein output protein from a respective output RNA, i.e. at least one or each of the output proteins that is/are encoded by a respective output RNA.

Mechanisms and/or factors that contribute to and/or regulate the stability of an output RNA and/or the translation of an output protein from an output RNA include antisense RNAs (e.g. microRNAs (miRNA; an illustrative example may be miR-1), small-interfering RNAs (siRNA; an illustrative example may be FF4), small-hairpin RNAs (shRNA) and/or antisense oligonucleotides (ASOs) (e.g. ARs from a respective group of ARs, as described herein), in particular wherein said antisense RNAs (e.g. miRNA) may be cell type and/or state specific, general RNA-binding proteins (e.g., inter alia, human pumilio 1, and SF2/ASF protein), cell type and/or state specific RNA-binding proteins, and/or riboswitches (i.e. the activation or inhibition of a riboswitch by an oligonucleotide, e.g. an antisense RNA).

A certain cell type and/or state and/or the presence of a certain post-transcriptional regulatory mechanism and/or factor (e.g. the presence of a certain antisense RNA) may be readily determined and/or defined for reference purposes by the person skilled in the art by any means known in the art, as further described herein in context of the determination of a certain cell type and/or state and/or the presence of a certain transcriptional regulatory mechanism and/or factor. For example, the detection of antisense RNAs, e.g. miRNAs, and/or RNA-binding proteins, does not involve any particular difficulties and can be also easily performed by methods known in the art, and/or methods described herein.

Thus, the person skilled in the art has not difficulties to define reference post-transcriptional regulatory programs and/or reference cell types and/or states.

Hence, it can be not only easily determined whether a certain promoter is active in a certain reference cell that comprises a certain transcriptional regulatory state (e.g. a reference cell of a certain type and/or in a certain state) by ordinary means, but it can be also easily determined whether a certain output RNA is degraded and/or the translation of an output protein from a certain output RNA is inhibited in a certain reference cell that comprises a certain post-transcriptional regulatory state.

Thus, the person skilled in the art can select and/or modify a unique sequence comprised in the inventive DNA construct provided herein (e.g. in a certain alternative first exon(s), 5′UTR or the 3′UTR) by routine experimentation such that the respective output RNA comprising a sequence corresponding to said unique sequence is degraded and/or translationally inhibited when a respective post-transcriptional regulatory state is present.

Furthermore, rational design principles can be applied for selecting and/or modifying unique sequences (e.g. in alternative first exons). For example, when it is desired or expected that a certain post-transcriptional regulatory state comprises a certain antisense RNA, e.g. an AR from a certain group of antisense RNAs as described herein, a respective unique sequence and/or alternative first exon comprised in the inventive DNA construct may comprise respective binding sites (target sites) to which said AR(s) can bind.

Thus, a certain post-transcriptional regulatory state, i.e. the presence of a certain transcriptional regulatory state, may comprise the presence of at least one antisense RNA (AR) from a respective group of antisense RNAs, e.g.,
wherein PTS₁ comprises the presence of at least one AR from a first group of ARs (AR₁), e.g. AR₁ₐ and/or AR_{1b}, and/or
a PTSₙ, e.g. PTS₂, comprises the presence of at least one AR from a further group of ARs (ARₙ, e.g., AR₂), for example, ARₙₐ and/or AR_{nb}, e.g. AR₂ₐ and/or AR_{2b}.

However, an individual AR may be contained in one or more of said groups of ARs.

For example, an antisense RNA may comprise a sequence of at least 10, 15 or 20 contiguous bases, wherein said sequence has at least 40%, 50%, 60%, 70%, 80%, 90% or 100% sequence identity to the complementary sequence of a respective target site (binding site), e.g. a target site in the respective alternative first exon contained in an output RNA.

Suitable tools that provide the percentage of the sequence identity between two sequences are well known in the art. For example, Nucleotide BLAST (e.g. at the NCBI webpage) may be used to check whether a sequence has at least 10, 15 or 20 contiguous bases with at least 40%, 50%, 60%, 70%, 80%, 90% or 100% sequence identity to the complementary sequence of a respective target site.

An antisense RNA, herein and in the context of the invention, may be, for example, a microRNA (miRNA) or a small interfering RNA (siRNA). Preferably said antisense RNA is a miRNA. In particular, an antisense RNA (AR) may promote the translational inhibition and/or degradation of an output RNA containing at least one target site (binding site), for said AR, as described herein. In particular, the translational inhibition of an output RNA, as used herein, refers to the inhibition of the production, i.e. the translation, of at least one output protein encoded by said output RNA, e.g. a reporter and/or effector protein, as described herein.

Furthermore, a certain post-transcriptional regulatory state, i.e. the presence of a certain transcriptional regulatory state, may comprise the presence of at least one RNA-binding protein (RB).

In particular, a certain AR is considered present when a higher amount of the AR compared to a control cell that does not contain and/or express said AR is present, and/or when adding a similar amount of the AR (e.g. by forced expression from a plasmid, and/or injection into the cell) affects the amount of the respective RNA that is obtained (positive control experiment).

Similarly, a certain RB may be considered present when a higher amount of the RB compared to a control cell that does not express said RB is present, and/or when adding a similar amount of the RB (e.g. by forced expression from a plasmid) affects the amount of the respective RNA that is obtained (positive control experiment).

In particular, the first alternative first exon (E1ₐ) may comprise at least one sequence (e.g. a unique sequence) corresponding to at least one target site, i.e. binding site, for at least one AR or a certain number of ARs from AR₁, e.g. AR₁ₐ, or AR₁ₐ and AR_{1b}, i.e., wherein said at least one target site is contained in a sequence corresponding to E1ₐ in an output RNA produced by P₁; and/or
wherein any of said further alternative first exons (E1ₙ), e.g. E1_{b}, may comprise at least one sequence (e.g. a unique sequence) corresponding to at least one target site, i.e. binding site, for at least one AR or a certain number of ARs from a respective ARₙ, e.g. AR₂, for example, ARₙₐ, or ARₙₐ and AR_{nb}, e.g. AR₂ₐ, or AR₂ₐ and AR_{2b}, i.e., wherein said at least one target site is contained in a sequence corresponding to said E1ₙ in an output RNA produced by a promoter that is 5′ of said E1ₙ, i.e. the closest promoter 5′ of said E1ₙ.

Thus, a unique sequence (e.g. within a certain alternative first exon) comprised in the DNA construct of the invention may be designed such that the respective produced RNA is degraded and/or translationally inhibited when more than one, e.g. 2, 3, or 4 ARs are present, e.g. when said unique sequence (and/or alternative first exon) comprises at least one binding site for each of said ARs. In other words, it is possible that a certain output RNA is degraded and/or translationally inhibited in a synergistic way by more than one AR.

However, this principle is not limited to ARs, but any two or more mechanisms and/or factors, as described herein, may act synergistically to a degrade and/or translationally inhibit a certain output RNA.

Thus, the degradation of any one of the output RNAs produced by the inventive DNA construct (e.g. by two or more mechanisms and/or factors of the same respective post-transcriptional regulatory state, e.g. comprising two or more ARs from the same respective group of ARs) may follow an AND gate-like logic. Thus, the herein described logic operations may be further combined with said additional AND gate-like logic at the post-transcriptional level.

For example, even more complex logic operations such as, inter alia, (TF₁ₐ AND TF_{1b} AND NOT AR₁ₐ AND NOT AR_{1b}) OR (TF₂ₐ AND TF_{2b} AND NOT AR₂ₐ AND NOT AR_{2b}) may be achieved.

Thus, the DNA construct of the invention can combine multiple promoters with alternative splicing, which may provide a powerful approach to obtain nearly full control of gene expression (i.e. to control in which conditions an effective amount of an output RNA is obtained).

As described herein, the DNA construct of the invention may yield an at least 1.5-, 2-, 4-, 6-, 8-, 10-, 15-, 20-, 30-, 40-, 60-, 80-, 100-, 150-, or 200-fold, preferably at least 10-fold, higher amount of an output RNA and/or corresponding output protein in at least one, preferably each, condition in which an effective amount of an output RNA should be produced compared to at least one, preferably each, condition in which no effective amount of an output RNA should be produced according to the respective logic formula.

In an illustrative Example, the DNA construct of the invention comprises
(i) two alternative promoters (P₁ and P₂), wherein
   P₁ is activated by the respective transcriptional regulatory state (TS₁) and
   P₂ is activated by the respective transcriptional regulatory state (TS₂);
and
(ii) two respective alternative first exons (E1ₐ and E1_{b}), wherein
   the output RNA comprising a sequence corresponding to E1ₐ (which is produced by P₁; RNA₁) is degraded and/or translationally inhibited by the respective post-transcriptional regulatory state (PTS₁), and
   the output RNA comprising a sequence corresponding to E1_{b} (which is produced by P₂; RNA₂) is degraded and/or translationally inhibited by the respective post-transcriptional regulatory state (PTS₂).

Thus, the logic formula for said illustrative Example is: (TS₁ AND NOT PTS₂) OR (TS₂ AND NOT PTS₂).

Thus, according to said logic formula, said exemplary DNA construct
should yield an effective amount of an output RNA in a eukaryotic cell, when
   (1) TS₁ is present, TS₂ is present, PTS₁ is absent, and PTS₂ is absent;
   (2) TS₁ is present, TS₂ is present, PTS₁ is present, and PTS₂ is absent;
   (3) TS₁ is present, TS₂ is present, PTS₁ is absent, and PTS₂ is present;
   (4) TS₁ is present, TS₂ is absent, PTS₁ is absent, and PTS₂ is absent;
   (5) TS₁ is present, TS₂ is absent, PTS₁ is absent, and PTS₂ is present;
   (6) TS₁ is absent, TS₂ is present, PTS₁ is absent, and PTS₂ is absent; or
   (7) TS₁ is absent, TS₂ is present, PTS₁ is present, and PTS₂ is absent;
and should not yield an effective amount of an output RNA in a eukaryotic cell, when
   (8) TS₁ is present, TS₂ is present, PTS₁ is present, and PTS₂ is present;
   (9) TS₁ is present, TS₂ is absent, PTS₁ is present, and PTS₂ is absent;
   (10) TS₁ is present, TS₂ is absent, PTS₁ is present, and PTS₂ is present;
   (11) TS₁ is absent, TS₂ is present, PTS₁ is absent, and PTS₂ is present;
   (12) TS₁ is absent, TS₂ is present, PTS₁ is present, and PTS₂ is present;
   (13) TS₁ is absent, TS₂ is absent, PTS₁ is present, and PTS₂ is present;
   (14) TS₁ is absent, TS₂ is absent, PTS₁ is present, and PTS₂ is absent;
   (15) TS₁ is absent, TS₂ is absent, PTS₁ is absent, and PTS₂ is present; or
   (16) TS₁ is absent, TS₂ is absent, PTS₁ is absent, and PTS₂ is absent.

Evidently, herein, “absent” does mean the same as “not present”; and “not yielding an effective amount” does mean the same as “yielding no effective amount”.

The 16 conditions that are possible in said example, may be further noted down in form of a truth table, wherein “1” means to “present” or “true”, and “0” means “absent” or “false”.

The truth table for said exemplary DNA construct is:

| | | | | | Output |
|---|---|---|---|---|---|
| | | | | | Effective |
| | Input | | | | amount of an |
| Condition | TS₁ | TS₂ | PTS₁ | PTS₂ | output RNA |
| 1 | 1 | 1 | 0 | 0 | 1 |
| 2 | 1 | 1 | 1 | 0 | 1 |
| 3 | 1 | 1 | 0 | 1 | 1 |
| 4 | 1 | 0 | 0 | 0 | 1 |
| 5 | 1 | 0 | 0 | 1 | 1 |
| 6 | 0 | 1 | 0 | 0 | 1 |
| 7 | 0 | 1 | 1 | 0 | 1 |
| 8 | 1 | 1 | 1 | 1 | 0 |
| 9 | 1 | 0 | 1 | 0 | 0 |
| 10 | 1 | 0 | 1 | 1 | 0 |
| 11 | 0 | 1 | 0 | 1 | 0 |
| 12 | 0 | 1 | 1 | 1 | 0 |
| 13 | 0 | 0 | 1 | 1 | 0 |
| 14 | 0 | 0 | 1 | 0 | 0 |
| 15 | 0 | 0 | 0 | 1 | 0 |
| 16 | 0 | 0 | 0 | 0 | 0 |

Thus, said exemplary DNA construct may yield, for example, an at least 1.5-, 2-, 4-, 6-, 8-, 10-, 15-, 20-, 30-, 40-, 60-, 80-, 100-, 150-, or 200-fold, preferably at least 10-fold, higher amount of an output RNA and/or corresponding output protein in at least one, preferably at least 5, more preferably all of conditions 1 to 7 compared to at least one, preferably at least 5, more preferably al of conditions 8 to 16.

In other words, said exemplary DNA construct may yield, for example, an at least 1.5-, 2-, 4-, 6-, 8-, 10-, 15-, 20-, 30-, 40-, 60-, 80-, 100-, 150-, or 200-fold, preferably at least 10-fold, higher amount of an output RNA and/or corresponding output protein in at least one, preferably at least 70%, more preferably all of conditions 1 to 7 compared to at least one, preferably at least 50%, more preferably all of conditions 8 to 16.

The person skilled in the art can easily determine the logic formula of any inventive DNA construct provided herein based on the present disclosure including the appended Examples and further common general knowledge. Furthermore, the person skilled in the art can also easily determine the truth table for any inventive DNA construct provided herein based on the present disclosure and further common general knowledge.

Furthermore, as described herein, the person skilled in the art can easily determine the amount of an output RNA and/or output protein present in a eukaryotic cell by means well known in the art, described herein and/or illustrated in the appended Examples.

Therefore, the person skilled in the art can further easily determine, whether the DNA construct of the invention yields an effective amount of an output RNA and/or output protein in a eukaryotic reference cell, for which it is known whether it comprises the respective transcriptional regulatory state(s) and/or post-transcriptional regulatory state(s).

Hence, the person skilled in the art can test and validate the performance of the DNA construct of the invention, for example, the person skilled in the art can determine how well the DNA construct of the invention can distinguish conditions in which an effective amount of an output RNA should be produced from conditions in which no effective amount of an output RNA should be produced according to the respective logic formula.

Thus, the present invention further provides a scalable approach to complex multi-input regulatory programs in mammalian cells that rely predominantly on transcriptional inputs. The present disclosure including the appended Examples provides design guidelines towards multi-promoter OR gates and their extension with AND and NOT logic which allow to overcome complications that may be caused by three distinct mechanisms and which may dictate quantitative performance of the DNA construct: (i) the alternative splicing per se, influenced by the choice of the alternative 5′-splice site sequence, and the length of the introns (e.g. the length between two alternative first exons); (ii) the transcriptional interference between different promoters, whereby upstream promoter activation may inhibit the expression from an activated downstream promoter via the mechanism of transcriptional run-through; and (iii) long-range transcriptional synergy between transcriptional inputs to the different promoters, which may result in an increase of expression from a downstream promoter upon TF binding to an upstream promoter. In addition, the NOT logic may rely on efficient knockdown of gene expression via 5′-UTR target sites, which may be less robust than the binding to 3′-UTRs (Gam et al., 2018). Moreover, the above mechanisms are not mutually independent, for example, the length of the introns may affect splicing but also the degree of synergy, and so on. However, the guidelines and design principles described herein and in the context of the present invention allow to overcome such complications and enable the generation of useful constructs that have, preferably, an at least acceptable performance. Furthermore, even full mapping of the most important genetic determinants into the logic performance of such complex constructs may be done, e.g., by big data acquisition and analysis via machine learning (Rosenberg et al., 2015), and is thus within reach of the currently available technologies and within the scope of the invention.

Ultimately, the present invention may allow to implement logic control of the form (TF1⁽¹⁾ and TF2⁽¹⁾ and . . . not (miRNA-a⁽¹⁾) and NOT(miRNA-b⁽¹⁾ . . . ) OR (TF1⁽²⁾ and TF2⁽²⁾ and . . . not (miRNA-a⁽²⁾) and NOT(miRNA-b⁽²⁾) . . . ), and being able to encode it, e.g., in viral vectors, as illustrated in the appended Examples, thus making the DNA constructs of the invention compatible with gene therapy, something that would have been impossible with multiple DNA constructs implementing the gate due to very large DNA footprint. Thus, the present invention provides DNA constructs and corresponding guidelines that may fully unleash the power of cell classification for specific cell state targeting in therapeutic applications.

Preferably, the inventive DNA construct provided herein has an at least acceptable performance.

In particular, the performance of a DNA construct of the invention may be considered acceptable when said DNA construct yields an at least 1.5-, 2-, 4-, 6-, 8-, 10-, 15-, 20-, 30-, 40-, 60-, 80-, 100-, 150-, or 200-fold, preferably at least 10-fold, higher amount of an output RNA and/or corresponding output protein in at least one, preferably at least 70%, more preferably each of the conditions in which an effective amount of an output RNA should be produced according to the respective logic formula, compared to at least one, preferably at least 50%, more preferably each of the conditions in which no effective amount of an output RNA should be produced according to said logic formula.

For example, the performance of a DNA construct of the invention may be considered acceptable when said DNA construct yields an at least 1.5-fold, preferably at least 10-fold, higher amount of an output RNA and/or corresponding output protein in at least one, preferably at least 70%, more preferably each of the conditions in which an effective amount of an output RNA should be produced according to the respective logic formula, compared to at least one, preferably at least 50%, more preferably each of the conditions in which no effective amount of an output RNA should be produced according to said logic formula.

Furthermore, the performance of a DNA construct of the invention may be considered acceptable when said DNA construct yields an at least 1.5-, 2-, 4-, 6-, 8-, 10-, 15-, 20-, 30-, 40-, 60-, 80-, 100-, 150-, or 200-fold, preferably at least 10-fold, higher amount of an output RNA and/or corresponding output protein in at least 70% of the conditions in which an effective amount of an output RNA should be produced according to the respective logic formula, compared to at least 50% of the conditions in which no effective amount of an output RNA should be produced according to said logic formula.

Preferably, the performance of a DNA construct of the invention may be considered acceptable when said DNA construct yields an at least 1.5-, 2-, 4-, 6-, 8-, 10-, 15-, 20-, 30-, 40-, 60-, 80-, 100-, 150-, or 200-fold, preferably at least 10-fold, higher amount of an output RNA and/or corresponding output protein in at least 70% of the conditions in which an effective amount of an output RNA should be produced according to the respective logic formula, compared to all conditions in which no effective amount of an output RNA should be produced according to said logic formula.

For example, the performance of a DNA construct of the invention may be considered acceptable when said DNA construct yields an at least 4-fold, preferably at least 10-fold, higher amount of an output RNA and/or corresponding output protein in at least 70% of the conditions in which an effective amount of an output RNA should be produced according to the respective logic formula, compared to all conditions in which no effective amount of an output RNA should be produced according to said logic formula.

More preferably, the performance of a DNA construct of the invention may be considered acceptable when said DNA construct yields an at least 1.5-, 2-, 4-, 6-, 8-, 10-, 15-, 20-, 30-, 40-, 60-, 80-, 100-, 150-, or 200-fold, preferably at least 10-fold, higher amount of an output RNA and/or corresponding output protein in all conditions in which an effective amount of an output RNA should be produced according to the respective logic formula, compared to all conditions in which no effective amount of an output RNA should be produced according to said logic formula.

For example, the performance of a DNA construct of the invention may be considered acceptable when said DNA construct yields an at least 1.5-fold, preferably at least 4-fold, preferably at least 10-fold, higher amount of an output RNA and/or corresponding output protein in all conditions in which an effective amount of an output RNA should be produced according to the respective logic formula, compared to all conditions in which no effective amount of an output RNA should be produced according to said logic formula.

Furthermore, the DNA construct of the invention may be designed such that two or more, e.g. all, conditions that should yield an effective amount of an output RNA according to the respective logic formula yield a similar amount of an output RNA, e.g., as illustrated in the appended Examples. For example, the amount of an output RNA between two conditions may be considered similar, when the fold-difference is less than 4-fold, preferably less than 2-fold, more preferably less than 1.5-fold.

However, the DNA construct of the invention may be further designed such that two or more conditions that should yield an effective amount of an output RNA according to the respective logic formula yield different amounts of an output RNA. For example, the amount of an output RNA between two conditions may be considered different, when the fold-difference is at least 1.5-fold, preferably at least 2-fold, more preferably at least 4-fold. This may allow to further fine tune the response to different cellular conditions, which might be relevant, e.g. for the medical uses provided herein.

However, the fold-difference between two or more conditions that should yield an effective amount of an output RNA (according to the logic formula) is preferably lower than the difference between any of these conditions compared to a condition in which no effective amount an output RNA (according to the logic formula) should be produced. Thus, in that case the difference between a condition that should yield an effective amount of an output RNA and that should not yield an effective amount of an output RNA is preferably at least 6-fold or, more preferably, at least 10-fold.

The maximum length of the DNA construct of the invention is not particularly limited.

Preferably, however, the DNA construct of the invention has a length of at most 150 kb, preferably at most 12 kb. Such a small length or size may be advantageous, e.g., for producing a viral vector comprising the DNA construct of the invention, and/or for therapeutic and/or diagnostic applications, as described herein.

The DNA construct of the invention can be easily assembled and/or synthesized by methods known in the art and as described herein, e.g. as illustrated in the appended Examples, for example, by cloning, oligonucleotide synthesis and/or a combination thereof.

Thus, the invention further relates to a method for producing the DNA construct of the invention, wherein said method comprises the steps of
(a) selecting and arranging the DNA sequence elements contained in the DNA construct, e.g. in silico, and
(b) assembling and/or synthesizing the selected and arranged DNA sequence elements, thereby producing said DNA construct.

Furthermore, the inventive production method provided herein may comprise prior to step (a), analyzing the transcriptional regulatory states and/or post-transcriptional regulatory states of target cells and/or non-target cells, as described herein, e.g. by determining the activity of at least one promoter in target cells and/or non-target cells, and/or by determining the presence of biomarkers, specific transcription factors, antisense RNAs such as miRNAs, the transcriptome and/or proteome in target cells and/or non-target cells.

This allows to design and/or test certain sequence elements of the DNA construct (e.g. promoters, antisense RNA binding sites, transcription factors binding sites etc.) prior to selecting and arranging all DNA sequence elements contained in the DNA construct, i.e. in a rational way.

Furthermore, the inventive production method provided herein may further comprise the steps of
(c) analyzing whether the DNA construct (i.e. produced by steps (a) and (b)) yields an effective amount of an output RNA in target cells and/or non-target cells, and
(d) selecting, and optionally amplifying or re-synthesizing, the DNA construct when said DNA construct yields an effective amount of an output RNA in at least two different types of target cells, and preferably does not yield an effective amount of an output RNA in non-target cells.

This may further ensure that the produced DNA construct has the desired functionality, e.g. that it can distinguish target cells from non-target cells.

As regards the target cells and/or non-target cells, the same applies as is described herein, e.g. in context of the inventive medical and/or diagnostic uses of the DNA construct of the invention.

Thus, the DNA construct of the invention may be obtainable and/or produced by the inventive production method provided herein.

Furthermore, the invention relates to a plasmid comprising the DNA construct of the invention. Any plasmid (i.e. a circular DNA molecule) that is used for cloning, and/or as an expression vector in the art is suitable in context of the invention. The plasmid of the invention can be easily produced by methods known in the art, e.g. by cloning the DNA construct into an existing plasmid, as illustrated, for example, in the appended Examples.

Furthermore, the invention relates to a virus comprising the DNA construct of the invention, which may be double-stranded DNA or single-stranded DNA (i.e. the coding strand of the DNA construct of the invention), a single-stranded DNA that comprises a sequence that is complementary to the sequence of the DNA construct of the invention (i.e. the antisense strand of the DNA construct of the invention), or an RNA (preferably a single-stranded RNA) that comprises a sequence corresponding to the DNA construct of the invention (i.e. corresponding to the coding strand of the DNA construct) and/or a sequence that is complementary to the sequence of the DNA construct of the invention (i.e. corresponding to the antisense strand of the DNA construct of the invention). In particular, said virus is a viral vector, e.g. an adeno-associated virus (AAV) vector, a lentiviral vector, an Adenoviral vector, a Herpes-Simplex Virus vector, or a VSV vector, preferably an adeno-associated virus vector or a lentiviral vector. The virus and/or viral vector of the invention can be easily produced by methods known in the art, as illustrated, for example, in the appended Examples. Such methods may comprise, e.g. generating a plasmid of the invention, wherein said plasmid is suitable for producing the corresponding virus and/or viral vector, and introducing said plasmid into a host cell (e.g. a HEK cell) that is suitable for producing said virus and/or viral vector.

Evidently, the plasmid of the invention and/or the virus or viral vector of the invention may comprise further sequences in addition to the DNA construct of the invention, as well known in the art and described in the appended Examples herein. These further sequences may, inter alia, enable the amplification of the plasmid in a bacterial cell, promote the production of the virus in vitro (e.g. in a eukaryotic cell), enable the formation of double-stranded DNA comprising the DNA construct of the invention in a eukaryotic cell into which the virus of the invention has been introduced and/or promote the integration of the DNA construct of the invention into the genome of a eukaryotic cell into which the virus of the invention has been introduced.

Thus, the invention further relates to a host cell comprising the DNA construct, plasmid and/or virus of the invention. In some embodiments, the host cell of the invention is a bacterium (e.g. E. coli) that is suitable for amplifying the plasmid of the invention. In some embodiments, the host cell of the invention is suitable for and/or used for producing the virus and/or viral vector of the invention. In other embodiments, the host cell of the invention is a target cell into which the DNA construct, plasmid, virus and/or viral vector has been transiently or stably introduced (e.g. by means of transfection and/transduction), for example, in the context of the medical and/or diagnostic uses of the invention.

Furthermore, the inventive DNA construct, plasmid, and/or virus (e.g. a viral vector) may be used for treating a disease in a subject, diagnosing a disease in a subject in vivo, and/or in an in vitro method for determining the cell type and/or state of a eukaryotic cell.

As used herein, “treatment” (and grammatical variations thereof such as “treat” or “treating”) refers to clinical intervention in an attempt to alter the natural course of the individual being treated. Desirable effects of treatment include, but are not limited to, prophylaxis, preventing occurrence or recurrence of disease or symptoms associated with disease, alleviation of symptoms, diminishment of any direct or indirect pathological consequences of the disease, decreasing the rate of disease progression, amelioration or palliation of the disease state, improved prognosis and cure.

Thus, the invention further relates to the DNA construct, plasmid, virus or host cell of the invention for use in treating a disease in a subject.

Thus, the invention also relates to a method for treating a disease in a subject, wherein said method comprises administering to said subject in need for therapy an effective amount of the DNA construct, plasmid, virus and/or host cell of the invention.

Thus, the invention further relates to a pharmaceutical composition comprising the DNA construct, plasmid, virus or host cell of the invention, and at least one further pharmaceutically acceptable substance.

Preferably herein and in the context of the invention, the eukaryotic cell, e.g. a target and/or a non-target cell, is a mammalian cell, preferably a human cell.

Thus, preferably herein and in the context of the invention, the subject is a mammal, preferably a human.

For example, the eukaryotic cell and/or the subject may be human, murine, equine, bovine, feline, canine etc., preferably human.

The DNA construct, plasmid, and/or virus of the invention, e.g. in the context of the inventive medical uses provided herein, may be introduced into a plurality of cells in a subject, in particular, wherein said plurality of cells may comprise target cells and non-target cells.

In particular, the DNA construct, plasmid, and/or virus of the invention may be introduced into a plurality of cells, e.g. into target cells and/or non-target cells, in a subject by systemic or locoregional delivery to said subject.

Furthermore, the DNA construct, plasmid, and/or virus of the invention may be delivered to a subject with single or repeated dosing.

Herein, and in the context of the invention, the disease may be associated with and/or caused by a heterogeneous mix of target cells (e.g. at least two different types of target cells). In particular, a target cell may correspond to a first abnormal and/or malignant cell type and/or state (AC₁), and/or another one or any of n further abnormal and/or malignant cell type(s) and/or state(s) (ACₙ), wherein n≥1 as described herein.

Herein, and in the context of the invention, treating the disease may comprise killing and/or manipulating target cells regardless whether a target cell corresponds to said first abnormal and/or malignant cell type and/or state (AC₁), and/or to another one or any of said further abnormal and/or malignant cell types and/or states (ACₙ). In particular, herein and in the context of the invention, the manipulated target cells may become harmless, less harmful or beneficial to the subject.

It is advantageous that the DNA construct of the invention may be clinically effective when one or different types of abnormal and/or malignant target cells (e.g. different subtypes of a cancer) are present in a subject that is in need of medical intervention.

For example, at least 1%, 5%, 10%, 20%, 30%, 40%, 50%, 60%, 70%, 80%, 90%, 95%, 99% or 100% of the target cells that correspond to AC₁, and at least 1%, 5%, 10%, 20%, 30%, 40%, 50%, 60%, 70%, 80%, 90%, 95%, 99% or 100% of the target cells that correspond to another AC (ACₙ), e.g. AC₂, may be killed and/or manipulated.

Preferably, at least 1%, 5%, 10%, 20%, 30%, 40%, 50%, 60%, 70%, 80%, 90%, 95%, 99% or 100%, e.g. about 1% to about 99%, about 10% to about 99% or about 10% to about 50% of the target cells that correspond to AC₁ and/or any of the ACₙ (e.g. AC₂ and/or AC₃) may be killed and/or manipulated.

Preferably herein, i.e. in the context of the medical uses, non-target cells, i.e. normal and/or benign cells, are not killed and/or manipulated.

For example, less than 30%, 20%, 10%, 5%, 2%, 1%, 0.5%, 0.1%, 0.05%, or 0.01%, preferably less than 10%, more preferably less than 10%, most preferably less than 0.10% of the non-target cells, i.e. normal and/or benign cells, are killed and/or manipulated.

In particular herein, i.e. the context of the inventive medical and/or diagnostic uses provided herein, a eukaryotic cell, e.g. a target cell and/or a non-target cell, may be a eukaryotic cell that comprises the DNA construct of the invention and/or in which the DNA construct of the invention is functional, as described herein.

In particular herein and in the context of the invention, (i) the AC₁ comprises the presence of the TS₁ described herein, and optionally the absence of the PTS₁ described herein, and/or (ii) an ACₙ, e.g. AC₂, comprises the presence of a respective TSₙ, e.g. TS₂, as described herein, and optionally the absence of a respective PTSₙ, e.g. PTP₂, as described herein.

In particular, as regards the transcriptional regulatory states, and the post-transcriptional regulatory state, the same applies as is described herein, e.g. in the context of the inventive DNA construct provided herein.

As described herein, the abnormal and/or malignant cell types and/or states (AC) may be respective features with respect to the features of the DNA construct of the invention (e.g. the promoters, the 5′ splice sites and/or the alternative first exons) and/or features of the cellular condition (e.g. the transcriptional regulatory states and/or the post-transcriptional regulatory states), as described herein. For example, when 2, 3, or 4 abnormal and/or malignant cell types and/or states should be killed and/or manipulated (e.g. according to the inventive medical uses provided herein), and/or detected (e.g. according to the inventive in vitro and/or in vivo diagnostic uses provided herein), the DNA construct of the invention may comprise 2, 3 or 4 respective promoters, and optionally 2, 3, or 4 respective 5′ss and respective alternative first exons that may comprises 2, 3 or 4 respective unique sequences.

In particular herein and in the context of the invention, an effective amount of an output RNA and/or at least one output protein encoded by an output RNA may be obtained
(i) in target cells that correspond to the first abnormal and/or malignant cell type and/or state (AC₁), and/or
(ii) in target cells that correspond to any of the further abnormal and/or malignant cell type(s) and/or state(s) (ACₙ, e.g. AC₂).

Furthermore, preferably no effective amount of an output RNA and/or an output protein encoded by the output RNA is obtained in the non-target cells.

As regards the effective amount of an output RNA, the same applies as is described herein, e.g. in the context of the inventive DNA construct provided herein.

Herein, e.g. in the context of the medical uses of the inventive DNA construct provided herein, the output RNA, e.g. at least one or each type of the output RNA, may be a long non-coding RNA (lncRNA) or a microRNA (miRNA) that is suitable for killing and/or manipulating a eukaryotic cell, e.g. a target cell, as described herein.

Preferably, in the context of the medical uses of the inventive DNA construct provided herein, the output RNA may comprise a sequence encoding at least one effector protein, e.g. a toxic protein, an enzyme, a cytokine, an immunomodulator, a membrane protein and/or a membrane-bound receptor.

Preferably, in the context of the medical uses of the DNA construct provided herein, the at least one output protein comprises at least one effector protein, e.g. a toxic protein, an enzyme, a cytokine, an immunomodulator, a membrane protein and/or a membrane-bound receptor.

In particular, the effector protein is suitable for killing and/or manipulating a eukaryotic cell, e.g. a target cell, as described herein.

Herein and in the context of the invention, the disease may be a cancer, a neurodegenerative disease, an immunodeficiency, and/or a genetic disease. Preferably, said disease is a cancer, e.g., inter alia, a liver cancer such as, inter alia, a hepatocellular carcinoma, a skin cancer such as, inter alia, a melanoma, a blood cancer such as, inter alia, a leukemia, a breast cancer, a prostate cancer, a lung cancer, a brain cancer such as, inter alia, a glioblastoma.

In particular, the cancer may comprise target cells that correspond to the first abnormal and/or malignant cell type and/or state (AC₁), and target cells that correspond to another one or any of said further abnormal and/or malignant cell type(s) and/or state(s) (ACₙ).

Furthermore, the DNA construct, plasmid and/or virus (e.g. a viral vector) of the invention may be used for analyzing a eukaryotic cell, i.e. for determining the cell type and/or state of a eukaryotic cell, as described herein.

Thus, the invention further relates to an in vitro method for determining the cell type and/or state of a eukaryotic cell, preferably a mammalian cell, wherein said method comprises
(a) introducing the DNA construct, plasmid or virus of the invention into said eukaryotic cell in vitro,
(b) measuring the amount of an output RNA and/or an output protein encoded by said output RNA in said cell, and
(c) determining that
   (i) said cell has a certain cell type and/or state when an effective amount of said output RNA and/or output protein is present in said cell, and/or
   (ii) said cell does not have said cell type and/or state when no effective amount of said output RNA and/or output protein is present in said cell.

Evidently, in context of the inventive in vitro methods provided herein, the eukaryotic cells, e.g. in a tissue sample, should be alive when the DNA construct, plasmid and/or virus of the invention is introduced into said cells, e.g. in said tissue sample, (i.e. in step a)). Furthermore, the cells should be alive long enough such that the DNA construct is functional, i.e. such that it is able to produce an effective amount of an output RNA in a condition under which it normally produces an effective amount of an output RNA (i.e., this may be a further step between steps a) and b)). However, for measuring the amount of an output RNA and/or output protein (i.e. in step b), the cells may be killed (e.g. fixed or lysed) or they may be kept alive (e.g. when the output protein is a reporter protein as described herein).

As regards an effective amount of the output RNA, the same applies as is described herein, e.g. in the context of the inventive DNA construct provided herein.

In particular, e.g. in context of the inventive in vitro methods provided herein, a certain cell type and/or state may comprise
(i) the presence of a certain transcriptional regulatory state, e.g. TS₁, for example, the presence of at least one or a certain number of transcription factor(s) (TFs) from a respective group of TFs, e.g., TF₁ₐ, or TF₁ₐ and TF_{1b}; and optionally
(ii) the absence of a respective post-transcriptional regulatory state, e.g. PTS₁, for example, the absence of at least one or a certain number of antisense RNA(s) (AR) from a respective group of ARs, e.g., AR₁, or AR₁ and AR₂.

As regards the cell types and/or states, the transcriptional regulatory states, and the post-transcriptional regulatory state, the same applies as is described herein, e.g. in the context of the inventive DNA construct provided herein.

Furthermore, the invention relates to an in vitro method for diagnosing a disease in a subject, wherein said method comprises
a) introducing the DNA construct, plasmid or virus of the invention into a tissue sample from said subject,
b) measuring the amount of an output RNA and/or an output protein encoded by the output RNA in said tissue sample, and
c) diagnosing whether said subject has said disease,
   wherein the diagnosis is positive when an effective amount of the output RNA and/or output protein is present in said tissue sample, and/or
   wherein the diagnosis is negative when no effective amount of the output RNA and/or output protein is present in said tissue sample.

As regards an effective amount of the output RNA, the same applies as is described herein, e.g. in the context of the inventive DNA construct provided herein.

In particular, said disease, e.g. in the context of the in vitro and/or in vivo diagnostic uses of the invention, may be associated with and/or caused by a heterogeneous mix of target cells (e.g. at least two different types of target cells). In particular, a target cell may correspond to a first abnormal and/or malignant cell type and/or state (AC₁), and/or another one or any of n further abnormal and/or malignant cell type(s) and/or state(s) (ACₙ), wherein n≥1 as described herein.

In particular, said tissue sample may be suspected to comprise said target cells. Furthermore, said tissue sample may comprise non-target cells, i.e. normal and/or benign cells.

In particular, diagnosing the disease (in vitro and/or in vivo) may comprise detecting target cells in said tissue sample regardless whether a target cell corresponds to said first abnormal and/or malignant cell type and/or cell state (AC₁), and/or to another one or any of said further abnormal and/or malignant cell types and/or cell states (ACₙ).

For example, at least 1%, 5%, 10%, 20%, 30%, 40%, 50%, 60%, 70%, 80%, 90%, 95%, 99% or 100% of the target cells that correspond to AC₁, and at least 1%, 5%, 10%, 20%, 30%, 40%, 50%, 60%, 70%, 80%, 90%, 95%, 99% or 100% of the target cells that correspond to another AC (ACₙ), e.g. AC₂, may be detected.

Preferably, at least 1%, 5%, 10%, 20%, 30%, 40%, 50%, 60%, 70%, 80%, 90%, 95%, 99% or 100%, e.g. about 1% to about 99%, about 10% to about 99% or about 10% to about 50% of the target cells that correspond to AC₁ and/or any of the ACₙ (e.g. AC₂ and/or AC₃) may detected.

Preferably, non-target cells, i.e. normal and/or benign cells, may not be detected.

For example, less than 30%, 20%, 10%, 5%, 2%, %, 0.5%, 0.1%, 0.05%, or 0.01%, preferably less than 10%, more preferably less than 1%, most preferably less than 0.1% of the non-target cells, i.e. normal and/or benign cells, are detected.

In particular, e.g. in the context of the in vitro and/or in vivo diagnostic uses of the invention, (i) the AC₁ may comprises the presence of said TS₁, and optionally the absence of said PTS₁, and/or (ii) an ACₙ, e.g. AC₂, may comprise the presence of a respective TSₙ, e.g. TS₂, and optionally the absence of a respective PTSₙ, e.g. PTP₂.

As regards the cell types and/or states, the transcriptional regulatory states, and the post-transcriptional regulatory state, the same applies as is described herein, e.g. in the context of the inventive DNA construct provided herein.

In particular, e.g. in the context of the in vitro and/or in vivo diagnostic uses of the invention, an effective amount of said output RNA and/or output protein may be obtained
(i) in target cells that correspond to said first abnormal and/or malignant cell type and/or state (AC₁), and/or
(ii) in target cells that correspond to any of said further abnormal and/or malignant cell type(s) and/or state(s) (ACₙ, e.g. AC₂).

Preferably, no effective amount of said output RNA and/or output protein may be obtained in non-target cells.

Furthermore, step b) of the inventive diagnostic method provided herein may further comprise measuring the percentage of cells in said tissue sample that have an effective amount of said output RNA and/or output protein, and/or the diagnosis in step c) may be considered positive when at least 0.01%, 0.05%, 0.1%, 0.5%, 1%, 5%, 10%, 20%, 30%, 40% or 50% of the cells in said tissue sample have an effective amount of said output RNA and/or output protein, and/or the diagnosis in step c) may be considered negative when less than 0.01%, 0.05%, 0.1%, 0.5%, 1%, 5%, 10%, 20%, 30%, 40% or 50% of the cells in said tissue sample have an effective amount of said output RNA and/or output protein.

Preferably, in the context of the diagnostic (in vitro and/or in vivo) uses of the inventive DNA construct provided herein, the output RNA may comprise a sequence encoding at least one reporter protein, e.g. a fluorescent protein or a luminogenic or chromogenic enzyme.

Thus, preferably, in the context of the diagnostic (in vitro and/or in vivo) uses and/or methods provided herein, the output protein may comprise at least one reporter protein, e.g. a fluorescent protein or a luminogenic or chromogenic enzyme.

Preferably, the reporter protein is suitable for determining the amount of an output RNA obtained and/or for detecting a target cell.

In particular, e.g. in the context of the in vitro and/or in vivo diagnostic uses of the invention, the disease may be a cancer, a neurodegenerative disease, an immunodeficiency, and/or a genetic disease, preferably a cancer.

As regards the cancer, the same applies as is described herein, e.g. in the context of the medical uses of the DNA construct of the invention. For example, said cancer may comprise cells that correspond to said first abnormal and/or malignant cell type and/or state (AC₁), and target cells that correspond to another one or any of said further abnormal and/or malignant cell type(s) and/or state(s) (ACₙ).

It is a further advantage of the DNA construct of the invention that it may be used for diagnosing a disease in a subject in vivo and/or in living cells (e.g. in an in vitro method of the invention). This allows to monitor the amount of the output RNA and/or protein in a subject, a tissue sample, and/or a single cell over time. Furthermore, this allows to monitor the number of target cells in a subject and/or tissue sample over time. For example, the amount of the output RNA and/or output proteins and/or the number of target cells in a subject may be monitored for several days, weeks, months or even years, e.g. by in vivo measurements (e.g. by optical methods), by analyzing body fluids (e.g. when the output protein is secreted into a body fluid), and/or by serial sampling of a tissue and subsequent in vitro measurements, e.g. as described herein. Thus, the DNA construct of the invention may be advantageous for many diagnostic applications.

Thus, the invention further relates to the DNA construct, plasmid, virus or host cell of the invention for use in diagnosing a disease in a subject in vivo, i.e. for use in an in vivo diagnostic method.

In particular, e.g. in the context of in vivo diagnostic uses and/or methods of the invention, the DNA construct, plasmid, and/or virus of the invention may be introduced into a plurality of cells in a subject (e.g. a tissue), wherein said plurality of cells may comprise target cells and non-target cells.

In particular, e.g. in the context of in vivo diagnostic uses and/or methods of the invention, the disease may be associated with and/or caused by a heterogeneous mix of target cells (e.g. at least two different types of target cells). In particular, a target cell may correspond to a first abnormal and/or malignant cell type and/or state (AC₁), and/or another one or any of n further abnormal and/or malignant cell type(s) and/or state(s) (ACₙ), wherein n≥1 as described herein.

Furthermore, e.g. in the context of in vivo diagnostic uses and/or methods of the invention, a sample of said plurality of cells (e.g. a tissue sample) may be obtained at one or more time point(s). Said cells and/or tissue sample(s) may be then analyzed in vitro, e.g. by the inventive in vitro methods provided herein, for example, by the inventive diagnostic in vitro methods provided herein.

For example, the in vivo diagnostic method may comprise:
a) introducing the DNA construct, plasmid or virus of the invention into a tissue of a subject,
b) measuring the amount of an output RNA and/or an output protein encoded by the output RNA in said tissue, and
c) diagnosing whether said subject has said disease,
   wherein the diagnosis is positive when an effective amount of the output RNA and/or output protein is present in said tissue, and/or
   wherein the diagnosis is negative when no effective amount of the output RNA and/or output protein is present in said tissue.

As regards the disease, the cancer, the target cells, the non-target cells, the abnormal and/or malignant cell type and/or state, an effective amount of the output RNA, the detection of target cells, the transcriptional regulatory states, the post-transcriptional regulatory states, the output protein, the reporter protein, and the diagnosis, the same applies, mutatis mutandis, as described herein, e.g. in the context of the inventive DNA construct of the invention and/or the inventive diagnostic methods provided herein.

For example, diagnosing the disease in vivo may comprise detecting target cells in said tissue regardless whether a target cell corresponds to said first abnormal and/or malignant cell type and/or cell state (AC₁), and/or to another one or any of said further abnormal and/or malignant cell types and/or cell states (ACₙ). Preferably, non-target cells, i.e. normal and/or benign cells, may not be detected.

For example, the diagnostic in vivo method may further comprise measuring the percentage of cells in said tissue that have an effective amount of said output RNA and/or output protein, and/or the diagnosis may be considered positive when at least 0.01%, 0.05%, 0.1%, 0.5%, 1%, 5%, 10%, 20%, 30%, 40% or 50% of the cells in said tissue have an effective amount of said output RNA and/or output protein, and/or the diagnosis may be considered negative when less than 0.01%, 0.05%, 0.1%, 0.5%, 1%, 5%, 10%, 20%, 30%, 40% or 50% of the cells in said tissue have an effective amount of said output RNA and/or output protein.

Thus, the DNA construct of the invention is particularly useful for many therapeutic and/or diagnostic applications. In particular, the DNA construct of the invention is particularly useful for detecting, killing and/or manipulating different types of eukaryotic target cells (e.g. a heterogenous population of target cells, such as, inter alia, different subtypes of a cancer), e.g. in a subject and/or in a tissue sample.

Furthermore, the invention relates to the following items:
1. A DNA construct comprising in 5′ to 3′ direction the following DNA sequence elements:
   a first promoter (P₁);
   n further promoter(s) (Pₙ), wherein n≥1; and
   an output sequence;
   wherein each of said promoters comprises a transcription start site and/or is suitable for initiating transcription;
   wherein initiation of transcription from P₁ is enabled by a first transcriptional regulatory state (TS₁) and initiation of transcription from each of said further promoter(s) (Pₙ) is enabled by a respective further transcriptional regulatory state (TSₙ) is enabled by TS₂; and
   wherein said DNA construct yields an effective amount of an output RNA in a eukaryotic cell, preferably a mammalian cell, more preferably a human cell, when said TS₁ and/or any of the respective TSₙ is present in said cell, wherein said output RNA comprises a sequence corresponding to said output sequence;
   in particular wherein a first type of said output RNA (RNA₁) is obtained when transcription is initiated from P₁, and a respective further type of said output RNA (RNAₙ) is obtained when transcription is initiated from a respective Pₙ, wherein each type of said output RNA comprises a sequence corresponding to said output sequence, and wherein the amount of said output RNA corresponds to the total amount of all types of said output RNA; and
   preferably wherein said DNA construct does not yield an effective amount of said output RNA when neither TS₁ nor any of TSₙ is present in said cell.
2. The DNA construct of item 1, further comprising in 5′ to 3′ direction between said first promoter (P₁) and the last promoter of said further promoter(s) (Pₙ) a first alternative first exon (E1ₐ) and a first alternative 5′ splice site (5′ss₁), and
   between said last promoter and said output sequence the last alternative first exon of n respective further alternative first exons (E1ₙ), the last alternative 5′ splice site of n respective further 5′ splice sites (5′ssₙ), a branch point (BP) and a 3′ splice site (3′ss), wherein said output sequence is a second Exon (E2), and
   preferably wherein said last alternative 5′ splice site is weaker than another one or any other 5′ splice site contained in the DNA construct.
3. The DNA construct of items 1 or 2, wherein said output RNA comprises at least one sequence encoding at least one output protein, wherein the coding sequence(s) of said at least one output protein is/are partially or fully contained in said output sequence, e.g. said second exon, preferably
   wherein said at least one output protein comprises a reporter protein, e.g., a fluorescent protein or a luminogenic or chromogenic enzyme, and/or an effector protein, e.g. a toxic protein, an enzyme, a cytokine, an immunomodulator, a membrane protein and/or a membrane-bound receptor.
4. The DNA construct of any one of items 1 to 3, wherein a certain transcriptional regulatory state
   (i) is associated with and/or reflects a certain cell type and/or cell state, e.g., a disease cell state, a differentiated cell state, or a stem cell state; and/or
   (ii) comprises the presence of at least one transcription factor (TF) from a respective group of transcription factors,
      in particular, wherein the first transcriptional regulatory state (TS₁) comprises the presence of at least two TFs from a first group of TFs, and/or wherein any of the further transcriptional regulatory state(s) (TSₙ) comprises the presence of at least two TFs from a respective further group of transcription factors,
      preferably, wherein P₁ comprises binding sites for at least two TFs from said first group of TFs, and/or wherein any of Pₙ comprises binding sites for at least two TFs from a respective further group of transcription factors.
5. The DNA construct of any one of items 3 to 6, wherein an output RNA comprising a sequence corresponding to the first alternative first exon (E1ₐ) is translationally inhibited and/or degraded by a first post-transcriptional regulatory state (PTS₁), and wherein an output RNA comprising a sequence corresponding to a further alternative first exon (E1ₙ) is translationally inhibited and/or degraded by a respective further post-transcriptional regulatory state (PTSₙ).
6. The DNA construct of item 5, wherein said DNA construct yields an effective amount of an output RNA and/or output protein encoded by an output RNA in said eukaryotic cell, when
   (i) at least one transcriptional regulatory state is present in said cell such that an output RNA from at least one respective promoter contained in said DNA construct is produced; and
   (ii) none of the respective post-transcriptional regulatory state(s) is present in said cell such that the respective produced output RNA comprising the respective alternative first exon is not translationally inhibited or degraded; and
   preferably wherein said DNA construct does not yield an effective amount of an output RNA and/or output protein encoded by an output RNA in said eukaryotic cell, when
   (i) no transcriptional regulatory state that is capable of inducing transcription from the respective promoter(s) contained in said DNA construct is present in said cell, and/or
   (ii) each post-transcriptional regulatory state that is capable of translationally inhibiting and/or degrading the respective output RNA produced by the respective promoters contained in said DNA construct is present in said cell.
7. The DNA construct of items 5 or 6, wherein a certain post-transcriptional regulatory state
   (i) is associated with and/or reflects a certain cell type and/or cell state;
   (ii) comprises the presence of at least one antisense RNA (AR) from a respective group of antisense RNAs,
      in particular wherein PTS₁ comprises the presence of at least one AR from a first group of ARs (AR₁), and/or a PTSₙ comprises the presence of at least one AR from a further group of ARs (ARₙ,),
      preferably wherein said E1ₐ comprises at least one sequence corresponding to at least one target site for at least one AR from AR₁, and/or wherein any of said E1ₙ comprises at least one sequence corresponding to at least one target site for at least one AR from a respective ARₙ;
   and/or
   (iii) comprises the presence of at least one RNA-binding protein,
8. The DNA construct of any one of items 1 to 7, wherein the yield of an effective amount of an output RNA in a cell corresponds to the presence of an at least 1.5-, 2-, 4-, 6-, 8-, 10-, 15-, 20-, 30-, 40-, 60-, 80-, 100-, 150-, or 200-fold, preferably at least 10-fold, higher amount of an output RNA and/or output protein that is encoded by an output RNA in a cell comprising said DNA construct compared to the amount of said output RNA and/or output protein present in a cell comprising the same DNA construct in a condition under which no effective amount of said output RNA is obtained, e.g. when none of the respective transcriptional regulatory states is present to initiate transcription from any of the promoters contained in said DNA construct.
9. A virus comprising the DNA construct of any one of items 1 to 8 or an RNA comprising a sequence corresponding to said DNA construct and/or a sequence that is complementary to the sequence of said DNA construct, wherein the DNA construct comprised in said virus is double-stranded or single-stranded, wherein the single-stranded DNA construct comprises a sequence that corresponds to the sense or antisense strand of the DNA construct of any one of the preceding items, in particular wherein said virus is an adeno-associated virus (AAV) vector, a lentiviral vector, an Adenoviral vector, a Herpes-Simplex Virus vector, or a VSV vector.
10. The DNA construct of any one of items 1 to 8 or the virus of item 9 for use in treating a disease, e.g. cancer, in a subject, preferably a mammal, more preferably a human.
11. The DNA construct or virus for use according to item 10, wherein
   (i) said DNA construct is to be introduced into a plurality of cells in a subject, wherein said plurality of cells may comprise target cells and non-target cells;
   (ii) said disease is associated with and/or caused by at least two different types of target cells, wherein a target cell may correspond to a first abnormal and/or malignant cell type and/or state (AC₁), and/or another one or any of n further abnormal and/or malignant cell type(s) and/or state(s) (ACₙ), wherein n≥1, in particular wherein a certain abnormal and/or malignant cell type and/or state comprises the presence of a certain transcriptional regulatory state and optionally the absence of a respective post-transcriptional regulatory state; and
   (iii) wherein treating said disease comprises killing and/or manipulating target cells regardless whether a target cell corresponds to said first abnormal and/or malignant cell type and/or state (AC₁), and/or to another one or any of said further abnormal and/or malignant cell types and/or states (ACₙ), preferably wherein non-target cells are not killed and/or manipulated; and
   in particular, wherein
   (iv) an effective amount of an output RNA and/or at least one output protein encoded by said output RNA is obtained
      in target cells that correspond to said first abnormal and/or malignant cell type and/or state (AC₁), and/or in target cells that correspond to another one or any of said further abnormal and/or malignant cell type(s) and/or state(s) (ACₙ), and preferably wherein no effective amount of the output RNA and/or output protein(s) encoded by said output RNA is obtained in the non-target cells.
12. An in vitro method for determining the cell type and/or state of a eukaryotic cell, preferably a mammalian cell, wherein said method comprises
   (a) introducing the DNA construct of any one of items 1 to 8 or the virus of item 9 into said eukaryotic cell in vitro,
   (b) measuring the amount of an output RNA and/or an output protein encoded by said output RNA in said cell, and
   (c) determining that
      (i) said cell has a certain cell type and/or state when an effective amount of said output RNA and/or output protein is present in said cell, and/or
      (ii) said cell does not have said cell type and/or state when no effective amount of said output RNA and/or output protein is present in said cell.
13. An in vitro method for diagnosing a disease, e.g. a cancer, in a subject, preferably a mammal, more preferably a human, wherein said method comprises
   a) introducing the DNA construct of any one of items 1 to 8 or the virus of item 9 into a tissue sample from said subject,
   b) measuring the amount of an output RNA and/or an output protein encoded by said output RNA in said tissue sample, and
   c) diagnosing whether said subject has said disease,
      wherein the diagnosis is positive when an effective amount of the output RNA and/or output protein is present in said tissue sample, and/or
      wherein the diagnosis is negative when no effective amount of the output RNA and/or output protein is present in said tissue sample.
14. The method of item 13, wherein said disease is
   (i) associated with and/or caused by a at least two different types of target cells, wherein a target cell may correspond to a first abnormal and/or malignant cell type and/or state (AC₁), and/or another one or any of n further abnormal and/or malignant cell type(s) and/or state(s) (ACₙ), wherein n≥1;
   (ii) wherein said tissue sample is suspected to comprise said target cells and may comprise non-target cells,
      in particular wherein a certain abnormal and/or malignant cell type and/or state comprises the presence of a certain transcriptional regulatory state and optionally the absence of a respective post-transcriptional regulatory state; and
   (iii) wherein diagnosing said disease comprises detecting target cells in said tissue sample regardless whether a target cell corresponds to said first abnormal and/or malignant cell type and/or cell state (AC₁), and/or to another one or any of said further abnormal and/or malignant cell types and/or cell states (ACₙ),
      preferably wherein non-target cells are not detected; and
   in particular wherein an effective amount of said output RNA and/or output protein is obtained in target cells that correspond to said first abnormal and/or malignant cell type and/or state (AC₁), and/or in target cells that correspond to another one or any of said further abnormal and/or malignant cell type(s) and/or state(s) (ACₙ), and
      preferably wherein no effective amount of said output RNA and/or output protein is obtained in non-target cells.
15. The DNA construct of any one of items 1 to 8 or the virus of item 9 for use in diagnosing a disease, e.g. cancer, in a subject, preferably a mammal, more preferably a human, in vivo.

The invention is also characterized by the following figures, figure legends and the following non-limiting examples.

### BRIEF DESCRIPTION OF THE FIGURES

FIG. 1. Schematics and initial characterization of multiple alternative promoter-based logic circuitry. (A) Exemplary layout of the multi-input programs enabled by the alternative promoter regulation. Alternative promoters P₁, P₂ and P₃ enable transcription of the respective alternative (first) exons: Ia, Ib and Ic, with a shared exon II. Each alternative (first) exon is followed by a 5′-splice signal (5′ ss1, 5′ ss2 or 5′ ss3). A branchpoint sequence (BP) and 3′-splice signal (3′ss), precede Exon II. Intronic sequence is represented by wavy boxes. Transcriptional (TP1, TP2, TP3) and post-transcriptional (PTP1, PTP2, PTP3) regulatory programs are represented by boxes that convert inputs to transcriptional and/or translational activity. “True” output corresponds to high transcriptional activity or high transcript concentration post-transcription. Alternative transcripts/isoforms are shown, with thick rectangles indicating exons and lines indicating introns. All transcripts result in same output protein. The logic formula corresponding to the general scheme is also shown. (B) The schematics of three-promoter polychromatic reporter. Various genetic components are labeled. Rectangles with jagged ends represent exons. Position of 5′-splice signals (5′ ss1, 5′ ss2 or 5′ ss3) and 3′-splice signal (3′ss) are shown. Dashed lines indicate alternative splicing. Wavy lines represent spliced transcripts. The output proteins translated from the spliced transcripts are shown. PIT: Pristinamycin I-dependent transactivator 2, PIR: pristinamycin I-repressible promoter, ET: Erythromycin dependent transactivator, ETR: erythromycin repressible promoter, tTA: tetracycline dependent transactivator, TRE: tetracycline repressible promoter, CMV: Human cytomegalovirus promoter. (C) Schematic and characterization of the initial three-promoter cassette (pKB01) based on mouse Ciita genetic sequences. On top, schematic highlights the 5′-splice sites used at each position with their compound score. The bar chart shows the expression of SBFP2, CFP, and mCitrine outputs in promoter-normalized units for different input combinations, indicated below. (D) Schematics and characterization of a modified cassette pJD31 showing qualitative agreement with OR logic. The genetic cassette schematic and chart notation are identical to panel C. mCerulean is used in this cassette instead of CFP. Each bar in (C) and (D) represents mean±SD of biological triplicates. Cassette features/modules and their DNA sequences are shown in FIG. 10A and Table 1 respectively.

FIG. 2. Exploration of the genetic landscape of the three-promoter polychromatic reporter system. (A) Schematics and characterization of the output response in a number of representative cassettes. Features that differ from pKB01 are indicated, including 5′-splice signals and their compound scores (Table 5), see FIG. 10A and Table 1 for details. The charts' notation is identical to FIG. 1C. Each bar represents mean±SD of biological triplicates of output expression in promoter-normalized units. (B) Micrographs illustrate the expression of three fluorescent outputs from pJD48 in HEK293 cells and induced with the indicated combinations of transactivators. SBFP2, mCerulean, mCitrine and transfection control mCherry are represented by pseudocolors (the SBFP2 pseudocolor is not well visible in greyscale mode). All micrographs are at 10× magnification. SBFP2: 500 ms exposure, LUT range of 0-32K; mCerulean: 500 ms, LUT range of 0-27K, mCitrine: 200 ms, LUT range of 0-14K; mCherry: 200 ms, LUT range of 0-32K. Scale bars, 100 μm. (C) Correlation between promoter-normalized units of SBFP2 (left), CFP/mCerulean (middle), and mCitrine (right) outputs with the compound score of 5′-splice signal at the third position for polychromatic cassettes. Outputs from single-input activation are used. Furthermore, linearly fitted trendlines are shown. (D) mRNA-seq data analysis (n=1) showing the relative abundance of reads (normalized) that map to exon-intron junctions and to post-splicing exon-exon junctions, shown for the constructs pKB01 and pJD49 under single-input conditions. Black arrows indicate major changes in junction frequency. X axis: junction type. ExNN-In indicates an unspliced sequence spanning an exon (NN=Ia, Ib, or Ic, FIG. 7B) and an adjacent intronic sequence; In-ExNN is an unspliced sequence spanning an intron and an adjacent exon; ExNN-ExII represents a properly-spliced junction between a first and the second exons. YFP stands for mCitrine, CFP for mCerulean and BFP for SBFP2. See the Examples, i.e. Example 1, for further details on data analysis workflow. Note that the frequency values should only be interpreted relative to each other as they do not reflect absolute isoform/junction abundance.

FIG. 3. Development of the two-promoter cassette. (A) Schematics of the two-promoter, two-color reporter. Notations are as in FIG. 1B. (B) Two-promoter cassette, pJD139. Top left, cassette schematics with 5′-splice site sequence, their compound score and intron length indicated. Bottom left, bar charts of output expression for different input combinations, indicated below, in promoter-normalized units. Right, visualization using micrographs. mCherry: 200 ms exposure, LUT range of 0-16K; SBFP2: 500 ms, LUT range of 0-18K; mCerulean: 500 ms, LUT range of 0-30K (the SBFP2 pseudocolor is not well visible in greyscale mode). (C) Two-input construct pJD145 with short introns. Top left, cassette schematics with 5′-splice signals their compound score and intron lengths indicated. Bottom left: output expression for different input combinations in promoter-normalized units. Right: visualization using micrographs. mCherry: 200 ms, LUT range of 0-30K; SBFP2 and mCerulean as in (B) (the SBFP2 pseudocolor is not well visible in greyscale mode). In (B) and (C), 10× magnification is used. Scale bars, 100 μm. Cassette details are in FIG. 10B and Table 1. Each bar represents mean±SD of biological triplicates.

FIG. 4. Multi-input OR logic circuits. (A) Schematics and characterization of stable and transient versions of an OR logic construct with two promoters. Top, construct schematics. Middle, transiently-transfected circuit data. Left to right: bar chart showing expression levels of mCitrine output in relative units obtained for different input combinations (shown below the chart); micrographs illustrating the expression of mCitrine output and transfection control in single cells; overlay of mCitrine expression histograms measured by flow cytometry. Bottom row, stably integrated circuit data. Left to right: bar chart plotting the mean expression of mCitrine positive cells on the left Y axis, and the percentage of mCitrine positive cells on the right Y axis for different input combinations (shown below the chart); micrographs illustrating mCitrine expression in single cells; overlay of mCitrine expression histograms measured by flow cytometry. Exposure time is 200 ms, LUT range is 0-16.5K for mCitrine. (B) Schematics and characterization of a three-input OR gate. The panel arrangement and visual items are similar to panel A. In the transiently-transfected circuit experiments, mCitrine exposure time is 200 ms, LUT range is 0-65K. For stably-integrated circuit, mCitrine exposure time is 200 ms and LUT range is 0-4.5K. All micrographs were taken at 10× magnification. Scale bars, 100 μm. Each bar represents mean±SD of biological triplicates.

FIG. 5. Disjunctive normal form-like (AND-OR) logic computation in mammalian cells. (A) Schematic representation of an AND-OR logic circuit implemented with four transcription factor inputs (TF-1 . . . TF-4). (B) A gene circuit implementing the concept in panel (A) and executing logic computation (PIT AND SOX10) OR (ET AND HFN1A) to control mCitrine output. Construct details are in FIG. 10D and Table 1. All the inputs are supplied in trans. Bidirectional TRE promoter controls SOX10 and HNF1A while CMV promoter drives PIT and ET. (C) AND-OR circuit performance. Top left: circuit schematic highlighting intron length and PIT binding sites. On bottom left, bar chart shows mCitrine expression levels in relative units, with each bar representing mean±SD of biological triplicates, for all input combinations numbered 1 to 16. Input presence is designated by the “+” sign and absence by the “−” sign; predicted outcome according to the logic formula is indicated by light grey (On) or dark grey (Off) bars. Micrographs on top right show the expression of mCitrine output and transfection control for selected input conditions. mCitrine and transfection control mCerulean are represented by pseudocolors. All micrographs were taken at 10× magnification. mCitrine: 200 ms exposure, LUT range of 1-5.5K; mCerulean: 500 ms exposure, LUT range of 0-21K. Scale bars, 100 μm. Bottom right, histogram of mCitrine expression levels for all input combinations highlighting worst-case and median On:Off ratios. X axis: mCitrine relative units, Y axis: number of input conditions/cases.

FIG. 6. Further disjunctive normal form (DNF)-like logic computation in mammalian cells. (A) Schematic representation of DNF logic circuit with TF (TF-1 . . . TF-4), and miRNA inputs. (B) Genetic circuit implementing logic computation (PIT AND SOX10 AND NOT(siRNA-FF4)) OR (ET AND HNF1A AND NOT(siRNA-FF5)) to control mCitrine output. Gray semi-ovals represent the AND gate implemented at the promoter level, and the blunt arrow represents repression by the siRNA. All the inputs are supplied in trans. Construct details are in FIG. 10E and Table 1 respectively. (C) DNF circuit performance. Left, the bar chart shows mCitrine expression levels in relative units for the indicated input combinations (numbered 1 to 16). The input presence and predicted outcome notation is identical to FIG. 5C. Each bar represents mean±SD of a biological triplicate. Right, micrographs illustrate the expression of mCitrine and transfection control for selected input conditions (numbered 5 to 16). mCitrine and transfection control mCerulean are represented by pseudocolors. All micrographs were taken at 10× magnification. mCitrine: 500 ms exposure, LUT range of 0-5K; mCerulean: 500 ms, LUT range of 0-4.65K. Scale bars, 100 μm.

FIG. 7. Schematic representation of sequences used in constructing the polychromatic reporter system (related to FIG. 1). (A) Amino acid sequence alignment of SBFP2, mCerulean and mCitrine fluorescent proteins. Exon1 (dashed rectangle) consisting of 210 amino acids, and Exon2 (solid rectangle) consisting of 29 amino acids, are depicted separately. (B) Initial polychromatic reporter construct (pKB01) aligned to the mouse Ciita gene sequence. Synthetic inducible promoter sequences (PIR, ETR, TRE) in conjunction with minimal TATA are driving expression of individual fluorescent proteins. Alternating first exons (Exons Ia, Ib, Ic) of Ciita gene are substituted with fluorescent protein Ex1 sequences while Exons2-19 of Ciita gene is substituted with second (shared) exon (Ex2) of fluorescent proteins. Entire 5′-UTR and partial intronic sequences are also obtained from Ciita gene. PIR: pristinamycin I-repressible promoter, ETR: erythromycin repressible promoter, TRE: tetracycline repressible promoter, TATA-YB: minimal promoter; * sign depicts exact homology between the genomic sequence and the synthetic construct.

FIG. 8. Control experiments, normalization controls and experimental data with a few three-input cassettes/constructs (related to FIG. 1). (A) Illustration of the need for functional splicing in context of the exemplified splicing-based constructs. On top, schematic shows promoter-reporter constructs (PIR-SBFP2, ETR-mCerulean, TRE-mCitrine) with either i) Exon1 and Exon2, or ii) Exon1 without Exon2, and without the 3′-splice signal in the intron. The bar charts below show the expression values obtained for these constructs with or without their transcriptional input. (B) Confirmation of the requirement for the second exon in context of the exemplified splicing-based constructs. On top, schematics of a control cassette/construct engineered with a mutated second exon (Myc tag sequence inserted), derived from pJD49. The bar chart below shows the negligible expression levels of fluorescent proteins upon induction with the respective inputs. (C) Cassettes/Constructs used for promoter normalization. Top, schematics showing promoter-reporter constructs (PIR-SBFP2, ETR-mCerulean, ETR-CFP, TRE-mCitrine) made for the purpose of promoter normalization. U1, U2, U4, U5, U6 are 5′-UTR codes whose sequences are mentioned in Table 1. The bar charts below show the expression values obtained for these constructs with their transcriptional input in relative units (see Example 1 for details). Each bar is labeled with a plasmid name used in a measurement. The font shade of grey of the 5′-UTR code matches to the bar shade of grey of the plasmid. 5′-UTR of mCitrine is identical across all the polychromatic reporter constructs. (D), (E) Characterization of a few key changes in three-promoter cassettes/constructs: the weakening of the third 5′-splice site (D) and the introduction of Kozak (E). Top, the schematic of constructs pKW17 and pJD23 (D) and pJD30 (E) with their 5′-splice sites and their compound score values (Table 5). Vertical line in the depicted sequences separates the exonic and intronic sequences of 5′-splice sites. ‘AAG’ sequence is used as a default exonic part of the 5′-splice site unless indicated. The bar charts below each schematic show expression values in promoter normalization units (Y axis) for different input combinations (shown below the chart) for each construct. Each set of bars represents the expression of alternative open reading frames encoding, respectively, SBFP2, mCerulean, and mCitrine outputs. Detailed description of calculation of the promoter normalized units and transfection details are provided in Example 1. Each bar in (A)-(D) represents mean±SD of biological triplicates. Construct features/modules and its related sequences can be found in FIG. 10 A,F,G and Table 1 respectively.

FIG. 9. Heatmaps displaying the expression profiles of the 3-promoter polychromatic reporter cassettes (related to FIGS. 1, 2). The mean promoter normalized units (n=3) of each fluorescent protein: SBFP2 (top), CFP/mCerulean (middle) and mCitrine (bottom) are depicted for all the polychromatic reporters (X axis) for all input combinations (Y axis). The scale bar for each fluorescent protein expression map is on its right side. Construct features/modules and its related sequences are in FIG. 10A and Table 1 respectively.

FIG. 10. Representation of modules (promoter, 5′-UTR, exon1a, 5′-splice site 1, intron, exon1b, 5′-splice site 2, exonic, 5′-splice site 3, 3′-splice site, exon2 and polyA) of all the plasmids (related to FIGS. 1-6, 8, 9, 11 and 12). Shown are all plasmids for (A) three-promoter polychromatic reporter cassettes/constructs, (B) two-promoter cassettes/constructs, (C) OR logic constructs, (D) AND-OR logic constructs, (E) AND-OR-NOT logic construct, (F) control constructs and (G) promoter normalization constructs. The sequences pertaining to the codes for each module are described in Table 1. Inclusion of kozak sequence in front of the exon is denoted by a star (*).

FIG. 11. OR logic circuit characterization. (Related to FIG. 4). (A) Alignment of pJD140 (2-color construct) with pJD145 (2-input OR logic construct) displaying the similarities and differences. Donor splice site and acceptor splice site sequence for both constructs are shown. PIR: pristinamycin I-repressible promoter, ETR: erythromycin repressible promoter, TRE: tetracycline repressible promoter, TATA-YB: minimal promoter. (B) Agarose gel showing integrity of the construct following stable integration in HEK293 cells. Gel lanes are divided in plasmid groups—pJD163 (1,2), pJD164 (3,4), pJ165 (5,6). Lanes 1,3,5 correspond to amplification of respective plasmid serving as positive control. Lanes 2,4,6 correspond to amplification of genomic DNA from stably transduced HEK293 with viral vectors derived from pJD163, pJD164 and pJD165 respectively. Lane 7 shows amplification of non-transduced HEK293 genomic DNA. Lane 8 is PCR control with no template. (C) Schematics of stable and transient version of the OR logic construct with two promoters. The same construct is integrated in a lentiviral transfer vector in an antisense strand, or in a regular plasmid for transient transfection. Construct features/modules and its related sequence can be found in FIG. 10C and Table 1 respectively. (D) Data related to transient circuit. Left to right: bar chart showing the expression levels of mCitrine in relative units obtained for different input combinations (shown below the chart). Micrographs illustrate expression of mCitrine output and transfection control in single cells. mCitrine exposure time is 200 ms and LUT range is 0-65K; overlay of histograms of mCitrine expression observed by flow cytometry. (E) Data obtained for stably-integrated circuit. Left to right: a bar chart plotting the mean expression of mCitrine positive cells on the left Y axis and percentage of mCitrine positive cells on the right Y axis for different input combinations (shown below the chart); micrographs illustrating expression of mCitrine output in single cells with exposure time of 200 ms and LUT range of 0-16.5K; and overlay of histograms of mCitrine expression observed by flow cytometry. Each bar represents mean±SD of biological triplicates. mCitrine and transfection control mCherry are represented by pseudocolors. All micrographs were taken at 10× magnification. Scale bars, 100 μm. Details of transfection are described in Example 1.

FIG. 12. AND-OR logic computation in mammalian cells (related to FIG. 5). Top left: Schematic representation of the circuit pJD178 (A) and pJD198 (B) highlighting the intron length between the two promoters. Bottom left: The bar chart shows mCitrine expression levels in relative units (each bar represents mean±SD of biological triplicates) obtained by flow cytometry for all possible input conditions (denoted 1-16). Input presence is designated by the “+” sign and absence by the “−” sign; predicted outcome according to the logic formula is indicated by light grey (On) or dark grey (Off) color bars. Micrographs on top right side illustrate the expression of mCitrine output and transfection control in single cells for selected input conditions. On the bottom right, histogram of mCitrine expression levels highlighting worst-case and median On:Off values is shown. All micrographs were taken at 10× magnification. mCitrine and transfection control mCerulean are represented by pseudocolors. mCitrine was imaged with exposure of 200 ms and shown with LUT range of 1-11K while mCerulean was imaged with 500 ms and shown with LUT range of 0-21K. Scale bars, 100 μm.

FIG. 13. Flow cytometry gating strategy. (related to Example 1). Representative flow cytometry dot-plots and histograms describing the gating strategy that was applied for the data analysis in the Examples, Figures and Figure legends herein. (A) The smoothened dot-plots showing the strategy for gating: live cells (left) based on Forward Scatter Area (FSC-A) vs Side Scatter Area (SSC-A), single cells (right) based on FSC-A vs FSC-W (width). Following this, compensation was performed manually based on the leakiness observed in single color control samples. (B) Histograms of singlets in non-transfected cells (compensated) was used for gating positive and negative population for a fluorescent channel: transfection control, mCherry (top left), SBFP2 (top right), mCerulean (bottom left), mCitrine (bottom right). This gating was then applied to all the samples in that experiment. Based on this gating, the percentage/frequency of positive cells for a fluorescent channel was obtained which was used for calculating relative and promoter normalized units. (C) Example dot-plots with adjunct histograms of pJD49 sample. mCherry is transfection control on X axis. Top left: non-induced condition, mCherry vs SSC-A. Top right: induced with PIT, mCherry vs SBFP2. Bottom left: induced with ET, mCherry vs mCerulean. Bottom right: induced with tTA, mCherry vs mCitrine.

### EXAMPLES

Methods and materials are described herein for use in the present disclosure; other, suitable methods and materials known in the art can also be used. The materials, methods, and examples are illustrative only and not intended to be limiting.

### Example 1: Materials and Methods

### Experimental Model and Subject Details

The experiments were performed in three different cell lines: HEK293 (Invitrogen, Cat #11631-017), HEK293T (ATCC, Cat #CRL-11268), and HeLa (ATCC, Cat #CCL-2, Lot #58930571). All cell lines, including stably transduced HEK293 cells, were cultured at 37° C., 5% CO₂, in 0.24 (TPP, Cat #99500) filtered DMEM (ThermoFisher, Cat #41966029) supplemented with 10% FBS (ThermoFisher, Cat #10270-106) and 1% Penicillin/Streptomycin solution (Corning, Cat #30-002CI). Splitting was performed every 3-4 days using 0.25% Trypsin-EDTA (ThermoFisher, Cat #25200-072). Mycoplasma tests were performed once every 4 weeks. For Mycoplasma detection, protocol from PCR Mycoplasma test kit (Promokine, Cat #PK-CA91-1024) was used with primers specific for contaminating mycoplasmas (Uphoff and Drexler, 2011). The thermocycler program used for detection was as follows: 1 cycle of 7 minutes at 95° C., 3 minutes at 72° C. and 2 minutes at 65° C.; 32 cycles of 4 seconds at 95° C., 8 seconds at 50° C. and 45 seconds at 68° C. Primers: PR1843, PR1844, PR1845, PR1846, PR1847 and PR1848 were used for detection reaction (see Table 2). For positive control, an extra set of primers (PR0673 and PR0674) apart from the ones mentioned above were used. Cell cultures were propagated for at most two months before being replaced by fresh cell stock.

### Method Details

### DNA Cassettes Design and Construction

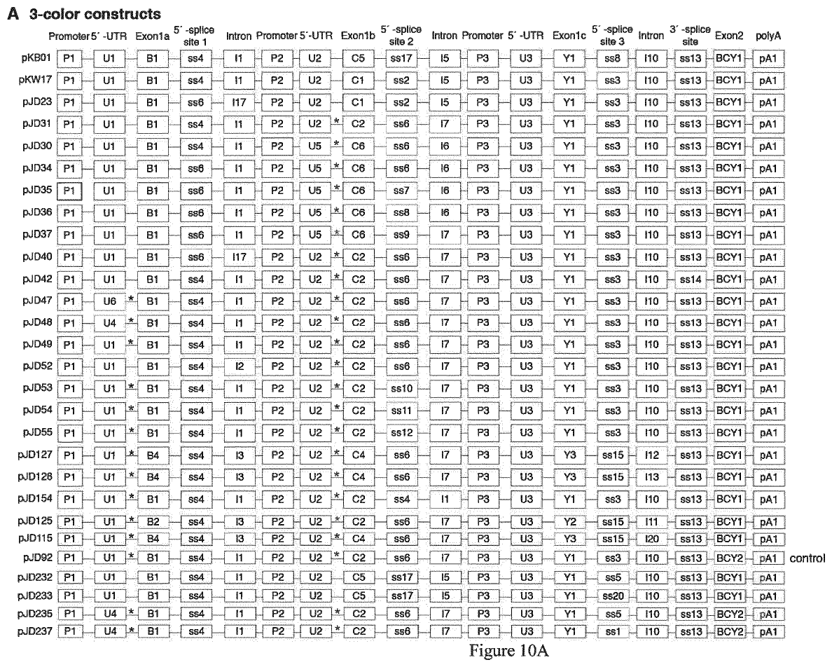

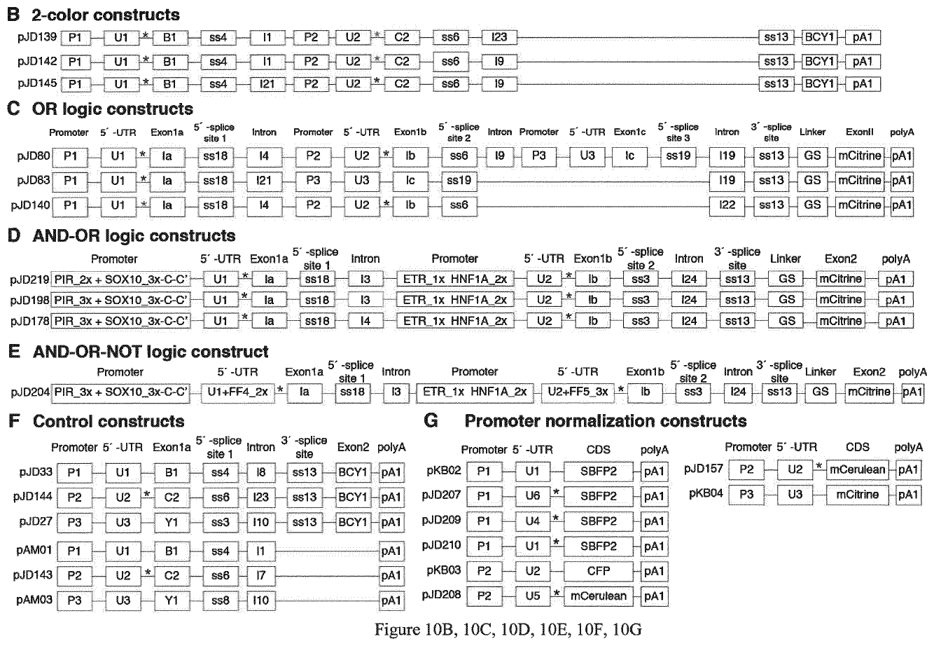

DNA cassettes are divided into several modules (FIG. 10). The sequences pertaining to each module are listed in Table 1. Primers used in cloning and other procedures are listed in Table 2 Gene fragments/synthetic DNA sequences/gBlocks used in cloning are listed in Table 3 All the plasmids and their related cloning procedure are listed in Table 4

### Polychromatic Reporter Constructs

GFP-derived fluorescent proteins (SBFP2, CFP, mCerulean and mCitrine) were used to construct polychromatic reporters. These fluorescent proteins were split into two exons (exon1—210 amino acids, exon2—29 amino acids) in such a way that the second exon remained identical. Pristinamycin I-dependent transactivator (PIT) 2/pristinamycin I-repressible promoter (PIR) system (Fussenegger et al., 2000) with three binding sites, Erythromycin dependent transactivator promoter system (Weber et al., 2002) with three binding sites, and Tetracycline dependent transactivator promoter system with 6 binding sites were placed in front of TATA-YB minimal sequence (Angelici et al., 2016) for driving expression of SBFP2, CFP/mCerulean and mCitrine fluorescent proteins respectively. 5′-UTR sequences were either obtained from mouse Ciita gene (NCBI #NC000082.6) or designed to prevent formation of secondary structures thereby allowing occupancy by different regulatory factors. Promoter and 5′-UTR sequences were placed upstream of the alternating exon sequences (SBFP2 Ex1, CFP/mCerulean Ex1 and mCitrine Ex1). Kozak sequence was inserted in front of the start codon in some cassettes. Intron sequences varying in length (50-516 bp) were obtained from mouse Ciita gene (NCBI #NC000082.6). 5′-splice site sequences were varied in different polychromatic constructs. 3′-splice site sequence used was either from mouse Ciita gene (NCBI #NC000082.6) or the following: 5′-ttttttaacttcctttattttccttacag-3′ (SEQ ID NO:1). Splicing enhancer sequences (Miyaso et al., 2003; Wang et al., 2012) were inserted within the intronic sequence downstream of SBFP2 exon 1 in pJD52 and pJD125. Rabbit globin polyadenylation signal placed downstream of stop codon was used for termination of transcription. The initial polychromatic cassette pKB01 was synthesized de novo by Genewiz.

### OR Logic Constructs

Promoters, 5′-UTRs, introns and transcription termination sequence were used as described in the section above. Promoter and 5′-UTR sequences were placed upstream of the alternating exon sequences. Alternative first exon sequences (Ia, Ib, and Ic) were obtained from mouse Ciita gene (NCBI #NC000082.6). The last codon of the alternating exons was split by placing the first base in the alternating part and the two bases in the shared second exon. This ensures that splicing has to happen for coding sequence to be in frame and hence the protein to be functional. Other than that, the second exon consisted of a linker (Shcherbakova et al., 2016) followed by mCitrine CDS. An intron splicing enhancer (5′-gttggtggtt-3′; SEQ ID NO:2) (Wang et al., 2012) that was inserted within 50 bp downstream of the first (most upstream) 5′ splice site was used to facilitate splicing of sequence between exon1 (Ia) and exon2 (linker with mCitrine) in all OR logic constructs.

### Normal Form-Like Logic Constructs

Alternative synergistic promoters were used to design AND-OR logic circuit. The architecture of the constructs remained similar to OR logic constructs with some changes in promoter architecture. Synergistic promoters were designed as described earlier (Angelici et al., 2016). The response elements (binding sites) for SOX10 transcription factor were placed in synergy with the response elements (binding sites) for PIT-VP16 transactivator while the response elements (binding sites) for HNF1A transcription factor were placed in synergy with the response elements (binding sites) for ET transactivator. Two or three response elements were used for PIT transactivator. One response element was used instead of three (in OR logic constructs) for ET transactivator. The sequence and placement of response elements of each transcription factor with respect to the transactivator is provided in Table 1. For NOT logic, 2× target site for siRNA-FF4 and 3× target site for siRNA-FF5 were cloned into 5′-UTRs of alternative first exons (Table 1).

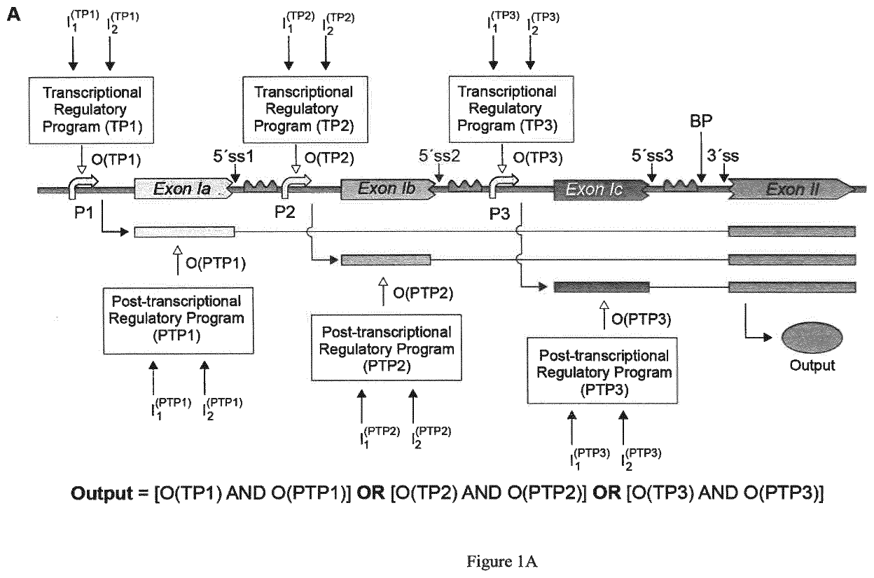

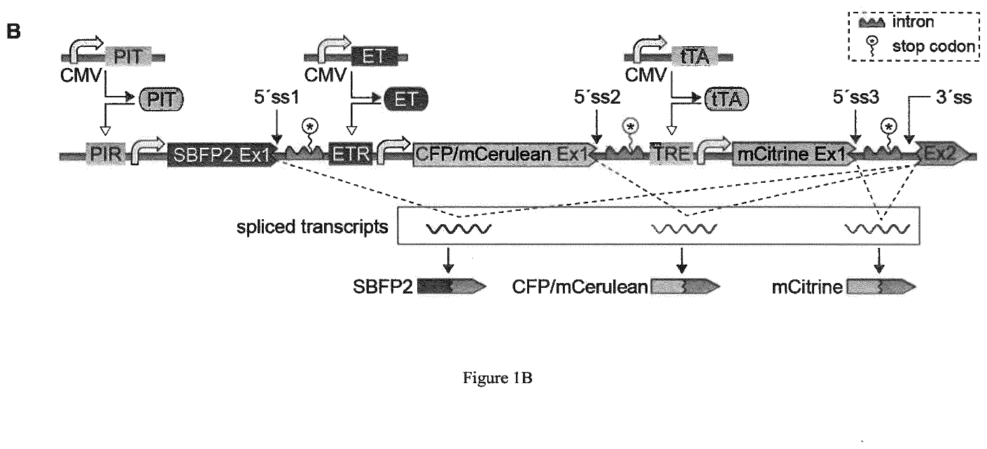

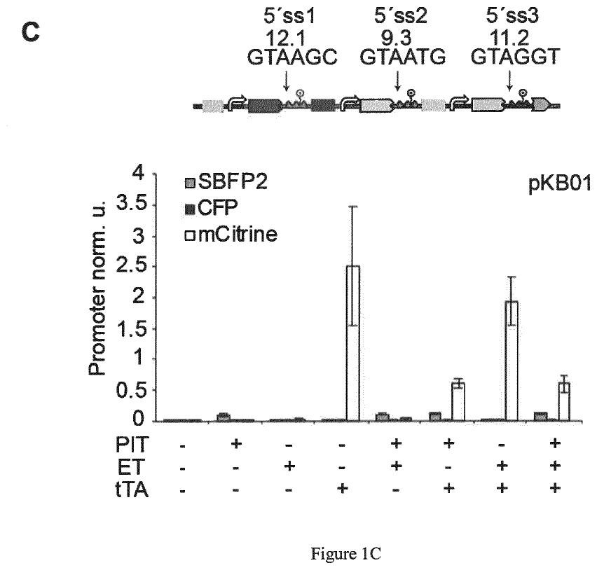

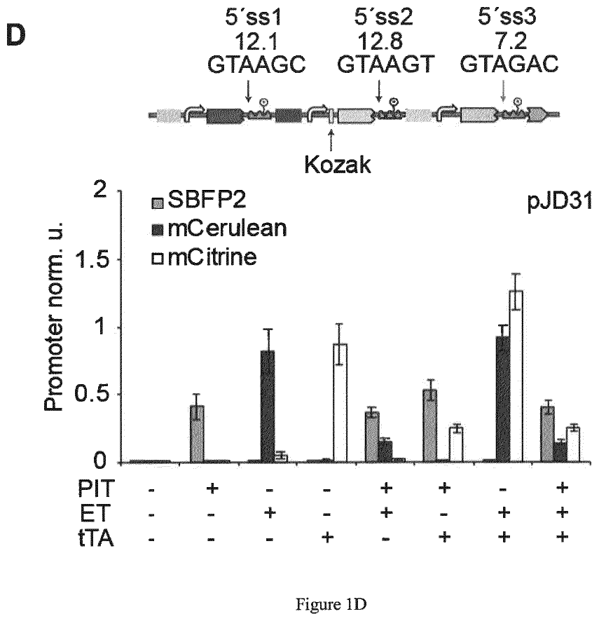

Table 1. DNA sequences of modules used to construct genetic logic circuits. Related to FIGS. 1-6, and 8-12. Described in the following are the DNA sequences of various structural modules of the logic gate constructs, sorted by the module type. See FIG. 10 for the schematics of the full constructs, shown as concatenations of the modules from Table 1. Note: i) In order to re-create the constructs, the exonic part of 5′-splice site sequence should not be concatenated because it is already included in the CDS exonic sequence. ii) GCC(G/A)CC nucleotides are added between 5′-UTR and exon1 CDS for making complete kozak sequence, iii) Also, 3′-splice site sequence is always part of the intronic sequence. Thus, the sequence should not be added separately in order to re-create the construct.

TABLE 1.1
Promoters

| Code | Description | Sequence (5′-3′) |
|---|---|---|
| P1 | PIR(3x) + TATA-YB | GAAATAGCGCTGTACAGCGTATGGGAATCT |
| | | CTTGTACGGTGTACGAGTATCTTCCCGTAC |
| | | ACCGTACAAGCTTACTAGAGGGTATATAAT |
| | | GGGGGCCAATCCT (SEQ ID NO: 3) |
| P2 | ETR(3x) + TATA-YB | GATTGAATATAACCGACGTGACTGTTACAT |
| | | TTAGGGATTGTATATAACCCACGTGAGTGT |
| | | TACAATTAGGGATTGAATATAACCGACGTG |
| | | ACTGTTACATTTAGGAAGCTTACTAGAGGG |
| | | TATATAATGGGGGCCA (SEQ ID NO: 4) |
| P3 | TRE(6x) + TATA-YB | TCCCTATCAGTGATAGAGAACGTATGTCGA |
| | | GTTTACTCCCTATCAGTGATAGAGAACGAT |
| | | GTCGAGTTTACTCCCTATCAGTGATAGAGA |
| | | ACGTATGTCGAGTTTACTCCCTATCAGTGA |
| | | TAGAGAACGTATGTCGAGTTTACTCCCTAT |
| | | CAGTGATAGAGAACGTATGTCGAGTTTATC |
| | | CCTATCAGTGATAGAGAACGTATGTCGAGT |
| | | TTACTCCCTATCAGTGATAGAGAAACGTTA |
| | | CTAGAGGGTATATAATGGGGGCCAGACTG |
| | | CCCGCCC (SEQ ID NO: 5) |
| PIR_3x + | PIR promoter (3 | GAAATAGCGCTGTACAGCGTATGGGAATCT |
| SOX10_3x-C- | binding sites) | CTTGTACGGTGTACGAGTATCTTCCCGTAC |
| C′ | alongside SOX10 | ACCGTACGAGCTCCTACACAAAGCCCTCTT |
| | binding sites and | TGTGAGACTACACAAAGCCCTCTTTGTGAG |
| | minimal TATA | ACTACACAAAGCCCTCTTTGTGAGAGGTAC |
| | sequence followed by a | CCTAGAGGGTATATAATGGGGGCCAATCCT |
| | restriction site | (SEQ ID NO: 6) |
| PIR_2x + | PIR promoter (2 | GAAATAGCGCTTAAAGTAAGCTGGGAATC |
| SOX10_3x-C- | binding sites with | TCTTGTACGGTGTACGAGTATCTTCCCGTA |
| C′ | distant mutated binding | CACCGTACGAGCTCCTACACAAAGCCCTCT |
| | site) alongside SOX10 | TTGTGAGACTACACAAAGCCCTCTTTGTGA |
| | binding sites and | GACTACACAAAGCCCTCTTTGTGAGAGGTA |
| | minimal TATA | CCCTAGAGGGTATATAATGGGGGCCAATCC |
| | sequence followed by a | TCT (SEQ ID NO: 7) |
| | restriction site | |
| ETR + | ETR promoter | GATTGAATATAACCGACGTGACTGTTACAT |
| HNF1A_2x | alongside HNF1A | TTAGGCAATTGAAGTTAATAATTAACTAGT |
| | binding sites and | TAATAATTAACTAAGCTTACTAGAGGGTAT |
| | minimal TATA | ATAATGGGGGCCA (SEQ ID NO: 8) |
| | sequence followed by a | |
| | restriction site | |

TABLE 1.2
5′ UTRs

| Code | Description | Sequence (5′-3′) |
|---|---|---|
| U1* | 5′UTR of Exon1a of | ACTTCATGTTTTGGATGCTGCAAGGCTGGATGA |
| | Mice Ciita gene; NCBI | GAGGCGACTCCAGGCAGCAGGCAGCCTCAGAGC |
| | id-NC000082.6, | ACTGCC (SEQ ID NO: 9) |
| | position: 10480072- | |
| | 10480143 | |
| U2 | 5′UTR of Exon1b of | GAATTTTCAGGTGGTCCCTTGCTCGCTTTCTTTG |
| | Mice Ciita gene; NCBI | CATGCTAGCTGAGCTTGGTAGGTTCTGGGCTCCT |
| | id-NC000082.6, | AACTGATAGACAGAGGCATGTGAGGGATGAGG |
| | position: 10488178- | CTGCCTGCTTCCCACCTGGGCATCTGAGGACCTT |
| | 10488372 | TTTGGAGACTTCCGGCACGCCAGGAGGGGCAGC |
| | | TGGACTACAGACGTTACTGCATCACTCTGCTCTC |
| | | TAAATC (SEQ ID NO: 10) |
| U3 | 5′UTR of Exon1c of | CAAGCTCCTAGGAGCCACGGAGCTGGCGGCAGG |
| | Mice Ciita gene; NCBI | GAGACTGC (SEQ ID NO: 11) |
| | id-NC000082.6, | |
| | position: 10489781- | |
| | 10489821 | |
| U4 | Synthetic 5′ UTR | CTGAATTCATCTTGGCTGAGGAATCTTCTAACAA |
| | | TTTAGAGCTTAAAAACGCCCACGAGGCGGAGAA |
| | | CGAAATATCCAGAGAGACGTTAGAAACGTTCAA |
| | | AAACGTTCACTAGTAGG (SEQ ID NO: 12) |
| U5 | Short 5′UTR of | GAATTTTCAGGTATCCTCTGGTCCCTTGCTCGCT |
| | Exon1b of Mice Ciita | TTCTTTGCATG (SEQ ID NO: 13) |
| | gene; NCBI id- | |
| | NC000082.6, | |
| | position: 10488178- | |
| | 10488203 | |
| U6 | short 5′UTR with | CTGAATTCTTATAAAGGCCTATTGTACTAGTAGG |
| | restriction sites | (SEQ ID NO: 14) |
| U1 + | target sites for siRNA- | ACTTCATGTTTTGGATGCTGCAAGGCTGGATGA |
| FF4_2x | FF4 added to Ul | GAGGCGACTCCAGGCAGCAGGCAGCCTCAGAGC |
| | | ACTGCCCCGCTTGAAGTCTTTAATTAAACCGCTT |
| | | GAAGTCTTTAATTAAAACCGGT (SEQ ID NO: 15) |
| U2 + | target sites for siRNA- | GGTCCCTTGCTCGCTTTCTTTGCATGCTAGCTGA |
| FF5_3x | FF5 added to U2 | GCTTGGTAGGTTCTGGGCTCCTAACTGATAGAC |
| | | AGAGGCATGTGAGGGATGAGGCTGCCTGCTTCC |
| | | CACCTGGGCATCTGAGGACCTTTTTGGAGACTTC |
| | | CGGCACGCCAGGAGGGGCAGCTGGACTACAGA |
| | | CGTTACTGCATCACTCTGCTCTCTAAATCAAGCA |
| | | CTCTGATTTGACAATTAAAGCACTCTGATTTGAC |
| | | AATTAAAGCACTCTGATTTGACAATTACTCGAG |
| | | (SEQ ID NO:16) |

*pJD49, pJD53, pJD54, pJD55, pJD92, pJD115, pJD125, pJD127, pJD128, pJD154, pJD235, pJD237, pJD139, pJD142, pJD145, pJD80, pJD83, pJD140, pJD178, pJD198, pJD219, pJD204 has extra bases (CTGAATTC) preceding and (ACTAGT) following the U1 5′UTR; pKB01, pKW17, pJD23, pJD31, pJD30, pJD34, pJD35, pJD36, pJD37, pJD40, pJD42, pJD52, pJD232, pJD233 has extra bases (GGGTCTC) preceding the U1 5′UTR.

TABLE 1.3
Exon1 CDS

| Code | Description | Sequence (5′-3′) |
|---|---|---|
| B1 | BFP exon1 | ATGGTGAGCAAGGGCGAGGAGCTGTTCACCGGCGTGGTG |
| | CDS | CCCATCCTGGTGGAGCTGGACGGCGACGTGAACGGCCAC |
| | | AAGTTCAGCGTGAGCGGCGAGGGCGAGGGCGACGCCAC |
| | | CTACGGCAAGCTGACCCTGAAGTTCATCTGCACCACCGG |
| | | CAAGCTGCCCGTGCCCTGGCCCACCCTGGTGACAACCCT |
| | | GAGCCACGGCGTGCAGTGCTTCGCCAGATACCCCGACCA |
| | | CATGAAGCAGCACGACTTCTTCAAGAGCGCCATGCCCGA |
| | | GGGCTACGTGCAGGAGAGAACCATCTTCTTCAAGGACGA |
| | | CGGCAACTACAAGACCAGAGCCGAGGTGAAGTTCGAGG |
| | | GCGACACCCTGGTGAACAGAATCGAGCTGAAGGGCATCG |
| | | ACTTCAAGGAGGACGGCAACATCCTGGGCCACAAGCTGG |
| | | AGTACAACTTCAACAGCCACAACGTGTACATCACCGCCG |
| | | ACAAGCAGAAGAACGGCATCAAGGCCAACTTCAAGATC |
| | | AGACACAACATCGAGGACGGCGGCGTGCAGCTGGCCGA |
| | | CCACTACCAGCAGAACACCCCCATCGGCGACGGCCCCGT |
| | | GCTGCTGCCCGACAACCACTACCTGAGCACCCAGAGCAA |
| | | GCTGAGCAAG (SEQ ID NO: 17) |
| B2 | BFP exon1 | ATGGTGAGCAAGGGCGAGGAGCTGTTCACCGGCGTGGTG |
| | CDS differing | CCCATCCTGGTGGAGCTGGACGGCGACGTGAACGGCCAC |
| | from B1 by | AAGTTCAGCGTGAGCGGCGAGGGCGAGGGCGACGCCAC |
| | two ′wobble′ | CTACGGCAAGCTGACCCTGAAGTTCATCTGCACCACCGG |
| | bases | CAAGCTGCCCGTGCCCTGGCCCACCCTGGTGACAACCCT |
| | | GAGCCACGGCGTGCAGTGCTTCGCCAGATACCCCGACCA |
| | | CATGAAGCAGCACGACTTCTTCAAGAGCGCCATGCCCGA |
| | | GGGCTACGTGCAGGAGAGAACCATCTTCTTCAAGGACGA |
| | | CGGCAACTACAAGACCAGAGCCGAGGTGAAGTTCGAGG |
| | | GCGACACCCTGGTGAACAGAATCGAGCTGAAGGGCATCG |
| | | ACTTCAAGGAGGACGGCAACATCCTGGGCCACAAGCTGG |
| | | AGTACAACTTCAACAGCCACAACGTGTACATCACCGCCG |
| | | ACAAGCAGAAGAACGGCATCAAGGCCAACTTCAAGATC |
| | | AGACACAACATCGAGGACGGCGGCGTGCAGCTGGCCGA |
| | | CCACTACCAGCAGAACACCCCCATCGGCGACGGCCCCGT |
| | | GCTGCTTCCGGACAACCACTACCTGAGCACCCAGAGCAA |
| | | GCTGAGCAAG (SEQ ID NO: 18) |
| B4 | BFP exon1 | ATGGTGAGCAAGGGCGAGGAGCTGTTCACCGGCGTGGTG |
| | CDS differing | CCCATCCTGGTGGAGCTGGACGGCGACGTGAACGGCCAC |
| | from B1 by | AAGTTCAGCGTGAGCGGCGAGGGCGAGGGCGACGCCAC |
| | several | CTACGGCAAGCTGACCCTGAAGTTCATCTGCACCACCGG |
| | ′wobble′ bases | CAAGCTGCCCGTGCCCTGGCCCACCCTGGTGACAACCCT |
| | | GAGCCACGGCGTGCAGTGCTTCGCCAGATACCCCGACCA |
| | | CATGAAGCAGCACGACTTCTTCAAGAGCGCCATGCCCGA |
| | | GGGCTACGTGCAGGAGAGAACCATCTTCTTCAAGGACGA |
| | | CGGCAACTACAAGACCAGAGCCGAGGTGAAGTTCGAGG |
| | | GCGACACCCTGGTGAACAGAATCGAGCTGAAGGGCATCG |
| | | ACTTCAAGGAGGACGGCAACATCCTGGGCCACAAGCTGG |
| | | AGTACAACTTCAACAGCCACAACGTGTACATCACCGCCG |
| | | ACAAGCAGAAGAACGGCATCAAGGCCAACTTCAAGATC |
| | | AGACACAACATCGAGGACGGCGGCGTGCAGCTGGCCGA |
| | | CCACTACCAGCAGAACACCCCCATCGGCGACGGCCCCGT |
| | | GCTGCTTCCGGACAATCATTATCTCAGTACCCAAAGCAA |
| | | ACTCAGCAAG (SEQ ID NO: 19) |
| C1 | mCerulean | ATGGTGAGCAAGGGCGAGGAGCTGTTCACCGGGGTGGTG |
| | exon1 CDS | CCCATCCTGGTCGAGCTGGACGGCGACGTAAACGGCCAC |
| | with non- | AAGTTCAGCGTGTCCGGCGAGGGCGAGGGCGATGCCACC |
| | consensus 5′ | TACGGCAAGCTGACCCTGAAGTTCATCTGCACCACCGGC |
| | splice site | AAGCTGCCCGTGCCCTGGCCCACCCTCGTGACCACCCTG |
| | sequence | ACCTGGGGCGTGCAGTGCTTCGCCCGCTACCCCGACCAC |
| | | ATGAAGCAGCACGACTTCTTCAAGTCCGCCATGCCCGAA |
| | | GGCTACGTCCAGGAGCGCACCATCTTCTTCAAGGACGAC |
| | | GGCAACTACAAGACCCGCGCCGAGGTGAAGTTCGAGGGC |
| | | GACACCCTGGTGAACCGCATCGAGCTGAAGGGCATCGAC |
| | | TTCAAGGAGGACGGCAACATCCTGGGGCACAAGCTGGAG |
| | | TACAACGCCATCAGCGACAACGTCTATATCACCGCCGAC |
| | | AAGCAGAAGAACGGCATCAAGGCCAACTTCAAGATCCGC |
| | | CACAACATCGAGGACGGCAGCGTGCAGCTCGCCGACCAC |
| | | TACCAGCAGAACACCCCCATCGGCGACGGCCCCGTGCTG |
| | | CTGCCCGACAACCACTACCTGAGCACCCAGTCCAAGCTG |
| | | AGCAAA (SEQ ID NO: 20) |
| C2 | mCerulean | ATGGTGAGCAAGGGCGAGGAGCTGTTCACCGGGGTGGTG |
| | exon1 CDS | CCCATCCTGGTCGAGCTGGACGGCGACGTAAACGGCCAC |
| | with a | AAGTTCAGCGTGTCCGGCGAGGGCGAGGGCGATGCCACC |
| | restriction | TACGGCAAGCTGACCCTGAAGTTCATCTGCACCACCGGC |
| | site | AAGCTGCCCGTGCCCTGGCCCACCCTCGTGACCACCCTG |
| | | ACCTGGGGCGTGCAGTGCTTCGCCCGCTACCCCGACCAC |
| | | ATGAAGCAGCACGACTTCTTCAAGTCCGCCATGCCCGAA |
| | | GGCTACGTCCAGGAGCGCACCATCTTCTTCAAGGACGAC |
| | | GGCAACTACAAGACCCGCGCCGAGGTGAAGTTCGAGGGC |
| | | GACACCCTGGTGAACCGCATCGAGCTGAAGGGCATCGAC |
| | | TTCAAGGAGGACGGCAACATCCTGGGGCACAAGCTGGAG |
| | | TACAACGCCATCAGCGACAACGTCTATATCACCGCCGAC |
| | | AAGCAGAAGAACGGCATCAAGGCCAACTTCAAGATCCGC |
| | | CACAACATCGAGGACGGCAGCGTGCAGCTCGCCGACCAC |
| | | TACCAGCAGAACACCCCAATTGGCGACGGCCCCGTGCTG |
| | | CTGCCCGACAACCACTACCTGAGCACCCAGTCCAAGCTT |
| | | TCGAAG (SEQ ID NO: 21) |
| C4 | mCerulean | ATGGTGAGCAAGGGCGAGGAGCTGTTCACCGGGGTGGTG |
| | exon1 CDS | CCCATCCTGGTCGAGCTGGACGGCGACGTAAACGGCCAC |
| | differing from | AAGTTCAGCGTGTCCGGCGAGGGCGAGGGCGATGCCACC |
| | C2 by several | TACGGCAAGCTGACCCTGAAGTTCATCTGCACCACCGGC |
| | ′wobble′ bases | AAGCTGCCCGTGCCCTGGCCCACCCTCGTGACCACCCTG |
| | | ACCTGGGGCGTGCAGTGCTTCGCCCGCTACCCCGACCAC |
| | | ATGAAGCAGCACGACTTCTTCAAGTCCGCCATGCCCGAA |
| | | GGCTACGTCCAGGAGCGCACCATCTTCTTCAAGGACGAC |
| | | GGCAACTACAAGACCCGCGCCGAGGTGAAGTTCGAGGGC |
| | | GACACCCTGGTGAACCGCATCGAGCTGAAGGGCATCGAC |
| | | TTCAAGGAGGACGGCAACATCCTGGGGCACAAGCTGGAG |
| | | TACAACGCCATCAGCGACAACGTCTATATCACCGCCGAC |
| | | AAGCAGAAGAACGGCATCAAGGCCAACTTCAAGATCCGC |
| | | CACAACATCGAGGACGGCAGCGTGCAGCTCGCCGACCAC |
| | | TACCAGCAGAACACCCCAATTGGAGATGGGCCTGTCCTC |
| | | CTACCTGATAATCACTATCTAAGTACGCAATCTAAACTTT |
| | | CGAAG (SEQ ID NO: 22) |
| C5 | CFP exon1 | ATGGTGTCCAAGGGTGAGGAACTGTTCACTGGGGTGGTG |
| | CDS | CCTATCCTTGTTGAACTCGACGGGGATGTGAATGGCCATC |
| | | GCTTCTCAGTTTCTGGCGAGGGGGAAGGCGACGCAACGT |
| | | ATGGCAAGCTTACACTGAAATTTATCTGTACTACCGGAA |
| | | AACTTCCCGTTCCCTGGCCTACGCTCGTCACTACTCTTAC |
| | | ATGGGGAGTACAGTGCTTCAGTAGGTACCCCGATCACAT |
| | | GAAACAACATGATTTCTTCAAATCTGCAATGCCTGAGGG |
| | | CTATGTACAGGAGAGAACAATATTTTTCAAGGATGACGG |
| | | AAACTATAAGACCAGGGCGGAGGTAAAGTTCGAGGGGG |
| | | ACACCCTGGTGAACCGAATAGAGCTGAAAGGGATAGACT |
| | | TCAAGGAAGATGGCAATATCCTTGGACACAAGTTGGAAT |
| | | ACAACTACATCAGCCACAACGTGTACATTACAGCAGATA |
| | | AGCAGAAAAACGGCATCAAAGCCCACTTCAAAATTAGAC |
| | | ACAATATCGAGGACGGGTCTGTTCAACTGGCCGACCACT |
| | | ATCAACAGAACACCCCAATTGGAGACGGACCTGTATTGC |
| | | TGCCCGACAATCATTATCTGAGCACTCAGTCTGCACTTTC |
| | | CAAG (SEQ ID NO: 23) |
| C6 | mCerulean | ATGGTGAGCAAGGGCGAGGAGCTGTTCACCGGGGTGGTG |
| | exon1 CDS | CCCATCCTGGTCGAGCTGGACGGCGACGTAAACGGCCAC |
| | with no | AAGTTCAGCGTGTCCGGCGAGGGCGAGGGCGATGCCACC |
| | restriction | TACGGCAAGCTGACCCTGAAGTTCATCTGCACCACCGGC |
| | site | AAGCTGCCCGTGCCCTGGCCCACCCTCGTGACCACCCTG |
| | | ACCTGGGGCGTGCAGTGCTTCGCCCGCTACCCCGACCAC |
| | | ATGAAGCAGCACGACTTCTTCAAGTCCGCCATGCCCGAA |
| | | GGCTACGTCCAGGAGCGCACCATCTTCTTCAAGGACGAC |
| | | GGCAACTACAAGACCCGCGCCGAGGTGAAGTTCGAGGGC |
| | | GACACCCTGGTGAACCGCATCGAGCTGAAGGGCATCGAC |
| | | TTCAAGGAGGACGGCAACATCCTGGGGCACAAGCTGGAG |
| | | TACAACGCCATCAGCGACAACGTCTATATCACCGCCGAC |
| | | AAGCAGAAGAACGGCATCAAGGCCAACTTCAAGATCCGC |
| | | CACAACATCGAGGACGGCAGCGTGCAGCTCGCCGACCAC |
| | | TACCAGCAGAACACCCCAATTGGCGACGGCCCCGTGCTG |
| | | CTGCCCGACAACCACTACCTGAGCACCCAGTCCAAGCTG |
| | | AGCAAG (SEQ ID NO: 24) |
| Y1 | mCitrine | ATGGTGAGCAAGGGCGAGGAGCTGTTCACCGGGGTGGTG |
| | exon1 CDS | CCCATCCTGGTCGAGCTGGACGGCGACGTAAACGGCCAC |
| | | AAGTTCAGCGTGTCCGGCGAGGGCGAGGGCGATGCCACC |
| | | TACGGCAAGCTGACCCTGAAGTTCATCTGCACCACCGGC |
| | | AAGCTGCCCGTGCCCTGGCCCACCCTCGTGACCACCTTCG |
| | | GCTACGGCCTGATGTGCTTCGCCCGCTACCCCGACCACAT |
| | | GAAGCAGCACGACTTCTTCAAGTCCGCCATGCCCGAAGG |
| | | CTACGTCCAGGAGCGCACCATCTTCTTCAAGGACGACGG |
| | | CAACTACAAGACCCGCGCCGAGGTGAAGTTCGAGGGCGA |
| | | CACCCTGGTGAACCGCATCGAGCTGAAGGGCATCGACTT |
| | | CAAGGAGGACGGCAACATCCTGGGGCACAAGCTGGAGT |
| | | ACAACTACAACAGCCACAACGTCTATATCATGGCCGACA |
| | | AGCAGAAGAACGGCATCAAGGTGAACTTCAAGATCCGCC |
| | | ACAACATCGAGGACGGCAGCGTGCAGCTCGCCGACCACT |
| | | ACCAGCAGAACACCCCCATCGGCGACGGCCCCGTGCTGC |
| | | TGCCCGACAACCACTACCTGAGCTACCAGTCCAAGCTGA |
| | | GCAAG (SEQ ID NO: 25) |
| Y2 | mCitrine | ATGGTGAGCAAGGGCGAGGAGCTGTTCACCGGGGTGGTG |
| | exon1 CDS | CCCATCCTGGTCGAGCTGGACGGCGACGTAAACGGCCAC |
| | differing from | AAGTTCAGCGTGTCCGGCGAGGGCGAGGGCGATGCCACC |
| | Y1 by two | TACGGCAAGCTGACCCTGAAGTTCATCTGCACCACCGGC |
| | ′wobble′ bases | AAGCTGCCCGTGCCCTGGCCCACCCTCGTGACCACCTTCG |
| | | GCTACGGCCTGATGTGCTTCGCCCGCTACCCCGACCACAT |
| | | GAAGCAGCACGACTTCTTCAAGTCCGCCATGCCCGAAGG |
| | | CTACGTCCAGGAGCGCACCATCTTCTTCAAGGACGACGG |
| | | CAACTACAAGACCCGCGCCGAGGTGAAGTTCGAGGGCGA |
| | | CACCCTGGTGAACCGCATCGAGCTGAAGGGCATCGACTT |
| | | CAAGGAGGACGGCAACATCCTGGGGCACAAGCTGGAGT |
| | | ACAACTACAACAGCCACAACGTCTATATCATGGCCGACA |
| | | AGCAGAAGAACGGCATCAAGGTGAACTTCAAGATCCGCC |
| | | ACAACATCGAGGACGGCAGCGTGCAGCTCGCCGACCACT |
| | | ACCAGCAGAACACCCCCATCGGCGACGGACCGGTGCTGC |
| | | TGCCCGACAACCACTACCTGAGCTACCAGTCCAAGCTGA |
| | | GCAAG (SEQ ID NO: 26) |
| Y3 | mCitrine | ATGGTGAGCAAGGGCGAGGAGCTGTTCACCGGGGTGGTG |
| | exon1 CDS | CCCATCCTGGTCGAGCTGGACGGCGACGTAAACGGCCAC |
| | differing from | AAGTTCAGCGTGTCCGGCGAGGGCGAGGGCGATGCCACT |
| | Y1 by three | TACGGCAAGCTGACCCTGAAGTTCATCTGCACCACCGGC |
| | ′wobble′ bases | AAGCTGCCCGTGCCCTGGCCCACCCTCGTGACCACCTTCG |
| | | GCTACGGCCTGATGTGCTTCGCCCGCTACCCCGACCACAT |
| | | GAAGCAGCACGACTTCTTCAAGTCCGCCATGCCCGAAGG |
| | | CTACGTCCAGGAGCGCACCATCTTCTTCAAGGACGACGG |
| | | CAACTACAAGACCCGCGCCGAGGTGAAGTTCGAGGGCGA |
| | | CACCCTGGTGAACCGCATCGAGCTGAAGGGCATCGACTT |
| | | CAAGGAGGACGGCAACATCCTGGGGCACAAGCTGGAGT |
| | | ACAACTACAACAGCCACAACGTCTATATCATGGCCGACA |
| | | AGCAGAAGAACGGCATCAAGGTGAACTTCAAGATCCGCC |
| | | ACAACATCGAGGACGGCAGCGTGCAGCTCGCCGACCACT |
| | | ACCAGCAGAACACCCCCATCGGCGACGGACCGGTGCTGC |
| | | TGCCCGACAACCACTACCTGAGCTACCAGTCCAAGCTGA |
| | | GCAAG (SEQ ID NO: 27) |
| Ia | Mice Ciita | ATGAACCACTTCCAGGCCATCCTGGCCCAAGTACAGACA |
| | gene exon1a; | CTGCTCTCCAGCCAGAAGCCCAGGCAGGTGCGGGCCCTC |
| | NCBI id- | CTGGATGGCCTGCTGGAAGAAGAGCTGCTCTCACGGGAA |
| | NC000082.6, | TACCACTGTGCCTTGCTGCATGAGCCTGATGGTGATGCCC |
| | position: | TGGCCCGGAAGATTTCCCTGACCCTGCTGGAGAAAGGGG |
| | 10480144- | ACTTAGACTTGACTTTCTTGAGCTGGGTCTGCAACAGTCT |
| | 10480426 | GCAGGCTCCCACGGTAGAGAGGGGCACCAGCTACAGGG |
| | | ACCATGGAG (SEQ ID NO: 28) |
| Ib | Mice Ciita | ATGCGCTGCCTGGTTCCTGGCCCTTCTGGGTCTTACCTGC |
| | gene exon1b; | CGGAGTTGCAAG (SEQ ID NO: 29) |
| | NCBI id- | |
| | NC000082.6, | |
| | position: | |
| | 10488373- | |
| | 10488424 | |
| Ic | Mice Ciita | ATGCAGGCAGCACTCAGAAGCACGGGGCACAGCCACAG |
| | gene exon1c; | CCGCG (SEQ ID NO: 30) |
| | NCBI id- | |
| | NC000082.6, | |
| | position: | |
| | 10489822- | |
| | 10489864 | |

TABLE 1.4
Splice sites

| Code | Description | Sequence (5′-3′)* |
|---|---|---|
| ss1 | 5′splice site | AAGGTCTCC |
| ss2 | 5′ splice site | AAAGTAATG |
| ss3 | 5′ splice site | AAGGTAGAC |
| ss4 | 5′ splice site (downstream of | AAGGTAAGC |
| | exonla of Ciita gene) | |
| ss5 | 5′splice site | AAGGTGATC |
| ss6 | 5′ splice site | AAGGTAAGT |
| ss7 | 5′ splice site | AAGGTAAGA |
| ss8 | 5′ splice site (downstream of | AAGGTAGGT |
| | exonlc of Ciita gene) | |
| ss9 | 5′ splice site | AAGGTAAGAATCTGC (SEQ ID NO: 39) |
| ss10 | 5′ splice site | AAGGTATGT |
| ss11 | 5′ splice site | AAGGTGGGT |
| ss12 | 5′ splice site | AAGGTTTGT |
| ss15 | 5′ splice site | AAGGTAGGC |
| ss17 | 5′ splice site (downstream of | AAGGTAATG |
| | exon1b of Ciita gene) | |
| ss18 | 5′splice site | GAGGTAAGC |
| ss19 | 5′splice site | GCGGTAGGC |
| ss20 | 5′splice site | AAGGTGCCC |
| ss13 | 3′ splice site preceding exon2 | ATCTGAGTGTATCTCTCCTCCCAGGA (SEQ |
| | of Ciita gene; NCBI id- | ID NO:4 8) |
| | NC000082.6, | |
| | position: 10501743-10501766 | |
| ss14 | 3′ splice site | TTTTTTAACTTCCTTTATTTTCCTTACAGGA |
| | | (SEQ ID NO: 49) |

*exonic sequence is underlined; intronic sequence is not underlined

TABLE 1.5
Introns

| Code | Description | Sequence (5′-3′) |
|---|---|---|
| I1* | Mice Ciita gene | TGGCATCCCTTTGAGTCAAGGCAACAATGTATGACC |
| | derived intron; | AACCCTAAGGTGTCCTTTCTCTGACAGGAAAATCTA |
| | 243 bp after exonla | TGTAAAGGATATAGGGTAAGCTTTTGGAAAACCTGG |
| | of Mice Ciita gene | GTGGCTGGGTGCTGCAAGCACAAAGCTTGTTTTCCA |
| | followed by | CATTGATCAGAGCATCCTCAGGGCACACTGCCGCCT |
| | restriction site | ACCTGCTTAGTCCATGCCTGAGCACCATCGACAATG |
| | | GACACCAGTGTACACTGTTCAGCGGCCGC (SEQ ID |
| | | NO: 50) |
| I2 | Mice Ciita gene | TGGCATCCCTTTGAGTCAAGGCAAGAATTCCAATGT |
| | derived intron-I1 | ATGACCAACGTGGAAAACAATGTTTTTGAACACCTA |
| | with an intron | AGGTGTCCTTTCTCTGACAGGAAAATCTATGTAAAG |
| | splicing enhancer1 | GATATAGGGTAAGCTTTTGGAAAACCTGGGTGGCTG |
| | (Miyaso et al, 2003) | GGTGCTGCAAGCACAAAGCTTGTTTTCCACATTGAT |
| | and a restriction site | CAGAGCATCCTCAGGGCACACTGCCGCCTACCTGCT |
| | | TAGTCCATGCCTGAGCACCATCGACAATGGACACCA |
| | | GTGTACACTGTTCAGCGGCCGC (SEQ ID NO: 51) |
| I3 | Mice Ciita gene | TGGCATCCCTTTGAGTCGTTGGTGGTTATCGATAAG |
| | derived intron-I1 | GCAACAATGTATGACCAACCCTAAGGTGTCCTTTCT |
| | with an intron | CTGACAGGAAAATCTATGTAAAGGATATAGGGTAA |
| | splicing enhancer2 | GCTTTTGGAAAACCTGGGTGGCTGGGTGCTGCAAGC |
| | (Wang et al, 2012) | ACAAAGCTTGTTTTCCACATTGATCAGAGCATCCTC |
| | and a restriction site | AGGGCACACTGCCGCCTACCTGCTTAGTCCATGCCT |
| | | GAGCACCATCGACAATGGACACCAGTGTACACTGTT |
| | | CAGCGGCCGC (SEQ ID NO: 52) |
| I17 | Mice Ciita gene | TGGCATCCCTTTGAGTCAAGAATTCAATGTATGACC |
| | derived intron | AACCCTAAGGTGTCCTTTCTCTGACAGGAAAATCTA |
| | differing from I1 by | TGTAAAGGATATAGGGTAAGCTTTTGGAAAACCTGG |
| | 5 bases | GTGGCTGGGTGCTGCAAGCACAAAGCTTGTTTTCCA |
| | | CATTGATCAGAGCATCCTCAGGGCACACTGCCGCCT |
| | | ACCTGCTTAGTCCATGCCTGAGCACCATCGACAATG |
| | | GACACCAGTGTACACTGTTCAGCGGCCGC (SEQ ID |
| | | NO: 53) |
| I8 | Mice Ciita gene | TGGCATCCCTTTGAGTCAAGGCAACAATGTATGACC |
| | derived intron-I1 + | AACCCTAAGGTGTCCTTTCTCTGACAGGAAAATCTA |
| | restriction site + | TGTAAAGGATATAGGGTAAGCTTTTGGAAAACCTGG |
| | terminal 200 bases | GTGGCTGGGTGCTGCAAGCACAAAGCTTGTTTTCCA |
| | of I10 | CATTGATCAGAGCATCCTCAGGGCACACTGCCGCCT |
| | | ACCTGCTTAGTCCATGCCTGAGCACCATCGACAATG |
| | | GACACCAGTGTACACTGTTCAGAGCTCAGAAAACG |
| | | GATACTTAAAGAGGATAAGTCACTCGCCTTGGGCCT |
| | | CACAGATGGGACTCCAACTCCTCTCTAGTGTCTGGA |
| | | ACCCTGTTGTTCATATTCTTAGGCTCCTCCCACCCCC |
| | | AGCCCTCAGCAGCAGTGCAAGTTCACTCTCTGCCTT |
| | | TGCCTACCATAATCCGATGACATATCTGAGTGTATC |
| | | TCTCCTCCCAG (SEQ ID NO: 54) |
| I4 | Shortened Mice | TGGCATCCCTTTGAGTCGTTGGTGGTTATCGATGTAC |
| | Ciita gene derived | ACTGTTCAGCGGCCGC (SEQ ID NO: 55) |
| | intron containing 5′ | |
| | splice site, NotI and | |
| | ClaI restriction sites | |
| | and intron splicing | |
| | enhancer2 (Wang et | |
| | al, 2012) | |
| I21 | Shorterned Mice | TGGCATCCCTTTGAGTCGTTGGTGGTTATCGATTGCG |
| | Ciita gene derived | GTGTATCTAATGCATGCGGCCGC (SEQ ID NO: 56) |
| | intron with 5′ splice | |
| | site, NsiI and ClaI | |
| | restriction sites and | |
| | intron splicing | |
| | enhancer2 (Wang et | |
| | al, 2012) | |
| I5 | Mice Ciita gene | GATGGGCTAGAGCCAATGGTTAAGACTAGAGCTTTC |
| | derived intron; | CTATGCACACTGAGGTCTTGGGTGGTTTCAGCATTC |
| | 353 bp after exon1b | CTAACAGAGTTTTTAATCTGCACCATGACTTCCAGG |
| | of Mice Ciita gene | GGCCTCTGCTGGCAGCTCTCAGCCCCACCAAGGTTT |
| | followed by | TGGGCTACTTAGACTATCATCTCTTCCTCTTCCTTCT |
| | restriction site | CCTCTTCCTTAAGGAGGAACCCTGTGGATGATTGAC |
| | | AAGCTCTCTACCCTTGAGCGACATCTCAAGCCTATT |
| | | TCTGTCTTTGAGTACACTGTGCTGTACTTCTAGGAGA |
| | | CCTCCATCCTGAGAAGCTTGGCACTCCCAGACTATA |
| | | AGCAAGCCTGCGGTGTATCTAATGCAT (SEQ ID |
| | | NO: 57) |
| I6 | Mice Ciita gene | GATGGGCTAGAGCCAATGGTGAAGACTAGAGCTTTC |
| | derived intron | CTATGCACACTGAGGTCTTGGGTGGTTTCAGCATTC |
| | differing from 15 by | CTAACAGAGTTTTTAATCTGCACCATGACTTCCAGG |
| | 3 bases | GGCCTCTGCTGGCAGCTCTCAGCCCCACCAAGGTTT |
| | | TGGGCTACTTAGACTATCATCTCTTCCTCTTCCTTCT |
| | | CCTCTTCCTTAAGGAGGAACCCTGTGGATGATTGAC |
| | | AAGCTCTCTACCCTTGAGCGACATCTCAAGCCTATT |
| | | TCTGTCTTTGAGTACACTGTGCTGTACTTCTAGGAGA |
| | | CCTCCATCCTGAGAAGCTTGGCACTCCCAGACTATA |
| | | AGCAAGCCTGCGGTGTATCTAATGCAT (SEQ ID |
| | | NO: 58) |
| I7** | Mice Ciita gene | ATCTGCTAGAGCCAATGGTGAAGACTAGAGCTTTCC |
| | derived intron | TATGCACACTGAGGTCTTGGGTGGTTTCAGCATTCC |
| | differing from 15 by | TAACAGAGTTTTTAATCTGCACAATGACTTCCAGGG |
| | 7 bases | GCCTCTGCTGGCAGCTCTCAGCCCCACCAAGGTTTT |
| | | GGGCTACTTAGACTATCATCTCTTCCTCTTCCTTCTC |
| | | CTCTTCCTTAAGGAGGAACCCTGTGGATGATTGACA |
| | | AGCTCTCTACCCTTGAGCGACATCTCAAGCCTATTTC |
| | | TGTCTTTGAGTACACTGTGCTGTACTTCTAGGAGAC |
| | | CTCCATCCTGAGAAGCTTGGCACTCCCAGACTATAA |
| | | GCAAGCCTGCGGTGTATCTAATGCAT (SEQ ID NO: 59) |
| I23 | Mice Ciita gene | ATCTGCTAGAGCCAATGGTGAAGACTAGAGCTTTCC |
| | derived intron | TATGCACACTGAGGTCTTGGGTGGTTTCAGCATTCC |
| | differing from 15 by | TAACAGAGTTTTTAATCTGCACAATGACTTCCAGGG |
| | 13 bases and an | GCCTCTGCTGGCAGCTCTCAGCCCCACCAAGGTTTT |
| | additional 230 bp | GGGCTACTTAGACTATCATCTCTTCCTCTTCCTTCTC |
| | containing 3′ splice | CTCTTCCTTAAGGAGGAACCCTGTGGATGATTGACA |
| | site obtained from | AGCTCTCTACCCTTGAGCGACATCTCAAGCCTATTTC |
| | sequence preceding | TGTCTTTGAGTACACTGTGCTGTACTTCTAGGAGAC |
| | Exon2 of Mice Ciita | CTCCATCCTGAGAAGCTTGGCACTCCCAGACTATAA |
| | gene | GCAAGCCTGCGGTGTATCTAATGCATTGGCACAGAG |
| | | ACAGCATTTTATTTGACTCTGAGCTCAGAAAACGGA |
| | | TACTTAAAGAGGATAAGTCACTCGCCTTGGGCCTCA |
| | | CAGATGGGACTCCAACTCCTCTCTAGTGTCTGGAAC |
| | | CCTGTTGTTCATATTCTTAGGCTCCTCCCACCCCCAG |
| | | CCCTCAGCAGCAGTGCAAGTTCACTCTCTGCCTTTG |
| | | CCTACCATAATCCGATGACATATCTGAGTGTATCTC |
| | | TCCTCCCAG (SEQ ID NO: 60) |
| I9 | Shortened Mice | ATCTGCTAGAGCCAATGGTGAAGACTAGAGCTTTCC |
| | Ciita gene derived | TGCGGTGTATCTAATGCAT (SEQ ID NO: 61) |
| | intron with 5′ splice | |
| | site, BbsI and NsiI | |
| | restriction sites | |
| I10 | Mice Ciita gene | GTCTCCAAGATCCCCTTTGCAGCCGCTAAAGTTAGG |
| | derived intron; | TTGGGAATCCGAGGCTCTGAGGCTGGCAACTATTTG |
| | 516 bp after exon1c | CCCGAAGTCAGACAGCAGGGCCGGAAAAAGCTGGA |
| | of Mice Ciita gene | TGGGTGTGCAGGAGGTTGAGAGGCTTCTTGGATCTT |
| | followed by | GGACGGACTGTATGCAGGACCTGGGAGCAGGGAGC |
| | restriction site and | TGGGGTCCAGAACACCGAGACTATGCACAGAACTG |
| | then 200 bp | GTAGTGAAGGCAAGTGGCGGGATAGCCTTGCAGGA |
| | preceding Exon2 of | GTGTCTGCAGCTCTGAGACTCAGACCGTGGGAGGCG |
| | Mice Ciita gene | CGGGGAGGCGGGGGAGGAGGGGGAGGAGGTGGGG |
| | | GGTGGGCAGACGTGCACTCTATTAATACCCAACATC |
| | | TTCAGAGAGCGCAGGGACCTTGGACCAGGGGCTGC |
| | | CCTTCACTGGACCTTCATTTGCGTGTTGGGACAGAC |
| | | CACCTCTGACTCTGGAGTCTGGGCACAGGGTCCTTC |
| | | TTGTGAGGGCTTGGCACAGAGACAGCATAGGGTGC |
| | | ATTTATTTGACTCTGAGCTCAGAAAACGGATACTTA |
| | | AAGAGGATAAGTCACTCGCCTTGGGCCTCACAGATG |
| | | GGACTCCAACTCCTCTCTAGTGTCTGGAACCCTGTT |
| | | GTTCATATTCTTAGGCTCCTCCCACCCCCAGCCCTCA |
| | | GCAGCAGTGCAAGTTCACTCTCTGCCTTTGCCTACC |
| | | ATAATCCGATGACATATCTGAGTGTATCTCTCCTCCC |
| | | AG (SEQ ID NO: 62) |
| I11 | Mice Ciita gene | GTCTCCAAGATCCCCTTTGGCGCCGCTAAAGTTAGG |
| | derived intron | TTGGGAATCCGAGGCTCTGAGGCTGGCAACTATTTG |
| | differing from I10 | CCCGAAGTCAGACAGCAGGGCCGGAAAAAGCTGGA |
| | by 3 bases | TGGGTGTGCAGGAGGTTGAGAGGCTTCTTGGATCTT |
| | | GGACGGACTGTATGCAGGACCTGGGAGCAGGGAGC |
| | | TGGGGTCCAGAACACCGAGACTATGCACAGAACTG |
| | | GTAGTGAAGGCAAGTGGCGGGATAGCCTTGCAGGA |
| | | GTGTCTGCAGCTCTGAGACTCAGACCGTGGGAGGCG |
| | | CGGGGAGGCGGGGGAGGAGGGGGAGGAGGTGGGG |
| | | GGTGGGCAGACGTGCACTCTATTAATACCCAACATC |
| | | TTCAGAGAGCGCAGGGACCTTGGACCAGGGGCTGC |
| | | CCTTCACTGGACCTTCATTTGCGTGTTGGGACAGAC |
| | | CACCTCTGACTCTGGAGTCTGGGCACAGGGTCCTTC |
| | | TTGTGAGGGCTTGGCACAGAGACAGCATAGGGTGC |
| | | ATTTATTTGACTCTGAGCTCAGAAAACGGATACTTA |
| | | AAGAGGATAAGTCACTCGCCTTGGGCCTCACAGATG |
| | | GGACTCCAACTCCTCTCTAGTGTCTGGAACCCTGTT |
| | | GTTCATATTCTTAGGCTCCTCCCACCCCCAGCCCTCA |
| | | GCAGCAGTGCAAGTTCACTCTCTGCCTTTGCCTACC |
| | | ATAATCCGATGACATATCTGAGTGTATCTCTCCTCCC |
| | | AG (SEQ ID NO: 63) |
| I20 | Mice Ciita gene | GTCTCCAAGATCCCCTTTGGCGCCGCTAAAGTTAGG |
| | derived intron | TTGGGAATCCGAGGCTCTGAGGCTGGCAACTATTTG |
| | differing from I10 | CCCGAAGTCAGACAGCAGGGCCGGAAAAAGCTGGA |
| | by 3 bases and a | TGGGTGTGCAGGAGGTTGAGGGCTTCTTGGATCTTG |
| | base less | GACGGACTGTATGCAGGACCTGGGAGCAGGGAGCT |
| | | GGGGTCCAGAACACCGAGACTATGCACAGAACTGG |
| | | TAGTGAAGGCAAGTGGCGGGATAGCCTTGCAGGAG |
| | | TGTCTGCAGCTCTGAGACTCAGACCGTGGGAGGCGC |
| | | GGGGAGGCGGGGGAGGAGGGGGAGGAGGTGGGGG |
| | | GTGGGCAGACGTGCACTCTATTAATACCCAACATCT |
| | | TCAGAGAGCGCAGGGACCTTGGACCAGGGGCTGCC |
| | | CTTCACTGGACCTTCATTTGCGTGTTGGGACAGACC |
| | | ACCTCTGACTCTGGAGTCTGGGCACAGGGTCCTTCT |
| | | TGTGAGGGCTTGGCACAGAGACAGCATAGGGTGCA |
| | | TTTATTTGACTCTGAGCTCAGAAAACGGATACTTAA |
| | | AGAGGATAAGTCACTCGCCTTGGGCCTCACAGATGG |
| | | GACTCCAACTCCTCTCTAGTGTCTGGAACCCTGTTGT |
| | | TCATATTCTTAGGCTCCTCCCACCCCCAGCCCTCAGC |
| | | AGCAGTGCAAGTTCACTCTCTGCCTTTGCCTACCAT |
| | | AATCCGATGACATATCTGAGTGTATCTCTCCTCCCA |
| | | G (SEQ ID NO: 64) |
| I12 | Shortened Mice | GTCTCCAAGATCCCCTTTGGCGCCGCTAAAGTTAGG |
| | Ciita gene derived | TTGGGAATATATCTGAGTGTATCTCTCCTCCCAG |
| | intron-50bases | (SEQ ID NO: 65) |
| | after exon1c of | |
| | Mice Ciita gene | |
| | with 2 base changes | |
| | for restriction site | |
| | and then 26 bp | |
| | preceding Exon2 of | |
| | Mice Ciita gene | |
| I13 | Shortened Mice | GTCTCCAAGATCCCCTTTGGCGCCGCTAAAGTTAGG |
| | Ciita gene derived | TTGGGAATGCCTACCATAATCCGATGACATATCTGA |
| | intron-I12 | GTGTATCTCTCCTCCCAG (SEQ ID NO: 66) |
| | sequence with an | |
| | additional 20 bases | |
| | coming from the | |
| | native intron | |
| | sequence | |
| I19 | Shorterned Mice | GTCTCCAAGATCCCCTTTGGCGCCCATAATCCGATG |
| | Ciita gene derived | ACATATCTGAGTGTATCTCTCCTCCCAG (SEQ ID |
| | intron with 5′ and | NO: 67) |
| | 3′ splice sites and | |
| | KasI and BamHI | |
| | (partial) restriction | |
| | sites | |
| I24 | Shorterned Mice | GTCTCCAAGATCCCCTTTGGCGCCCATAATCCGATG |
| | Ciita gene derived | ACATATCTGAGTGTATCTCTCCTCCCAG (SEQ ID |
| | intron differing | NO: 68) |
| | from I19 by 1 base | |
| | in 5′ splice site | |
| I22 | Shorterned Mice | ATCTGCTAGAGCCAATGGTGAAGACTAGAGCTTTCC |
| | Ciita gene derived | TGCGGTGTATCTAATGCATCATAATCCGATGACATA |
| | intron with 5′ and | TCTGAGTGTATCTCTCCTCCCAG (SEQ ID NO: 69) |
| | 3′ splice sites, BbsI, | |
| | NsiI and BamHI | |
| | restriction sites | |

*I1 sequence without NotI restriction site (GCGGCCGC) is part of intronic sequence of pAM01 and pJD154 (sequence following mCerulean exon1 CDS);
**pJD143 has the entire I7 sequence except for partial NsiI restriction site recognition sequence (TGCAT)

TABLE 1.6
Exon2 CDS

| Code | Description | Sequence (5′-3′) |
|---|---|---|
| BCY1 | Exon2 sequence | GATCCCAACGAGAAGAGAGATCA |
| | common among | CATGGTGCTTCTTGAGTTCGTGACCGCAGCCGG |
| | fluorescent proteins | AATTACCCTGGGTATGGACGAGTTGTACAAGTAG |
| | | (SEQ ID NO: 70) |
| BCY2 | Exon2 sequence | GATCCCAACGAGAAGAGAGATC |
| | disrupted using | ACGAGCAGAAACTCATCTCAGAAGAGG |
| | Myc tag* | ATCTGGAGTTGTACAAGTAG (SEQ ID NO: 71) |

*Myc tag is in italics and underlined

TABLE 1.7
Others

| Code | Description | Sequence (5′-3′) |
|---|---|---|
| GS | 1x GS linker (Shcherbakova et al, 2016) | GATCCGGTGGAGGAGGT |
| | | (SEQ ID NO: 72) |
| mCitrine | mCitrine fluorescent protein | SEQ ID NO: 73 |
| pA1 | rabbit globin polyadenylation signal sequence | SEQ ID NO: 74 |

TABLE 2
List of primers and siRNAs

| | SEQ ID NO |
|---|---|
| Primer id | |
| PR4317 | SEQ ID NO: 75 |
| PR4356 | SEQ ID NO: 76 |
| PR4330 | SEQ ID NO: 77 |
| PR4364 | SEQ ID NO: 78 |
| PR4325 | SEQ ID NO: 79 |
| PR4326 | SEQ ID NO: 80 |
| PR5070 | SEQ ID NO: 81 |
| PR5071 | SEQ ID NO: 82 |
| prKB0001 | SEQ ID NO: 83 |
| prKB0002 | SEQ ID NO: 84 |
| prKB0003 | SEQ ID NO: 85 |
| prKB0004 | SEQ ID NO: 86 |
| PR7360 | SEQ ID NO: 87 |
| PR7361 | SEQ ID NO: 88 |
| PR7358 | SEQ ID NO: 89 |
| PR7359 | SEQ ID NO: 90 |
| PR7362 | SEQ ID NO: 91 |
| PR7363 | SEQ ID NO: 92 |
| PR7364 | SEQ ID NO: 93 |
| PR7365 | SEQ ID NO: 94 |
| PR6812 | SEQ ID NO: 95 |
| PR6813 | SEQ ID NO: 97 |
| PR4310 | SEQ ID NO: 98 |
| PR4311 | SEQ ID NO: 99 |
| PR4312 | SEQ ID NO: 100 |
| PR4313 | SEQ ID NO: 101 |
| PR4528 | SEQ ID NO: 102 |
| PR4529 | SEQ ID NO: 103 |
| PR4490 | SEQ ID NO: 104 |
| PR4491 | SEQ ID NO: 105 |
| PR4494 | SEQ ID NO: 106 |
| PR4495 | SEQ ID NO: 107 |
| PR4532 | SEQ ID NO: 108 |
| PR4533 | SEQ ID NO: 109 |
| PR4492 | SEQ ID NO: 110 |
| PR4493 | SEQ ID NO: 111 |
| PR6085 | SEQ ID NO: 112 |
| PR6086 | SEQ ID NO: 113 |
| PR6113 | SEQ ID NO: 114 |
| PR6114 | SEQ ID NO: 115 |
| PR5701 | SEQ ID NO: 116 |
| PR5702 | SEQ ID NO: 117 |
| PR5703 | SEQ ID NO: 118 |
| PR5704 | SEQ ID NO: 119 |
| PR5705 | SEQ ID NO: 120 |
| PR5706 | SEQ ID NO: 121 |
| PR5707 | SEQ ID NO: 122 |
| PR5708 | SEQ ID NO: 123 |
| PR5709 | SEQ ID NO: 125 |
| PR6238 | SEQ ID NO: 126 |
| PR6087 | SEQ ID NO: 127 |
| PR6088 | SEQ ID NO: 128 |
| PR6259 | SEQ ID NO: 129 |
| PR6260 | SEQ ID NO: 130 |
| PR2818 | SEQ ID NO: 131 |
| PR2819 | SEQ ID NO: 132 |
| PR2820 | SEQ ID NO: 133 |
| PR2821 | SEQ ID NO: 134 |
| PR5008 | SEQ ID NO: 135 |
| PR5009 | SEQ ID NO: 136 |
| PR5350 | SEQ ID NO: 137 |
| PR5351 | SEQ ID NO: 138 |
| PR5354 | SEQ ID NO: 139 |
| PR5355 | SEQ ID NO: 140 |
| PR5452 | SEQ ID NO: 141 |
| PR2762 | SEQ ID NO: 142 |
| PR4365 | SEQ ID NO: 143 |
| PR4366 | SEQ ID NO: 144 |
| PR6107 | SEQ ID NO: 145 |
| PR6108 | SEQ ID NO: 146 |
| PR6109 | SEQ ID NO: 147 |
| PR6110 | SEQ ID NO: 148 |
| PR6810 | SEQ ID NO: 149 |
| PR6811 | SEQ ID NO: 150 |
| PR6814 | SEQ ID NO: 151 |
| PR6815 | SEQ ID NO: 152 |
| PR6844 | SEQ ID NO: 153 |
| PR6845 | SEQ ID NO: 154 |
| PR6846 | SEQ ID NO: 155 |
| PR6847 | SEQ ID NO: 156 |
| PR6931 | SEQ ID NO: 157 |
| PR6932 | SEQ ID NO: 158 |
| PR6937 | SEQ ID NO: 159 |
| PR6938 | SEQ ID NO: 160 |
| PR4860 | SEQ ID NO: 161 |
| PR4842 | SEQ ID NO: 162 |
| PR6250 | SEQ ID NO: 163 |
| PR6251 | SEQ ID NO: 164 |
| PR6252 | SEQ ID NO: 165 |
| PR6253 | SEQ ID NO: 167 |
| PR4429 | SEQ ID NO: 168 |
| PR4430 | SEQ ID NO: 169 |
| PR4431 | SEQ ID NO: 170 |
| PR4432 | SEQ ID NO: 171 |
| PR4433 | SEQ ID NO: 172 |
| PR4434 | SEQ ID NO: 173 |
| PR4478 | SEQ ID NO: 174 |
| PR4479 | SEQ ID NO: 175 |
| PR4526 | SEQ ID NO: 176 |
| PR4527 | SEQ ID NO: 177 |
| PR4331 | SEQ ID NO: 178 |
| PR4530 | SEQ ID NO: 179 |
| PR4531 | SEQ ID NO: 180 |
| PR5667 | SEQ ID NO: 181 |
| PR5668 | SEQ ID NO: 182 |
| PR5669 | SEQ ID NO: 183 |
| PR5670 | SEQ ID NO: 184 |
| PR6290 | SEQ ID NO: 185 |
| PR6291 | SEQ ID NO: 186 |
| PR4314 | SEQ ID NO: 187 |
| PR4315 | SEQ ID NO: 188 |
| PR4316 | SEQ ID NO: 189 |
| PR1843 | SEQ ID NO: 190 |
| PR1844 | SEQ ID NO: 191 |
| PR1845 | SEQ ID NO: 192 |
| PR1846 | SEQ ID NO: 193 |
| PR1847 | SEQ ID NO: 194 |
| PR1848 | SEQ ID NO: 195 |
| PR0673 | SEQ ID NO: 196 |
| PR0674 | SEQ ID NO: 197 |
| PR3163 | SEQ ID NO: 198 |
| PR6129 | SEQ ID NO: 199 |
| PR4308 | SEQ ID NO: 200 |
| siRNA | |
| siRNA FF4 sense | SEQ ID NO: 201 |
| siRNA FF4 antisense | SEQ ID NO: 202 |
| siRNA FF5 sense | SEQ ID NO: 203 |
| siRNA FF5 antisense | SEQ ID NO: 204 |
| miRIDIAN negative | SEQ ID NO: 205 |
| control #2 | |

TABLE 3
List of sequences of gene fragments/synthetic
DNA sequences/gBlocks

| Fragment name | SEQ ID NO |
|---|---|
| gBlock242 | SEQ ID NO: 206 |
| gBlock109 | SEQ ID NO: 207 |
| gBlock107 | SEQ ID NO: 208 |
| gBlock241 | SEQ ID NO: 209 |
| gBlock273 | SEQ ID NO: 210 |
| gBlock278 | SEQ ID NO: 211 |
| gBlock284 | SEQ ID NO: 212 |
| JD6.69.1 | SEQ ID NO: 213 |
| JD6.134.1 | SEQ ID NO: 214 |
| gBlock249 | SEQ ID NO: 215 |
| JD7.48.1 | SEQ ID NO: 216 |
| JD7.48.2 | SEQ ID NO: 217 |
| JD7.48.3 | SEQ ID NO: 218 |

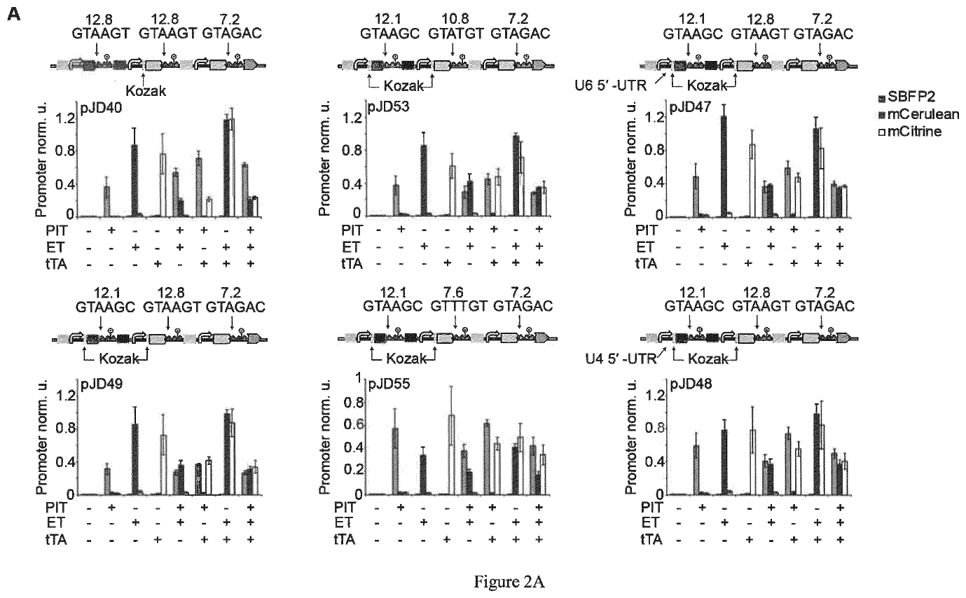

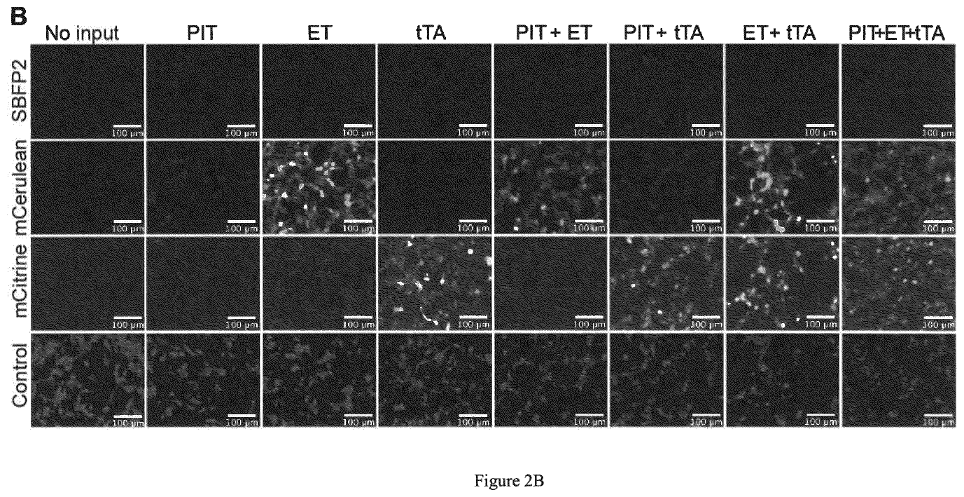

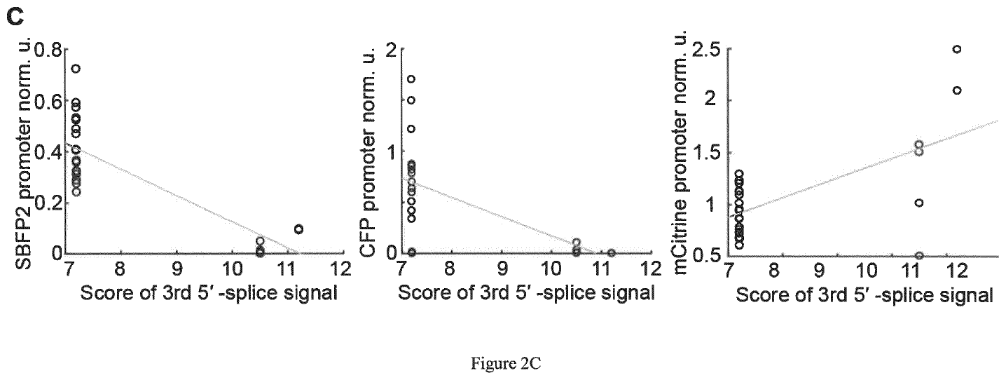

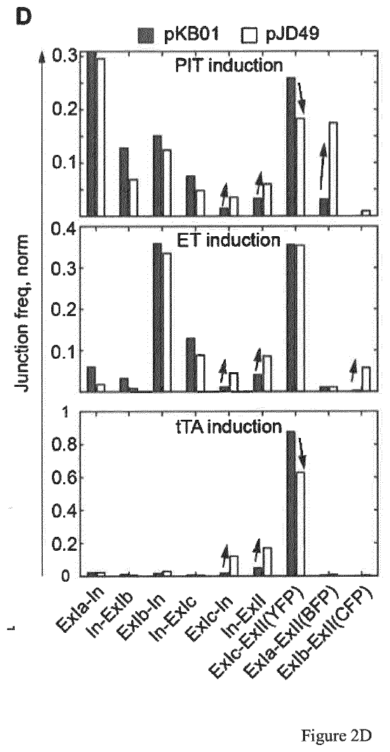

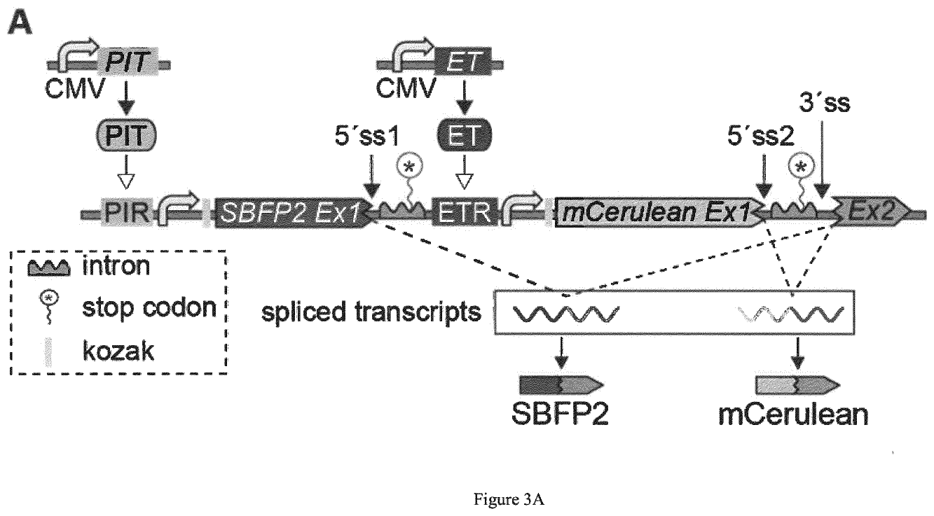

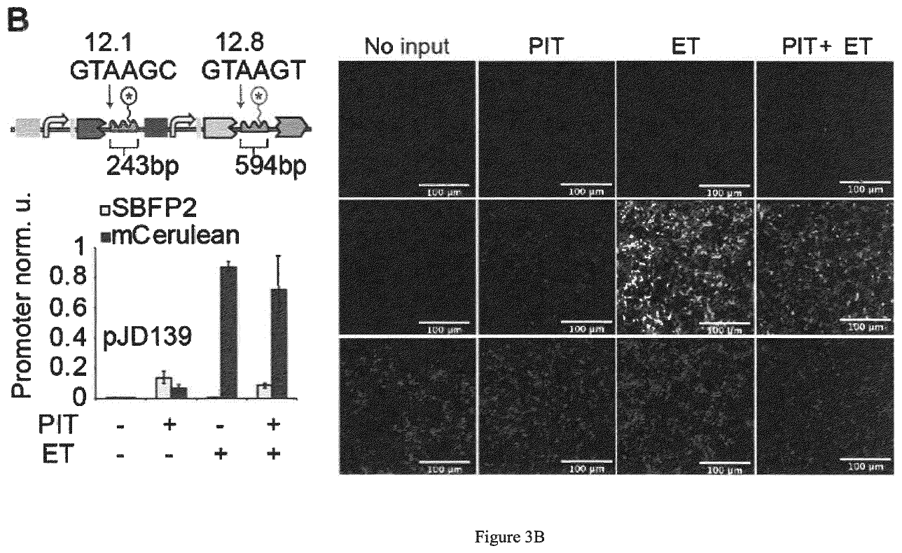

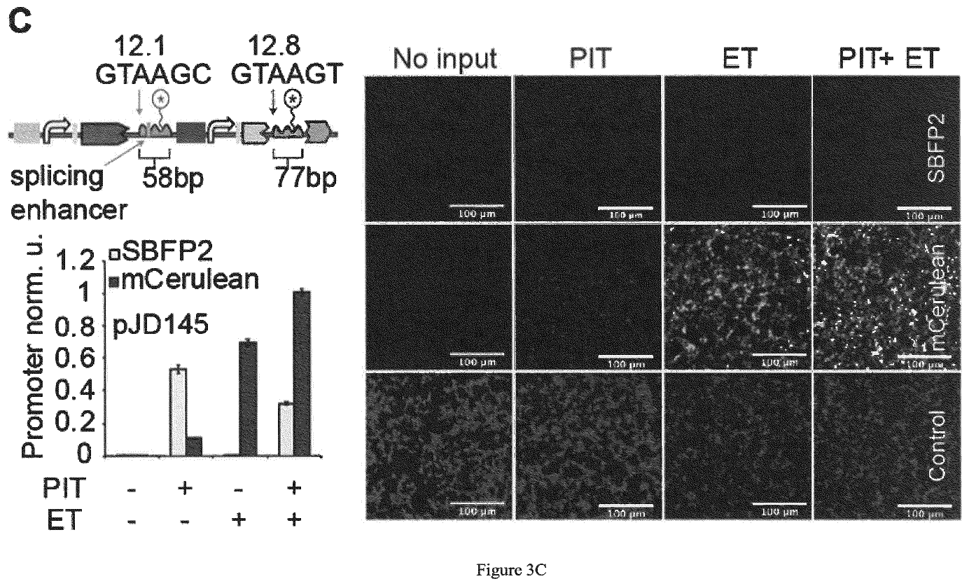

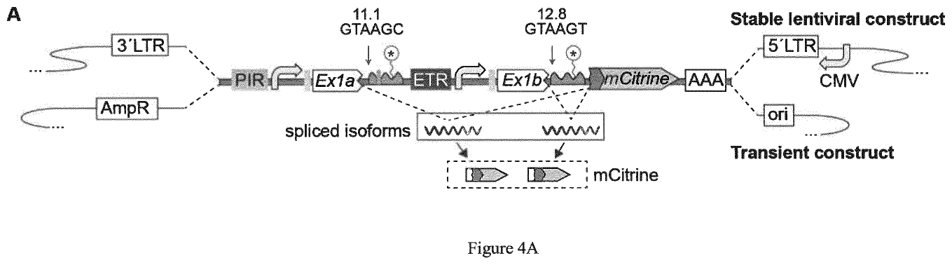

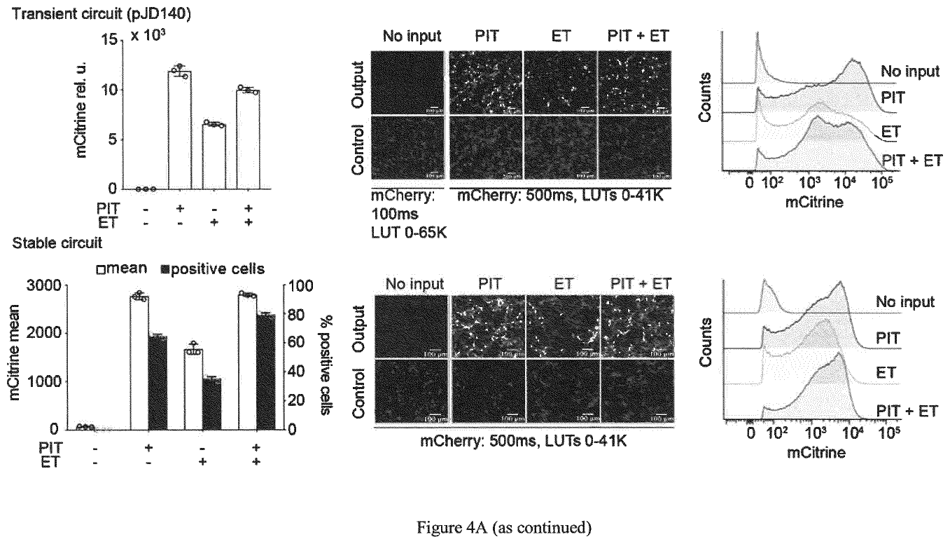

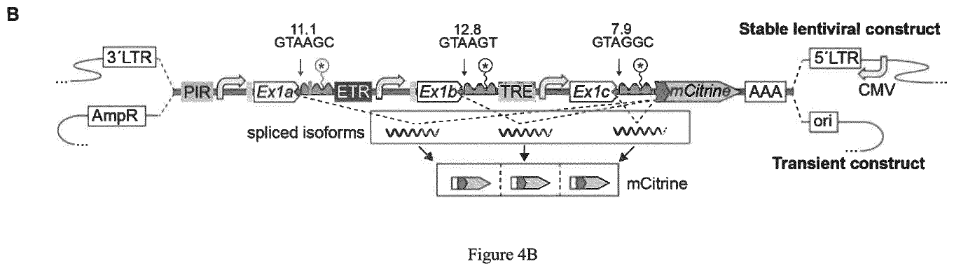

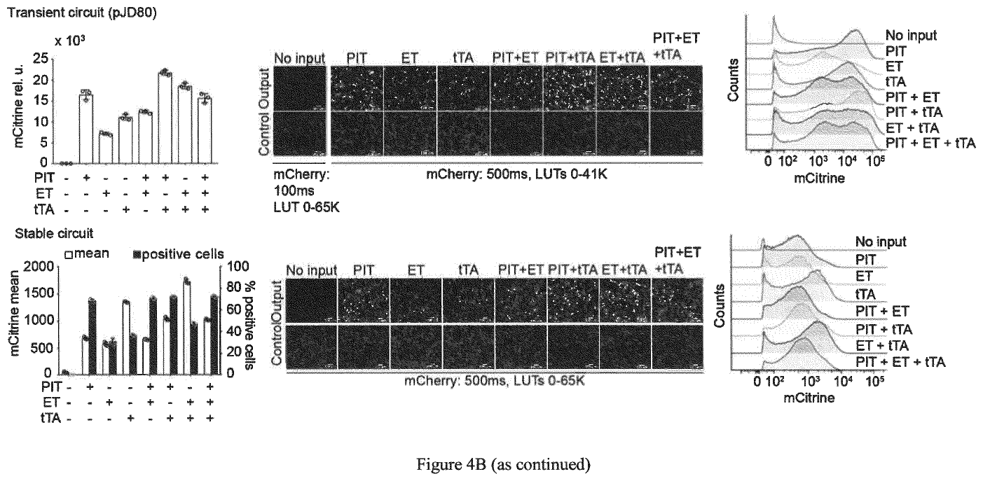

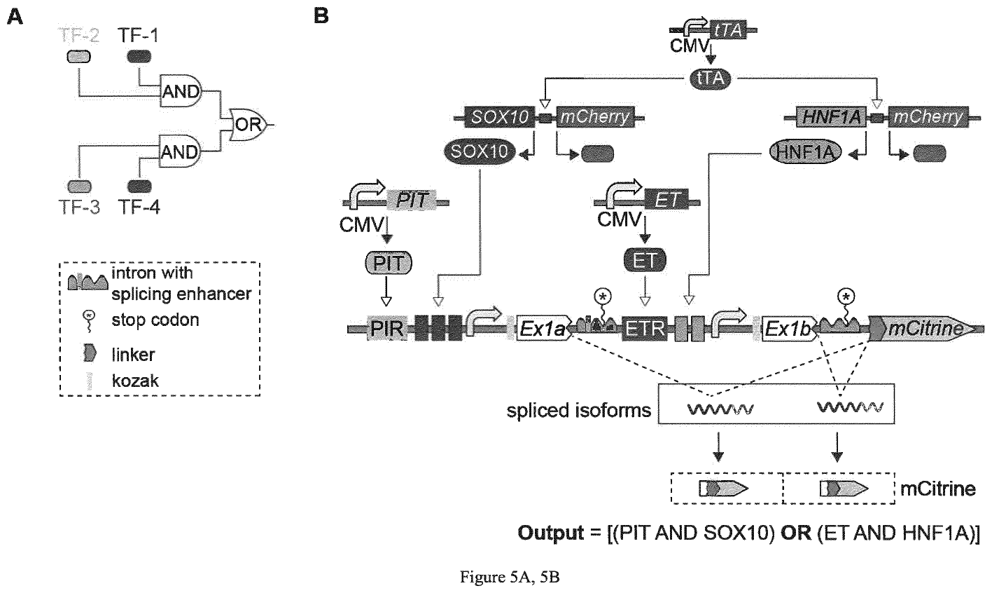

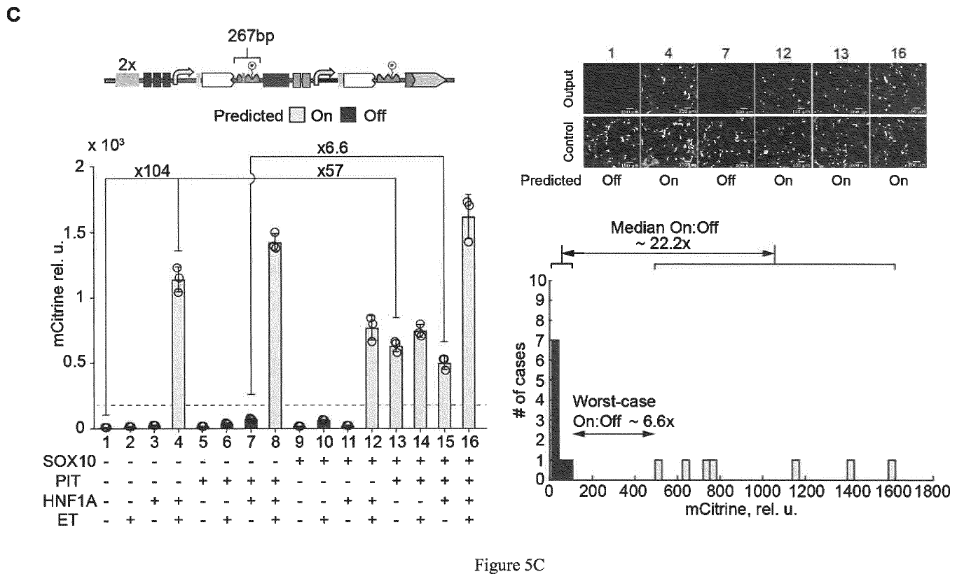

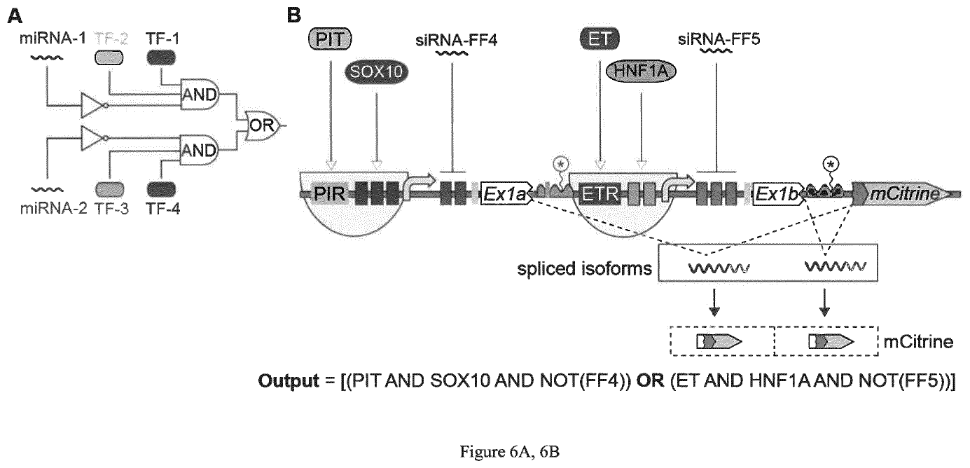

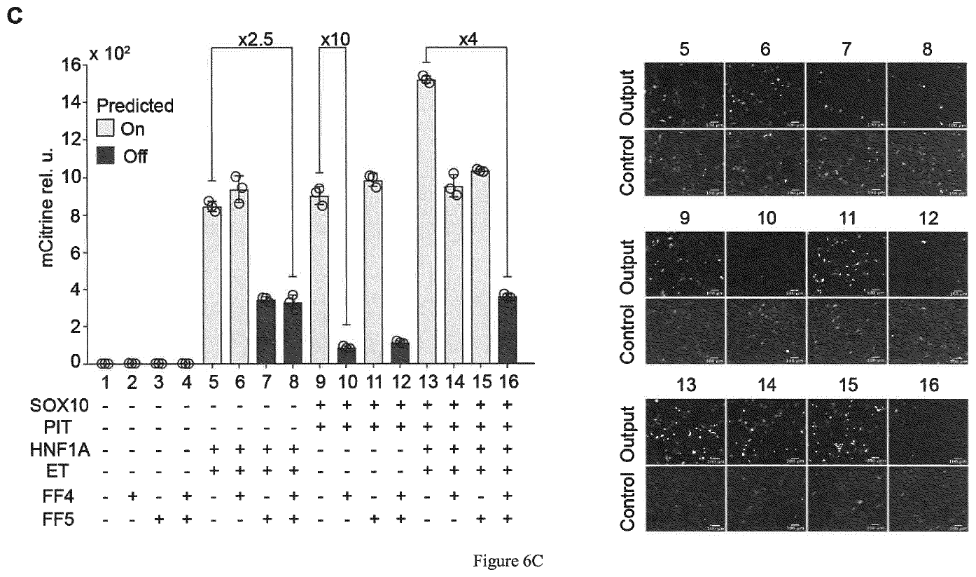

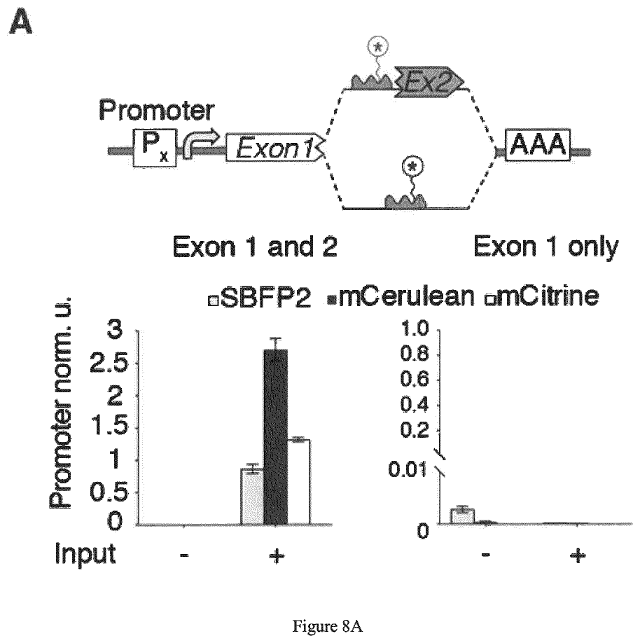

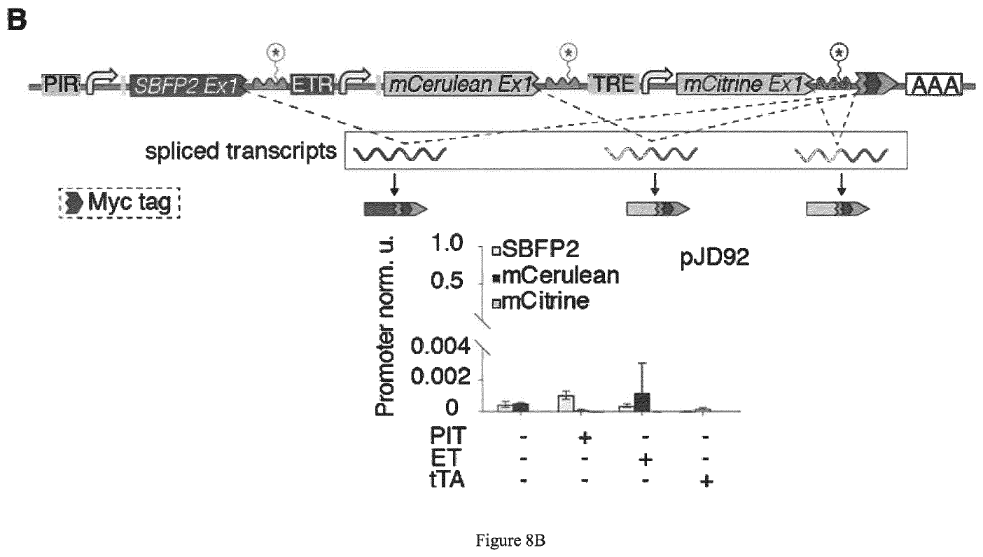

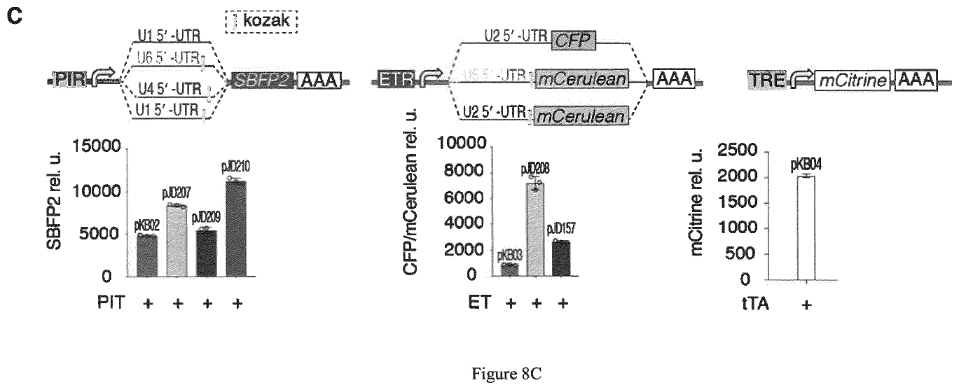

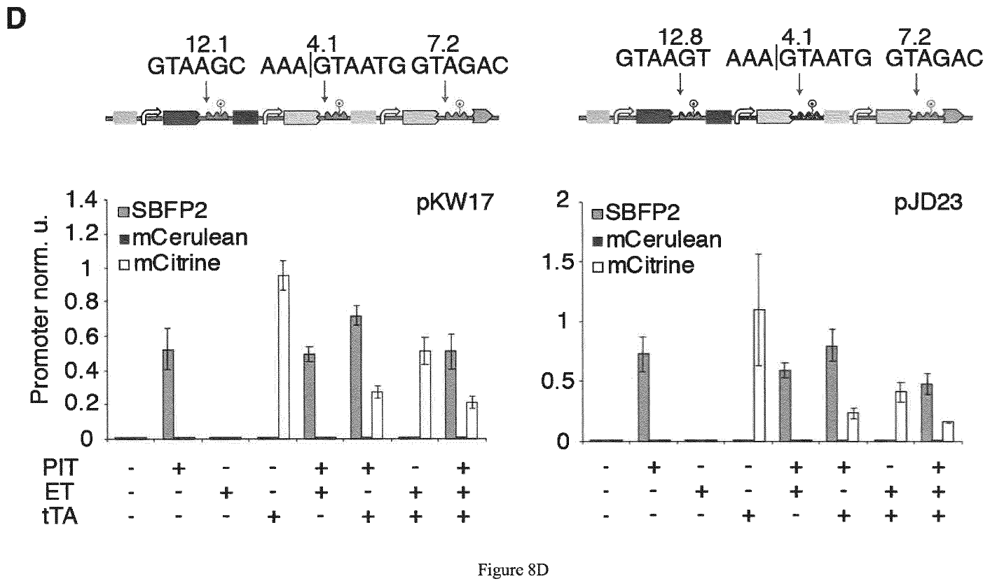

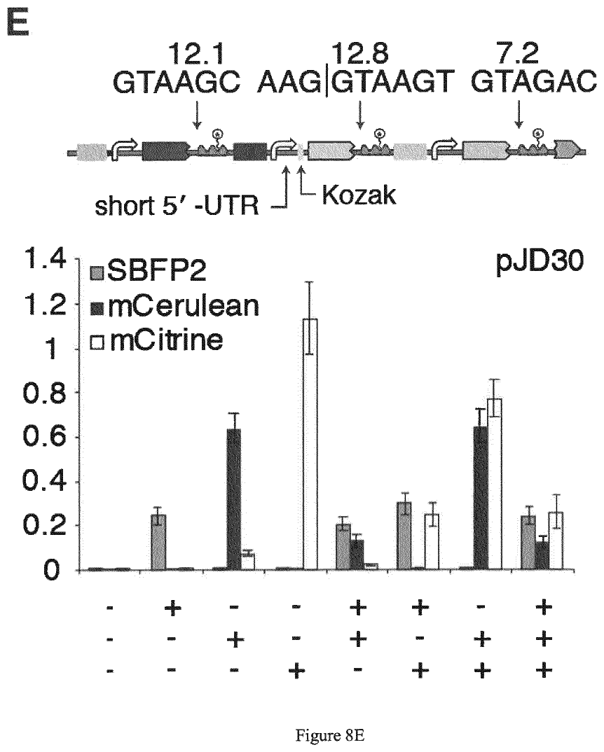

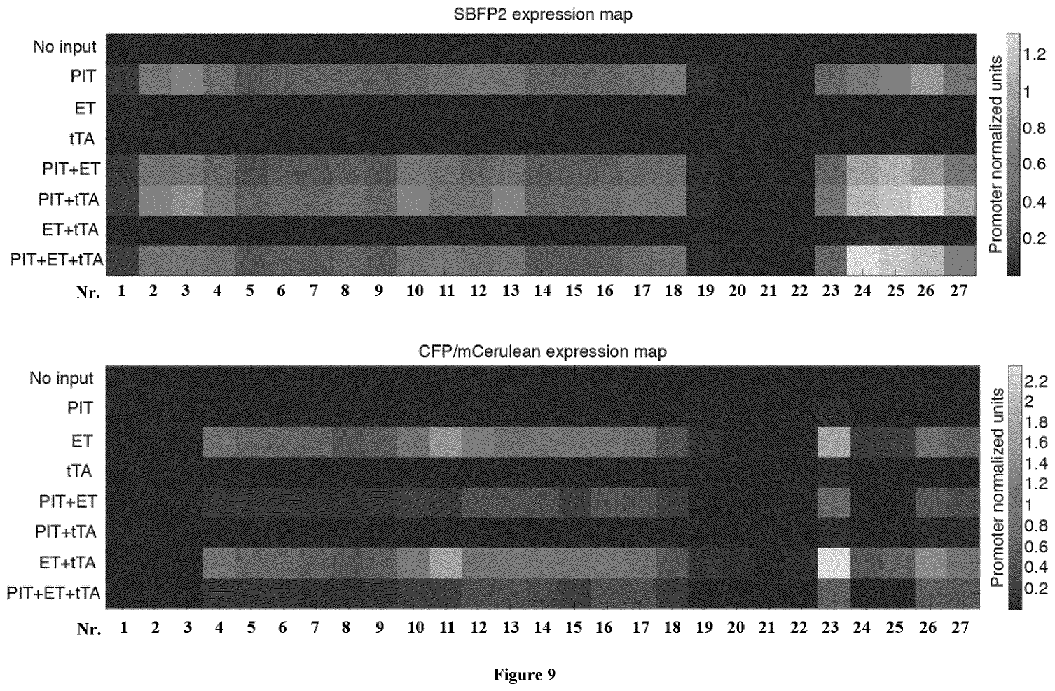

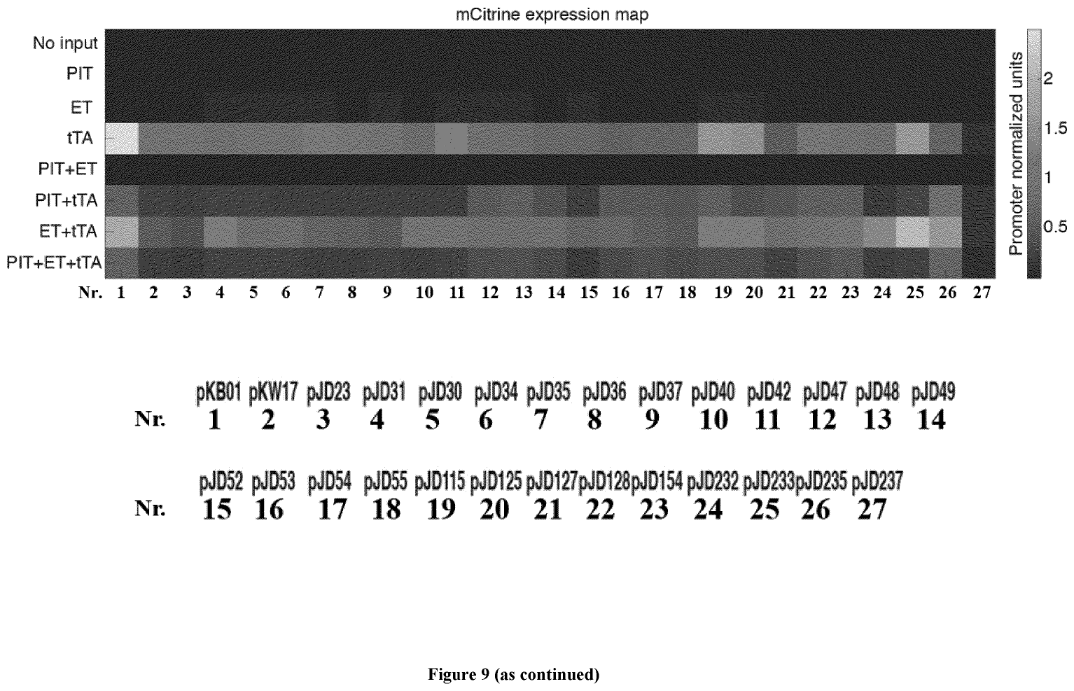

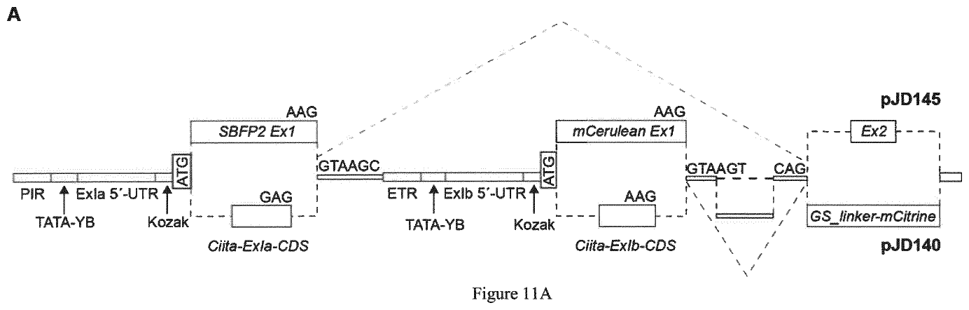

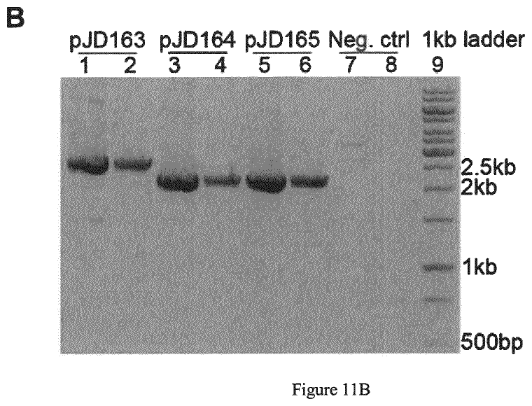

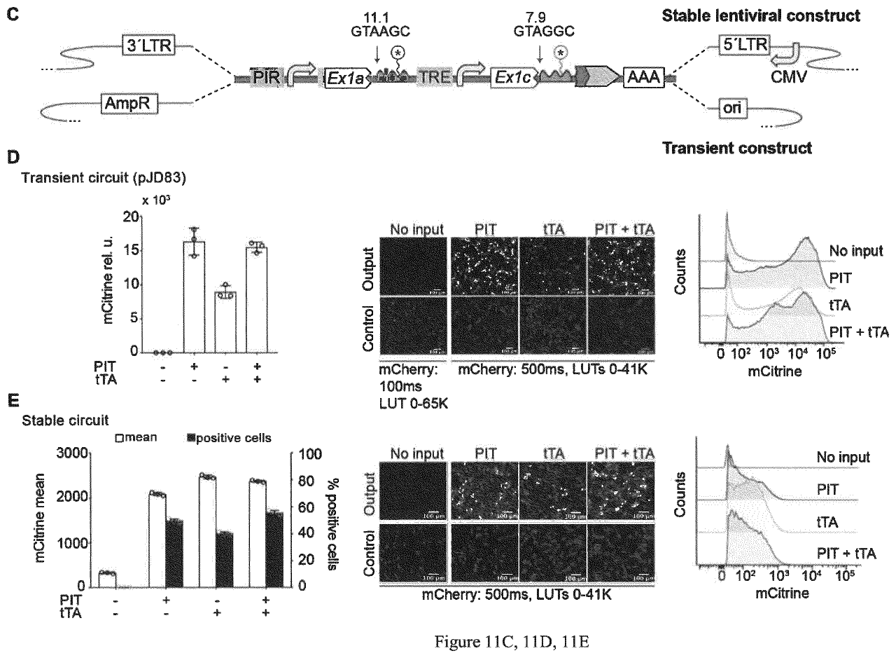

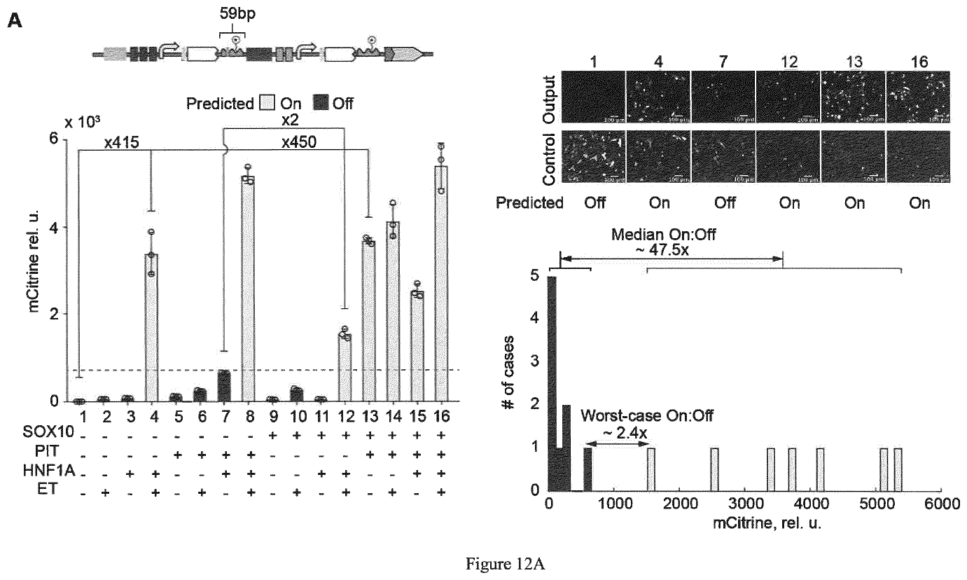

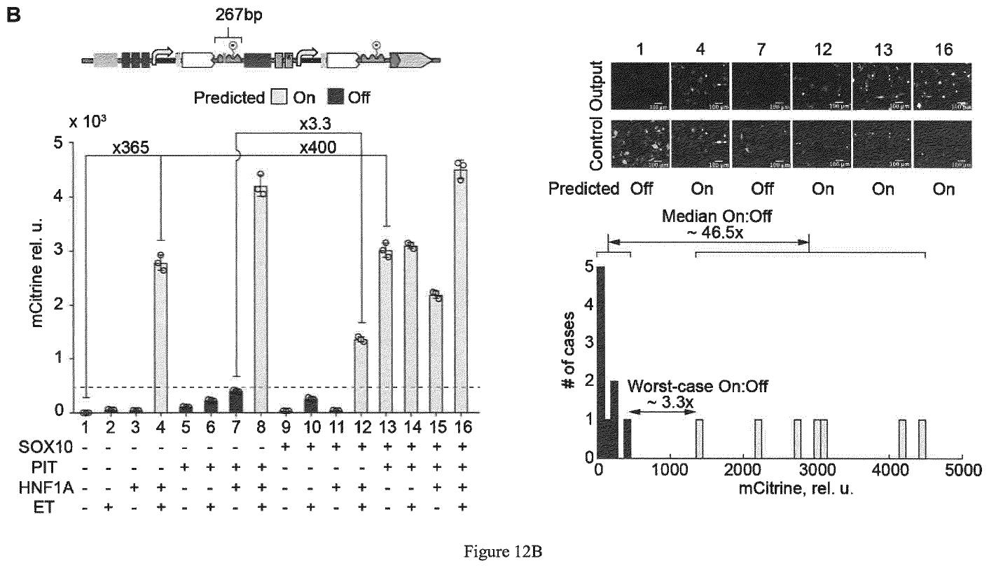

TABLE 4
List of plasmids with its cloning procedure

| Plasmid | Cloning procedure | Used in/for |
|---|---|---|
| pJD31 | pKW17 was digested with NheI, NsiI & CIP followed by gel | FIG. 1D |
| | purification of DNA (5583 bp). mCerulean_exon1, 5′ splice site | |
| | (ss6), intron (I7) (1190 bp) was amplified from gBlock242 using | |
| | primers PR4317/PR4356, gel extracted, digested with NheI & | |
| | NsiI, purified and ligated with the backbone. | |
| pJD33 | pKW17 was digested with NdeI, SacI & CIP followed by gel | FIG. 8A |
| | purification of DNA (2852 bp). PIR (P1) with SBFP2 (B1) (1100 bp) | (first bar |
| | was amplified from pKW17 using primers PR4330/PR4364, gel | chart) |
| | extracted, digested with NdeI & SacI, purified and ligated with | |
| | backbone. | |
| pJD144 | pJD139 was digested with AscI, NotI & CIP followed by gel | FIG. 8A |
| | purification of DNA (4243 bp). Blunting of the digested backbone | (first bar |
| | was performed using Fast DNA End Repair kit (ThermoFisher, | chart) |
| | Cat#K0771) followed by purification and ligation. | |
| pJD27 | pJD49 was digested with BamHI, NsiI & CIP followed by gel | FIG. 8A |
| | purification of DNA (1670 bp). Exon2 with backbone (2695 bp) was | (first bar |
| | amplified from pKB03 using primers PR4325/PR4326, gel | chart) |
| | extracted, digested with BamHI & NsiI, purified and ligated with | |
| | backbone. | |
| pAM01 | pKB01 was digested with MluI, NotI & CIP followed by gel | FIG. 8A |
| | purification of DNA (3642 bp). Blunting of the digested backbone | (second bar |
| | was performed using Fast DNA End Repair kit (ThermoFisher, | chart) |
| | Cat#K0771) followed by purification and ligation to clone pAM04 | |
| pJD143 | pJD139 was digested with MluI, NsiI & CIP followed by gel | FIG. 8A |
| | purification of DNA (5000 bp). Blunting of the digested backbone | (second bar |
| | was performed using Fast DNA End Repair kit (ThermoFisher, | chart) |
| | Cat#K0771) followed by purification and ligation to clone pJD143 | |
| pAM03 | pKB01 was digested with PstI, MluI & CIP followed by gel | FIG. 8A |
| | purification of DNA (6190 bp). Blunting of the digested backbone | (second bar |
| | was performed using Fast DNA End Repair kit (ThermoFisher, | chart) |
| | Cat#K0771) followed by purification and ligation to clone pAM03 | |
| pJD92 | pJD49 was digested with MluI, BamHI & CIP followed by | FIG. 8B |
| | purification of DNA (6670 bp) and ligated with annealed oligos | |
| | PR5070 & PR5071. | |
| pKB02 | PIR(P1)-SBFP2-polyA(pA1) (3488 bp) was amplified from pKB01 | FIG. 8C |
| | using primers prKB0001/prKB0002, loaded on agarose gel, | (first bar |
| | extracted, purified and circularized using T4 DNA ligase. | chart) |
| pKB03 | ETR(P2)-CFP-polyA(pA1) (4712 bp) was amplified from pKB01 | FIG. 8C |
| | using primers prKB0003/prKB0002, loaded on agarose gel, | (Second bar |
| | extracted, purified and circularized using T4 DNA ligase. | chart) |
| pKB04 | TRE(P3)-mCitrine-polyA(pA1) (6018 bp) was amplified from | FIG. 8C |
| | pKB01 using primers prKB0004/prKB0002, loaded on agarose | (third bar |
| | gel, extracted, purified and circularized using T4 DNA ligase. | chart) |
| pJD207 | pKB01 was digested with HindIII, NcoI & CIP followed by gel | FIG. 8C |
| | purification of DNA (3375 bp) and ligated with annealed oligos | (first bar |
| | PR7360 and PR7361. | chart) |
| pJD208 | pJD30 was digested with BamHI, MfeI & CIP followed by gel | FIG. 8C |
| | purification of DNA (4481 bp) and ligated with annealed oligos | (Second bar |
| | PR7358 and PR7359. | chart) |
| pJD209 | pKB02 was digested with NcoI, HindIII & CIP, purified, loaded on | FIG. 8C |
| | agarose gel, extracted and purified DNA (3375 bp) and ligated | (first bar |
| | with annealed oligos PR7362 and PR7363. | chart) |
| pJD210 | pKB02 was digested with NcoI, HindIII & CIP, purified, loaded on | FIG. 8C |
| | agarose gel, extracted and purified DNA (3375 bp) and ligated | (first bar |
| | with annealed oligos PR7364 and PR7365. | chart) |
| pJD157 | pJD144 was digested with MluI & CIP followed by digestion with | FIG. 8C |
| | BstBI. The digestion product was loaded on agarose gel to | (Second bar |
| | extract and purify DNA (3557 bp) and ligated with annealed oligos | chart) |
| | PR6812 & PR6813. | |
| pKW17 | This construct was obtained by a series cloning steps as | FIG. 8D |
| | described below. pKB01 was digested with AflII, BmtI & CIP | |
| | followed by gel purification of DNA (5750 bp). Gibson assembly | |
| | was performed of mCerulean containing gBlock109 with the | |
| | backbone to clone pKW14. pKW14 was digested with AvrII, PstI | |
| | & CIP followed by gel purification of DNA (5807 bp). pKW13 was | |
| | digested with AvrII, PstI & CIP followed by gel purification of DNA | |
| | (927 bp) and ligated with the backbone to clone pKW17 | |
| pKW13 | This construct was obtained by a series cloning steps as | pKW17 and |
| | described below. b) pKB01 was digested with NsiI, NotI & CIP | pJD122 |
| | followed by gel purification of DNA (5403 bp). Blunting of the | cloning |
| | digested backbone was performed using Fast DNA End Repair | |
| | kit (ThermoFisher, Cat#K0771) followed by purification and | |
| | ligation to clone pAM04. pAM04 was digested with BtgZI, AlfI & | |
| | CIP followed by gel purification of DNA (4838 bp). Gibson | |
| | assembly was performed of gBlock107 with the backbone to | |
| | clone pKW13. | |
| pJD23 | pKW17 was digested with NcoI, NotI & CIP followed by gel | FIG. 8D |
| | purification of DNA (5786 bp). SBFP2_exon1 (B1) with 5′ splice | |
| | site (ss6) (675 bp) was amplified from pKB02 using primers | |
| | PR4310/PR4311, gel extracted, digested with NcoI & EcoRI and | |
| | purified. Intron (I17) (239 bp) was amplified from pKW17 using | |
| | primers PR4312/PR4313, gel extracted, digested with NotI & | |
| | EcoRI and purified. Backbone was ligated with fragments. | |
| pJD30 | pKW17 was digested with NheI, NsiI & CIP followed by gel | FIG. 8E |
| | purification of DNA (5583 bp). mCerulean_exon1 (C6), 5′ splice | |
| | site (ss6), intron (I6) (1190 bp) was amplified from gBlock241 | |
| | using primers PR4317/PR4356, gel extracted, digested with NheI | |
| | & NsiI, purified and ligated with the backbone. | |
| pJD40 | pJD31 was digested with NotI, PvuI & CIP followed by gel | FIG. 2A |
| | purification of DNA (4862 bp). pJD23 was digested with NotI, PvuI | |
| | & CIP followed by gel purification of DNA (1883 bp) and ligated | |
| | with the backbone. | |
| pJD53 | pJD49 was digested with BbsI, CIP & BstBI followed by gel | FIG. 2A |
| | purification of DNA (6707 bp) and ligated with annealed oligos | |
| | PR4528 & PR4529. | |
| pJD47 | pJD31 was digested with NdeI, NotI & CIP followed by gel | FIG. 2A |
| | purification of DNA (5663 bp). PIR-TATA (P1) (150 bp) was | |
| | amplified from pJD33 using primers PR4491/PR4330, gel | |
| | extracted, digested with NdeI & EcoRI and purified. | |
| | SBFP2_exon1 (B1) with intron (I1) (931 bp) was amplified from | |
| | pJD33 using primers PR4490/PR4313, gel extracted, digested | |
| | with EcoRI & NotI and purified. The backbone was ligated with | |
| | the fragments. | |
| pJD49 | pJD47 was digested with SpeI, EcoRI & CIP followed by | FIG. 2A |
| | purification of DNA (6683 bp) and ligated with annealed oligos | |
| | PR4494 & PR4495. | |
| pJD55 | pJD49 was digested with BbsI, CIP & BstBI followed by gel | FIG. 2A |
| | purification of DNA (6707 bp) and ligated with annealed oligos | |
| | PR4532 & PR4533. | |
| pJD48 | pJD47 was digested with EcoRI, SpeI & CIP followed gel | FIG. 2A |
| | purification of DNA (6683 bp) and ligated with annealed oligos | |
| | PR4492 & PR4493. | |
| pJD139 | pJD49 was digested with SacI, NsiI & CIP followed by gel | FIG. 3B |
| | purification of DNA (5292 bp) and ligated with annealed oligos | |
| | PR6085 & PR6086. | |
| pJD145 | pJD142 was digested with SpeI, NotI & CIP followed by gel | FIG. 3C |
| | purification of DNA (3922 bp). SBFP2_ex1 with splice site ss4 and | |
| | intron I21 (722 bp) was amplified from pJD33 using primers | |
| | PR6113/PR6114, gel extracted, digested with SpeI & NotI, | |
| | purified and ligated with the backbone. | |
| pJD142 | pJD49 was digested with BbsI, BamHI & CIP, purified followed by | pJD145 |
| | gel purification of DNA (4767 bp). Gibson assembly was | cloning |
| | performed of ssDNA oligo PR6238 with the backbone. | |
| pJD140 | pJD80 was digested with BamHI, NsiI & CIP followed by gel | FIG. 4A |
| | purification of DNA (4321 bp) and ligated with annealed oligos | (transient |
| | PR6087 & PR6088. | version) |
| pJD80 | pJD49 was digested with MluI, SpeI & CIP followed by gel | FIG. 4B |
| | purification of DNA (4008 bp) gBlock273 containing Ciita_exon1a | (transient |
| | with 5′UTR (U1) was digested with NsiI, purified, | version) |
| | amplified(358 bp) using PR5701 & PR5702 and purified. ETR | |
| | (P2) with 5′UTR (U2) (490 bp) was amplified from pJD32 using | |
| | PR5703 & PR5704, DpnI digested and purified. TRE (P3) with | |
| | 5′UTR (U3) (451 bp) was amplified from pJD115 using PR5705 & | |
| | PR5706, DpnI digested and purified. mCitrine (842 bp) was | |
| | amplified from pKH025 using PR5707 & PR5708, DpnI digested | |
| | and purified. Gibson assembly was performed for all fragments | |
| | with digested backbone. | |
| pJD83 | pJD122 was digested with MluI, NcoI & CIP followed by gel | FIG. 11D |
| | purification of DNA (4008 bp). gBlock273 containing Ciita_exon1a | (transient |
| | with 5′UTR (U1) was digested with NsiI, purified, amplified | version) |
| | (358 bp) using PR5701 & PR5702 and purified. TRE (P3) with | |
| | 5′UTR (U3) (448 bp) was amplified from pJD115 using PR5709 | |
| | and PR5706, DpnI digested and purified. mCitrine (842 bp) was | |
| | amplified from pKH25 using PR5707 & PR5708, DpnI digested | |
| | and purified. Gibson assembly was performed for all fragments | |
| | with digested backbone. | |
| pJD163 | pJD17 was digested with BstBI, PacI & CIP followed by gel | FIG. 4B |
| | purification of DNA (7930 bp). PIR-Ciita_ex1a-intron-ETR- | (stable |
| | Ciita_ex1b-intron2-TRE-Ciita_ex1c-intron3-GSlinker-mCitrine- | version) |
| | polyA (2430 bp) was amplified from pJD80 using primers | |
| | PR6260/PR6259, DpnI digested and purified. Gibson assembly | |
| | was performed of the fragment with the backbone. | |
| pJD164 | pJD17 was digested with BstBI, PacI & CIP followed by gel | FIG. 11E |
| | purification of DNA (7930 bp). PIR-Ciita_ex1a-intron1-TRE- | (stable |
| | Ciita_ex1c-intron3-GSlinker-mCitrine-polyA(2002 bp) was | version) |
| | amplified from pJD83 using primers PR6260/PR6259, DpnI | |
| | digested and purified. Gibson assembly was performed of the | |
| | fragment with the backbone. | |
| pJD165 | pJD17 was digested with BstBI, PacI & CIP followed by gel | FIG. 4A |
| | purification of DNA (7930 bp). PIR-Ciita_ex1a-intron1-ETR- | (stable |
| | Ciita_ex1b-intron2-GSlinker-mCitrine-polyA (2040 bp) was | version) |
| | amplified from pJD140 using primers PR6260/PR6259, DpnI | |
| | digested and purified. Gibson assembly was performed of the | |
| | fragment with the backbone. | |
| pJD17 | pJD13 was digested with PacI, EcoRI & CIP followed by gel | pJD163, |
| | purification of DNA (7930 bp). Ef1a promoter with 5′UTR | pJD164, |
| | (1395 bp) was amplified from pRA16 (described in Altamura et al, | pJD165 |
| | in preparation) using primers PR2818/PR2819, loaded on | cloning |
| | agarose gel and purified. mCherry (850 bp) was amplified from | |
| | pKH026 using primers PR2820/PR2821, gel extracted and | |
| | purified. Gibson assembly was performed of the fragments with | |
| | the backbone. | |
| pJD115 | pJD49 was digested with NotI, SpeI & CIP followed by gel | FIG. 9 |
| | purification of DNA (5872 bp). SBFP2_exon (B4) with intron (I16) | |
| | (924 bp) was amplified from gBlock278 using primers | |
| | PR5008/PR5009, gel extracted, digested with NotI, SpeI, purified | |
| | and ligated with the backbone to clone pJD106. pJD106 was | |
| | digested with BspEI, ClaI & CIP followed by purification of DNA | |
| | (6700 bp) and ligated with annealed oligos PR5350 & PR5351 to | |
| | clone pJD109. pJD109 was digested with MfeI, CIP & BstBI | |
| | followed by purification of DNA (6715 bp) and ligated with | |
| | annealed oligos PR5354 & PR5355 to clone pJD111. pJD111 | |
| | was digested with AvrII, PstI & CIP followed by gel purification of | |
| | DNA (5850 bp). mCitrine_exon1 (Y3) with intron (I20) (954 bp) | |
| | was amplified from gBlock284 using primers PR5452/PR2762, | |
| | gel purified, digested with AvrII & PstI, purified and ligated with | |
| | the backbone to clone pJD115. | |
| pJD122 | pKW13 was digested with AvrII and PstI followed by gel | pJD83 cloning |
| | purification of DNA (4476 bp) and ligated, with 927 bp DNA | |
| | obtained by digestion and gel purification of pJD115 with AvrII | |
| | and PstI. | |
| pJD32 | pKW17 was digested with NdeI, SacI & CIP followed by gel | pJD80 cloning |
| | purification of DNA (2852 bp). ETR (P2) with mCerulean (C1) | |
| | (1100 bp) was amplified from pKW17 using primers | |
| | PR4365/PR4366, gel extracted, digested with NdeI & SacI, | |
| | purified and ligated with backbone. | |
| pJD198 | pJD178 was digested with ClaI, NotI & CIP followed by gel | FIG. 12B |
| | purification of DNA (4351 bp) and ligated with DNA (226 bp) | |
| | obtained by digestion of pJD125 with ClaI and NotI followed by | |
| | gel purification. | |
| pJD178 | This construct was obtained by a series cloning steps as | FIG. 12A |
| | described below. pJD140 was digested with AscI, EcoRI & CIP, | |
| | purified and ligated with annealed oligos PR6107 and PR6108 to | |
| | clone pJD155. pJD155 was digested with NotI, HindIII & CIP, | |
| | purified and ligated with annealed oligos PR6109 and PR6110 to | |
| | clone pJD156. pJD156 was digested with SacI, KpnI & CIP, | |
| | purified and ligated with annealed oligos PR6810 and PR6811 to | |
| | clone pJD158. pJD158 was digested with PpuMI, BamHI & CIP, | |
| | purified, loaded on agarose gel and DNA (4251 bp) was extracted | |
| | and purified. JD6.69.1 fragment was digested with PpuMI & | |
| | BamHI, purified and ligated with the backbone to clone pJD160. | |
| | pJD160 was digested with MfeI, HindIII & CIP, purified and | |
| | ligated with annealed oligos PR6814 and PR6815 to clone | |
| | pJD161. pJD161 was digested with NotI, MfeI & CIP, purified and | |
| | ligated with annealed oligos PR6846 and PR6847 to clone | |
| | pJD173. pJD173 was digested with KpnI, SacI & CIP, purified | |
| | and ligated with annealed oligos PR6844 and PR6845 to clone | |
| | pJD178. | |
| pJD125 | pJD49 was digested with AvrII, PstI & CIP followed by gel | FIG. 9 |
| | purification of DNA (5834 bp). mCitrine_exon1 (Y3) with intron | |
| | (I20) (954 bp) was amplified from gBlock284 using | |
| | PR5452/PR2762, gel purified, digested with AvrII & PstI, purified | |
| | and ligated with the backbone to clone pJD114. pJD106 was | |
| | digested with AvrII, PstI & CIP followed by gel purification of DNA | |
| | (5850 bp) and ligated with 927 bp DNA obtained by digestion of | |
| | pJD114 with AvrII, PstI & CIP and gel purification to clone | |
| | pJD125. | |
| pJD204 | This construct was obtained by a series cloning steps as | FIG. 6C |
| | described below. pJD178 was digested with EcoRI, KasI & CIP, | |
| | loaded on agarose gel and DNA(~3.5 kb) was extracted and | |
| | purified. JD6.134.1 gene fragment containing ff4 and ff5 target | |
| | sites in 5′UTRs was digested with EcoRI & KasI, purified and | |
| | ligated with the backbone to clone pJD194. pJD194 was digested | |
| | with ClaI, NotI & CIP followed by gel purification of DNA (4401 bp) | |
| | and ligated with DNA (226 bp) obtained by digestion of pJD125 | |
| | with ClaI and NotI and gel purification to clone pJD199. pJD199 | |
| | was digested with PpuMI, XhoI & CIP, purified and ligated with | |
| | annealed oligos PR6937 and PR6938 to clone pJD201. pJD201 | |
| | was digested with EcoRI, AgeI & CIP, purified and ligated with | |
| | annealed oligos PR6931 and PR6932 to clone pJD204. | |
| pJD70 | pJD69 (CMV-mScarlet; Addgene #85042; (Bindels et al., 2017)) | FIG. 5, 6C & |
| | was digested with NheI, BamHI & CIP followed by gel purification | 12 |
| | of DNA (3932 bp). PITVP16(1209 bp) was amplified from pBA481 | |
| | (Angelici et al., 2016)using primers PR4860/PR4842, gel | |
| | extracted, digested with BamHI, NheI, purified and ligated with | |
| | the backbone. | |
| pJD146 | pKH024 was digested with EcoRI, NotI & CIP followed by gel | Compensation |
| | purification of DNA (4279 bp). mCerulean_exon1(681 bp) was | control/ |
| | amplified from pJD144 using primers PR6250/PR6251, DpnI | transfection |
| | digested, purified. Gibson assembly was performed of ssDNA | control |
| | oligo PR6252, mCerulean_ex1 fragment with the backbone. | |
| pJD147 | pKH025 was digested with BlpI, XbaI & CIP followed by | Compensation |
| | purification to get DNA (4958 bp). Gibson assembly was | control |
| | performed of ssDNA oligo PR6253 with the backbone. | |
| pJD35 | pJD34 was digested with MfeI, BbsI & CIP followed by gel | FIG. 9 |
| | purification of DNA (6481 bp) and ligated with annealed oligos | |
| | PR4429 & PR4430. | |
| pJD36 | pJD34 was digested with MfeI, BbsI & CIP followed by gel | FIG. 9 |
| | purification of DNA (6481 bp) and ligated with annealed oligos | |
| | PR4431 & PR4432. | |
| pJD37 | pJD34 was digested with MfeI, BbsI & CIP followed by gel | FIG. 9 |
| | purification of DNA (6481 bp) and ligated with annealed oligos | |
| | PR4433 & PR4434. | |
| pJD34 | pJD30 was digested with NotI, PvuI & CIP followed by gel | FIG. 9 |
| | purification of DNA (4699 bp). pJD23 was digested with NotI, PvuI | |
| | & CIP followed by gel purification of DNA (1883 bp) and ligated | |
| | with the backbone. | |
| pJD42 | pJD31 was digested with BbvCI, BamHI & CIP followed by gel | FIG. 9 |
| | purification of DNA (6667 bp) and ligated with annealed oligos | |
| | PR4478 & PR4479. | |
| pJD52 | pJD33 was digested with BlpI, SacI & CIP followed by gel | FIG. 9 |
| | purification of DNA (3681 bp). Intron (I2) (300 bp) was amplified | |
| | from gBlock249 using primers PR4526/PR4527, gel extracted, | |
| | digested with BlpI, SacI, purified and ligated with the backbone to | |
| | clone pJD51. pJD49 was digested with NotI, SalI & CIP followed | |
| | by gel purification of DNA (5668 bp). PIR (P1), SBFP2_exon1 | |
| | (B1) with intron (I2) (1131 bp) was amplified from pJD51 using | |
| | primers PR4330/PR4331, gel extracted, digested with NotI, SalI, | |
| | purified and ligated with the backbone. | |
| pJD54 | pJD49 was digested with BbsI, CIP & BstBI followed by gel | FIG. 9 |
| | purification of DNA (6707 bp) and ligated with annealed oligos | |
| | PR4530 & PR4531. | |
| pJD127 | pJD115 was digested with KasI, BamHI & CIP followed by gel | FIG. 9 |
| | purification of DNA (6080 bp) and ligated with annealed oligos | |
| | PR5667 & PR5668. | |
| pJD128 | pJD115 was digested with KasI, BamHI & CIP followed by gel | FIG. 9 |
| | purification of DNA (6080 bp) and ligated with annealed oligos | |
| | PR5669 & PR5670. | |
| pJD154 | pJD49 was digested with BstBI, NsiI & CIP followed by gel | FIG. 9 |
| | purification of DNA (6393 bp). Intron (I1) from Ciita mouse gene | |
| | (299 bp) was amplified using 3T3 cells' genomic DNA as template | |
| | using primers PR6290/PR6291 and purified. Gibson assembly | |
| | was performed of the fragment with the backbone. | |
| pJD232 | pKB01 was digested with AvrII, PstI & CIP, loaded on agarose | FIG. 9 |
| | gel and DNA (5813 bp) was extracted and purified. mCitrine with | |
| | weaker 5′ splice site (2043 bp) gene fragment (JD7.48.1) was | |
| | digested with AvrII & PstI, purified and ligated with the backbone. | |
| pJD233 | pKB01 was digested with AvrII, PstI & CIP, loaded on agarose | FIG. 9 |
| | gel and DNA (5813 bp) was extracted and purified. mCitrine with | |
| | weaker 5′ splice site (2043 bp) gene fragment (JD7.48.2) was | |
| | digested with AvrII & PstI, purified and ligated with the backbone. | |
| pJD235 | pJD48 was digested with AvrII, PstI & CIP, loaded on agarose gel | FIG. 9 |
| | and DNA (5856 bp) was extracted and purified. mCitrine with | |
| | weaker 5′ splice site (2043 bp) gene fragment (JD7.48.1) was | |
| | digested with AvrII & PstI, purified and ligated with the backbone. | |
| pJD237 | pJD48 was digested with AvrII, PstI & CIP, loaded on agarose gel | FIG. 9 |
| | and DNA (5856 bp) was extracted and purified. mCitrine with | |
| | weaker 5′ splice site (2043 bp) gene fragment (JD7.48.3) was | |
| | digested with AvrII & PstI, purified and ligated with the backbone. | |
| pJD219 | pJD198 was digested with AscI, SacI & CIP followed by | FIG. 5C |
| | purification of DNA (4499 bp) and ligated with annealed oligos | |
| | PR8218 & PR8219. | |
| pKB01 | Synthesized by Genewiz | FIG. 1C |
| pMF206 (CMV- | Mentioned in Weber et al., 2002 | FIG. 1C, |
| PIT2) | | 1D, 8, 9, 11, |
| | | 2A, 3B, 3C, 4 |
| pEL190 (CMV- | Mentioned in Prochazka et al., 2014 | FIG. 1C, |
| ET1) | | 1D, 8, 9, 11, |
| | | 2A, 3B, 3C, 4, |
| | | 5, 6 |
| pBA166 (CMV- | Synthesized by Clonetech Laboratories | FIG. 1C, |
| tTA) | | 1D, 8, 9, 11, |
| | | 2A, 4B, 5, 6 |
| pKH026 (Ef1a- | Mentioned in Prochazka et al., 2014 | transfection |
| mCherry) | | control/ |
| | | compensation |
| | | control |
| pKH025 (Ef1a- | Mentioned in Prochazka et al., 2014 | compensation |
| mCitrine) | | control |
| pKH024 (Ef1a- | Mentioned in Prochazka et al., 2014 | compensation |
| mCerulean) | | control |
| pCS187 (Ef1a- | Mentioned in Stelzer et al (in preparation) | compensation |
| SBFP2) | | control |
| pBH265 (junk | Mentioned in Haefliger et al., 2016 | junk DNA |
| DNA) | | |
| pJD13 (5′LTR- | Mentioned in Lois et al., 2002 | pJD17 cloning |
| UbC-GFP- | | |
| WPRE-3′LTR) | | |
| pJD14 (CMV- | Mentioned in Dull et al., 1998 | lentivirus |
| Gag/Pol) | | production |
| pJD15 (CMV- | Mentioned in Dull et al., 1998 | lentivirus |
| VSV-G) | | production |
| pJD16 (REV- | Mentioned in Dull et al., 1998 | lentivirus |
| Rev) | | production |
| pRA16 (Ef1a- | Mentioned in Altamura et al (in preparation) | pJD17 cloning |
| mCitrine) | | |
| pEM003 | Mentioned in Angelici et al., 2016 | FIG. 5, 6C & |
| (mCherry- | | 12 |
| TREbidirectional- | | |
| HNF1A) | | |
| pBA417 | Mentioned in Angelici et al., 2016 | FIG. 5, 6C & |
| (mCherry- | | 12 |
| TREbidirectional- | | |
| SOX10) | | |
| pBA481 | Mentioned in Angelici et al., 2016 | pJD70 cloning |
| (mCherry- | | |
| TREbidirectional- | | |
| PIT.VP16) | | |
| pJD69 (CMV- | Mentioned in Bindels et al., 2017 | pJD70 cloning |
| mScarlet) | | |

### Recombinant DNA Methods

For different kits used, manufacturer's instructions were followed unless indicated otherwise. Standard cloning techniques were used to generate plasmids. DNA amplification was performed using Phusion High Fidelity DNA Polymerase (NEB, Cat #M0530). De-salted primers/oligonucleotides (Table 2) were ordered from IDT/Sigma Aldrich. De-salted gene fragments/synthetic DNA sequences/gBlocks (Table 3) were ordered from IDT/Twist Bioscience. Digestion fragments were purified using MinElute PCR purification kit (Qiagen, Cat #28006) or Qiaquick PCR purification kit (Qiagen, Cat #28106). Gel extraction and purification was performed using MinElute Gel purification kit (Qiagen, Cat #28606) or Qiaquick Gel Extraction kit (Qiagen, Cat #28706). Restriction digestion was performed for BstBI at 65° C., SfiI at 50° C., BtgZI at 70° C. and for all other enzymes at 37° C. Ligation reaction was performed using T4 DNA ligase (NEB, Cat #M0202). Mix and Go E. coli transformation kit (Zymo, Cat #T3001) was used for preparing chemically-competent cells—Top10 (ThermoFisher, Cat #C404010) and JM109 (Zymo, Cat #T3003). In-house prepared Mach1 electro-competent cells (ThermoFisher, Cat #C862003) and chemically competent Stbl3 cells (ThermoFisher, Cat #C737303) were also used for cloning. Screening of positive clones was either performed using restriction digestion or performing colony PCR with Quick-Load Taq 2× Master Mix (NEB, Cat #M0271). Plasmid isolation from positive clones was performed using GenElute Plasmid Mini-prep kit (Sigma Aldrich, Cat #PLN350-1KT). All the plasmids were verified using Sanger sequencing service provided by Microsynth AG (Switzerland). Transformed bacteria were cultured in Difco LB broth, Miller (BD, Cat #244610) supplemented with appropriate antibiotics (Ampicillin 100 μg/mL (Sigma Aldrich, Cat #A9518) and Kanamycin 50 pg/mL (Sigma Aldrich, Cat #K4000)). HiPure Plasmid Filter Midi-prep kit (Invitrogen, Cat #K210014) was used for plasmid isolation and purification. Endotoxin Removal kit (Norgen, Cat #52200) was used for removing endotoxins from purified plasmids. Gibson assembly (Gibson et al., 2009) was performed at 50° C. for 1 hour in 20 μL final volume by mixing vector (50 ng) and inserts (5 molar equivalent) in 1× Gibson assembly buffer (0.1 M Tris-HCl, pH 7.5, 0.01M MgCl₂, 0.2 mM dGTP, 0.2 mM dATP, 0.2 mM dTTP, 0.2 mM dCTP, 0.01 M DTT, 5% (w/v) PEG-8000, 1 mM NAD), 0.04 units of T5 exonuclease (NEB, Cat #M0363), 0.25 units of Phusion DNA polymerase (NEB, Cat #M0530) and 40 units of Taq DNA ligase (NEB, Cat #M0208). Negative controls for Gibson assemblies included vectors alone. Oligo cloning comprised phosphorylation and annealing of oligonucleotides prior to ligation with the backbone fragment. Phosphorylation of oligonucleotides was performed by adding together 3 μL olignucleotide (100 μM), 5 μL 10×PNK buffer, 5 μL ATP (10 mM), 1.5 μL of T4 PNK (10 U/μL) (NEB, Cat #M0201) and 34 μL ddH₂O followed by incubation at 37° C. for 30 minutes. Annealing was performed by mixing 25 μL each of the phosphorylated oligonucleotide and then incubating in a thermocycler at 95° C. for 3 minutes followed by a decrease of 0.5° C. every minute for the next 170 minutes. 1 μL of 1:20 diluted (with ddH₂O) annealed oligonucleotides was used for ligation reaction.

### Transfection

All transfections were performed using Lipofectamine 2000 transfection reagent (ThermoFisher, Cat #11668-027) according to the suggested guidelines. Transfections were performed either in 24-well plate (Cat #142475, ThermoFisher) or 6-well plate (Cat #140675, ThermoFisher) (for RNA-sequencing). The cells were seeded in each well 24 hours prior to transfection at a density of 7.5*10⁴ for HEK293, stably transduced HEK293 in 24-well plate, 3.5*10⁵ for HEK293 in 6-well plate and 5.5*10⁴ for HeLa in 24-well plate in order to have around 70-80% of confluency at the time of transfection. DMEM supplemented with 10% FBS and 1% Penicillin/Streptomycin solution was used for seeding HEK293 cells while DMEM supplemented with 10% FBS and no antibiotic was used for seeding HeLa cells. Appropriate amounts of plasmids used for each transfection were mixed together and Opti-MEM (ThermoFisher, Cat #31985-062) was used to make final volume of 50 μL (24-well) or 250 μL (6-well) (DNA Opti-MEM mix). Ratio of DNA (pg) to lipofectamine 2000 (μL) used for HEK293 and HeLa cells was 1:3 and 1:2.5 respectively. Appropriate volume of lipofectamine 2000 was taken and Opti-MEM was used to make final volume of 50 μL (24-well) or 250 μL (6-well) (lipo Opti-MEM mix). Lipofectamine 2000 Opti-MEM mix was incubated at room temperature for 5 minutes. Following incubation, the mix was added to DNA-OptiMEM mix and incubated for 15-20 minutes before adding it dropwise to the cells. Experiments shown in FIGS. 1-4, 8, 9, 11 were performed in HEK293 cells. FIGS. 4 and 11 also have data that was obtained from experiments on stably transduced HEK293 cells. Experiments shown in FIGS. 5, 6 and 12 were performed in HeLa cells.

To obtain the output expression values for FIGS. 1, 2, 8 and 9, 200 ng of appropriate plasmid was transfected with 50 ng of transfection control (pKH026, Ef1a-mCherry) and 50 ng of inducer plasmid (pMF206 CMV-PIT2 (Weber et al., 2002); pEL190 CMV-ET1 (Prochazka et al., 2014); pBA166 CMV-tTA (Clonetech, Cat #631070)) where required. In wells without transactivator, (‘no input’ condition), 50 ng of junk DNA (pBH265 (Haefliger et al., 2016)) was used.

To obtain the output expression values for the transient constructs in FIGS. 3, 4 & 11, 100 ng of appropriate plasmid (pJD139 or pJD145 or pJD140 or pJD80 or pJD83) was transfected with 50 ng of transfection control (pKH026, Ef1a-mCherry) and 50 ng of inducer plasmid (pMF206 CMV-PIT2; pEL190 CMV-ET1; pBA166 CMV-tTA) where required. To obtain the output expression values for the stable constructs in FIGS. 4 and 11, 50 ng of transfection control (pKH026, Ef1a-mCherry) was transfected with 50 ng of inducer plasmid (pMF206 CMV-PIT2; pEL190 CMV-ET1; pBA166 CMV-tTA) where required. For FIGS. 5, 12 and 6, 100 ng of appropriate plasmid (pJD219 or pJD198 or pJD178 or pJD204) was transfected with appropriate amount of inducer plasmid (5.5 ng of pJD70 CMV-PIT-VP16; 50 ng of pBA417 mCherry-TRE_{bidirectional}-SOX10 (Angelici et al., 2016); 22 ng of pEL190 CM-ET1; 50 ng of pEM003 mCherry-TRE_{bidirectional}-SOX10 (Angelici et al., 2016)) where required. Appropriate amount of junk DNA (pBH265) was added to keep constant the amount of DNA transfected across different input conditions in the above-mentioned experiments.

For RNA-sequencing experiment, 1000 ng of appropriate plasmid (pKB01 or pJD49) was transfected with 250 ng of transfection control (pKH026, Ef1a-mCherry) and 250 ng of inducer plasmid (pMF206 CMV-PIT2; pEL190 CMV-ET1; pBA166 CMV-tTA) where required. In wells without transactivator, (‘no input’ condition), 250 ng of junk DNA (pBH265) was used. 5 pmol of siRNA FF4 (Dharmacon) and 10 pmol of siRNA FF5 (Dharmacon) were added to the transfection mix where required. 5/10 pmol of miRIDIAN negative control #2 (Dharmacon, Cat #CN-002000-01-05) was added to keep the amount of siRNA constant across different input conditions. Sequences of siRNAs are mentioned in Table 2. siRNAs were added to the DNA-OptiMEM mix. Appropriate amounts of lipofectamine 2000 was added for siRNAs. For transfection of wells with siRNAs, pre-warmed fresh media was used to replace the existing media 12-15 hours post-transfection.

### Flow Cytometry

Cells were analyzed on flow cytometer 48 hours post transfection. HEK293 cells were prepared for flow cytometry by removing the media and supplying the cells with 1:1 mix of PBS 1×, pH 7.4 (ThermoFisher, Cat #10010-015) and Accutase (ThermoFisher, Cat #A11105-01) in a total volume of 100 μL, while HeLa cells were prepared by removing the media and supplying the cells with 100 μL of Accutase. The cells were incubated for 5-8 minutes at 37° C., 5% CO2. Cells were then re-suspended and transferred to micro-dilution tubes (Cat #02-1412-0000, Life Systems Design) which were kept on ice. Following this, cells were analyzed using BD LSR Fortessa II Cell Analyzer (BD Biosciences). The machine was calibrated with Sphero Rainbow Calibration Particles 8-peak beads (Spherotech, Cat #PCP-30-5A) prior to use. The excitation lasers (Ex) and emission filters (Em) used for respective fluorescent protein measurements are as follows: SBFP2 (Ex: 405 nm, Em: 445/20 nm), mCerulean/CFP (Ex: 445 nm, Em: 473/10 nm), mCitrine (Ex: 488 nm, Em: 530/11 nm, longpass filter 505 nm), and mCherry (Ex: 561 nm, Em: 610/20 nm, longpass filter 600 nm). Photo multiplier tube (PMT) voltage for different fluorescent channels were adjusted in a way that the mean values for 8-peak beads remained constant across different experiments. To provide a reference, measurement of polychromatic reporters and OR logic constructs was done at 200 mV for mCitrine, 220 mV for mCherry (transfection control), 250 mV for SBFP2 and 210 mV for CFP/mCerulean. In case of DNF-like logic testing, measurements were done at 230 mV for mCitrine, 225 mV for mCerulean (transfection control).

### Data Analysis

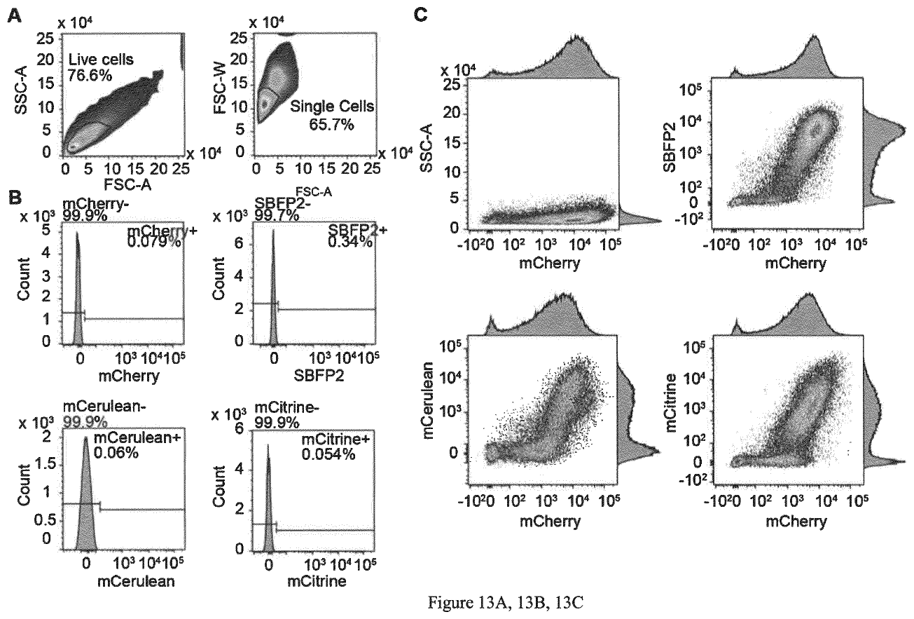

Flow cytometry data analysis for bar charts was performed using FlowJo software (BD Biosciences). In this work, the inventors used relative expression units and promoter normalized units for representing fluorescence values obtained from flow cytometry. Promoter normalized units are utilized in bar charts for polychromatic reporter cassettes. Relative expression units are utilized in bar charts for OR logic and DNF-like logic (AND-OR, AND-OR-NOT) constructs. The gating strategy performed using FlowJo is shown in FIG. 13.

The following steps are identical for calculating both metrics. (i) Live cells were gated based on forward scatter area vs side-scatter area plot. (ii) Within the live cells population, single cells were gated based on forward scatter area vs forward scatter width. (iii) To account for the cross-talk between fluorescent protein channels, a compensation matrix was defined based on the cells transfected individually with constitutively expressed fluorescent proteins—SBFP2, mCerulean, mCitrine and mCherry. The cross-talk from one fluorescent channel to the other was observed and manually compensated. The resultant matrix was then applied to all samples. (iv) Within the single cells population, cells positive for a given fluorescence channel were gated based on a negative control (non-transfected) sample such that 99.9% of the control cells fell outside of the selected gate. (v) For each positive cell population in a given fluorescence channel, Flowjo was used to calculate mean value of fluorescence and the frequency of positive cells. Multiplying these two values gives absolute intensity which is a direct measure of the fluorescent protein signal. (vi) The absolute intensity of a given fluorescent protein (Y) when normalized by the frequency of positive cells for transfection control in that sample gives relative expression units (rel. u.). It can be represented by the following formula:

$$Relative expression units ( rel . u . ) = \frac{frequency of positive cells for fluorescent protein Y \times mean of fluorescent protein Y}{freq uency of positive cells for transfection control}$$

Additional steps undertaken to calculate promoter normalized units are as follows: vii) To compare expression values of fluorescent proteins across different polychromatic reporter cassettes as well as to observe the actual effect of splicing, normalization of expression strength differences in fluorescent proteins arising from promoter strength and other regulatory features (5′-UTRs and ribosome binding site) was performed. To this end, a set of ‘control’ constructs (pKB02, pJD207, pJD209, pJD210, pKB03, pJD157, pJD208 and pKB04) were produced that were utilized for normalization (FIG. 8C). Fluorescent protein expression values were measured from cells transfected with these ‘control’ constructs. Further, relative expression units were obtained as mentioned above.
viii) Relative expression units for each polychromatic reporter cassette for each fluorescent protein was calculated as mentioned above. Promoter normalization units (norm. u.) were obtained as per the following formula:

$$Promoter norm . u . ( {P}_{n} ) = \frac{average of rel . u . of the polychromatic reporter cassette ( {R}_{a} )}{average of rel . u . of the ‘ control construct ’ ( {R}_{b} )}$$

In the above equation, the numerator and denominator both possess standard deviation values. Hence, error propagation was performed (https://www.eoas.ubc.ca/courses/eosc252/error-propagation-calculator-fj.htm) using the formula below. Let SDₐ and SD_{b} be the standard deviation values of Rₐ and R_{b}.

$$Standard deviation after error propagation = {P}_{n} \times \sqrt{{( \frac{{R}_{a}}{S {D}_{a}} )}^{2} + {( \frac{{R}_{b}}{S {D}_{b}} )}^{2}}$$

### Microscopy

Fluorescent protein expression was imaged using fluorescence microscopy at 48 hours post transfection. Images were acquired utilizing Nikon Eclipse Ti microscope equipped with a mechanized stage and temperature control chamber held at 37° C. The excitation light was generated by a Nikon IntensiLight C-HGFI mercury lamp or LED source and filtered through a set of optimized Semrock filter cubes. The resulting images were collected by a Hammamatsu, ORCA R2 or Flash4 camera using a 10× objective. The following optimal excitation (Ex), emission (Em) and dichroic (Dc) filter sets were used to minimize the cross-talk between different fluorescent channels mCitrine (Ex 500/24 nm or 513 nm LED with 20% intensity, Em 542/27 nm, Dc 520 nm), mCherry (Ex 562/40 nm, Em 624/40 nm, Dc 593 nm), CFP/mCerulean (Ex 438/24 or 438 nm LED with 20% intensity, Em 483/32 nm, Dc 458 nm) and SBFP2 (Ex 370/36 nm, Em 483/32 nm, Dc 458 nm). Exposure time, look-up tables (LUTs) and magnification for all experiments are indicated in the figure legends. Image processing for figure preparation was performed using Fiji software (http://imagej.net/).

### Lentivirus Production and Transduction

Lentivirus production protocol was adapted from Addgene (https://www.addgene.org/protocols/lentivirus-production/). HEK293T cells were seeded at 3.8*10⁶ cells per 60 cm² plate (Cat #93100, TPP) and incubated at 37° C., 5% CO₂ for ˜20 hours. DMEM supplemented with 10% FBS and no antibiotic was used in culturing cells for lentivirus production. After 20 hours, the media was gently aspirated and supplied with pre-warmed 10 mL media containing 10 μL of 25 mM chloroquine diphosphate (Sigma Aldrich, Cat #C6628-25G). The cells were incubated for 5 hours before replacing the media with no chloroquine diphosphate. DNA-Opti-MEM mix was prepared by mixing the following components: 15 pg of transfer plasmid (pJD163 or pJD164 or pJD165), 10 pg of pJD14, 2 pg of pJD15, 1 pg of pJD16 and final volume was made to 500 μL using Opti-MEM. On the side, 500 μL of PEI-Opti-MEM mix was prepared by adding 84 pg of PEI (Polysciences, Cat #24765-1) to Opti-MEM such that the DNA (μg): PEI (μg) ratio remained 1:3. Then, PEI-Opti-MEM mix was gently added dropwise to DNA-Opti-MEM mix and incubated at room temperature for 15-20 minutes. The transfection mix was added dropwise to the HEK293T packaging cells and incubated for 18 hours. Then, the media was gently aspirated and supplied with 15 mL pre-warmed fresh media. The lentivirus present in the supernatant (media) was harvested at 48 hours and the cells were supplied with 15 mL pre-warmed fresh media. The same was repeated at 72 hours post transfection. The lentiviral harvests from 48 and 72 hours were pooled together. The pooled lentivirus was centrifuged at 500×g for 5 minutes and then filtered using 0.45 μm filter (Sartorius, Cat #16555-K). The viral supernatant was loaded on Amicon Ultra-15 centrifugal filter units (MerckMillipore, Cat #UFC910096) for concentration and buffer exchange by following manufacturer's instructions. Lentivirus titration was performed using qPCR lentivirus complete titration kit (abm, Cat #LV900-S) by following manufacturer's instructions. Infectious units per mL (IU/mL) for the three lentiviruses are as follows: pJD163—1.21E+08 IU/mL, pJD164—1.26E+08 IU/mL and pJD165—1.89E+08 IU/mL. The virus was aliquoted (200 μL aliquots) and stored at −80° C.

For transduction, HEK293 cells were seeded at a density of 3*10⁵ cells per well in a 6-well plate (Cat #NC140675, ThermoFisher). Pre-thawed lentivirus (200 μL) was immediately added to the cells after seeding to get a MOI of 80, 84 and 126 respectively for lentivirus generated from constructs pJD163, pJD164 and pJD165. DMEM supplemented with 10% FBS and no antibiotic was used as media for the cells. Cells were cultured at 37° C., 5% CO2. The cells were split when required. Media with antibiotic was used once cells were split. The transduced, unsorted cells were seeded at 7.5*10⁴ cells per well for transfection and further analysis.

Transgene integrity following genomic integration was checked using PCR. Firstly, genomic DNA was extracted from the transduced and non-transduced cells using DNEasy Blood and Tissue kit (Qiagen, Cat #69504) following manufacturer's instructions. PCR was performed on the extracted genomic DNA (200 ng) samples using primers PR3163 and PR6129. The thermocycler program was as follows: 45 seconds at 98° C. for; 30 cycles of 10 seconds at 98° C., 30 seconds at 57° C., 2 minutes at 72° C.; 5 minutes at 72° C. The PCR product was loaded on 1% agarose gel for analysis. 40 ng of plasmids—pJD163, pJD164, pJD165 were used as templates for positive control and genomic DNA of non-transduced cells was used as a template for the negative control.

### RNA-Seq Sample Preparation and Data Analysis

Briefly, HEK293 cells were seeded in a 6-well plate at a density of 3.5*10⁵ cells per well. After 24 hours, the cells were transfected with 3-promoter polychromatic reporter (pKB01 or pJD49) with relevant inputs (PIT, ET, tTA). DNA amounts and other related information is in ‘Transfection’ section. Non-transfected cells were also included as a sample in the experiment. No biological replicate was made for this experiment. After 48 hours of transfection, the media from the wells was removed and the cells were detached from the well surface by supplying the cells with 1:1 mix of PBS 1×, pH 7.4 and Trypsin in a total volume of 500 μL. The cells were incubated for 5-8 minutes at 37° C., 5% CO2. Trypsin was inactivated by adding 500 μL media. The cells were re-suspended and counted for each sample. Equal number of cells (1.54*10⁶) were taken for each sample for cytoplasmic RNA extraction process. The extraction was performed using RNeasy Mini kit (Qiagen, Cat #74104) as per manufacturer's instructions. 100 mL RLN buffer was prepared for cytoplasmic RNA extraction by mixing the following components: 5 mL Tris·Cl pH 8.0 (AMResco, Cat #E199-500 ML), 0.81 g NaCl (Sigma Aldrich, Cat #53014-1KG), 3 mL of 50 mM MgCl₂ (Sigma Aldrich, Cat #13512), 2.5 mL IGEPAL CA-630 (10%). The buffer was filtered using 0.2μ filter (Sartorius, Cat #16534-K). For 10% IGEPAL CA-630 preparation, the IGEPAL bottle (Sigma Aldrich, Cat #I8896) was pre-warmed at 37° C. Then, 10 mL of 100% IGEPAL CA-630 was mixed with 90 mL ddH₂O. The dissolution was performed by vigorous mixing. On-column DNase digestion was also performed using RNase-free DNAse set kit (Qiagen, Cat #79254) according to manufacturer's instructions. Following RNA extraction, 100 ng of RNA for each sample was used for library preparation. The next-generation sequencing was performed by Microsynth AG (Switzerland). TruSeq stranded RNA library preparation method was used with polyA enrichment step. The 75 bp paired-end sequencing was performed on Illumina NextSeq platform to obtain (10+10) million reads per sample.

De-multiplexing of reads and trimming of Illumina adaptor residuals was performed by Microsynth. In order to draw conclusions from the RNA-seq data, and due to the fact that the sequences of the alternative transcript are extremely homologous in the exon regions that makes standard tools for transcript calling difficult to implement, the inventors developed a procedure for data analysis using MATLAB (Mathworks) scripts. Sequences of 50 nucleotides representing all possible unresolved exon-intron junctions (6 in total) and sequences representing correct splicing junctions (3 in total) were chosen for plasmids pKB01 and pJD49. Every sequence spanned 25 bases on either side of the junction. The fastq files were searched for reads that included these sequences in their entirety, and the total number of reads containing a junction were determined. These numbers were normalized to the total number of reads in each dataset to enable comparison between samples. Further, for every condition, all the counts mapped to the junctions were normalized such that their sum equals one. While the junctions are not mutually exclusive, this facilitates the comparison.

The following junction sequences were used for pKB01:

J1 (ExIa-Intron junction):
(SEQ ID NO: 219)
GAGCACCCAGAGCAAGCTGAGCAAGGTAAGCTGGCATCCCTTTGAGTCA
A
J2 (Intron-ExIb junction):
(SEQ ID NO: 220)
ATAATGGGGGCCAGAATTTTCAGGTGGTCCCTTGCTCGCTTTCTTTGCA
T;

The presence of these junctions in the transcript would imply the failure to remove the first intron.

J3 (ExIb-Intron junction):
(SEQ ID NO: 221)
GAGCACTCAGTCTGCACTTTCCAAGGTAATGGATGGGCTAGAGCCAATG
G
J4 (Intron-ExIc junction):
(SEQ ID NO: 222)
ATAATGGGGGCCAGACTGCCCGCCCCAAGCTCCTAGGAGCCACGGAGCT
G;

The presence of these junctions in the transcript would imply the failure to remove the second intron.

J5 (ExIc-Intron junction):
(SEQ ID NO: 223)
GAGCTACCAGTCCAAGCTGAGCAAGGTAGGTGTCTCCAAGATCCCCTTT
G
J6 (Intron-ExII junction):
(SEQ ID NO: 224)
TATCTGAGTGTATCTCTCCTCCCAGGATCCCAACGAGAAGAGAGATCAC
A;

The presence of J5 would imply the failure to splice the third intron, while the presence of J6 implies the failure to remove any of the three introns.

J7: ExIc-ExII junction:
(SEQ ID NO: 225)
GAGCTACCAGTCCAAGCTGAAG|GATCCCAACGAGAAGAGAGATCACA;

J7 indicates correct splicing of the third intron.

J8: ExIa-ExII junction:
(SEQ ID NO: 226)
AGCACCCAGAGCAAGCTGAGCAAG|
GATCCCAACGAGAAGAGAGATCACA;

J8 indicates correct splicing of the first intron.

J9: ExIb-ExII junction:
(SEQ ID NO: 227)
GAGCACTCAGTCTGCACTTTCCAAG|
GATCCCAACGAGAAGAGAGATCACA;

J9 indicates correct splicing of the second intron.

Corresponding junction sequences for pJD49 are as follows:

J1:
(SEQ ID NO: 228)
GAGCACCCAGAGCAAGCTGAGCAAGGTAAGCTGGCATCCCTTTGAGTCA
A;
(SEQ ID NO: 229)
J2: ATAATGGGGGCCAGAATTTTCAGGTGGTCCCTTGCTCGCTTTCTT
TGCAT;
J3:
(SEQ ID NO: 230)
GAGCACCCAGTCCAAGCTTTCGAAGGTAAGTATCTGCTAGAGCCAATGG
T;
J4:
(SEQ ID NO: 231)
ATAATGGGGGCCAGACTGCCCGCCCCAAGCTCCTAGGAGCCACGGAGCT
G;
J5:
(SEQ ID NO: 232)
GAGCTACCAGTCCAAGCTGAGCAAGGTAGACGTCTCCAAGATCCCCTTT
G;
J6:
(SEQ ID NO: 233)
TATCTGAGTGTATCTCTCCTCCCAGGATCCCAACGAGAAGAGAGATCAC
A;
J7:
(SEQ ID NO: 234)
GAGCTACCAGTCCAAGCTGAGCAAG|GATCCCAACGAGAAGAGAGATCA
CA;
J8:
(SEQ ID NO: 235)
GAGCACCCAGAGCAAGCTGAGCAAG|GATCCCAACGAGAAGAGAGATCA
CA;
J9:
(SEQ ID NO: 236)
GAGCACCCAGTCCAAGCTTTCGAAG|GATCCCAACGAGAAGAGAGATCA
CA

### Quantification and Statistical Analysis

Each biological replicate (n=3) for polychromatic reporter constructs (in FIGS. 1, 2, 3, 8, 9) was obtained from a separate experiment. Experiments for FIGS. 4, 5, 6, 11, 12 were at least repeated once. The number of biological replicates (n) used for the experiment is indicated in the corresponding figure legends. The data are plotted as a mean±SD as indicated.

The experimental results obtained with the disclosed DNA constructs are further described in the following Examples.

### Example 2. Alternative Promoter Based Multi-Input OR Circuits

In one example, which is not understood to be limiting, an alternative promoter based multi-input OR circuit comprises a number of individually-controlled promoter sequences, each with its own regulatory program, a Pol II binding site, and a transcriptional start site; every promoter transcribes an mRNA comprising a first exon unique to this promoter followed by a 5′-splice signal, intronic sequence, downstream promoter regions and alternative first exons, etc., until it reaches the shared second exon and transcription termination site. The transcriptional program of a promoter controls the production of mRNA. In addition, each mRNA isoform can also be controlled by its own post-transcriptional program directed towards an isoform-specific first exon sequence.

In this example, individual transcripts from a given promoter will only be generated at high levels if the transcriptional program at this promoter induces transcription, and the post-transcriptional program at the first exon is consistent with high transcript concentration, e.g. because the produced mRNA is not degraded or inhibited; in other words, if the outputs (high mRNA yield) of both programs are “On”, comprising AND logic at a single transcript level (FIG. 1A). Additional expression fine-tuning may be implemented at the translation stage, via modulating ribosomal binding sites of transcripts, etc. The “OR” logic relationship between regulatory programs of different transcripts can be hypothesized but not a priori assumed. The presence of alternatively-spliced mRNA variants might in principle, based on prior art knowledge only, also lead to mutual exclusion or mutual inhibition between transcripts, as implied by the word “alternative”. Mutual exclusion would imply “exclusive OR” (XOR), rather than OR, logic (Culler et al., 2010; Mathur et al., 2019).

Although several eukaryotic genes are regulated by alternative promoters in nature (Ayoubi and VanDeVen, 1996), this design principle has neither been assessed with respect to its regulatory logic, nor was it used as an inspiration for producing synthetic DNA constructs and/or designing DNA cassettes and/or synthetic gene circuits. It was thus a surprising finding that alternative promoters and/or alternative splicing could be employed to generate DNA constructs that function as OR gates and/or as normal form logic circuits, as described in more detail in the following Examples.

### Example 3. Polychromatic Reporter System to Visualize and Fine-Tune Multiple Alternative Promoter Architectures

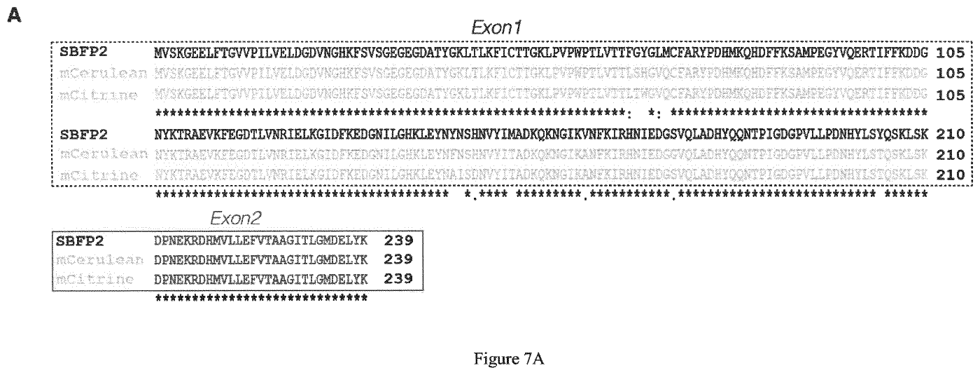

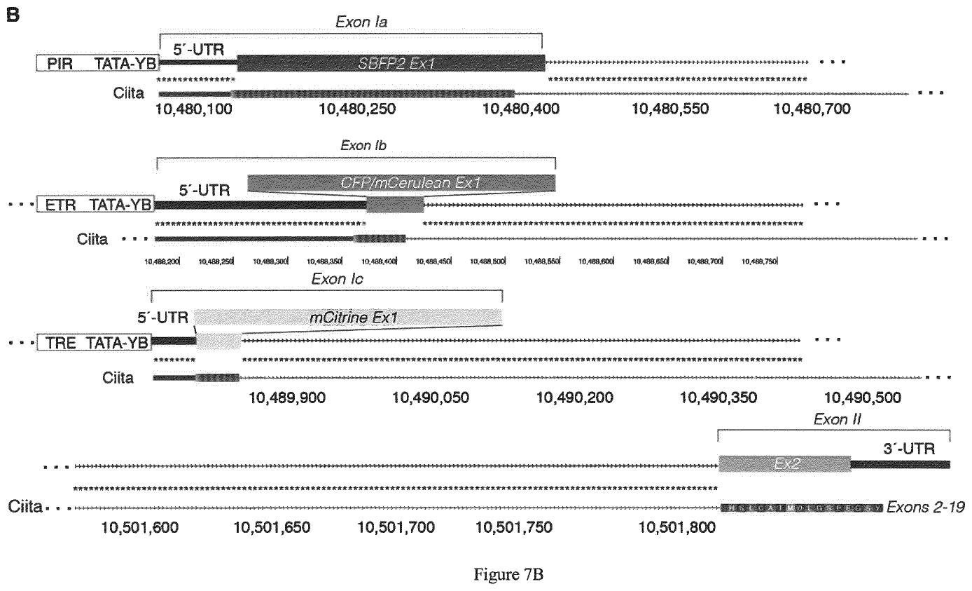

The inventors created a genetic scaffold modelled based on the mouse Ciita gene described in Example 1. The inventors utilized GFP-derived fluorescent proteins (SBFP2, CFP/mCerulean and mCitrine), all slight variations of each other and identical at their C-terminal (29 amino acids). The C-terminal sequence formed the second (shared) exon (FIG. 7A), while the 210 N-terminal amino acid sequences of each protein form the unique first exons. The alternative first exons were driven by inducible promoters, PIR, ETR and TRE, regulated respectively by the transactivators PIT2, as described in Example 1 (Fussenegger et al., 2000), ET (Weber et al., 2002) and tTA (Boger and Gruss, 1999). The architecture of the mouse Ciita locus was followed where possible, according to the “minigene” approach (Gaildrat et al., 2010): the 5′-UTR of the alternative first exons were identical to the 5′-UTR of the alternative Ciita exons. Further, the intronic sequences following the alternative first exons were identical to anywhere between 250 and 450 base pairs of the corresponding genomic sequence of the Ciita gene; and the 3′-region, shared to all introns, was likewise identical to the 200 base pairs of genomic sequence upstream of the second (shared) exon of the Ciita gene (FIG. 7B). For transcription termination, the rabbit ß-globin polyadenylation signal (Gil and Proudfoot, 1987) was used in the 3′-UTR instead of the ˜2,000 nt-long 3′-UTR of the Ciita gene due to practical reasons and to avoid cryptic effects that might result from multiple polyadenylation sites.

Upon activation of different promoters by their cognate transactivators, the DNA cassette is expected to express SBFP2 upon PIR induction by PIT2, CFP (or mCerulean) upon ETR induction by ET, and mCitrine upon TRE induction by tTA (FIG. 1B). Simultaneous induction by more than one transactivator should result in co-expression of different fluorescent proteins, as expected from the OR-like behavior. An in-frame stop codon was present in every intron to ensure lack of fluorescent protein expression upon mis-splicing. Eight different input combinations corresponding to all possible subsets of the three transactivators were tested to evaluate circuit response. A number of control experiments were performed to confirm the requirement for fully-functional splicing for fluorescent output expression by the polychromatic constructs that are used for optimizing the construct/cassette design, e.g. the splice sites. Of note, however, other constructs disclosed herein may not require fully-functional splicing or splicing at all (i.e. non-splicing DNA constructs), e.g. when they do not contain a first and a second exon but merely a common output sequence (e.g. one common exon).

In context of the polychromatic constructs, first, each fluorescent output was properly expressed when cloned individually with an intervening intron whose 5′- and 3′-splicing sequences are identical to a three-promoter construct, while no fluorescence was observed in constructs lacking the 3′-splicing signal and the second exon (FIG. 8A). Second, no activator-dependent induction of any fluorescent reporter was observed when a second exon was mutated in a three-promoter cassette (FIG. 8B). Hence, correct splicing was necessary and sufficient, in this context, to observe output fluorescence for all three proteins, consistent with the fact that GFP can tolerate truncation of only up to 15 amino acids at the C-terminus without disrupting fluorescence (Li et al., 1997). In order to disentangle the effects of splicing from the intrinsic differences between promoter strength and translation-affecting features such as ribosome binding site, the inventors measured the expression levels of intron-less fluorescent proteins from these promoters and used these values for normalization purposes, as described in Example 1 (FIG. 8C). The reported “promoter-normalized” units, whereby the expression of a fluorescent output is normalized by the expression of the output from the same promoter in the absence of splicing, highlights differences in expression that are mainly due to the presence of multiple promoters and alternative splicing (see Example 1).

The performance criteria for the polychromatic circuit were as follows: i) upon single-input promoter activation, the fluorescent protein output expression levels obtained should preferably be close to the expression generated with promoter-reporter cassettes in the absence of splicing (the latter corresponding to 1 promoter-normalized unit) while eliciting minimal concurrent activation of other fluorescent outputs; and ii) avoiding additive expression upon multiple promoter activation. In addition, the inventors strove to ensure strong absolute expression of all outputs. The inventors hypothesized that several design elements could affect the performance: i) the strength of the 5′-splice site sequences, due to the competition between these sites in the course of alternative splicing; ii) 3′-splice site, polypyrimidine tract and 3′-UTR sequences; iii) intron sequence, in particular the presence of splicing enhancer/silencer sequences; iv) promoter type and sequence, and distance between promoters, due to the fact that adjacent promoters may both enhance and inhibit each other via long-range interactions that are not related to splicing; v) exonic sequence affecting transcript stability and translational initiation efficiency and thus influencing absolute output expression. Note that the polychromatic reporter system is, in strictu sensu, not a bona fide OR gate as it generates multiple outputs. It serves mainly as a model system to investigate design variables, to be implemented in the next step of building bonafide OR logic circuit with a single output protein. The above criteria and their modulation methods notwithstanding, the actual desired performance specification may vary depending on the application and a reporter system will behave differently from an application-relevant cassette. Because specific applications of OR gates may address heterogenous cell populations, the desired output expression in different cell types may not necessarily be identical. The results that follow show a number of trends that can be used as guidelines to achieve desired performance goals. Based on the guidelines provided herein and further common general knowledge, the person skilled in the art can routinely generate and/or modify DNA constructs/cassettes such that they have the desired properties described herein. Thus, a construct/cassette described herein which has not immediately any or all of the desired properties can easily be modified to have the desired properties based on the present disclosure and further common general knowledge.

For example, the initial three-promoter cassette was only able to express mCitrine following TRE induction in HEK293 cells (FIG. 1C, pKB01). Induction of PIR or ETR failed to generate significant levels of SBFP2 and CFP, although the induction of PIR alongside TRE reduced the expression of mCitrine by three-fold, suggesting that the different transcriptional frames interfered with each other. Neither SBFP2 nor CFP were expressed despite following Ciita gene architecture, possibly because (i) the intronic sequences in the native Ciita were much longer and/or (ii) cell-type specific trans factors that aid in proper splicing of Ciita gene in hematopoietic lineages were absent in HEK293 cells. The inventors contemplated that the 5′-splice site downstream of mCitrine was too strong, preventing the engagement of the upstream splice sites. The strength of 5′-splice sites was estimated using MaxEnt scan tool (Yeo and Burge, 2004) with various supported models to generate a compound score (Table 5; a higher score corresponds to a stronger site. By weakening the 5′-splice site of the third intron downstream of the mCitrine first exon to varying degrees, the inventors observed correct splicing of the PIT-driven transcript resulting in SBFP2 expression (FIG. 8D, pKW17 and pJD23; FIG. 9 pJD232, pJD233). However, transcriptional interference were still observed and CFP expression remained very low. Thus, the inventors replaced CFP with brighter mCerulean (Rizzo and Piston, 2005), introduced Kozak sequence (Kozak, 1986) in the first exon of mCerulean, and strengthened the 5′-splice site of the intron downstream of this exon. These modifications resulted in cassettes that generated significant expression of every output, qualitatively consistent with the predicted expression but at quantitatively non-uniform levels (FIG. 1D, pJD31; FIG. 8E, pJD30). This already illustrates how initially suboptimal constructs can be improved, e.g. by modifying the various 5′-splice site strengths and/or adding Kozak sequence(s).

Interestingly, in pKB01 the 5′-splice sites of the first and the third intron (ss8 and ss4 in Table 5, corresponding to SBFP2 and mCitrine outputs) had similar splicing ‘capacity’, and yet SBFP2 failed to splice at all, presumably due to the longer distance to the 3′-splice site, agreeing with earlier observations that the proximal 5′-splice site is chosen among two alternative 5′-splice sites of similar strength (Eperon et al., 1993). In the present example, despite transcription from the PIR promoter, the third (mCitrine) 5′-splice site was engaged, resulting in mRNA that can translate neither SBFP2 nor mCitrine (see FIG. 2D). Thus, in pJD31, the 5′-splice signal of the third intron (ss3 in Table 5) was weakened by around 1.5-fold, which allowed SBFP2 to splice at a comparable level to mCitrine while reducing mCitrine expression by a three-fold, consistent with the observation that a distant 5′-site can be chosen when the proximal site is weak (Eperon et al., 1993).

The reduction in mCitrine expression, when PIR promoter was activated alongside TRE, occurred regardless of the expression of the SBFP2 protein, and therefore it was not due to translational burden (Ceroni et al., 2018). Neither is it likely to be splicing-related but rather a manifestation of transcriptional interference from an upstream toward a downstream promoter (Eszterhas et al., 2002). In fact, however, such inhibitory interference is advantageous in the context of an OR gate as it prevents additive output expression upon simultaneous multi-input activation. The inventors also noted that increasing the strength of acceptor splice site did not significantly change the expression levels (FIG. 9, pJD42).

TABLE 5
Scoring of 5′-splice sites using MaxEntScan tool (Yeo and Burge, 2004). A 5′-splice
site sequence comprises 9 bases (three exonic bases followed by six intronic bases).

| | | Maximum | Maximum | | | |
|---|---|---|---|---|---|---|
| | 5′-splice | Entropy | Decomposition | Markov | Weight | Compound |
| Code* | site sequence | Model | Model | Model | Matrix Model | Score |
| ss6 | AAGGTAAGT | 11 | 15.48 | 12.19 | 12.71 | 12.8 |
| ss4 | AAGGTAAGC | 10.22 | 16.08 | 10.62 | 11.48 | 12.1 |
| ss7 | AAGGTAAGA | 10.57 | 15.58 | 10.55 | 11.36 | 12.0 |
| ss9 | AAGGTAAGAATCTGC | 10.57 | 15.58 | 10.55 | 11.36 | 12.0 |
| ss8 | AAGGTAGGT | 10.29 | 13.98 | 10.17 | 10.33 | 11.2 |
| ss18 | GAGGTAAGC | 9.85 | 13.88 | 10.2 | 10.64 | 11.1 |
| ss10 | AAGGTATGT | 9.79 | 13.48 | 10.03 | 9.76 | 10.8 |
| ss15 | AAGGTAGGC | 10.08 | 14.18 | 8.6 | 9.11 | 10.5 |
| ss11 | AAGGTGGGT | 8.23 | 13.78 | 7.98 | 8.95 | 9.7 |
| ss17 | AAGGTAATG | 8.99 | 12.98 | 7.42 | 7.87 | 9.3 |
| ss19 | GCGGTAGGC | 7.2 | 10.58 | 7.7 | 5.93 | 7.9 |
| ss12 | AAGGTTTGT | 7.81 | 11.58 | 6.11 | 4.81 | 7.6 |
| ss3 | AAGGTAGAC | 7.07 | 10.88 | 5.3 | 5.66 | 7.2 |
| ss2 | AAAGTAATG | 1.16 | 6.88 | 3.74 | 4.7 | 4.1 |
| ss5 | AAGGTGATC | 4.41 | 9.28 | 5.14 | 6.20 | 6.3 |
| ss20 | AAGGTGCCC | 5.96 | 8.38 | 4.71 | 3.28 | 5.6 |
| ss1 | AAGGTCTCC | 0.87 | 5.28 | 1.42 | 0.18 | 1.9 |

*Refer to FIG. 10 for its usage in different cassettes.

To further evaluate multiple splice site configurations, the inventors mapped the design space by varying the sequences of different modules (FIG. 10A, Table 1). The initial challenge was to increase the expression of SBFP2 that went down once mCerulean exon was furnished with stronger splice site (e.g., compare pJD23 to pJD31 in FIG. 9). The inventors sought to achieve this by increasing the strength of SBFP2 5′-splice site, weakening the 5′-splice site of mCerulean (FIGS. 2A and 9, pJD35-37, pJD40, pJD53-55), adding a canonical Kozak sequence in front of SBFP2 exon and modifying its 5′-UTR (FIG. 2A, pJD47-49), including an intron splicing enhancer (Wang et al., 2012) close to SBFP2 donor splice site (FIG. 9, pJD52). In this small library, cassettes pJD47-49 and pJD53 fulfilled performance criteria better than others (see FIG. 2B for visualization of pJD48 performance). The increase in SBFP2 by weakening of mCerulean donor splice site was only observed with very strong disparity between the sites, as in pJD55, which, however, led to weak mCerulean expression.

This further demonstrates how the DNA constructs can be further optimized, e.g. by further modifying the 5′-splice sites, and/or introducing splicing enhancers.

Additional manipulations did not result in substantial improvement in performance. For example, using identical intron and 5′-splice signal downstream of mCerulean and SBFP2 first exons (FIG. 9, pJD154) did not affect SBFP2 while increasing both mCitrine and mCerulean. A systematic increase of the 5′-splice signal strength downstream of mCitrine exon resulted in decrease of both SBFP2 and mCerulean, consistent with the observation made with pKB01 (FIG. 9, pJD115, pJD125, pJD127, pJD128). Lastly, embedding a splicing enhancer (Miyaso et al., 2003) in the intron downstream of SBFP2 exon did not result in rescue of its expression (pJD125, FIG. 9).

Among the various explored parameters, the absolute strength of the 5′-splicing signal downstream of the mCitrine exon had the most impact on the output levels. A trend is observed whereby decreasing the strength of this site results in increase of expression from both the first and the second promoters, and a decrease in expression from the third promoter (FIG. 2C). Since this is an important parameter, the inventors checked to which extent this weakening can sustain while fulfilling the performance goals. The inventors reduced the strength of donor splice site downstream of mCitrine exon in pJD48 from 7.2 to 6.3 and 1.9. The reduction to 6.3 resulted in SBFP2 levels close to ‘1’ (pJD235, FIG. 9), with only slight reduction in mCitrine. However, weakening to 1.9 (pJD237, FIG. 9) drastically reduced mCitrine expression. Thus, weakening to this extent was not favorable in view of the performance goals. In order to further understand the different processes at play, the inventors performed deep sequencing of the transcriptome from pKB01 and the optimized pJD49 and assessed the presence of unspliced exon-intron junctions as well as properly-spliced junctions. In brief (FIG. 2D), deep sequencing data confirmed that PIT or ET activation in pKB01 resulted in transcription from these promoters but did not lead to the expected splicing. Instead, the third intron was spliced, resulting in a properly formed mCitrine coding sequence that nevertheless failed to translate due to its long distance from the mRNA cap and the presence of in-frame stop codon in the preceding introns. The weakening of the third exon's 5′-splice site in pJD49 resulted in less efficient processing of the third intron, and was accompanied by the increase in the correct splicing of the first and second introns. Of note, even in pJD49, failed splicing comprised a substantial portion of the transcriptome, but it is unclear if some of the nuclear pre-mRNA was carried over to the sample despite efforts to isolate cytoplasmic mRNA. Be it as it may, incomplete splicing is not a problem for the functionality of the DNA constructs, and may only, at most, somewhat reduce the amount of the desired output protein, because these unspliced mRNAs do not generate functional proteins. Furthermore, data in FIG. 2D should only be interpreted in relative terms (pKB01 vs. pJD49) because the absolute mapping frequency was highly sequence-dependent and cannot be used to make any claims about absolute abundance.

### Example 4. Implementing OR Logic Using Alternative Promoter Architecture

Towards constructing OR logic gates, the inventors first reduced the system to two promoters. The initial two-color cassette was obtained from pJD49 (FIG. 2A) by removing the TRE promoter and its downstream exon, as well as part of the intron associated with it (FIG. 3A). The proximal 5′-splice site was stronger than the distal site, and there was a two-fold decrease in the expression from the first promoter in comparison to pJD49 (FIG. 3B). Additionally, SBFP2 expression decreased by another 1.6-fold when ET promoter was activated together with PIT. As a side remark, weak expression of mCerulean was now observed upon PIR activation, something that did not happen in the three-promoter progenitor cassette described in Example 3. In order to reduce cassette size and make it even more suitable for viral vector packaging, 220 bp were removed from the first intron upstream of the ETR promoter, and about 500 bp removed from the second intron, given that introns as short as ˜65 bases can be still efficiently spliced (Piovesan et al., 2015; Sasaki-Haraguchi et al., 2012). The short introns still possessed in-frame stop codons in case mis-spliced or unspliced transcripts are formed. Furthermore, in a bid to increase SBFP2 expression, instead of weakening the proximal splice site the inventors introduced an intron splicing enhancer sequence (Wang et al., 2012) downstream of the distal 5′-splice site. These modifications resulted in around four-fold increase in SBFP2 expression with the leakiness of mCerulean expression increasing around 1.6-fold (FIG. 3C) and an overall more balanced expression of two outputs. Of note, in this context the activation of the PIR promoter resulted in increased expression from the ETR promoter compared to the ETR alone, contrary to the previously-observed negative influence. The inventors think that this has to do with transcriptional synergy between the two promoters (Angelici et al., 2016) due to reduced distance between promoters, rather than with splicing-related effects. The person skilled in the art can readily select the promoters and their positions in a way that no or only little synergy occurs, based on the design guidelines provided herein, and further common general knowledge, e.g. by increasing the distance between the promoters. Next, the inventors adapted the architecture to a bona fide OR-gate, generating the same functional output from both promoters. The inventors opted for alternative coding first exons, split by introns in the middle of a codon, thereby requiring proper splicing for in-frame translation. However, it is also possible to utilize non-coding exons instead as alternative 5′-UTRs, albeit this might lead to protein translation from the second exon even when the splicing fails. Going back to the genomic template of Ciita gene, the inventors used the coding sequence from its alternative first exons. The second exon included the remaining bases of a split codon, followed by a GS linker, and an mCitrine coding sequence. While this two-input OR gate was based on pJD145 (FIG. 3C), the structure of the exons was changed substantially, with the second (shared) exon becoming much longer and the alternative first exons becoming much shorter, altering the distances between the splicing signals as well as between the promoters. The schematics of the relationship between the two cassettes is shown in FIG. 11A.

The resulting OR-gate construct was evaluated in two configurations, transient transfection and stable integration via a lentiviral transduction (FIG. 4A). In order to construct the latter, the construct was embedded in an inverse orientation in relation to lentiviral transfer plasmid, to avoid interference with the viral mRNA transcription process (Poling et al., 2017). Analysis of the stable integrants showed that the original construct was intact (FIG. 11B) post-integration. The inducer plasmids were transfected in stable setup as well. The flow cytometry data and the fluorescent micrographs revealed that the two-input OR circuit was qualitatively similar in transient and stable versions (FIG. 4A), consistent with the OR logic behavior. For the stably-integrated circuit, the mean expression values remained consistent across different input conditions as seen in the histogram of mCitrine positive population. The variation in percentage of positive cells in the stable setup can be attributed to the usage of unsorted cells and differences in the promoter strength. Note that the normalization for promoter strength could not be performed in this case because there was a single output protein. Another two-input construct using PIT and tTA inputs was engineered and characterized, generating qualitatively similar data (FIG. 11C-E). Overall, two constructs using different promoter combinations have been shown to behave in a similar fashion, indicating the feasibility of this approach to implement OR logic in transient and stable settings.

Lastly, the inventors scaled the system up to three inputs (FIG. 4B). Overall, the data were also consistent with the OR logic despite some variability. In both transient and stable setting, the peak mCitrine values under multi-input conditions did not exceed the peak values with single input condition, comprising a non-additive OR-like response. Remarkably, mean output levels in cells bearing the stably integrated cassette were very consistent, varying within only about two-fold range. In summary, this dataset further supports the multi-promoter architecture as the basis for OR-logic both on plasmids and lentiviral vectors. Since the normalization by promoter strength was no longer possible, the balance of promoter strength becomes more important for obtaining more uniform outputs. Additionally, the precise output levels also depend on the complex interplay between alternative splicing, transcriptional interference, as well as synergy between neighboring promoters, especially as the intron sizes and mutual distances are reduced. Based on the design principles disclosed herein, satisfactory OR-like response can be achieved.

### Example 5. Normal Form Logic Circuits

In the next step, the inventors asked if the basic OR architecture described in Example 5 could be expanded to perform complex Boolean logic as envisioned in FIG. 1A and Example 2. For the inputs to the transcriptional program, the inventors relied on the AND-gate approach developed earlier (Angelici et al., 2016). The inventors coupled this approach with the OR architecture and designed a disjunctive normal form-like (AND-OR) logic circuit (FIG. 5A). As both PIT and ET inputs were shown to function as AND-gate inputs on individual promoters under certain design constraints (Angelici et al., 2016), the inventors converted single-input PIR and ETR promoters from FIG. 4A to two-input AND gates using SOX10 and HNF1A transcription factor (TF) inputs, respectively. For this, the inventors cloned SOX10 binding sites downstream of PIR, and HNF1A/B sites downstream of ETR. The number of ET binding sites was reduced from three to one. Accordingly, the logic program of this circuit was (PIT AND SOX10) OR (ET AND HNF1A) (FIG. 5B). To increase On-to-Off ratio, PIT2 was replaced with PIT-VP16 (Angelici et al., 2016). All possible input combinations were provided in trans either from constitutive promoters (PIT and ET) or tTA-induced promoters (SOX10 and HNF1A), and circuit response measured in HeLa cells that expressed neither SOX10 nor HNF1A/B. In the initial construct containing short introns, either input to the first promoter (PIT or SOX10) showed synergistic activation with either input to the second promoter (ET or HNF1A). The activation was strongest with PIT+HNF1A combination, making it the worst case OFF state in this Example (FIG. 12A), with a synergy score (Angelici et al., 2016) of 3.3. To counteract this problem, the distance between the two promoters was increased by adding intronic sequence of 200 bases, reducing the synergy score to 2.4 (FIG. 12B). Additionally, reducing PIT binding sites from 3 to 2 brought the synergy to 1.6. Qualitatively, the response of the fine-tuned circuit (FIG. 5C) was consistent with the logic truth table. However, there was variability among the ON and the OFF outputs, with the median dynamic range of about 22-fold, and the worst case of 6.6-fold (FIG. 5C). As a side note, for input combination HNF1A+ET+SOX10, the output was reduced in comparison to ET+HNF1A and the opposite was observed when PIT was added in the presence of ET and HNF1A. Although, the presence of TF inputs SOX10 or HNF1A had an impact on the output levels of the opposite promoter branches, the constructs achieved a satisfactory normal form logic-like behavior. Further fine tuning may be performed based on the design principles disclosed herein, in particular with respect to the intron length and individual AND promoter composition, to balance the various effects and further improve the normal form logic performance.

Lastly, the inventors explored whether the logic control of each individual transcript of the OR gate could be extended by NOT operations at post-transcriptional level (FIG. 6A), for example, via microRNA inputs and RNA interference (RNAi) pathway. It was contemplated that the unique 5′-UTRs of the first and second promoter-driven transcripts could serve as access points for transcript-specific RNAi; for example, it was shown that microRNA were able to regulate gene expression via target sites in their 5′-UTRs (Lytle et al., 2007). To analyse this possibility, the 5′-UTR of the first transcript was augmented with the target sites for an artificial siRNA-FF4 and the second with the target sites for siRNA-FF5 (Leisner et al., 2010). The full circuit (FIG. 6B) received six inputs, corresponding to 64 possible input combinations. Instead of testing all the 64 input combinations, the inventors tested the most informative ones focusing on the siRNA contribution to circuit response. siRNA inputs (four combinations) were superimposed on the transcriptional input combinations that resulted in none, one, or both activated promoter branches (four conditions), resulting in the total of 16 input sets. mCitrine expression (FIG. 6C) patterns agreed qualitatively with the truth table. When the promoter of one of the transcripts was activated but the siRNA input targets another transcript, there was no effect on output expression. However, there was clear knockdown of output expression when the siRNA input targeted the transcript generated by its designated promoter branch. The effect was particularly strong in case of siRNA-FF4 (condition 9 vs condition 10, 10× difference), and somewhat less so in the case of siRNA-FF5 (condition 5 vs condition 7, 2.5-3× difference). When both transcripts were activated but only one of the siRNA inputs was present, the output was strongly expressed (condition 14 and 15), as expected from the logic formula and/or the truth table, and only when both siRNA inputs were present did the output get repressed (condition 16). However, the lower efficiency of siRNA-FF5 acted as a bottleneck that prevented achieving more substantial knockdown. Nonetheless, repression via 5′-UTR can be efficient as exemplified by the strong effect of siRNA-FF4, showing a clear path to further optimization, e.g. by further improving the siRNA binding efficiency.

### REFERENCES

The following detailed references relate to the short references indicated herein:
Angelici, B., Mailand, E., Haefliger, B., and Benenson, Y. (2016). Synthetic biology platform for sensing and integrating endogenous transcriptional inputs in mammalian cells. Cell Reports 16, 2525-2537.
Ayoubi, T. A. Y., and VanDeVen, W. J. M. (1996). Regulation of gene expression by alternative promoters. Faseb Journal 10, 453-460.
Bashor, C. J., Patel, N., Choubey, S., Beyzavi, A., Kondev, J., Collins, J. J., and Khalil, A. S. (2019). Complex signal processing in synthetic gene circuits using cooperative regulatory assemblies. Science 364, 593-+.
Benenson, Y. (2012). Biomolecular computing systems: principles, progress and potential. Nature Reviews Genetics 13, 455-468.
Bindels, D. S., Haarbosch, L., van Weeren, L., Postma, M., Wieser, K. E., Mastop, M., Aumonier, S., Gotthard, G., Royant, A., Hink, M. A., et al. (2017). mScarlet: a bright monomeric red fluorescent protein for cellular imaging. Nature Methods 14, 53-56.
Boger, H., and Gruss, P. (1999). Functional determinants for the tetracycline-dependent transactivator tTA in transgenic mouse embryos. Mechanisms of Development 83, 141-153.
Buchler, N. E., Gerland, U., and Hwa, T. (2003). On schemes of combinatorial transcription logic. Proceedings of the National Academy of Sciences of the United States of America 100, 5136-5141.
Ceroni, F., Boo, A., Furini, S., Gorochowski, T. E., Borkowski, O., Ladak, Y. N., Awan, A. R., Gilbert, C., Stan, G. B., and Ellis, T. (2018). Burden-driven feedback control of gene expression. Nature Methods 15, 387-+.
Cox, R. S., Surette, M. G., and Elowitz, M. B. (2007). Programming gene expression with combinatorial promoters. Molecular Systems Biology 3, 11.
Culler, S. J., Hoff, K. G., and Smolke, C. D. (2010). Reprogramming Cellular Behavior with RNA Controllers Responsive to Endogenous Proteins. Science 330, 1251-1255.
Donahue, P. S., Draut, J. W., Muldoon, J. J., Edelstein, H. I., Bagheri, N., and Leonard, J. N. (2020). The COMET toolkit for composing customizable genetic programs in mammalian cells. Nature Communications 11.
Dull, T., Zufferey, R., Kelly, M., Mandel, R. J., Nguyen, M., Trono, D., and Naldini, L. (1998). A third-generation lentivirus vector with a conditional packaging system. Journal of Virology 72, 8463-8471.
Elowitz, M. B., and Leibler, S. (2000). A synthetic oscillatory network of transcriptional regulators. Nature 403, 335-338.
Eperon, I. C., Ireland, D. C., Smith, R. A., Mayeda, A., and Kramer, A. R. (1993). Pathways for selection of 5′ splice sites by U1 SNRNPS and SF2/ASF. Embo Journal 12, 3607-3617.
Eszterhas, S. K., Bouhassira, E. E., Martin, D. I. K., and Fiering, S. (2002). Transcriptional interference by independently regulated genes occurs in any relative arrangement of the genes and is influenced by chromosomal integration position. Molecular and Cellular Biology 22, 469-479.
Fussenegger, M., Morris, R. P., Fux, C., Rimann, M., von Stockar, B., Thompson, C. J., and Bailey, J. E. (2000). Streptogramin-based gene regulation systems for mammalian cells. Nature Biotechnology 18, 1203-1208.
Gaildrat, P., Killian, A., Martins, A., Toumier, I., Frebourg, T., and Tosi, M. (2010). Use of splicing reporter minigene assay to evaluate the effect on splicing of unclassified genetic variants. In Cancer Susceptibility: Methods and Protocols, M. Webb, ed. (Totowa: Humana Press Inc), pp. 249-257.
Gam, J. J., Babb, J., and Weiss, R. (2018). A mixed antagonistic/synergistic miRNA repression model enables accurate predictions of multi-input miRNA sensor activity (vol 9, 2430, 2018). Nature Communications 9.
Gao, X. J., Chong, L. S., Kim, M. S., and Elowitz, M. B. (2018). Programmable protein circuits in living cells. Science 361, 1252-1258.
Gardner, T. S., Cantor, C. R., and Collins, J. J. (2000). Construction of a genetic toggle switch in Escherichia coli. Nature 403, 339-342.
Gibson, D. G., Young, L., Chuang, R. Y., Venter, J. C., Hutchison, C. A., and Smith, H. O. (2009). Enzymatic assembly of DNA molecules up to several hundred kilobases. Nature Methods 6, 343-U341.
Gil, A., and Proudfoot, N.J. (1987). Position-dependent sequence elements downstream of AAUAAA are required for efficient rabbit beta-globin messenger-ma 3′ end formation. Cell 49, 399-406.
Green, A. A., Kim, J. M., Ma, D., Ilver, P. A. S., Collins, J. J., and Yin, P. (2017). Complex cellular logic computation using ribocomputing devices. Nature 548, 117-+.
Haefliger, B., Prochazka, L., Angelici, B., and Benenson, Y. (2016). Precision multidimensional assay for high-throughput microRNA drug discovery. Nature Communications 7, 12.
Ham, T. S., Lee, S. K., Keasling, J. D., and Arkin, A. P. (2008). Design and Construction of a Double Inversion Recombination Switch for Heritable Sequential Genetic Memory. Plos One 3, 9.
Kozak, M. (1986). Point mutations define a sequence flanking the AUG initiator codon that modulates translation by eukaryotic ribosomes. Cell 44, 283-292.
Kramer, B. P., Fischer, C., and Fussenegger, M. (2004). BioLogic gates enable logical transcription control in mammalian cells. Biotechnology and Bioengineering 87, 478-484.
Lapique, N., and Benenson, Y. (2018). Genetic programs can be compressed and autonomously decompressed in live cells. Nature Nanotechnology 13, 309-315.
Leisner, M., Bleris, L., Lohmueller, J., Xie, Z., and Benenson, Y. (2010). Rationally designed logic integration of regulatory signals in mammalian cells. Nature Nanotechnology 5, 666-670.
Li, X. Q., Zhang, G. H., Ngo, N., Zhao, X. N., Kain, S. R., and Huang, C. C. (1997). Deletions of the Aequorea victoria green fluorescent protein define the minimal domain required for fluorescence. Journal of Biological Chemistry 272, 28545-28549.
Lin, Y. S., Carey, M., Ptashne, M., and Green, M. R. (1990). How different eukaryotic transcriptional activators can cooperate promiscuously. Nature 345, 359-361.
Lohmueller, J. J., Armel, T. Z., and Silver, P. A. (2012). A tunable zinc finger-based framework for Boolean logic computation in mammalian cells. Nucleic Acids Research 40, 5180-5187.
Lois, C., Hong, E. J., Pease, S., Brown, E. J., and Baltimore, D. (2002). Germline transmission and tissue-specific expression of transgenes delivered by lentiviral vectors. Science 295, 868-872.
Lytle, J. R., Yario, T. A., and Steitz, J. A. (2007). Target mRNAs are repressed as efficiently by microRNA-binding sites in the 5′ UTR as in the 3′ UTR. Proceedings of the National Academy of Sciences of the United States of America 104, 9667-9672.
Mathur, M., Kim, C. M., Munro, S. A., Rudina, S. S., Sawyen, E. M., and Smolke, C. D. (2019). Programmable mutually exclusive alternative splicing for generating RNA and protein diversity. Nature Communications 10, 13.
Misirly, G., Nguyen, T., McLaughlin, J. A., Vaidyanathan, P., Jones, T. S., Densmore, D., Myers, C., and Wipat, A. (2019). A Computational Workflow for the Automated Generation of Models of Genetic Designs. Acs Synthetic Biology 8, 1548-1559.
Miyaso, H., Okumura, M., Kondo, S., Higashide, S., Miyajima, H., and Imaizumi, K. (2003). An intronic splicing enhancer element in survival motor neuron (SMN) pre-mRNA. Journal of Biological Chemistry 278, 15825-15831.
Mohammadi, P., Beerenwinkel, N., and Benenson, Y. (2017). Automated design of synthetic cell classifier circuits using a two-step optimization strategy. Cell Systems 4, 207-218.
Muhlethaler-Mottet, A., Otten, L. A., Steimle, V., and Mach, B. (1997). Expression of MHC class II molecules in different cellular and functional compartments is controlled by differential usage of multiple promoters of the transactivator CIITA. EMBO Journal 16, 2851-2860.
Nickerson, K., Sisk, T. J., Inohara, N., Yee, C. S. K., Kennell, J., Cho, M. C., Yannie, P. J., Nunez, G., and Chang, C. H. (2001). Dendritic cell-specific MHC class II transactivator contains a caspase recruitment domain that confers potent transactivation activity. Journal of Biological Chemistry 276, 19089-19093.
Nielsen, A. A. K., Der, B. S., Shin, J., Vaidyanathan, P., Paralanov, V., Strychalski, E. A., Ross, D., Densmore, D., and Voigt, C. A. (2016). Genetic circuit design automation. Science 352, 11.
Piovesan, A., Caracausi, M., Ricci, M., Strippoli, P., Vitale, L., and Pelleri, M. C. (2015). Identification of minimal eukaryotic introns through GeneBase, a user-friendly tool for parsing the NCBI Gene databank. DNA Research 22, 495-503.
Poling, B. C., Tsai, K., Kang, D., Ren, L., Kennedy, E. M., and Cullen, B. R. (2017). A lentiviral vector bearing a reverse intron demonstrates superior expression of both proteins and microRNAs. Rna Biology 14, 1570-1579.
Prochazka, L., Angelici, B., Haefliger, B., and Benenson, Y. (2014). Highly modular bow-tie gene circuits with programmable dynamic behaviour. Nature Communications 5, 12.
Rinaudo, K., Bleris, L., Maddamsetti, R., Subramanian, S., Weiss, R., and Benenson, Y. (2007). A universal RNAi-based logic evaluator that operates in mammalian cells. Nature Biotechnology 25, 795-801.
Rizzo, M. A., and Piston, D. W. (2005). High-contrast imaging of fluorescent protein FRET by fluorescence polarization microscopy. Biophysical Journal 88, L14-L16.
Rosenberg, A. B., Patwardhan, R. P., Shendure, J., and Seelig, G. (2015). Learning the Sequence Determinants of Alternative Splicing from Millions of Random Sequences. Cell 163, 698-711.
Sasaki-Haraguchi, N., Shimada, M. K., Taniguchi, I., Ohno, M., and Mayeda, A. (2012). Mechanistic insights into human pre-mRNA splicing of human ultra-short introns: Potential unusual mechanism identifies G-rich introns. Biochemical and Biophysical Research Communications 423, 289-294.
Schreiber, J., Arter, M., Lapique, N., Haefliger, B., and Benenson, Y. (2016). Model-guided combinatorial optimization of complex synthetic gene networks. Molecular Systems Biology 12, 14.
Shcherbakova, D. M., Baloban, M., Emelyanov, A. V., Brenowitz, M., Guo, P., and Verkhusha, V. V. (2016). Bright monomeric near-infrared fluorescent proteins as tags and biosensors for multiscale imaging. Nature Communications 7, 12.
Tamsir, A., Tabor, J. J., and Voigt, C. A. (2011). Robust multicellular computing using genetically encoded NOR gates and chemical ‘wires’. Nature 469, 212-215.
Uphoff, C. C., and Drexler, H. G. (2011). Detecting Mycoplasma Contamination in Cell Cultures by Polymerase Chain Reaction. In Cancer Cell Culture: Methods and Protocols, Second Edition, I. A. Cree, ed. (Totowa: Humana Press Inc), pp. 93-103.
Wang, Y., Ma, M., Xiao, X. S., and Wang, Z. F. (2012). Intronic splicing enhancers, cognate splicing factors and context-dependent regulation rules. Nature Structural & Molecular Biology 19, 1044-U1104.
Weber, W., Fux, C., Daoud-El Baba, M., Keller, B., Weber, C. C., Kramer, B. P., Heinzen, C., Aubel, D., Bailey, J. E., and Fussenegger, M. (2002). Macrolide-based transgene control in mammalian cells and mice. Nature Biotechnology 20, 901-907.
Weinberg, B. H., Pham, N. T. H., Caraballo, L. D., Lozanoski, T., Engel, A., Bhatia, S., and Wong, W. W. (2017). Large-scale design of robust genetic circuits with multiple inputs and outputs for mammalian cells. Nature Biotechnology 35, 453-+.
Xie, M. Q., and Fussenegger, M. (2018). Designing cell function: assembly of synthetic gene circuits for cell biology applications. Nature Reviews Molecular Cell Biology 19, 507-525.
Xie, Z., Wroblewska, L., Prochazka, L., Weiss, R., and Benenson, Y. (2011). Multi-Input RNAi-Based Logic Circuit for Identification of Specific Cancer Cells. Science 333, 1307-1311.
Yeo, G., and Burge, C. B. (2004). Maximum entropy modeling of short sequence motifs with applications to RNA splicing signals. Journal of Computational Biology 11, 377-394.
You, L. C., Cox, R. S., Weiss, R., and Arnold, F. H. (2004). Programmed population control by cell-cell communication and regulated killing. Nature 428, 868-871.

1. A DNA construct comprising in 5′ to 3′ direction the following DNA sequence elements:
a first promoter (P₁);
n amount of further promoter(s) (Pₙ), wherein n≥1; and
an output sequence,
wherein each of said promoters comprises a transcription start site and/or is suitable for initiating transcription,
wherein initiation of transcription from P₁ is enabled by a first transcriptional regulatory state (TS₁) and initiation of transcription from each of said further promoter(s) (Pₙ) is enabled by a respective further transcriptional regulatory state (TSₙ), and
wherein said DNA construct yields an effective amount of an output RNA produced by any of said promoters in a eukaryotic cell, preferably a mammalian cell, when said TS₁ and/or any of the respective TSₙ is present in said cell, and
wherein said output RNA comprises a sequence corresponding to said output sequence.

2. The DNA construct of claim 1, wherein said DNA construct does not yield an effective amount of said output RNA when neither TS₁ nor any of TSₙ is present in said cell.

3. The DNA construct of claim 1, further comprising in 5′ to 3′ direction between said first promoter (P₁) and a last promoter of said further promoter(s) (Pₙ)
a first alternative first exon (E1ₐ) and a first alternative 5′ splice site (5′ss1), and
between said last promoter and said output sequence a branch point (BP) and a 3′ splice site (3′ss), wherein said output sequence is or comprises a second Exon (E2),
wherein the branch point (BP) and the 3′ splice site (3′ss) of the DNA construct enable removal of one or more sequence(s) between at least one 5′ splice site and a 3′ splice site (3′ss) contained in an output RNA produced by any of the promoters contained in said DNA construct, and wherein the first alternative 5′ splice site (5′ss₁) enables the removal of a sequence between a first alternative 5′ splice site (5′ss₁) and a 3′ splice site (3′ss) contained in an output RNA.

4. The DNA construct of claim 3, further comprising, in 5′ to 3′ direction between said first alternative 5′ splice site (5′ss₁) and said last promoter, at least one further group of elements, wherein each of said groups of elements comprises in 5; to 3′ direction:
a further one of n amount of further promoter(s) (Pₙ),
a further one of n amount of respective further alternative first exon (E1ₙ), and
a further one of n amount of respective further alternative 5′ splice sites (5′ssₙ);
wherein a last alternative first exon of the n amount of respective further alternative 5′ splice sites (5′ssₙ) is between said last promoter and said branch point (BP); and
wherein each of said further alternative 5′ splice sites enables the removal of a sequence between a respective further 5′ splice site and the 3′ss contained in an output RNA produced by a promoter that is 5′ of said further 5′ splice site.

5-9. (canceled)

10. The DNA construct of claim 1, wherein said output RNA comprises at least one coding sequence (CDS) encoding at least one output protein, wherein the CDS of said at least one output protein is/are partially or fully contained in said output sequence.

11. The DNA construct of claim 10, wherein said at least one output protein comprises a reporter protein selected from the group consisting of a fluorescent protein, a luminogenic or chromogenic enzyme, and an effector protein, wherein the effector protein is selected from the group consisting of a toxic protein, an enzyme, a cytokine, an immunomodulator, a membrane protein and a membrane-bound receptor.

12. The DNA construct of claim 10,
(i) wherein each of said CDS is fully contained in said output sequence, when said DNA construct does not contain a first exon and a second exon; and
(ii) wherein an CDS that is partially comprised in the second exon corresponds to or is part of an open reading frame (ORF), wherein the start codon of said ORF is contained in at least one alternative first exon comprised in said DNA construct and the stop codon of said ORF is contained in said second exon.

13. (canceled)

14. The DNA construct of claim 1, wherein a certain transcriptional regulatory state (TS)
(i) is associated with and/or reflects a certain cell type and/or cell state; and/or
(ii) comprises the presence of at least one transcription factor (TF).

15. The DNA construct of claim 1, wherein the first transcriptional regulatory state (TS₁) comprises the presence of two TFs from a first group of TFs (TF₁) and/or wherein any of the further transcriptional regulatory state(s) (TSₙ) comprises the presence of at least two TFs from a respective further group of transcription factors (TFₙ.

16. The DNA construct of claim 15, wherein P₁ comprises binding sites for the TFs from TF₁; and/or
wherein any of said Pₙ comprises binding sites for the TFs from a respective TFₙ.

17. (canceled)

18. The DNA construct of claim 4, wherein an output RNA comprising a sequence corresponding to the first alternative first exon (E1ₐ) is translationally inhibited and/or degraded by a first post-transcriptional regulatory state (PTS₁), and/or
wherein an output RNA comprising a sequence corresponding to a further alternative first exon (E1ₙ) is translationally inhibited and/or degraded by a respective further post-transcriptional regulatory state (PTSₙ).

19. The DNA construct of claim 18, wherein
(I) said DNA construct yields an effective amount of an output RNA and/or output protein encoded by an output RNA in said eukaryotic cell, when
(i) at least one transcriptional regulatory state TS₁ and/or any of TSₙ is present in said cell such that an output RNA from at least one respective promoter contained in said DNA construct is produced; and
(ii) at least one of the respective post-transcriptional regulatory state(s) is absent in said cell such that the output RNA comprising the respective alternative first exon(s) produced from other respective promoter(s) is not translationally inhibited or degraded; and
(II) said DNA construct does not yield an effective amount of an output RNA and/or output protein encoded by an output RNA in said eukaryotic cell, when
(i) no transcriptional regulatory state that is capable of inducing transcription from the respective promoter(s) contained in said DNA construct is present in said cell, and/or
(ii) each post-transcriptional regulatory state that is capable of translationally inhibiting and/or degrading the output RNA produced by the respective promoters contained in said DNA construct is present in said cell.

20. (canceled)

21. The DNA construct of claim 18, wherein a certain post-transcriptional regulatory state
(i) is associated with and/or reflects a certain cell type and/or cell state; and/or
(ii) comprises the presence of at least one antisense RNA (AR),
and/or
(iii) comprises the presence of at least one RNA-binding protein.

22-23. (canceled)

24. The DNA construct of claim 21, wherein said first alternative first exon (E1ₐ) comprises at least one sequence corresponding to at least one target site for at least one AR from a first group of ARs (AR₁); and/or
wherein any of said first further first alternative first exons (E1ₙ) comprises at least one sequence corresponding to at least one target site, for at least one AR from a respective further group of Ars (ARₙ).

25. (canceled)

26. The DNA construct of claim 21, wherein the antisense RNA is a microRNA (miRNA) or a small interfering RNA (siRNA).

27. (canceled)

28. The DNA construct of claim 1, wherein the yield of an effective amount of an output RNA in a cell corresponds to the presence of an at least 1.5-, 2-, 4-, 6-, 8-, 10-, 15-, 20-, 30-, 40-, 60-, 80-, 100-, 150-, or 200-fold higher amount of the output RNA in said cell comprising said DNA construct compared to the amount of the output RNA present in a cell comprising the same DNA construct when none of the respective transcriptional regulatory states is present to initiate transcription from any of the promoters contained in said DNA construct.

29. (canceled)

30. The DNA construct of claim 1, wherein the yield of an effective amount of an output RNA and/or output protein in a cell corresponds to the presence of an at least 1.5-, 2-, 4-, 6-, 8-, 10-, 15-, 20-, 30-, 40-, 60-, 80-, 100-, 150-, or 200-fold higher amount of an output protein that is encoded by said output RNA in a cell comprising said DNA construct compared to the amount of said output protein present in a cell comprising the same DNA construct when none of the respective transcriptional regulatory states is present to initiate transcription from any of the promoters contained in said DNA construct.

31. The DNA construct of claim 1, which has a length of at most 150 kb.

32. A plasmid comprising the DNA construct of claim 1.

33. A viral vector comprising the DNA construct of claim 1, wherein the DNA construct is double-stranded or single-stranded comprising a coding strand or an antisense strand, or an RNA comprising a sequence corresponding to any one of said DNA constructs.

34. A host cell comprising the DNA construct of claim 1.

35-65. (canceled)

66. The DNA construct of claim 14, wherein said P₁ comprises at least one binding site for at least one TF from a first group of TFs (TF₁); and/or wherein any of Pₙ comprises at least one binding site for at least one TF from a respective further group of TFs (TF₁).

67. The DNA construct of claim 1, wherein said eukaryotic cell is a human cell.

68. The viral vector of claim 33, wherein said viral vector is selected from a group consisting of: an adeno-associated virus (AAV) vector, a lentiviral vector, an Adenoviral vector, a Herpes-Simplex Virus vector, and a VSV vector.

69. A pharmaceutical composition comprising the DNA construct of claim 1.

70. A method of treating a disease in a subject, comprising introducing the DNA construct of claim 1 into a plurality of cells in said subject, wherein said disease is associated with and/or caused by a heterogeneous mix of different types of target cells, wherein a target cell of the target cells may correspond to a first abnormal and/or malignant cell type and/or state (AC₁), and/or any of n amount of further abnormal and/or malignant cell type(s) and/or state(s) (ACₙ), wherein n≥1.

71. The method of claim 70, wherein treating said disease comprises killing and/or manipulating the heterogeneous mix of target cells regardless of whether the target cell corresponds to said first abnormal and/or malignant cell type and/or state (AC₁), and/or to any of said further abnormal and/or malignant cell types and/or states (ACₙ).

72. The method of the claim 71, wherein non-target cells are not killed and/or manipulated.

73. The method of claim 70, wherein an effective amount of the output RNA and/or at least one output protein encoded by said output RNA is obtained
(i) in target cells that correspond to said first abnormal and/or malignant cell type and/or state (AC₁), and/or
(ii) in target cells that correspond to any of said further abnormal and/or malignant cell type(s) and/or state(s) (ACₙ).

74. The method of claim 73, wherein said at least one output protein comprises at least one effector protein selected from the group consisting of: a toxic protein, an enzyme, a cytokine, an immunomodulator, a membrane protein and a membrane-bound receptor.

75. The method of claim 70, wherein said disease is a cancer, a neurodegenerative disease, an immunodeficiency, and/or a genetic disease.

76. The method of claim 70, wherein said subject is a human.

77. A method for determining the cell type and/or state of a eukaryotic cell, wherein said method comprises
(a) introducing the DNA construct of claim 1 into said eukaryotic cell,
(b) measuring the amount of an output RNA and/or an output protein encoded by said output RNA in said cell, and
(c) determining that
(i) said eukaryotic cell has a certain cell type and/or state when an effective amount of said output RNA and/or output protein is present in said eukaryotic cell, and/or
(ii) said eukaryotic cell does not have said cell type and/or state when no effective amount of said output RNA and/or output protein is present in said eukaryotic cell.

78. A method for diagnosing a disease in a subject, wherein said method comprises
(a) introducing the DNA construct of claim 1 into a tissue of said subject or into a tissue sample from said subject,
(b) measuring the amount of an output RNA and/or an output protein encoded by said output RNA in said tissue or tissue sample, and
(c) diagnosing whether said subject has said disease,
wherein the diagnosis is positive when an effective amount of the output RNA and/or output protein is present in said tissue or tissue sample, and/or
wherein the diagnosis is negative when no effective amount of the output RNA and/or output protein is present in said tissue or tissue sample.

79. The method of claim 78, wherein said disease is associated with and/or caused by a heterogeneous mix of different types of target cells, wherein a target cell of the target cells may correspond to a first abnormal and/or malignant cell type and/or state (AC₁), and/or any of n amount of further abnormal and/or malignant cell type(s) and/or state(s) (ACₙ), wherein n≥1.

80. The method of claim 79, wherein an effective amount of said output RNA and/or output protein is obtained
(i) in target cells that correspond to said first abnormal and/or malignant cell type and/or state (AC₁), and/or
(ii) in target cells that correspond to any of said further abnormal and/or malignant cell type(s) and/or state(s) (ACₙ).

81. The method of claim 78, wherein said output protein comprises at least one reporter protein selected from the group consisting of: a fluorescent protein, a luminogenic enzyme and a chromogenic enzyme.

82. The method of claim 78, wherein said disease is a cancer, a neurodegenerative disease, an immunodeficiency, and/or a genetic disease.

83. A method for producing the DNA construct of claim 1, wherein said method comprises the steps of
(a) selecting and arranging the DNA sequence elements contained in the DNA construct, and
(b) assembling and/or synthesizing the selected and arranged DNA sequence elements, thereby producing said DNA construct.

84. The method of claim 83, wherein prior to step (a) the transcriptional regulatory states and/or post-transcriptional regulatory states of target cells and/or non-target cells are analyzed.


SEQUENCE LISTING (236 sequences)

SEQ ID NO: 1 - DNA, 29 bp, Artificial Sequence
  3 prime -splice site
ttttttaacttcctttattttccttacag

SEQ ID NO: 2 - DNA, 10 bp, Artificial Sequence
  intron splicing enhancer
gttggtggtt

SEQ ID NO: 3 - DNA, 103 bp, Artificial Sequence
  P1
gaaatagcgctgtacagcgtatgggaatctcttgtacggtgtacgagtatcttcccgtacaccgtacaagcttactagagggtatataatgggggccaatcct

SEQ ID NO: 4 - DNA, 136 bp, Artificial Sequence
  P2
gattgaatataaccgacgtgactgttacatttagggattgtatataacccacgtgagtgttacaattagggattgaatataaccgacgtgactgttacatttaggaagcttactagagggtatataatgggggcca

SEQ ID NO: 5 - DNA, 276 bp, Artificial Sequence
  P3
tccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaaacgttactagagggtatataatgggggccagactgcccgccc

SEQ ID NO: 6 - DNA, 180 bp, Artificial Sequence
  PIR_3x + SOX10_3x-C-C′
gaaatagcgctgtacagcgtatgggaatctcttgtacggtgtacgagtatcttcccgtacaccgtacgagctcctacacaaagccctctttgtgagactacacaaagccctctttgtgagactacacaaagccctctttgtgagaggtaccctagagggtatataatgggggccaatcct

SEQ ID NO: 7 - DNA, 182 bp, Artificial Sequence
  PIR_2x + SOX10_3x-C-C′
gaaatagcgcttaaagtaagctgggaatctcttgtacggtgtacgagtatcttcccgtacaccgtacgagctcctacacaaagccctctttgtgagactacacaaagccctctttgtgagactacacaaagccctctttgtgagaggtaccctagagggtatataatgggggccaatcctct

SEQ ID NO: 8 - DNA, 103 bp, Artificial Sequence
  ETR + HNF1A_2x
gattgaatataaccgacgtgactgttacatttaggcaattgaagttaataattaactagttaataattaactaagcttactagagggtatataatgggggcca

SEQ ID NO: 9 - DNA, 72 bp, Mus musculus
  UTR U1
acttcatgttttggatgctgcaaggctggatgagaggcgactccaggcagcaggcagcctcagagcactgcc

SEQ ID NO: 10 - DNA, 207 bp, Mus musculus
  UTR U2
gaattttcaggtggtcccttgctcgctttctttgcatgctagctgagcttggtaggttctgggctcctaactgatagacagaggcatgtgagggatgaggctgcctgcttcccacctgggcatctgaggacctttttggagacttccggcacgccaggaggggcagctggactacagacgttactgcatcactctgctctctaaatc

SEQ ID NO: 11 - DNA, 41 bp, Mus musculus
  UTR U3
caagctcctaggagccacggagctggcggcagggagactgc

SEQ ID NO: 12 - DNA, 117 bp, Artificial Sequence
  UTR U4
ctgaattcatcttggctgaggaatcttctaacaatttagagcttaaaaacgcccacgaggcggagaacgaaatatccagagagacgttagaaacgttcaaaaacgttcactagtagg

SEQ ID NO: 13 - DNA, 45 bp, Mus musculus
  UTR U5
gaattttcaggtatcctctggtcccttgctcgctttctttgcatg

SEQ ID NO: 14 - DNA, 34 bp, Artificial Sequence
  UTR U6
ctgaattcttataaaggcctattgtactagtagg

SEQ ID NO: 15 - DNA, 122 bp, Artificial Sequence
  UTR U1+FF4_2x
acttcatgttttggatgctgcaaggctggatgagaggcgactccaggcagcaggcagcctcagagcactgccccgcttgaagtctttaattaaaccgcttgaagtctttaattaaaaccggt

SEQ ID NO: 16 - DNA, 267 bp, Artificial Sequence
  UTR U2+FF5_3x
ggtcccttgctcgctttctttgcatgctagctgagcttggtaggttctgggctcctaactgatagacagaggcatgtgagggatgaggctgcctgcttcccacctgggcatctgaggacctttttggagacttccggcacgccaggaggggcagctggactacagacgttactgcatcactctgctctctaaatcaagcactctgatttgacaattaaagcactctgatttgacaattaaagcactctgatttgacaattactcgag

SEQ ID NO: 17 - DNA, 630 bp, Artificial Sequence
  Exon1 CDS B1
atggtgagcaagggcgaggagctgttcaccggcgtggtgcccatcctggtggagctggacggcgacgtgaacggccacaagttcagcgtgagcggcgagggcgagggcgacgccacctacggcaagctgaccctgaagttcatctgcaccaccggcaagctgcccgtgccctggcccaccctggtgacaaccctgagccacggcgtgcagtgcttcgccagataccccgaccacatgaagcagcacgacttcttcaagagcgccatgcccgagggctacgtgcaggagagaaccatcttcttcaaggacgacggcaactacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggccaacttcaagatcagacacaacatcgaggacggcggcgtgcagctggccgaccactaccagcagaacacccccatcggcgacggccccgtgctgctgcccgacaaccactacctgagcacccagagcaagctgagcaag

SEQ ID NO: 18 - DNA, 630 bp, Artificial Sequence
  Exon1 CDS B2
atggtgagcaagggcgaggagctgttcaccggcgtggtgcccatcctggtggagctggacggcgacgtgaacggccacaagttcagcgtgagcggcgagggcgagggcgacgccacctacggcaagctgaccctgaagttcatctgcaccaccggcaagctgcccgtgccctggcccaccctggtgacaaccctgagccacggcgtgcagtgcttcgccagataccccgaccacatgaagcagcacgacttcttcaagagcgccatgcccgagggctacgtgcaggagagaaccatcttcttcaaggacgacggcaactacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggccaacttcaagatcagacacaacatcgaggacggcggcgtgcagctggccgaccactaccagcagaacacccccatcggcgacggccccgtgctgcttccggacaaccactacctgagcacccagagcaagctgagcaag

SEQ ID NO: 19 - DNA, 630 bp, Artificial Sequence
  Exon1 CDS B4
atggtgagcaagggcgaggagctgttcaccggcgtggtgcccatcctggtggagctggacggcgacgtgaacggccacaagttcagcgtgagcggcgagggcgagggcgacgccacctacggcaagctgaccctgaagttcatctgcaccaccggcaagctgcccgtgccctggcccaccctggtgacaaccctgagccacggcgtgcagtgcttcgccagataccccgaccacatgaagcagcacgacttcttcaagagcgccatgcccgagggctacgtgcaggagagaaccatcttcttcaaggacgacggcaactacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggccaacttcaagatcagacacaacatcgaggacggcggcgtgcagctggccgaccactaccagcagaacacccccatcggcgacggccccgtgctgcttccggacaatcattatctcagtacccaaagcaaactcagcaag

SEQ ID NO: 20 - DNA, 630 bp, Artificial Sequence
  Exon1 CDS C1
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaa

SEQ ID NO: 21 - DNA, 630 bp, Artificial Sequence
  Exon1 CDS C2
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacaccccaattggcgacggccccgtgctgctgcccgacaaccactacctgagcacccagtccaagctttcgaag

SEQ ID NO: 22 - DNA, 630 bp, Artificial Sequence
  Exon1 CDS C4
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacaccccaattggagatgggcctgtcctcctacctgataatcactatctaagtacgcaatctaaactttcgaag

SEQ ID NO: 23 - DNA, 630 bp, Artificial Sequence
  Exon1 CDS C5
atggtgtccaagggtgaggaactgttcactggggtggtgcctatccttgttgaactcgacggggatgtgaatggccatcgcttctcagtttctggcgagggggaaggcgacgcaacgtatggcaagcttacactgaaatttatctgtactaccggaaaacttcccgttccctggcctacgctcgtcactactcttacatggggagtacagtgcttcagtaggtaccccgatcacatgaaacaacatgatttcttcaaatctgcaatgcctgagggctatgtacaggagagaacaatatttttcaaggatgacggaaactataagaccagggcggaggtaaagttcgagggggacaccctggtgaaccgaatagagctgaaagggatagacttcaaggaagatggcaatatccttggacacaagttggaatacaactacatcagccacaacgtgtacattacagcagataagcagaaaaacggcatcaaagcccacttcaaaattagacacaatatcgaggacgggtctgttcaactggccgaccactatcaacagaacaccccaattggagacggacctgtattgctgcccgacaatcattatctgagcactcagtctgcactttccaag

SEQ ID NO: 24 - DNA, 630 bp, Artificial Sequence
  Exon1 CDS C6
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacaccccaattggcgacggccccgtgctgctgcccgacaaccactacctgagcacccagtccaagctgagcaag

SEQ ID NO: 25 - DNA, 630 bp, Artificial Sequence
  Exon1 CDS Y1
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaag

SEQ ID NO: 26 - DNA, 630 bp, Artificial Sequence
  Exon1 CDS Y2
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggaccggtgctgctgcccgacaaccactacctgagctaccagtccaagctgagcaag

SEQ ID NO: 27 - DNA, 630 bp, Artificial Sequence
  Exon1 CDS Y3
atggtgagcaagggcgaggagctgttcaccggggtggtgcccatcctggtcgagctggacggcgacgtaaacggccacaagttcagcgtgtccggcgagggcgagggcgatgccacttacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggaccggtgctgctgcccgacaaccactacctgagctaccagtccaagctgagcaag

SEQ ID NO: 28 - DNA, 283 bp, Mus musculus
  Exon1 CDS Ia
atgaaccacttccaggccatcctggcccaagtacagacactgctctccagccagaagcccaggcaggtgcgggccctcctggatggcctgctggaagaagagctgctctcacgggaataccactgtgccttgctgcatgagcctgatggtgatgccctggcccggaagatttccctgaccctgctggagaaaggggacttagacttgactttcttgagctgggtctgcaacagtctgcaggctcccacggtagagaggggcaccagctacagggaccatggag

SEQ ID NO: 29 - DNA, 52 bp, Mus musculus
  Exon1 CDS Ib
atgcgctgcctggttcctggcccttctgggtcttacctgccggagttgcaag

SEQ ID NO: 30 - DNA, 43 bp, Mus musculus
  Exon1 CDS Ic
atgcaggcagcactcagaagcacggggcacagccacagccgcg

SEQ ID NO: 31-38 - skipped (8 entries)

SEQ ID NO: 39 - DNA, 15 bp, Artificial Sequence
  splice site ss9
aaggtaagaatctgc

SEQ ID NO: 40-47 - skipped (8 entries)

SEQ ID NO: 48 - DNA, 26 bp, Mus musculus
  splice site ss13
atctgagtgtatctctcctcccagga

SEQ ID NO: 49 - DNA, 31 bp, Artificial Sequence
  splice site ss14
ttttttaacttcctttattttccttacagga

SEQ ID NO: 50 - DNA, 245 bp, Artificial Sequence
  intron I1
tggcatccctttgagtcaaggcaacaatgtatgaccaaccctaaggtgtcctttctctgacaggaaaatctatgtaaaggatatagggtaagcttttggaaaacctgggtggctgggtgctgcaagcacaaagcttgttttccacattgatcagagcatcctcagggcacactgccgcctacctgcttagtccatgcctgagcaccatcgacaatggacaccagtgtacactgttcagcggccgc

SEQ ID NO: 51 - DNA, 274 bp, Artificial Sequence
  intron I2
tggcatccctttgagtcaaggcaagaattccaatgtatgaccaacgtggaaaacaatgtttttgaacacctaaggtgtcctttctctgacaggaaaatctatgtaaaggatatagggtaagcttttggaaaacctgggtggctgggtgctgcaagcacaaagcttgttttccacattgatcagagcatcctcagggcacactgccgcctacctgcttagtccatgcctgagcaccatcgacaatggacaccagtgtacactgttcagcggccgc

SEQ ID NO: 52 - DNA, 261 bp, Artificial Sequence
  intron I3
tggcatccctttgagtcgttggtggttatcgataaggcaacaatgtatgaccaaccctaaggtgtcctttctctgacaggaaaatctatgtaaaggatatagggtaagcttttggaaaacctgggtggctgggtgctgcaagcacaaagcttgttttccacattgatcagagcatcctcagggcacactgccgcctacctgcttagtccatgcctgagcaccatcgacaatggacaccagtgtacactgttcagcggccgc

SEQ ID NO: 53 - DNA, 245 bp, Artificial Sequence
  intron I17
tggcatccctttgagtcaagaattcaatgtatgaccaaccctaaggtgtcctttctctgacaggaaaatctatgtaaaggatatagggtaagcttttggaaaacctgggtggctgggtgctgcaagcacaaagcttgttttccacattgatcagagcatcctcagggcacactgccgcctacctgcttagtccatgcctgagcaccatcgacaatggacaccagtgtacactgttcagcggccgc

SEQ ID NO: 54 - DNA, 443 bp, Artificial Sequence
  intron I8
tggcatccctttgagtcaaggcaacaatgtatgaccaaccctaaggtgtcctttctctgacaggaaaatctatgtaaaggatatagggtaagcttttggaaaacctgggtggctgggtgctgcaagcacaaagcttgttttccacattgatcagagcatcctcagggcacactgccgcctacctgcttagtccatgcctgagcaccatcgacaatggacaccagtgtacactgttcagagctcagaaaacggatacttaaagaggataagtcactcgccttgggcctcacagatgggactccaactcctctctagtgtctggaaccctgttgttcatattcttaggctcctcccacccccagccctcagcagcagtgcaagttcactctctgcctttgcctaccataatccgatgacatatctgagtgtatctctcctcccag

SEQ ID NO: 55 - DNA, 53 bp, Artificial Sequence
  intron I4
tggcatccctttgagtcgttggtggttatcgatgtacactgttcagcggccgc

SEQ ID NO: 56 - DNA, 60 bp, Artificial Sequence
  intron I21
tggcatccctttgagtcgttggtggttatcgattgcggtgtatctaatgcatgcggccgc

SEQ ID NO: 57 - DNA, 353 bp, Artificial Sequence
  intron I5
gatgggctagagccaatggttaagactagagctttcctatgcacactgaggtcttgggtggtttcagcattcctaacagagtttttaatctgcaccatgacttccaggggcctctgctggcagctctcagccccaccaaggttttgggctacttagactatcatctcttcctcttccttctcctcttccttaaggaggaaccctgtggatgattgacaagctctctacccttgagcgacatctcaagcctatttctgtctttgagtacactgtgctgtacttctaggagacctccatcctgagaagcttggcactcccagactataagcaagcctgcggtgtatctaatgcat

SEQ ID NO: 58 - DNA, 353 bp, Artificial Sequence
  intron I6
gatgggctagagccaatggtgaagactagagctttcctatgcacactgaggtcttgggtggtttcagcattcctaacagagtttttaatctgcaccatgacttccaggggcctctgctggcagctctcagccccaccaaggttttgggctacttagactatcatctcttcctcttccttctcctcttccttaaggaggaaccctgtggatgattgacaagctctctacccttgagcgacatctcaagcctatttctgtctttgagtacactgtgctgtacttctaggagacctccatcctgagaagcttggcactcccagactataagcaagcctgcggtgtatctaatgcat

SEQ ID NO: 59 - DNA, 352 bp, Artificial Sequence
  intron I7
atctgctagagccaatggtgaagactagagctttcctatgcacactgaggtcttgggtggtttcagcattcctaacagagtttttaatctgcacaatgacttccaggggcctctgctggcagctctcagccccaccaaggttttgggctacttagactatcatctcttcctcttccttctcctcttccttaaggaggaaccctgtggatgattgacaagctctctacccttgagcgacatctcaagcctatttctgtctttgagtacactgtgctgtacttctaggagacctccatcctgagaagcttggcactcccagactataagcaagcctgcggtgtatctaatgcat

SEQ ID NO: 60 - DNA, 588 bp, Artificial Sequence
  intron I23
atctgctagagccaatggtgaagactagagctttcctatgcacactgaggtcttgggtggtttcagcattcctaacagagtttttaatctgcacaatgacttccaggggcctctgctggcagctctcagccccaccaaggttttgggctacttagactatcatctcttcctcttccttctcctcttccttaaggaggaaccctgtggatgattgacaagctctctacccttgagcgacatctcaagcctatttctgtctttgagtacactgtgctgtacttctaggagacctccatcctgagaagcttggcactcccagactataagcaagcctgcggtgtatctaatgcattggcacagagacagcattttatttgactctgagctcagaaaacggatacttaaagaggataagtcactcgccttgggcctcacagatgggactccaactcctctctagtgtctggaaccctgttgttcatattcttaggctcctcccacccccagccctcagcagcagtgcaagttcactctctgcctttgcctaccataatccgatgacatatctgagtgtatctctcctcccag

SEQ ID NO: 61 - DNA, 55 bp, Artificial Sequence
  intron I9
atctgctagagccaatggtgaagactagagctttcctgcggtgtatctaatgcat

SEQ ID NO: 62 - DNA, 716 bp, Artificial Sequence
  intron I10
gtctccaagatcccctttgcagccgctaaagttaggttgggaatccgaggctctgaggctggcaactatttgcccgaagtcagacagcagggccggaaaaagctggatgggtgtgcaggaggttgagaggcttcttggatcttggacggactgtatgcaggacctgggagcagggagctggggtccagaacaccgagactatgcacagaactggtagtgaaggcaagtggcgggatagccttgcaggagtgtctgcagctctgagactcagaccgtgggaggcgcggggaggcgggggaggagggggaggaggtggggggtgggcagacgtgcactctattaatacccaacatcttcagagagcgcagggaccttggaccaggggctgcccttcactggaccttcatttgcgtgttgggacagaccacctctgactctggagtctgggcacagggtccttcttgtgagggcttggcacagagacagcatagggtgcatttatttgactctgagctcagaaaacggatacttaaagaggataagtcactcgccttgggcctcacagatgggactccaactcctctctagtgtctggaaccctgttgttcatattcttaggctcctcccacccccagccctcagcagcagtgcaagttcactctctgcctttgcctaccataatccgatgacatatctgagtgtatctctcctcccag

SEQ ID NO: 63 - DNA, 716 bp, Artificial Sequence
  intron I11
gtctccaagatcccctttggcgccgctaaagttaggttgggaatccgaggctctgaggctggcaactatttgcccgaagtcagacagcagggccggaaaaagctggatgggtgtgcaggaggttgagaggcttcttggatcttggacggactgtatgcaggacctgggagcagggagctggggtccagaacaccgagactatgcacagaactggtagtgaaggcaagtggcgggatagccttgcaggagtgtctgcagctctgagactcagaccgtgggaggcgcggggaggcgggggaggagggggaggaggtggggggtgggcagacgtgcactctattaatacccaacatcttcagagagcgcagggaccttggaccaggggctgcccttcactggaccttcatttgcgtgttgggacagaccacctctgactctggagtctgggcacagggtccttcttgtgagggcttggcacagagacagcatagggtgcatttatttgactctgagctcagaaaacggatacttaaagaggataagtcactcgccttgggcctcacagatgggactccaactcctctctagtgtctggaaccctgttgttcatattcttaggctcctcccacccccagccctcagcagcagtgcaagttcactctctgcctttgcctaccataatccgatgacatatctgagtgtatctctcctcccag

SEQ ID NO: 64 - DNA, 715 bp, Artificial Sequence
  intron I20
gtctccaagatcccctttggcgccgctaaagttaggttgggaatccgaggctctgaggctggcaactatttgcccgaagtcagacagcagggccggaaaaagctggatgggtgtgcaggaggttgagggcttcttggatcttggacggactgtatgcaggacctgggagcagggagctggggtccagaacaccgagactatgcacagaactggtagtgaaggcaagtggcgggatagccttgcaggagtgtctgcagctctgagactcagaccgtgggaggcgcggggaggcgggggaggagggggaggaggtggggggtgggcagacgtgcactctattaatacccaacatcttcagagagcgcagggaccttggaccaggggctgcccttcactggaccttcatttgcgtgttgggacagaccacctctgactctggagtctgggcacagggtccttcttgtgagggcttggcacagagacagcatagggtgcatttatttgactctgagctcagaaaacggatacttaaagaggataagtcactcgccttgggcctcacagatgggactccaactcctctctagtgtctggaaccctgttgttcatattcttaggctcctcccacccccagccctcagcagcagtgcaagttcactctctgcctttgcctaccataatccgatgacatatctgagtgtatctctcctcccag

SEQ ID NO: 65 - DNA, 70 bp, Artificial Sequence
  intron I12
gtctccaagatcccctttggcgccgctaaagttaggttgggaatatatctgagtgtatctctcctcccag

SEQ ID NO: 66 - DNA, 90 bp, Artificial Sequence
  intron I13
gtctccaagatcccctttggcgccgctaaagttaggttgggaatgcctaccataatccgatgacatatctgagtgtatctctcctcccag

SEQ ID NO: 67 - DNA, 64 bp, Artificial Sequence
  intron I19
gtctccaagatcccctttggcgcccataatccgatgacatatctgagtgtatctctcctcccag

SEQ ID NO: 68 - DNA, 64 bp, Artificial Sequence; identical to SEQ ID NO: 67
  intron I24

SEQ ID NO: 69 - DNA, 95 bp, Artificial Sequence
  intron I22
atctgctagagccaatggtgaagactagagctttcctgcggtgtatctaatgcatcataatccgatgacatatctgagtgtatctctcctcccag

SEQ ID NO: 70 - DNA, 90 bp, Artificial Sequence
  Exon2 CDS BCY1
gatcccaacgagaagagagatcacatggtgcttcttgagttcgtgaccgcagccggaattaccctgggtatggacgagttgtacaagtag

SEQ ID NO: 71 - DNA, 69 bp, Artificial Sequence
  Exon2 CDS BCY2
gatcccaacgagaagagagatcacgagcagaaactcatctcagaagaggatctggagttgtacaagtag

SEQ ID NO: 72 - DNA, 17 bp, Artificial Sequence
  GS linker
gatccggtggaggaggt

SEQ ID NO: 73 - DNA, 720 bp, Artificial Sequence
  mCitrine
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtag

SEQ ID NO: 74 - DNA, 349 bp, Artificial Sequence
  pA1
acgcgtgcactcctcaggtgcaggctgcctatcagaaggtggtggctggtgtggccaatgccctggctcacaaataccactgagaactttttccctctgccaaaaattatggggacatcatgaagccccttgagcatctgacttctggctaataaaggaaatttattttcattgcaatagtgtgttggaattttttgtgtctctcactcggaaggacatttgggagggcaaatcatttaaaacatcagaatgagtatttggtttagagtttggcaacatctgccatctgctggctgccatgaacaaaggtggctataaagaggtcatcagtatatgaaacagccccctg

SEQ ID NO: 75 - DNA, 26 bp, Artificial Sequence
  primer PR4317
ctgatcatgcattagatacaccgcag

SEQ ID NO: 76 - DNA, 18 bp, Artificial Sequence
  primer PR4356
cttgctcgctttctttgc

SEQ ID NO: 77 - DNA, 25 bp, Artificial Sequence
  primer PR4330
gtattacatatggtcgacggtcacc

SEQ ID NO: 78 - DNA, 34 bp, Artificial Sequence
  primer PR4364
atggtcgagctctgaacagtgtacactggtgtcc

SEQ ID NO: 79 - DNA, 27 bp, Artificial Sequence
  primer PR4325
atggtcggatcccaacgagaagagaga

SEQ ID NO: 80 - DNA, 32 bp, Artificial Sequence
  primer PR4326
attgctatgcatggcgcgccggtgaccgtcga

SEQ ID NO: 81 - DNA, 70 bp, Artificial Sequence
  primer PR5070
gatcccaacgagaagagagatcacgagcagaaactcatctcagaagaggatctggagttgtacaagtaga

SEQ ID NO: 82 - DNA, 70 bp, Artificial Sequence
  primer PR5071
cgcgtctacttgtacaactccagatcctcttctgagatgagtttctgctcgtgatctctcttctcgttgg

SEQ ID NO: 83 - DNA, 19 bp, Artificial Sequence
  primer prKB0001
cttgctcagcttgctctgg

SEQ ID NO: 84 - DNA, 27 bp, Artificial Sequence
  primer prKB0002
gatcccaacgagaagagagatcacatg

SEQ ID NO: 85 - DNA, 22 bp, Artificial Sequence
  primer prKB0003
cttggaaagtgcagactgagtg

SEQ ID NO: 86 - DNA, 22 bp, Artificial Sequence
  primer prKB0004
cttgctcagcttggactggtag

SEQ ID NO: 87 - DNA, 74 bp, Artificial Sequence
  primer PR7360
agcttactagagggtatataatgggggccaatcctctgaattcttataaaggcctattgtactagtagggccgc

SEQ ID NO: 88 - DNA, 74 bp, Artificial Sequence
  primer PR7361
catggcggccctactagtacaataggcctttataagaattcagaggattggcccccattatataccctctagta

SEQ ID NO: 89 - DNA, 67 bp, Artificial Sequence
  primer PR7358
aattggcgacggccccgtgctgctgcccgacaaccactacctgagcacccagtccaagctgagcaag

SEQ ID NO: 90 - DNA, 67 bp, Artificial Sequence
  primer PR7359
gatccttgctcagcttggactgggtgctcaggtagtggttgtcgggcagcagcacggggccgtcgcc

SEQ ID NO: 91 - DNA, 157 bp, Artificial Sequence
  primer PR7362
agcttactagagggtatataatgggggccaatcctctgaattcatcttggctgaggaatcttctaacaatttagagcttaaaaacgcccacgaggcggagaacgaaatatccagagagacgttagaaacgttcaaaaacgttcactagtagggccgc

SEQ ID NO: 92 - DNA, 157 bp, Artificial Sequence
  primer PR7363
catggcggccctactagtgaacgtttttgaacgtttctaacgtctctctggatatttcgttctccgcctcgtgggcgtttttaagctctaaattgttagaagattcctcagccaagatgaattcagaggattggcccccattatataccctctagta

SEQ ID NO: 93 - DNA, 129 bp, Artificial Sequence
  primer PR7364
agcttactagagggtatataatgggggccaatcctctgaattcacttcatgttttggatgctgcaaggctggatgagaggcgactccaggcagcaggcagcctcagagcactgccactagtagggccgc

SEQ ID NO: 94 - DNA, 129 bp, Artificial Sequence
  primer PR7365
catggcggccctactagtggcagtgctctgaggctgcctgctgcctggagtcgcctctcatccagccttgcagcatccaaaacatgaagtgaattcagaggattggcccccattatataccctctagta

SEQ ID NO: 95 - DNA, 96 bp, Artificial Sequence
  primer PR6812
cgaaggatcccaacgagaagagagatcacatggtgcttcttgagttcgtgaccgcagccggaattaccctgggtatggacgagttgtacaagtaga

SEQ ID NO: 96 - skipped

SEQ ID NO: 97 - DNA, 52 bp, Artificial Sequence
  primer PR6813
cgcgtctacttgtacaactcgtccatacccagggtaattccggctgcggtca

SEQ ID NO: 98 - DNA, 25 bp, Artificial Sequence
  primer PR4310
atgttgccatggtgagcaagggcga

SEQ ID NO: 99 - DNA, 59 bp, Artificial Sequence
  primer PR4311
tagtctgaattcttgactcaaagggatgccaacttaccttgctcagcttgctctgggtg

SEQ ID NO: 100 - DNA, 33 bp, Artificial Sequence
  primer PR4312
gtctatggaattcaatgtatgaccaaccctaag

SEQ ID NO: 101 - DNA, 34 bp, Artificial Sequence
  primer PR4313
aatctggcggccgctgaacagtgtacactggtgt

SEQ ID NO: 102 - DNA, 38 bp, Artificial Sequence
  primer PR4528
cgaaggtatgtatctgctagagccaatggtgaagacta

SEQ ID NO: 103 - DNA, 40 bp, Artificial Sequence
  primer PR4529
gctctagtcttcaccattggctctagcagatacatacctt

SEQ ID NO: 104 - DNA, 59 bp, Artificial Sequence
  primer PR4490
attctggaattcttataaaggcctattgtactagtagggccgccatggtgagcaagggc

SEQ ID NO: 105 - DNA, 34 bp, Artificial Sequence
  primer PR4491
attgtcgaattcagaggattggcccccattatat

SEQ ID NO: 106 - DNA, 78 bp, Artificial Sequence
  primer PR4494
aattcacttcatgttttggatgctgcaaggctggatgagaggcgactccaggcagcaggcagcctcagagcactgcca

SEQ ID NO: 107 - DNA, 78 bp, Artificial Sequence
  primer PR4495
ctagtggcagtgctctgaggctgcctgctgcctggagtcgcctctcatccagccttgcagcatccaaaacatgaagtg

SEQ ID NO: 108 - DNA, 38 bp, Artificial Sequence
  primer PR4532
cgaaggtttgtatctgctagagccaatggtgaagacta

SEQ ID NO: 109 - DNA, 40 bp, Artificial Sequence
  primer PR4533
gctctagtcttcaccattggctctagcagatacaaacctt

SEQ ID NO: 110 - DNA, 106 bp, Artificial Sequence
  primer PR4492
aattcatcttggctgaggaatcttctaacaatttagagcttaaaaacgcccacgaggcggagaacgaaatatccagagagacgttagaaacgttcaaaaacgttca

SEQ ID NO: 111 - DNA, 106 bp, Artificial Sequence
  primer PR4493
ctagtgaacgtttttgaacgtttctaacgtctctctggatatttcgttctccgcctcgtgggcgtttttaagctctaaattgttagaagattcctcagccaagatg

SEQ ID NO: 112 - DNA, 36 bp, Artificial Sequence
  primer PR6085
ttggcacagagacagcattttatttgactctgagct

SEQ ID NO: 113 - DNA, 36 bp, Artificial Sequence
  primer PR6086
cagagtcaaataaaatgctgtctctgtgccaatgca

SEQ ID NO: 114 - DNA, 35 bp, Artificial Sequence
  primer PR6113
cctgattactagtgccaccatggtgagcaagggcg

SEQ ID NO: 115 - DNA, 73 bp, Artificial Sequence
  primer PR6114
tgcattagcggccgcatgcattagatacaccgcaatcgataaccaccaacgactcaaagggatgccagcttac

SEQ ID NO: 116 - DNA, 56 bp, Artificial Sequence
  primer PR5701
agcaggcagcctcagagcactgccactagtgccaccatgaaccacttccaggccat

SEQ ID NO: 117 - DNA, 59 bp, Artificial Sequence
  primer PR5702
atcgataaccaccaacgactcaaagggatgccagcttacctccatggtccctgtagctg

SEQ ID NO: 118 - DNA, 59 bp, Artificial Sequence
  primer PR5703
gtaagctggcatccctttgagtcgttggtggttatcgatgtacactgttcagcggccgc

SEQ ID NO: 119 - DNA, 115 bp, Artificial Sequence
  primer PR5704
tcttcaccattggctctagcagatacttaccttgcaactccggcaggtaagacccagaagggccaggaaccaggcagcgcatggtggcgatttagagagcagagtgatgcagtaa

SEQ ID NO: 120 - DNA, 63 bp, Artificial Sequence
  primer PR5705
gtaagtatctgctagagccaatggtgaagactagagctttcctgcggtgtatctaatgcattc

SEQ ID NO: 121 - DNA, 88 bp, Artificial Sequence
  primer PR5706
ggcgccaaaggggatcttggagacgcctaccgcggctgtggctgtgccccgtgcttctgagtgctgcctgcatgcagtctccctgccg

SEQ ID NO: 122 - DNA, 106 bp, Artificial Sequence
  primer PR5707
gtaggcgtctccaagatcccctttggcgcccataatccgatgacatatctgagtgtatctctcctcccaggatccggtggaggaggtatggtgagcaagggcgagg

SEQ ID NO: 123 - DNA, 60 bp, Artificial Sequence
  primer PR5708
ctgataggcagcctgcacctgaggagtgcacgcgtctacttgtacagctcgtccatgccg

SEQ ID NO: 124 - skipped

SEQ ID NO: 125 - DNA, 62 bp, Artificial Sequence
  primer PR5709
gtaagctggcatccctttgagtcgttggtggttatcgattgcggtgtatctaatgcattccc

SEQ ID NO: 126 - DNA, 99 bp, Artificial Sequence
  primer PR6238
tctgctagagccaatggtgaagactagagccataatccgatgacatatctgagtgtatctctcctcccaggatcccaacgagaagagagatcacatggt

SEQ ID NO: 127 - DNA, 41 bp, Artificial Sequence
  primer PR6087
tcataatccgatgacatatctgagtgtatctctcctcccag

SEQ ID NO: 128 - DNA, 49 bp, Artificial Sequence
  primer PR6088
gatcctgggaggagagatacactcagatatgtcatcggattatgatgca

SEQ ID NO: 129 - DNA, 54 bp, Artificial Sequence
  primer PR6259
aggttgattatcgataagcttgatatcgttgaaatagcgctgtacagcgtatgg

SEQ ID NO: 130 - DNA, 60 bp, Artificial Sequence
  primer PR6260
acagcagagatccagtttggttaattaatgagagacacaaaaaattccaacacactattg

SEQ ID NO: 131 - DNA, 43 bp, Artificial Sequence
  primer PR2818
ggacagcagagatccagtttggttaattaagctccggtgcccg

SEQ ID NO: 132 - DNA, 91 bp, Artificial Sequence
  primer PR2819
tatcctgcaggcgcgacttagggtttgtaccgtacaccactgagaccgcggtggttgaccagacaaaccacgctagcgaacgtttttgaac

SEQ ID NO: 133 - DNA, 38 bp, Artificial Sequence
  primer PR2820
taagtcgcgcctgcaggatagccaccatggtgagcaag

SEQ ID NO: 134 - DNA, 106 bp, Artificial Sequence
  primer PR2821
ttgattatcgataagcttgatatcgttcgaagtggtttgtctggtcaaccaccgcggtctcagtggtgtacggtacaaacccgttaacagcctgcacctgaggagt

SEQ ID NO: 135 - DNA, 26 bp, Artificial Sequence
  primer PR5008
acttgcactagtagggccgccatggt

SEQ ID NO: 136 - DNA, 26 bp, Artificial Sequence
  primer PR5009
attctagcggccgctgaacagtgtac

SEQ ID NO: 137 - DNA, 77 bp, Artificial Sequence
  primer PR5350
ccggacaatcattatctcagtacccaaagcaaactcagcaaggtaagctggcatccctttgagtcgttggtggttat

SEQ ID NO: 138 - DNA, 75 bp, Artificial Sequence
  primer PR5351
cgataaccaccaacgactcaaagggatgccagcttaccttgctgagtttgctttgggtactgagataatgattgt

SEQ ID NO: 139 - DNA, 62 bp, Artificial Sequence
  primer PR5354
aattggagatgggcctgtcctcctacctgataatcactatctaagtacgcaatctaaacttt

SEQ ID NO: 140 - DNA, 60 bp, Artificial Sequence
  primer PR5355
cgaaagtttagattgcgtacttagatagtgattatcaggtaggaggacaggcccatctcc

SEQ ID NO: 141 - DNA, 20 bp, Artificial Sequence
  primer PR5452
tcagagctgcagacactcct

SEQ ID NO: 142 - DNA, 20 bp, Artificial Sequence
  primer PR2762
agactgcccgccccaagctc

SEQ ID NO: 143 - DNA, 32 bp, Artificial Sequence
  primer PR4365
attgtccatatggtacactgttcagcggccgc

SEQ ID NO: 144 - DNA, 32 bp, Artificial Sequence
  primer PR4366
atgattgagctctagatacaccgcaggcttgc

SEQ ID NO: 145 - DNA, 162 bp, Artificial Sequence
  primer PR6107
cgcgccgaaatagcgctgtacagcgtatgggaatctcttgtacggtgtacgagtatcttcccgtacaccgtacgagctcagttaataattaacgagttaataattaacgagttaataattaactggtaccctagagggtatataatgggggccaatcctctg

SEQ ID NO: 146 - DNA, 162 bp, Artificial Sequence
  primer PR6108
aattcagaggattggcccccattatataccctctagggtaccagttaattattaactcgttaattattaactcgttaattattaactgagctcgtacggtgtacgggaagatactcgtacaccgtacaagagattcccatacgctgtacagcgctatttcgg

SEQ ID NO: 147 - DNA, 191 bp, Artificial Sequence
  primer PR6109
ggccgcgattgaatataaccgacgtgactgttacatttagggattgtatataacccacgtgagtgttacaattagggattgaatataaccgacgtgactgttacatttaggcaattgactacacaaagccctctttgtgagactacacaaagccctctttgtgagactacacaaagccctctttgtgagaa

SEQ ID NO: 148 - DNA, 191 bp, Artificial Sequence
  primer PR6110
agctttctcacaaagagggctttgtgtagtctcacaaagagggctttgtgtagtctcacaaagagggctttgtgtagtcaattgcctaaatgtaacagtcacgtcggttatattcaatccctaattgtaacactcacgtgggttatatacaatccctaaatgtaacagtcacgtcggttatattcaatcgc

SEQ ID NO: 149 - DNA, 55 bp, Artificial Sequence
  primer PR6810
cctacacaaagccctctgtgtaagactacacaaagccctctgtgtaagatggtac

SEQ ID NO: 150 - DNA, 55 bp, Artificial Sequence
  primer PR6811
catcttacacagagggctttgtgtagtcttacacagagggctttgtgtaggagct

SEQ ID NO: 151 - DNA, 37 bp, Artificial Sequence
  primer PR6814
aattgaagttaataattaactagttaataattaacta

SEQ ID NO: 152 - DNA, 37 bp, Artificial Sequence
  primer PR6815
agcttagttaattattaactagttaattattaacttc

SEQ ID NO: 153 - DNA, 78 bp, Artificial Sequence
  primer PR6844
cctacacaaagccctctttgtgagactacacaaagccctctttgtgagactacacaaagccctctttgtgagaggtac

SEQ ID NO: 154 - DNA, 78 bp, Artificial Sequence
  primer PR6845
ctctcacaaagagggctttgtgtagtctcacaaagagggctttgtgtagtctcacaaagagggctttgtgtaggagct

SEQ ID NO: 155 - DNA, 42 bp, Artificial Sequence
  primer PR6846
ggccgcgattgaatataaccgacgtgactgttacatttaggc

SEQ ID NO: 156 - DNA, 42 bp, Artificial Sequence
  primer PR6847
aattgcctaaatgtaacagtcacgtcggttatattcaatcgc

SEQ ID NO: 157 - DNA, 122 bp, Artificial Sequence
  primer PR6931
aattcacttcatgttttggatgctgcaaggctggatgagaggcgactccaggcagcaggcagcctcagagcactgccccgcttgaagtctttaattaaaccgcttgaagtctttaattaaaa

SEQ ID NO: 158 - DNA, 122 bp, Artificial Sequence
  primer PR6932
ccggttttaattaaagacttcaagcggtttaattaaagacttcaagcggggcagtgctctgaggctgcctgctgcctggagtcgcctctcatccagccttgcagcatccaaaacatgaagtg

SEQ ID NO: 159 - DNA, 146 bp, Artificial Sequence
  primer PR6937
gacctttttggagacttccggcacgccaggaggggcagctggactacagacgttactgcatcactctgctctctaaatcaagcactctgatttgacaattaaagcactctgatttgacaattaaagcactctgatttgacaattac

SEQ ID NO: 160 - DNA, 147 bp, Artificial Sequence
  primer PR6938
tcgagtaattgtcaaatcagagtgctttaattgtcaaatcagagtgctttaattgtcaaatcagagtgcttgatttagagagcagagtgatgcagtaacgtctgtagtccagctgcccctcctggcgtgccggaagtctccaaaaag

SEQ ID NO: 161 - DNA, 63 bp, Artificial Sequence
  primer PR4860
cgtattaccgccatgcattagttattaatttgatcatgcattgagagtgtttggtgtggcacc

SEQ ID NO: 162 - DNA, 49 bp, Artificial Sequence
  primer PR4842
ctcaccatggtggcgaccggtagcgctagcgcagtctccctgccgccag

SEQ ID NO: 163 - DNA, 43 bp, Artificial Sequence
  primer PR6250
ccatttcaggtgtcgtgaggaattcgccaccatggtgagcaag

SEQ ID NO: 164 - DNA, 40 bp, Artificial Sequence
  primer PR6251
tctctcttctcgttgggatccttcgaaagcttggactggg

SEQ ID NO: 165 - DNA, 112 bp, Artificial Sequence
  primer PR6252
gatcccaacgagaagagagatcacatggtgcttcttgagttcgtgaccgcagccggaattaccctgggtatggacgagttgtacaagtaggcggccgcactcctcaggtgca

SEQ ID NO: 166 - skipped

SEQ ID NO: 167 - DNA, 140 bp, Artificial Sequence
  primer PR6253
tacctgagctaccagtccaagctgagcaaggatcccaacgagaagagagatcacatggtgcttcttgagttcgtgaccgcagccggaattaccctgggtatggacgagttgtacaagtagtctagagggccctattctat

SEQ ID NO: 168 - DNA, 101 bp, Artificial Sequence
  primer PR4429
aattggcgacggccccgtgctgctgcccgacaaccactacctgagcacccagtccaagctgagcaaggtaagagatgggctagagccaatggtgaagacta

SEQ ID NO: 169 - DNA, 101 bp, Artificial Sequence
  primer PR4430
gctctagtcttcaccattggctctagcccatctcttaccttgctcagcttggactgggtgctcaggtagtggttgtcgggcagcagcacggggccgtcgcc

SEQ ID NO: 170 - DNA, 101 bp, Artificial Sequence
  primer PR4431
aattggcgacggccccgtgctgctgcccgacaaccactacctgagcacccagtccaagctgagcaaggtaggtgatgggctagagccaatggtgaagacta

SEQ ID NO: 171 - DNA, 101 bp, Artificial Sequence
  primer PR4432
gctctagtcttcaccattggctctagcccatcacctaccttgctcagcttggactgggtgctcaggtagtggttgtcgggcagcagcacggggccgtcgcc

SEQ ID NO: 172 - DNA, 100 bp, Artificial Sequence
  primer PR4433
aattggcgacggccccgtgctgctgcccgacaaccactacctgagcacccagtccaagctgagcaaggtaagaatctgctagagccaatggtgaagacta

SEQ ID NO: 173 - DNA, 100 bp, Artificial Sequence
  primer PR4434
gctctagtcttcaccattggctctagcagattcttaccttgctcagcttggactgggtgctcaggtagtggttgtcgggcagcagcacggggccgtcgcc

SEQ ID NO: 174 - DNA, 78 bp, Artificial Sequence
  primer PR4478
tcagcagcagtgcaagttcactctctgcctttgcctaccataatccgatttttttaacttcctttattttccttacag

SEQ ID NO: 175 - DNA, 79 bp, Artificial Sequence
  primer PR4479
gatcctgtaaggaaaataaaggaagttaaaaaaatcggattatggtaggcaaaggcagagagtgaacttgcactgctgc

SEQ ID NO: 176 - DNA, 20 bp, Artificial Sequence
  primer PR4526
gttcgtgctgagcaaggtaa

SEQ ID NO: 177 - DNA, 22 bp, Artificial Sequence
  primer PR4527
cagaatgagctctgaacagtgt

SEQ ID NO: 178 - DNA, 32 bp, Artificial Sequence
  primer PR4331
atggtcggatcctgaacagtgtacactggtgt

SEQ ID NO: 179 - DNA, 38 bp, Artificial Sequence
  primer PR4530
cgaaggtgggtatctgctagagccaatggtgaagacta

SEQ ID NO: 180 - DNA, 40 bp, Artificial Sequence
  primer PR4531
gctctagtcttcaccattggctctagcagatacccacctt

SEQ ID NO: 181 - DNA, 51 bp, Artificial Sequence
  primer PR5667
gcgccgctaaagttaggttgggaatatatctgagtgtatctctcctcccag

SEQ ID NO: 182 - DNA, 51 bp, Artificial Sequence
  primer PR5668
gatcctgggaggagagatacactcagatatattcccaacctaactttagcg

SEQ ID NO: 183 - DNA, 71 bp, Artificial Sequence
  primer PR5669
gcgccgctaaagttaggttgggaatgcctaccataatccgatgacatatctgagtgtatctctcctcccag

SEQ ID NO: 184 - DNA, 71 bp, Artificial Sequence
  primer PR5670
gatcctgggaggagagatacactcagatatgtcatcggattatggtaggcattcccaacctaactttagcg

SEQ ID NO: 185 - DNA, 52 bp, Artificial Sequence
  primer PR6290
tacctgagcacccagtccaagctttcgaaggtaagctggcatccctttgagt

SEQ ID NO: 186 - DNA, 52 bp, Artificial Sequence
  primer PR6291
ttctctatcactgatagggaatgcattgaacagtgtacactggtgtccattg

SEQ ID NO: 187 - DNA, 26 bp, Artificial Sequence
  primer PR4314
atctgggctagctgagcttggtaggt

SEQ ID NO: 188 - DNA, 57 bp, Artificial Sequence
  primer PR4315
ttagtcaccggtccattggctctagcccatcgattaccttggaaagtgcagactgag

SEQ ID NO: 189 - DNA, 35 bp, Artificial Sequence
  primer PR4316
cttgtaaccggtttaagactagagctttcctatgc

SEQ ID NO: 190 - DNA, 20 bp, Artificial Sequence
  primer PR1843
cgcctgagtagtacgtwcgc

SEQ ID NO: 191 - DNA, 20 bp, Artificial Sequence
  primer PR1844
tgcctgrgtagtacattcgc

SEQ ID NO: 192 - DNA, 20 bp, Artificial Sequence
  primer PR1845
cgcctgagtagtatgctcgc

SEQ ID NO: 193 - DNA, 20 bp, Artificial Sequence
  primer PR1846
cgcctgggtagtacattcgc

SEQ ID NO: 194 - DNA, 20 bp, Artificial Sequence
  primer PR1847
gcggtgtgtacaaracccga

SEQ ID NO: 195 - DNA, 20 bp, Artificial Sequence
  primer PR1848
gcggtgtgtacaaaccccga

SEQ ID NO: 196 - DNA, 20 bp, Artificial Sequence
  primer PR0673
tcccacaacgaggactacac

SEQ ID NO: 197 - DNA, 20 bp, Artificial Sequence
  primer PR0674
cgagtcagtgagcgaggaag

SEQ ID NO: 198 - DNA, 20 bp, Artificial Sequence
  primer PR3163
agcagcgtatccacatagcg

SEQ ID NO: 199 - DNA, 21 bp, Artificial Sequence
  primer PR6129
gcaggggaaagaatagtagac

SEQ ID NO: 200 - DNA, 20 bp, Artificial Sequence
  primer PR4308
acttcatgttttggatgctg

SEQ ID NO: 201 - RNA, 22 nt, Artificial Sequence
  siRNA FF4 Sense
gcuugaagucuuuaauuaaauu

SEQ ID NO: 202 - RNA, 22 nt, Artificial Sequence
  siRNA FF4 anti-sense
uuuaauuaaagacuucaagcgg

SEQ ID NO: 203 - RNA, 22 nt, Artificial Sequence
  siRNA FF5 Sense
gcacucugauuugacaauuauu

SEQ ID NO: 204 - RNA, 22 nt, Artificial Sequence
  siRNA FF5 anti-sense
uaauugucaaaucagagugcuu

SEQ ID NO: 205 - RNA, 22 nt, Artificial Sequence
  siRNA FF5 miRIDIAN negative control #2
aguacuuuuguguaguacaauu

SEQ ID NO: 206 - DNA, 1195 bp, Artificial Sequence
  gene fragment gBlock242
ggtcccttgctcgctttctttgcatgctagctgagcttggtaggttctgggctcctaactgatagacagaggcatgtgagggatgaggctgcctgcttcccacctgggcatctgaggacctttttggagacttccggcacgccaggaggggcagctggactacagacgttactgcatcactctgctctctaaatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacaccccaattggcgacggccccgtgctgctgcccgacaaccactacctgagcacccagtccaagctttcgaaggtaagtatctgctagagccaatggtgaagactagagctttcctatgcacactgaggtcttgggtggtttcagcattcctaacagagtttttaatctgcaccatgacttccaggggcctctgctggcagctctcagccccaccaaggttttgggctacttagactatcatctcttcctcttccttctcctcttccttaaggaggaaccctgtggatgattgacaagctctctacccttgagcgacatctcaagcctatttctgtctttgagtacactgtgctgtacttctaggagacctccatcctgagaagcttggcactcccagactataagcaagcctgcggtgtatctaatgcattcccta

SEQ ID NO: 207 - DNA, 1034 bp, Artificial Sequence
  gene fragment gBlock109
gctcgctttctttgcatgctagctgagcttggtaggttctgggctcctaactgatagacagaggcatgtgagggatgaggctgcctgcttcccacctgggcatctgaggacctttttggagacttccggcacgccaggaggggcagctggactacagacgttactgcatcactctgctctctaaatcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaagtaatggatgggctagagccaatggttaagactagagctttcctatgcacactgaggtcttgggtggtttcagcattcctaacagagtttttaatctgcaccatgacttccaggggcctctgctggcagctctcagccccaccaaggttttgggctacttagactatcatctcttcctcttccttctcctcttccttaaggaggaaccctgtggatg

SEQ ID NO: 208 - DNA, 633 bp, Artificial Sequence
  gene fragment gBlock107
ggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaggtagacgtctccaagatcccctttgcagccgctaaagttaggttgggaatccgaggctctgaggctggcaactatttgcccgaagtcagacagcagggccggaaaaag

SEQ ID NO: 209 - DNA, 1032 bp, Artificial Sequence
  gene fragment gBlock241
ggtcccttgctcgctttctttgcatgctagcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacaccccaattggcgacggccccgtgctgctgcccgacaaccactacctgagcacccagtccaagctgagcaaggtaagtgatgggctagagccaatggtgaagactagagctttcctatgcacactgaggtcttgggtggtttcagcattcctaacagagtttttaatctgcaccatgacttccaggggcctctgctggcagctctcagccccaccaaggttttgggctacttagactatcatctcttcctcttccttctcctcttccttaaggaggaaccctgtggatgattgacaagctctctacccttgagcgacatctcaagcctatttctgtctttgagtacactgtgctgtacttctaggagacctccatcctgagaagcttggcactcccagactataagcaagcctgcggtgtatctaatgcattcccta

SEQ ID NO: 210 - DNA, 387 bp, Artificial Sequence
  gene fragment gBlock273
acttcatgttttggatgctgcaaggctggatgagaggcgactccaggcagcaggcagcctcagagcactgccatgcatatgaaccacttccaggccatcctggcccaagtacagacactgctctccagccagaagcccaggcaggtgcgggccctcctggatggcctgctggaagaagagctgctctcacgggaataccactgtgccttgctgcatgagcctgatggtgatgccctggcccggaagatttccctgaccctgctggagaaaggggacttagacttgactttcttgagctgggtctgcaacagtctgcaggctcccacggtagagaggggcaccagctacagggaccatggaggacgtcaccatagtctgtgtgccacc

SEQ ID NO: 211 - DNA, 924 bp, Artificial Sequence
  gene fragment gBlock278
acttgcactagtagggccgccatggtgagcaagggcgaggagctgttcaccggcgtggtgcccatcctggtggagctggacggcgacgtgaacggccacaagttcagcgtgagcggcgagggcgagggcgacgccacctacggcaagctgaccctgaagttcatctgcaccaccggcaagctgcccgtgccctggcccaccctggtgacaaccctgagccacggcgtgcagtgcttcgccagataccccgaccacatgaagcagcacgacttcttcaagagcgccatgcccgagggctacgtgcaggagagaaccatcttcttcaaggacgacggcaactacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggccaacttcaagatcagacacaacatcgaggacggcggcgtgcagctggccgaccactaccagcagaacacccccatcggcgacggccccgtgctgcttccggacaaccactacctgagcacccagagcaagctgagcaaggtaagctggcatccctttgagtcgttggtggttatcgataaggcaacaatgtatgaccaaccctaaggtgtcctttctctgacaggaaaatctatgtaaaggatatagggtaagcttttggaaaacctgggtggctgggtgctgcaagcacaaagcttgttttccacattgatcagagcatcctcagggcacactgccgcctacctgcttagtccatgcctgagcaccatcgacaatggacaccagtgtacactgttcagcggccgctagaat

SEQ ID NO: 212 - DNA, 954 bp, Artificial Sequence
  gene fragment gBlock284
agactgcccgccccaagctcctaggagccacggagctggcggcagggagactgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggaccggtgctgctgcccgacaaccactacctgagctaccagtccaagctgagcaaggtaggcgtctccaagatcccctttggcgccgctaaagttaggttgggaatccgaggctctgaggctggcaactatttgcccgaagtcagacagcagggccggaaaaagctggatgggtgtgcaggaggttgagaggcttcttggatcttggacggactgtatgcaggacctgggagcagggagctggggtccagaacaccgagactatgcacagaactggtagtgaaggcaagtggcgggatagccttgcaggagtgtctgcagctctga

SEQ ID NO: 213 - DNA, 230 bp, Artificial Sequence
  gene fragment JD6.69.1
agttcgacaggacctttttggagacttccggcacgccaggaggggcagctggactacagacgttactgcatcactctgctctctaaatcgccaccatgcgctgcctggttcctggcccttctgggtcttacctgccggagttgcaaggtagacgtctccaagatcccctttggcgcccataatccgatgacatatctgagtgtatctctcctcccaggatcctcaccatc

SEQ ID NO: 214 - DNA, 896 bp, Artificial Sequence
  gene fragment JD6.134.1
tgactattgaattcacttcatgttttggatgctgcaaggctggatgagaggcgactccaggcagcaggcagcctcagagcactgccccgcttgaagtctttaattaaaaccggtgccaccatgaaccacttccaggccatcctggcccaagtacagacactgctctccagccagaagcccaggcaggtgcgggccctcctggatggcctgctggaagaagagctgctctcacgggaataccactgtgccttgctgcatgagcctgatggtgatgccctggcccggaagatttccctgaccctgctggagaaaggggacttagacttgactttcttgagctgggtctgcaacagtctgcaggctcccacggtagagaggggcaccagctacagggaccatggaggtaagctggcatccctttgagtcgttggtggttatcgatgtacactgttcagcggccgcgattgaatataaccgacgtgactgttacatttaggcaattgaagttaataattaactagttaataattaactaagcttactagagggtatataatgggggccagaattttcaggtggtcccttgctcgctttctttgcatgctagctgagcttggtaggttctgggctcctaactgatagacagaggcatgtgagggatgaggctgcctgcttcccacctgggcatctgaggacctttttggagacttccggcacgccaggaggggcagctggactacagacgttactgcatcactctgctctctaaatcaagcactctgatttgacaattactcgaggccaccatgcgctgcctggttcctggcccttctgggtcttacctgccggagttgcaaggtagacgtctccaagatcccctttggcgcccataatcc

SEQ ID NO: 215 - DNA, 300 bp, Artificial Sequence
  gene fragment gBlock249
gttcgtgctgagcaaggtaagctggcatccctttgagtcaaggcaagaattccaatgtatgaccaacgtggaaaacaatgtttttgaacacctaaggtgtcctttctctgacaggaaaatctatgtaaaggatatagggtaagcttttggaaaacctgggtggctgggtgctgcaagcacaaagcttgttttccacattgatcagagcatcctcagggcacactgccgcctacctgcttagtccatgcctgagcaccatcgacaatggacaccagtgtacactgttcagagctcattctg

SEQ ID NO: 216 - DNA, 948 bp, Artificial Sequence
  gene fragment JD7.48.1
aacggtcttacctaggagccacggagctggcggcagggagactgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaggtgatcgtctccaagatcccctttgcagccgctaaagttaggttgggaatccgaggctctgaggctggcaactatttgcccgaagtcagacagcagggccggaaaaagctggatgggtgtgcaggaggttgagaggcttcttggatcttggacggactgtatgcaggacctgggagcagggagctggggtccagaacaccgagactatgcacagaactggtagtgaaggcaagtggcgggatagccttgcaggagtgtctgcagtcttgaagt

SEQ ID NO: 217 - DNA, 948 bp, Artificial Sequence
  gene fragment JD7.48.2
aacggtcttacctaggagccacggagctggcggcagggagactgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaggtgcccgtctccaagatcccctttgcagccgctaaagttaggttgggaatccgaggctctgaggctggcaactatttgcccgaagtcagacagcagggccggaaaaagctggatgggtgtgcaggaggttgagaggcttcttggatcttggacggactgtatgcaggacctgggagcagggagctggggtccagaacaccgagactatgcacagaactggtagtgaaggcaagtggcgggatagccttgcaggagtgtctgcagtcttgaagt

SEQ ID NO: 218 - DNA, 948 bp, Artificial Sequence
  gene fragment JD7.48.3
aacggtcttacctaggagccacggagctggcggcagggagactgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaggtctccgtctccaagatcccctttgcagccgctaaagttaggttgggaatccgaggctctgaggctggcaactatttgcccgaagtcagacagcagggccggaaaaagctggatgggtgtgcaggaggttgagaggcttcttggatcttggacggactgtatgcaggacctgggagcagggagctggggtccagaacaccgagactatgcacagaactggtagtgaaggcaagtggcgggatagccttgcaggagtgtctgcagtcttgaagt

SEQ ID NO: 219 - DNA, 50 bp, Artificial Sequence
  pKB01 junction J1
gagcacccagagcaagctgagcaaggtaagctggcatccctttgagtcaa

SEQ ID NO: 220 - DNA, 50 bp, Artificial Sequence
  pKB01 junction J2
ataatgggggccagaattttcaggtggtcccttgctcgctttctttgcat

SEQ ID NO: 221 - DNA, 50 bp, Artificial Sequence
  pKB01 junction J3
gagcactcagtctgcactttccaaggtaatggatgggctagagccaatgg

SEQ ID NO: 222 - DNA, 50 bp, Artificial Sequence
  pKB01 junction J4
ataatgggggccagactgcccgccccaagctcctaggagccacggagctg

SEQ ID NO: 223 - DNA, 50 bp, Artificial Sequence
  pKB01 junction J5
gagctaccagtccaagctgagcaaggtaggtgtctccaagatcccctttg

SEQ ID NO: 224 - DNA, 50 bp, Artificial Sequence
  pKB01 junction J6
tatctgagtgtatctctcctcccaggatcccaacgagaagagagatcaca

SEQ ID NO: 225 - DNA, 50 bp, Artificial Sequence
  pKB01 junction J7
gagctaccagtccaagctgagcaaggatcccaacgagaagagagatcaca

SEQ ID NO: 226 - DNA, 49 bp, Artificial Sequence
  pKB01 junction J8
agcacccagagcaagctgagcaaggatcccaacgagaagagagatcaca

SEQ ID NO: 227 - DNA, 50 bp, Artificial Sequence
  pKB01 junction J9
gagcactcagtctgcactttccaaggatcccaacgagaagagagatcaca

SEQ ID NO: 228 - DNA, 50 bp, Artificial Sequence; identical to SEQ ID NO: 219
  pJD49 junction J1

SEQ ID NO: 229 - DNA, 50 bp, Artificial Sequence; identical to SEQ ID NO: 220
  pJD49 junction J2

SEQ ID NO: 230 - DNA, 50 bp, Artificial Sequence
  pJD49 junction J3
gagcacccagtccaagctttcgaaggtaagtatctgctagagccaatggt

SEQ ID NO: 231 - DNA, 50 bp, Artificial Sequence; identical to SEQ ID NO: 222
  pJD49 junction J4

SEQ ID NO: 232 - DNA, 50 bp, Artificial Sequence
  pJD49 junction J5
gagctaccagtccaagctgagcaaggtagacgtctccaagatcccctttg

SEQ ID NO: 233 - DNA, 50 bp, Artificial Sequence; identical to SEQ ID NO: 224
  pJD49 junction J6

SEQ ID NO: 234 - DNA, 50 bp, Artificial Sequence; identical to SEQ ID NO: 225
  pJD49 junction J7

SEQ ID NO: 235 - DNA, 50 bp, Artificial Sequence
  pJD49 junction J8
gagcacccagagcaagctgagcaaggatcccaacgagaagagagatcaca

SEQ ID NO: 236 - DNA, 50 bp, Artificial Sequence
  pJD49 junction J9
gagcacccagtccaagctttcgaaggatcccaacgagaagagagatcaca